(19) United States
(12) Patent Application Publication
(10) Pub. No.: US 2021/0219550 A1
(43) Pub. Date: Jul. 22, 2021

(54) PEST CONTROL COMPOSITIONS AND USES THEREOF

(71) Applicant: Flagship Pioneering Innovations VI, LLC, Cambridge, MA (US)

(72) Inventors: Maria Helena Christine VAN ROOIJEN, Cambridge, MA (US); Barry Andrew MARTIN, Boston, MA (US); Hok Hei TAM, Newton, MA (US); Jonathan FRIEDLANDER, Cambridge, MA (US); Ignacio MARTINEZ, Lexington, MA (US); Nataliya Vladimirovna NUKOLOVA, Cambridge, MA (US); Simon SCHWIZER, Boston, MA (US); Daniel Garcia CABANILLAS, Boston, MA (US)

(21) Appl. No.: 17/054,846

(22) PCT Filed: May 15, 2019
(86) PCT No.: PCT/US2019/032460

§ 371 Date: Nov. 12, 2020

Related U.S. Application Data
(60) Provisional application No. 62/676,142, filed on May 24, 2018.
(60) Provisional application No. 62/671,942, filed on May 15, 2018.

(51) Int. Cl. A01N 43/16; A01N 25/00; A01N 37/46; A01N 25/22

(52) U.S. Cl. CPC A01N 43/16; A01N 25/22; A01N 37/46; A01N 25/008

(57) ABSTRACT

Disclosed herein are pest control, e.g., biopesticide or biorepellent, compositions including a plurality of plant messenger packs (e.g., including a plant extracellular vesicle (EV), or segment, portion, or extract thereof) that are useful in methods for decreasing the fitness of pests (e.g., agriculture pests) and/or increasing the fitness of a plant.

### BACKGROUND

Plant pests, including plant pathogens (e.g., bacteria or fungi), invertebrate pests (e.g., insects, mollusks, and nematodes), and weeds are pervasive in the human environment. Although a multitude of means have been utilized for attempting to control infestations by these pests, the demand for safe and effective pest control strategies is increasing. Thus, there is need in the art for new methods and compositions to control plant pests.

### SUMMARY OF THE INVENTION

Disclosed herein are pest control (e.g., biopesticide or biorepellent) compositions including a plurality of plant messenger packs (PMPs) that are useful in methods for decreasing the fitness of pests (e.g., agriculture pests) and/or increasing the fitness of a plant.

In one aspect, the disclosure features a pest control composition including a plurality of plant messenger packs (PMPs), wherein the composition is formulated for delivery to a plant and wherein the composition includes at least 5% PMPs as measured by wt/vol, percent PMP protein composition, and/or percent lipid composition (e.g., by measuring fluorescently labelled lipids) In another aspect, the disclosure features a pest control composition including a plurality of PMPs, wherein the composition is formulated for delivery to a plant pest and wherein the composition includes at least 5% PMPs.

In some embodiments of the pest control composition, the composition is stable for at least one day at room temperature, and/or stable for at least one week at 4° C. In other embodiments, the composition is stable for at least one day at room temperature, and/or stable for at least one week at 4° C.

In some embodiments, the composition is formulated for delivery to a plant. In some embodiments, the composition is formulated for delivery to a plant pest. In some embodiments, the PMPs are stable for at least 24 hours, 48 hours, seven days, or 30 days. In other embodiments, the PMPs are stable at a temperature of at least 24° C., 20° C., or 4° C. In still other embodiments, the plurality of PMPs in the composition is at a concentration effective to decrease the fitness of a plant pest.

In another aspect, the disclosure features a pest control composition including a plurality of PMPs, wherein the plurality of PMPs in the composition is at a concentration effective to decrease the fitness of a plant pest.

In some embodiments, the composition is formulated for delivery to a plant. In some embodiments, the composition is formulated for delivery to a plant pest. In some embodiments, the composition is stable for at least one day at room temperature, and/or stable for at least one week at 4° C.

In some embodiments, the PMP includes a plurality of PMP proteins, and the concentration of PMPs is the concentration of PMP proteins therein. In other embodiments, the plurality of PMPs in the composition is at a concentration of at least 0.01 ng, 0.1 ng, 1 ng, 2 ng, 3 ng, 4 ng, 5 ng, 10 ng, 50 ng, 100 ng, 250 ng, 500 ng, 750 ng, 1 μg, 10 μg, 50 μg, 100 μg, or 250 μg PMP protein/ml. In yet other embodiments, the composition is stable for at least one day at room temperature, and/or stable for at least one week at 4° C.

In some embodiments, the plurality of PMPs in the composition is at a concentration effective to decrease the fitness of a plant pest. In some embodiments, the plant EV is a modified plant extracellular vesicle (EV). In some embodiments, the plant EV is a plant exosome or a plant microvesicle. In some embodiments, the plurality of PMPs further includes a pest repellent.

In another aspect, the disclosure features a pest control composition including a plurality of PMPs, wherein each of the plurality of PMPs includes a heterologous pesticidal agent and wherein the composition is formulated for delivery to a plant or a plant pest.

In some embodiments, the heterologous pesticidal agent is an herbicidal agent, an antibacterial agent, an antifungal agent, an insecticidal agent, a molluscicidal agent, or a nematicidal agent.

In some embodiments, the herbicidal agent is doxorubicin. In other embodiments, the herbicidal agent is glufosinate, glyphosate, propaquizafop, metamitron, metazachlor, pendimethalin, flufenacet, diflufenican, clomazone, nicosulfuron, mesotrione, pinoxaden, sulcotrione, prosulfocarb, sulfentrazone, bifenox, quinmerac, triallate, terbuthylazine, atrazine, oxyfluorfen, diuron, trifluralin, or chlorotoluron.

In some embodiments, the antibacterial agent is doxorubicin. In some embodiments, the antibacterial agent is an antibiotic. In some embodiments, the antibiotic is vancomycin. In other embodiments, the antibiotic is a penicillin, a cephalosporin, a tetracycline, a macrolide, a sulfonamide, vancomycin, polymixin, gramicidin, chloramphenicol, clindamycin, spectinomycin, ciprofloxacin, isoniazid, rifampicin, pyrazinamide, ethambutol, myambutol, or streptomycin. In some embodiments, the antifungal agent is azoxystrobin, mancozeb, prothioconazole, folpet, tebuconazole, difenoconazole, captan, bupirimate, or fosetyl-Al. In some embodiments, the insecticidal agent is a chloronicotinyl, a neonicotinoid, a carbamate, an organophosphate, a pyrethroid, an oxadiazine, a spinosyn, a cyclodiene, an organochlorine, a fiprole, a mectin, a diacylhydrazine, a benzoylurea, an organotin, a pyrrole, a dinitroterpenol, a METI, a tetronic acid, a tetramic acid, or a pthalamide. In some embodiments, the heterologous pesticidal agent is a small molecule, a nucleic acid, or a polypeptide. In some embodiments, the small molecule is an antibiotic or a secondary metabolite. In some embodiments, the nucleic acid is an inhibitory RNA. In some embodiments, the heterologous pesticidal agent is encapsulated by each of the plurality of PMPs; embedded on the surface of each of the plurality of PMPs; or conjugated to the surface of each of the plurality of PMPs.

In some embodiments, each of the plurality of PMPs further includes a pest repellent. In some embodiments, each of the plurality of PMPs further includes an additional heterologous pesticidal agent.

In some embodiments, the plant pest is a bacterium or a fungus. In some embodiments, the bacterium is a Pseudomonas species, e.g., Pseudomonas aeruginosa or Pseudomonas syringae. In some embodiments, the fungus is a Sclerotinia species, a Botrytis species, an Aspergillus species, a Fusarium species, or a Penicillium species. In other embodiments, the plant pest is an insect, e.g. an aphid or a lepidopteran; a mollusk; or a nematode, e.g., a corn root-knot nematode.

In some embodiments, the composition is stable for at least one day at room temperature, and/or stable for at least one week at 4° C. In some embodiments, the PMPs are stable for at least 24 hours, 48 hours, seven days, or 30 days at 4° C. In other embodiments, the PMPs are stable at a temperature of at least 20° C., 24° C., or 37° C.

In some embodiments, the plurality of PMPs in the composition is at a concentration effective to decrease the fitness of a plant pest.

In some embodiments, the plurality of PMPs in the composition is at a concentration of at least least 0.01 ng, 0.1 ng, 1 ng, 2 ng, 3 ng, 4 ng, 5 ng, 10 ng, 50 ng, 100 ng, 250 ng, 500 ng, 750 ng, 1 μg, 10 μg, 50 μg, 100 μg, or 250 μg PMP protein/mL.

In some embodiments, the composition includes an agriculturally acceptable carrier; formulated to stabilize the PMPs; or formulated as a liquid, a solid, an aerosol, a paste, a gel, or a gas composition.

In some embodiments, the composition includes at least 5% PMPs.

In another aspect, the disclosure features a pest control composition including a plurality of PMPs, wherein the PMPs are isolated from a plant by a process which includes the steps of (a) providing an initial sample from a plant, or a part thereof, wherein the plant or part thereof includes EVs; (b) isolating a crude PMP fraction from the initial sample, wherein the crude PMP fraction has a decreased level of at least one contaminant or undesired component from the plant or part thereof relative to the level in the initial sample; (c) purifying the crude PMP fraction, thereby producing a plurality of pure PMPs, wherein the plurality of pure PMPs have a decreased level of at least one contaminant or undesired component from the plant or part thereof relative to the level in the crude EV fraction; (d) loading the plurality of pure PMPs with a pest control agent; and (e) formulating the PMPs of step (d) for delivery to a plant or a plant pest.

In another aspect, the disclosure features a plant including any one of the pest control compositions provided herein.

In yet another aspect, the disclosure features a plant pest including any one of the pest control compositions provided herein.

In still another aspect, the disclosure features a method of delivering a pest control composition to a plant including contacting the plant with any one of the compositions described herein.

And in still another aspect, the disclosure features a method of increasing the fitness of a plant, the method including delivering to the plant any one of the compositions described herein, wherein the method increases the fitness of the plant relative to an untreated plant.

In some embodiments, the plant has an infestation by a plant pest. In some embodiments, the method decreases the infestation relative to the infestation in an untreated plant. In some embodiments, the method substantially eliminates the infestation relative to the infestation in an untreated plant.

In some embodiments, the plant is susceptible to infestation by a plant pest. In some embodiments, the method decreases the likelihood of infestation in the plant relative to the likelihood of infestation in an untreated plant.

In some embodiments, the plant pest is a bacterium, e.g., a Pseudomonas species; or a fungus, e.g., a Sclerotinia species, a Botrytis species, an Aspergillus species, a Fusarium species, or a Penicillium species.

In other embodiments, the plant pest is an insect, e.g., an aphid or a lepidopteran; a mollusk; or a nematode, e.g., a corn root-knot nematode.

In some embodiments, the pest control composition is delivered as a liquid, a solid, an aerosol, a paste, a gel, or a gas.

In another aspect, the disclosure features a method of delivering a pest control composition to a plant pest including contacting the plant pest with any one of the compositions described herein.

In another aspect, the disclosure features a method of decreasing the fitness of a plant pest, the method including delivering to the plant pest any one of the compositions described herein, wherein the method decreases the fitness of the plant pest relative to an untreated plant pest.

In some embodiments, the method includes delivering the composition to at least one habitat where the plant pest grows, lives, reproduces, feeds, or infests. In some embodiments, the composition is delivered as a plant pest comestible composition for ingestion by the plant pest.

In some embodiments, the plant pest is a bacterium or a fungus. In other embodiments, the plant pest is an insect, e.g., an aphid or a lepidopteran; a mollusk; or a nematode, e.g., a corn root-knot nematode. In some embodiments, the composition is delivered as a liquid, a solid, an aerosol, a paste, a gel, or a gas.

In another aspect, the disclosure features a method of treating a plant having a fungal infection, wherein the method includes delivering to the plant a pest control composition including a plurality of PMPs.

In still another aspect, the disclosure features a method of treating a plant having a fungal infection, wherein the method includes delivering to the plant a pest control composition including a plurality of PMPs, and wherein each of the plurality of PMPs includes an antifungal agent. In some embodiments, the antifungal agent is a nucleic acid that inhibits expression of a gene in a fungus that causes the fungal infection. In some embodiments, the gene is dcl1 and/or dcl2. In some embodiments, the fungal infection is caused by a fungus belonging to a Sclerotinia species, e.g., Sclerotinia sclerotiorum; a Botrytis species, e.g., Botrytis cinerea; an Aspergillus species; a Fusarium species; or a Penicillium species. In some embodiments, the composition includes a PMP derived from Arabidopsis. In some embodiments, the method decreases or substantially eliminates the fungal infection.

In another aspect, the disclosure features a method of treating a plant having a bacterial infection, wherein the method includes delivering to the plant a pest control composition including a plurality of PMPs.

In still another aspect, the disclosure features a method of treating a plant having a bacterial infection, wherein the method includes delivering to the plant a pest control composition including a plurality of PMPs, and wherein each of the plurality of PMPs includes an antibacterial agent. In some embodiments, the antibacterial agent is doxorubicin. In some embodiments, the bacterial infection is caused by a bacterium belonging to a Pseudomonas species, e.g., Pseudomonas syringae. In some embodiments, the composition includes a PMP derived from Arabidopsis. In some embodiments, the method decreases or substantially eliminates the bacterial infection.

In another aspect, the disclosure features a method of decreasing the fitness of an insect plant pest, wherein the method includes delivering to the insect plant pest a pest control composition including a plurality of PMPs.

In yet another aspect, the disclosure features a method of decreasing the fitness of an insect plant pest, wherein the method includes delivering to the insect plant pest a pest control composition including a plurality of PMPs, and wherein each of the plurality of PMPs includes an insecticidal agent. In some embodiments, the insecticidal agent is a peptide nucleic acid.

In some embodiments, the insect plant pest is an aphid. In some embodiments, the insect plant pest is a lepidopteran, e.g., Spodoptera frugiperda. In some embodiments, the method decreases the fitness of the insect plant pest relative to an untreated insect plant pest.

In another aspect, the disclosure features a method of decreasing the fitness of a nematode plant pest, wherein the method includes delivering to the nematode plant pest a pest control composition including a plurality of PMPs.

In another aspect, the disclosure features a method of decreasing the fitness of a nematode plant pest, wherein the method includes delivering to the nematode plant pest a pest control composition including a plurality of PMPs, and wherein each of the plurality of PMPs includes a nematicidal agent.

In some embodiments, the nematicidal agent is a peptide, e.g., Mi-NLP-15b. In some embodiments, the nematode plant pest is a corn root-knot nematode. In some embodiments, the method decreases the fitness of the nematode plant pest relative to an untreated nematode plant pest.

In another aspect, the disclosure features a method of decreasing the fitness of a weed, wherein the method includes delivering to the weed a pest control composition including a plurality of PMPs.

In another aspect, the disclosure features a method of decreasing the fitness of a weed, wherein the method includes delivering to the weed a pest control composition including a plurality of PMPs, and wherein each of the plurality of PMPs includes an herbicidal agent. In some embodiments, the method decreases the fitness of the weed relative to an untreated weed. Other features and advantages of the invention will be apparent from the following Detailed Description and the Claims.

### Definitions

As used herein, the term “pest control composition” refers to a biopesticide or biorepellent composition that includes a plurality of plant messenger (PMP) packs. Each of the plurality of PMPs may include a pesticidal agent, e.g., a heterologous pesticidal agent.

As used herein, the term “biopesticide composition” refers to a pesticidal composition that includes a plurality of plant messenger (PMP) packs.

As used herein, the term “biorepellent composition” refers to a pest repellent composition that includes a plurality of plant messenger (PMP) packs.

As used herein, “delivering” or “contacting” refers to applying to a plant or plant pest, a pest control (e.g., biopesticide or biorepellent) composition either directly on the plant or plant pest, or adjacent to the plant or plant pest, in a region where the composition is effective to alter the fitness of the plant or plant pest. In methods where the composition is directly contacted with a plant, the composition may be contacted with the entire plant or with only a portion of the plant.

As used herein, “decreasing the fitness of a plant pest” refers to any disruption to pest physiology, or any activity carried out by said pest, as a consequence of administration of a pest control (e.g., biopesticide or biorepellent) composition described herein, including, but not limited to, any one or more of the following desired effects: (1) decreasing a population of a pest by about 10%, 20%, 30%, 40%, 50%, 60%, 70%, 80%, 90%, 95%, 99%, 100% or more; (2) decreasing the reproductive rate of a pest (e.g., insect) by about 10%, 20%, 30%, 40%, 50%, 60%, 70%, 80%, 90%, 95%, 99%, 100% or more; (3) decreasing the mobility of a pest by about 10%, 20%, 30%, 40%, 50%, 60%, 70%, 80%, 90%, 95%, 99%, 100% or more; (4) decreasing the body weight of a pest by about 10%, 20%, 30%, 40%, 50%, 60%, 70%, 80%, 90%, 95%, 99%, 100% or more; (5) decreasing the metabolic rate or activity of a pest by about 10%, 20%, 30%, 40%, 50%, 60%, 70%, 80%, 90%, 95%, 99%, 100% or more; or (6) decreasing plant infestation by a pest by about 10%, 20%, 30%, 40%, 50%, 60%, 70%, 80%, 90%, 95%, 99%, 100% or more. A decrease in pest fitness can be determined in comparison to a pest to which the pest control (e.g., biopesticide or biorepellent) composition has not been administered.

As used herein, the term “formulated for delivery to a plant or a plant pest” refers to a pest control (e.g., biopesticide or biorepellent) composition that includes an agriculturally acceptable carrier.

As used herein, the term “infestation” refers to the presence of unwanted pests on a plant, e.g., colonization or infection of a plant, a part thereof, or the habitat surrounding a plant, by a plant pest, particularly where the infestation decreases the fitness of the plant. A “decrease in infestation” or “treatment of an infestation” refers to a decrease in the number of pests on or around the plant (e.g., by about 1%, 2%, 5%, 10%, 20%, 30%, 40%, 50%, 60%, 70%, 80%, 90%, or %100) or a decrease in symptoms or signs in the plant that are directly or indirectly caused by the pest (e.g., by about 1%, 2%, 5%, 10%, 20%, 30%, 40%, 50%, 60%, 70%, 80%, 90%, or %100) relative to an untreated plant. Infestation or associated symptoms can be identified by any means of identifying infestation or related symptoms. For example, the decrease in infestation in one or more parts of the plant may be in an amount sufficient to “substantially eliminate” an infestation, which refers to a decrease in the infestation in an amount sufficient to sustainably resolve symptoms and/or increase plant fitness relative to an untreated plant.

As used herein, “increasing the fitness of a plant” refers to an increase in the production of the plant, for example, an improved yield, improved vigor of the plant or quality of the harvested product from the plant. An improved yield of a plant relates to an increase in the yield of a product (e.g., as measured by plant biomass, grain, seed or fruit yield, protein content, carbohydrate or oil content or leaf area) of the plant by a measurable amount over the yield of the same product of the plant produced under the same conditions, but without the application of the instant compositions or compared with application of conventional pesticides. For example, yield can be increased by at least about 0.5%, about 1%, about 2%, about 3%, about 4%, about 5%, about 10%, about 20%, about 30%, about 40%, about 50%, about 60%, about 70%, about 80%, about 90%, about 100%, or more than 100%. Yield can be expressed in terms of an amount by weight or volume of the plant or a product of the plant on some basis. The basis can be expressed in terms of time, growing area, weight of plants produced, or amount of a raw material used. An increase in the fitness of plant can also be measured by other means, such as an increase or improvement of the vigor rating, the stand (the number of plants per unit of area), plant height, stalk circumference, plant canopy, visual appearance (such as greener leaf color), root rating, emergence, protein content, increased tillering, bigger leaves, more leaves, less dead basal leaves, stronger tillers, less fertilizer needed, less seeds needed, more productive tillers, earlier flowering, early grain or seed maturity, less plant verse (lodging), increased shoot growth, earlier germination, or any combination of these factors, by a measurable or noticeable amount over the same factor of the plant produced under the same conditions, but without the administration of the instant compositions or with application of conventional pesticides.

As defined herein, the term “nucleic acid” and “polynucleotide” are interchangeable and refer to RNA or DNA that is linear or branched, single or double stranded, or a hybrid thereof, regardless of length (e.g., at least 2, 3, 4, 5, 6, 7, 8, 9, 10, 20, 30, 40, 50, 100, 150, 200, 250, 500, 1000, or more nucleic acids). The term also encompasses RNA/DNA hybrids. Nucleotides are typically linked in a nucleic acid by phosphodiester bonds, although the term “nucleic acid” also encompasses nucleic acid analogs having other types of linkages or backbones (e.g., phosphoramide, phosphorothioate, phosphorodithioate, O-methylphosphoroamidate, morpholino, locked nucleic acid (LNA), glycerol nucleic acid (GNA), threose nucleic acid (TNA), and peptide nucleic acid (PNA) linkages or backbones, among others). The nucleic acids may be single-stranded, double-stranded, or contain portions of both single-stranded and double-stranded sequence. A nucleic acid can contain any combination of deoxyribonucleotides and ribonucleotides, as well as any combination of bases, including, for example, adenine, thymine, cytosine, guanine, uracil, and modified or non-canonical bases (including, e.g., hypoxanthine, xanthine, 7-methylguanine, 5,6-dihydrouracil, 5-methylcytosine, and 5 hydroxymethylcytosine).

As used herein, the term “pest” refers to organisms that cause damage to plants or other organisms, are present where they are not wanted, or otherwise are detrimental to humans, for example, by impacting human agricultural methods or products. Pests may include, for example, invertebrates (e.g., insects, nematodes, or mollusks), microorganisms (e.g., phytopathogens, endophytes, obligate parasites, facultative parasites, or facultative saprophytes), such as bacteria, fungi, or viruses; or weeds.

As used herein, the term “pesticidal agent” or “pesticide” refers to an agent, composition, or substance therein, that controls or decreases the fitness (e.g., kills or inhibits the growth, proliferation, division, reproduction, or spread) of an agricultural, environmental, or domestic/household pest, such as an insect, mollusk, nematode, fungus, bacterium, or virus. Pesticides are understood to encompass naturally occurring or synthetic insecticides (larvicides or adulticides), insect growth regulators, acaricides (miticides), molluscicides, nematicides, ectoparasiticides, bactericides, fungicides, or herbicides. The term “pesticidal agent” may further encompass other bioactive molecules such as antibiotics, antivirals, pesticides, antifungals, antihelminthics, nutrients, and/or agents that stun or slow insect movement. In some instances, the pesticide is an allelochemical. As used herein, “allelochemical” or “allelochemical agent” is a substance produced by an organism (e.g., a plant) that can effect a physiological function (e.g., the germination, growth, survival, or reproduction) of another organism (e.g., a pest).

The pesticidal agent may be heterologous. As used herein, the term “heterologous” refers to an agent (e.g., a pesticidal agent) that is either (1) exogenous to the plant (e.g., originating from a source that is not the plant or plant part from which the PMP is produced) (e.g., added the PMP using loading approaches described herein) or (2) endogenous to the plant cell or tissue from which the PMP is produced, but present in the PMP (e.g., added to the PMP using loading approaches described herein, genetic engineering, in vitro or in vivo approaches) at a concentration that is higher than that found in nature (e.g., higher than a concentration found in a naturally-occurring plant extracellular vesicle).

As used herein, the term “repellent” refers to an agent, composition, or substance therein, that deters pests from approaching or remaining on a plant. A repellent may, for example, decrease the number of pests on or in the vicinity of a plant, but may not necessarily kill or decreasing the fitness of the pest.

As used herein, the term “peptide,” “protein,” or “polypeptide” encompasses any chain of naturally or non-naturally occurring amino acids (either D- or L-amino acids), regardless of length (e.g., at least 2, 3, 4, 5, 6, 7, 10, 12, 14, 16, 18, 20, 25, 30, 40, 50, 100, or more amino acids), the presence or absence of post-translational modifications (e.g., glycosylation or phosphorylation), or the presence of, e.g., one or more non-amino acyl groups (for example, sugar, lipid, etc.) covalently linked to the peptide, and includes, for example, natural proteins, synthetic, or recombinant polypeptides and peptides, hybrid molecules, peptoids, or peptidomimetics.

As used herein, “percent identity” between two sequences is determined by the BLAST 2.0 algorithm, which is described in Altschul et al., (1990) J. Mol. Biol. 215:403-410. Software for performing BLAST analyses is publicly available through the National Center for Biotechnology Information.

As used herein, the term “plant” refers to whole plants, plant organs, plant tissues, seeds, plant cells, seeds, and progeny of the same. Plant cells include, without limitation, cells from seeds, suspension cultures, embryos, meristematic regions, callus tissue, leaves, roots, shoots, gametophytes, sporophytes, pollen, and microspores. Plant parts include differentiated and undifferentiated tissues including, but not limited to the following: roots, stems, shoots, leaves, pollen, seeds, fruit, harvested produce, tumor tissue, sap (e.g., xylem sap and phloem sap), and various forms of cells and culture (e.g., single cells, protoplasts, embryos, and callus tissue). The plant tissue may be in a plant or in a plant organ, tissue, or cell culture. In addition, a plant may be genetically engineered to produce a heterologous protein or RNA, for example, of any of the pest control (e.g., biopesticide or biorepellent) compositions in the methods or compositions described herein.

As used herein, the term “plant extracellular vesicle”, “plant EV”, or “EV” refers to an enclosed lipid-bilayer structure naturally occurring in a plant. Optionally, the plant EV includes one or more plant EV markers. As used herein, the term “plant EV marker” refers to a component that is naturally associated with a plant, such as a plant protein, a plant nucleic acid, a plant small molecule, a plant lipid, or a combination thereof, including but not limited to any of the plant EV markers listed in the Appendix. In some instances, the plant EV marker is an identifying marker of a plant EV but is not a pesticidal agent. In some instances, the plant EV marker is an identifying marker of a plant EV and also a pesticidal agent (e.g., either associated with or encapsulated by the plurality of PMPs, or not directly associated with or encapsulated by the plurality of PMPs).

As used herein, the term “plant messenger pack” or “PMP” refers to a lipid structure (e.g., a lipid bilayer, unilamellar, multilamellar structure; e.g., a vesicular lipid structure), that is about 5-2000 nm (e.g., at least 5-1000 nm, at least 5-500 nm, at least 400-500 nm, at least 25-250 nm, at least 50-150 nm, or at least 70-120 nm) in diameter that is derived from (e.g., enriched, isolated or purified from) a plant source or segment, portion, or extract thereof, including lipid or non-lipid components (e.g., peptides, nucleic acids, or small molecules) associated therewith and that has been enriched, isolated or purified from a plant, a plant part, or a plant cell, the enrichment or isolation removing one or more contaminants or undesired components from the source plant. PMPs may be highly purified preparations of naturally occurring EVs. Preferably, at least 1% of contaminants or undesired components from the source plant are removed (e.g., at least 2%, 5%, 10%, 15%, 20%, 25%, 30%, 40%, 45%, 50%, 55%, 60%, 70%, 80%, 90%, 95%, 96%, 98%, 99%, or 100%) of one or more contaminants or undesired components from the source plant, e.g., plant cell wall components; pectin; plant organelles (e.g., mitochondria; plastids such as chloroplasts, leucoplasts or amyloplasts; and nuclei); plant chromatin (e.g., a plant chromosome); or plant molecular aggregates (e.g., protein aggregates, protein-nucleic acid aggregates, lipoprotein aggregates, or lipido-proteic structures). Preferably, a PMP is at least 30% pure (e.g., at least 40% pure, at least 50% pure, at least 60% pure, at least 70% pure, at least 80% pure, at least 90% pure, at least 99% pure, or 100% pure) relative to the one or more contaminants or undesired components from the source plant as measured by weight (w/w), spectral imaging (% transmittance), or conductivity (S/m).

PMPs may optionally include additional agents, such as heterologous functional agents, e.g., pesticidal agents, fertilizing agents, plant-modifying agents, therapeutic agents, polynucleotides, polypeptides, or small molecules. The PMPs can carry or associate with additional agents (e.g., heterologous functional agents) in a variety of ways to enable delivery of the agent to a target plant, e.g., by encapsulating the agent, incorporation of the agent in the lipid bilayer structure, or association of the agent (e.g., by conjugation) with the surface of the lipid bilayer structure. Heterologous functional agents can be incorporated into the PMPs either in vivo (e.g., in planta) or in vitro (e.g., in tissue culture, in cell culture, or synthetically incorporated).

As used herein, the term “stable PMP composition” (e.g., a composition including loaded or non-loaded PMPs) refers to a PMP composition that over a period of time (e.g., at least 24 hours, at least 48 hours, at least 1 week, at least 2 weeks, at least 3 weeks, at least 4 weeks, at least 30 days, at least 60 days, or at least 90 days) retains at least 5% (e.g., at least 5%, 10%, 15%, 20%, 25%, 30%, 35%, 40%, 45%, 50%, 55%, 60%, 65%, 70%, 75%, 80%, 85%, 90%, 95%, or 100%) of the initial number of PMPs (e.g., PMPs per mL of solution) relative to the number of PMPs in the PMP composition (e.g., at the time of production or formulation) optionally at a defined temperature range (e.g., a temperature of at least 24° C. (e.g., at least 24° C., 25° C., 26° C., 27° C., 28° C., 29° C., or 30° C.), at least 20° C. (e.g., at least 20° C., 21° C., 22° C., or 23° C.), at least 4° C. (e.g., at least 5° C., 10° C., or 15° C.), at least −20° C. (e.g., at least −20° C., −15° C., −10° C., −5° C., or 0° C.), or −80° C. (e.g., at least −80° C., −70° C., −60° C., −50° C., −40° C., or −30° C.)); or retains at least 5% (e.g., at least 5%, 10%, 15%, 20%, 25%, 30%, 35%, 40%, 45%, 50%, 55%, 60%, 65%, 70%, 75%, 80%, 85%, 90%, 95%, or 100%) of its activity (e.g., pesticidal and/or repellent activity) relative to the initial activity of the PMP (e.g., at the time of production or formulation) optionally at a defined temperature range (e.g., a temperature of at least 24° C. (e.g., at least 24° C., 25° C., 26° C., 27° C., 28° C., 29° C., or 30° C.), at least 20° C. (e.g., at least 20° C., 21° C., 22° C., or 23° C.), at least 4° C. (e.g., at least 5° C., 10° C., or 15° C.), at least −20° C. (e.g., at least −20° C., −15° C., −10° C., −5° C., or 0° C.), or −80° C. (e.g., at least −80° C., −70° C., −60° C., −50° C., −40° C., or −30° C.)).

As used herein, the term “untreated” refers to a plant or plant pest that has not been contacted with or delivered a pest control (e.g., biopesticide or biorepellent) composition, including a separate plant that has not been delivered the pest control (e.g., biopesticide or biorepellent) composition, the same plant undergoing treatment assessed at a time point prior to delivery of the pest control (e.g., biopesticide or biorepellent) compositions, or the same plant undergoing treatment assessed at an untreated part of the plant.

As used herein, the term “juice sac” or “juice vesicle” refers to a juice-containing membrane-bound component of the endocarp (carpel) of a hesperidium, e.g., a citrus fruit. In some aspects, the juice sacs are separated from other portions of the fruit, e.g., the rind (exocarp or flavedo), the inner rind (mesocarp, albedo, or pith), the central column (placenta), the segment walls, or the seeds. In some aspects, the juice sacs are juice sacs of a grapefruit, a lemon, a lime, or an orange.

### BRIEF DESCRIPTION OF THE DRAWINGS

FIG. 1A is a schematic diagram showing a protocol for grapefruit PMP production using a destructive juicing step involving the use of a blender, followed by ultracentrifugation and sucrose gradient purification. Images are included of the grapefruit juice after centrifugation at 1000×g for 10 min and the sucrose gradient band pattern after ultracentrifugation at 150,000×g for 2 hours.

FIG. 1B is a plot of the PMP particle distribution measured by the Spectradyne NCS1.

FIG. 2 is a schematic diagram showing a protocol for grapefruit PMP production using a mild juicing step involving use of a mesh filter, followed by ultracentrifugation and sucrose gradient purification. Images are included of the grapefruit juice after centrifugation at 1000×g for 10 min and the sucrose gradient band pattern after ultracentrifugation at 150,000×g for 2 hours.

FIG. 3A is a schematic diagram showing a protocol for grapefruit PMP production using ultracentrifugation, followed by size exclusion chromatography (SEC) to isolate the PMP-containing fractions. The eluted SEC fractions are analyzed for particle concentration (NanoFCM), median particle size (NanoFCM), and protein concentration (BCA).

FIG. 3B is a graph showing particle concentration per mL in eluted size exclusion chromatography (SEC) fractions (NanoFCM). The fractions containing the majority of PMPs (“PMP fraction”) are indicated with an arrow. PMPs are eluted in fractions 2-4.

FIG. 3C is a set of graphs and a table showing particle size in nm for selected SEC fractions, as measured using NanoFCM. The graphs show PMP size distribution in fractions 1, 3, 5, and 8.

FIG. 3D is a graph showing protein concentration in μg/mL in SEC fractions, as measured using a BCA assay. The fraction containing the majority of PMPs (“PMP fraction”) is labeled, and an arrow indicates a fraction containing contaminants.

FIG. 4A is a schematic diagram showing a protocol for scaled PMP production from 1 liter of grapefruit juice (˜7 grapefruits) using a juice press, followed by differential centrifugation to remove large debris, 100× concentration of the juice using TFF, and size exclusion chromatography (SEC) to isolate the PMP containing fractions. The SEC elution fractions are analyzed for particle concentration (NanoFCM), median particle size (NanoFCM) and protein concentration (BCA).

FIG. 4B is a pair of graphs showing protein concentration (BCA assay, top panel) and particle concentration (NanoFCM, bottom panel) of SEC eluate volume (ml) from a scaled starting material of 1000 ml of grapefruit juice, showing a high amount of contaminants in the late SEC elution volumes.

FIG. 4C is a graph showing that incubation of the crude grapefruit PMP fraction with a final concentration of 50 mM EDTA, pH 7.15 followed by overnight dialysis using a 300 kDa membrane, successfully removed contaminants present in the late SEC elution fractions, as shown by absorbance at 280 nm. There was no difference in the dialysis buffers used (PBS without calcium/magnesium pH 7.4, MES pH 6, Tris pH 8.6).

FIG. 4D is a graph showing that incubation of the crude grapefruit PMP fraction with a final concentration of 50 mM EDTA, pH 7.15, followed by overnight dialysis using a 300 kDa membrane, successfully removed contaminants present in the late elution fractions after SEC, as shown by BCA protein analysis, which, besides detecting protein, is sensitive to the presence of sugars and pectins. There was no difference in the dialysis buffers used (PBS without calcium/magnesium pH 7.4, MES pH 6, Tris pH 8.6).

FIG. 5A is a schematic diagram showing a protocol for PMP production from grapefruit juice using a juice press, followed by differential centrifugation to remove large debris, incubation with EDTA to reduce the formation of pectin macromolecules, sequential filtration to remove large particles, 5× concentration/wash by TFF, dialysis overnight to remove contaminants, further concentration by TFF (20× final), and SEC to isolate the PMP-containing fractions.

FIG. 5B is a graph showing the absorbance at 280 nm (A.U.) of eluted grapefruit SEC fractions using multiple SEC columns. PMPs are eluted in early fractions 4-6, and contaminants are eluted in late fractions.

FIG. 5C is a graph showing the protein concentration (μg/ml) of eluted grapefruit SEC fractions using multiple SEC columns. PMPs are eluted in early fractions 4-6, and contaminants are eluted in late fractions.

FIG. 5D is a graph showing the absorbance at 280 nm (A.U.) of eluted lemon SEC fractions using multiple SEC columns. PMPs are eluted in early fractions 4-6, and contaminants are eluted in late fractions.

FIG. 5E is a graph showing the protein concentration (μg/ml) of eluted lemon SEC fractions using multiple SEC columns. PMPs were eluted in early fractions 4-6, and contaminants were eluted in late fractions.

FIG. 5F is a scatter plot and a graph showing particle size in grapefruit PMP-containing SEC fractions after 0.22 um filter sterilization. The top panel is a scatter plot of particles in the combined SEC fractions, as measured by nano-flow cytometry (NanoFCM). The bottom panel is a size (nm) distribution graph of the gated particles (background subtracted). PMP concentration (particles/ml) and median size (nm) were determined using bead standards according to NanoFCM's instructions.

FIG. 5G is a scatter plot and a graph showing particle size in lemon PMP-containing SEC fractions after 0.22 um filter sterilization. The top panel is a scatter plot of particles in the combined SEC fractions, as measured by nano-flow cytometry (NanoFCM). The bottom panel is a size (nm) distribution graph of the gated particles (background subtracted). PMP concentration (particles/ml) and median size (nm) were determined using bead standards according to NanoFCM's instructions.

FIG. 5H is a graph showing grapefruit and lemon PMP stability at 4° Celsius, determined by the PMP concentration (PMP particles/ml) at different time points (days after production), as measured by NanoFCM.

FIG. 5I is a bar graph showing the stability of lemon (LM) PMPs after one freeze-thaw cycle at −20° Celsius and −20° Celsius compared to lemon PMPs stored at 4° Celsius, as determined by the PMP concentration (PMP particles/ml) after one week storage at the indicated temperatures, as measured by NanoFCM.

FIG. 6A is a graph showing particle concentration (particles/ml) in eluted BMS plant cell culture SEC fractions, as measured by nano-flow cytometry (NanoFCM). PMPs were eluted in SEC fractions 4-6.

FIG. 6B is a graph showing absorbance at 280 nm (A.U.) in eluted BMS SEC fractions, measured on a SpectraMax® spectrophotometer. PMPs were eluted in fractions 4-6; fractions 9-13 contained contaminants.

FIG. 6C is a graph showing protein concentration (μg/ml) in eluted BMS SEC fractions, as determined by BCA analysis. PMPs were eluted in fractions 4-6; fractions 9-13 contained contaminants.

FIG. 6D is a scatter plot showing particles in the combined BMS PMP-containing SEC fractions as measured by nano-flow cytometry (NanoFCM). PMP concentration (particles/ml) was determined using a bead standard according to NanoFCM's instructions.

FIG. 6E is a graph showing the size distribution of BMS PMPs (nm) for the gated particles (background subtracted) of FIG. 6D. Median PMP size (nm) was determined using Exo bead standards according to NanoFCM's instructions.

FIG. 7A is a scatter plot and a graph showing DyLight800 nm-labeled grapefruit PMPs as measured by Nano flow cytometry (NanoFCM). The top panel is a scatter plot of particles in the combined SEC fractions. The PMP concentration (4.44×10¹² PMPs/ml) was determined using a bead standard according to NanoFCM's instructions. The bottom panel is a size (nm) distribution graph of grapefruit DyLight800-PMPs. The median PMP size was determined using Exo bead standards according to NanoFCM's instructions. The median grapefruit DyLight800-PMPs size was 72.6 nm+/−14.6 nm (SD).

FIG. 7B is a scatter plot and a graph showing DyLight800 nm-labeled lemon PMPs as measured by Nano flow cytometry (NanoFCM). The median PMP concentration (5.18E×10¹² PMPs/ml) was determined using a bead standard according to NanoFCM's instructions. The bottom panel is a size (nm) distribution graph of grapefruit DyLight800-PMPs. The PMP size was determined using Exo bead standards according to NanoFCM's instructions. The median lemon DyLight800-PMPs size was 68.5 nm+/−14 nm (SD).

FIG. 7C is a bar graph showing the uptake of grapefruit and lemon-derived DyL800 nm-labeled PMPs by bacteria (E. coli, P. aeruginosa, and P. syringae) and yeast (S. cerevisiae) 2 hours post-treatment. Uptake is defined in relative fluorescence intensity (A.U.), normalized to the relative fluorescence intensity of dye-only treated microbe controls.

FIG. 8A is a scatter plot and a graph showing purified lemon PMPs (combined and pelleted PMP SEC fractions), as measured by nano flow cytometry (NanoFCM). The top panel is a scatter plot of particles in the combined SEC fractions. The final lemon PMP concentration (1.53×10¹³ PMPs/ml) was determined using a bead standard according to NanoFCM's instructions. The bottom panel is a size (nm) distribution graph of purified lemon PMPs. The bottom panel is a size (nm) distribution graph of the gated particles. The median PMP size was determined using Exo bead standards according to NanoFCM's instructions. The median lemon PMP size was 72.4 nm+/−19.8 nm (SD).

FIG. 8B is a scatter plot and a graph showing Alexa Fluor® 488-(AF488)-labeled lemon PMPs as measured by nano flow cytometry (NanoFCM). The top panel is a scatter plot. Particles were gated on the FITC fluorescence signal, relative to unlabeled particles and background signal. The labeling efficiency was 99%, as determined by the number of fluorescent particles relative to the total number of particles detected. The final AF488-PMP concentration (1.34×10¹³ PMPs/ml) was determined from the number of fluorescent particles and using a bead standard with a known concentration according to NanoFCM's instructions. The bottom panel is a size (nm) distribution graph of AF488-labeled lemon PMPs. The median PMP size was determined using Exo bead standards according to NanoFCM's instructions. The median lemon PMPs size was 72.1 nm+/−15.9 nm (SD).

FIG. 9A is a graph showing the absorbance at 280 nm (A.U.) in eluted grapefruit SEC fractions produced from different SEC columns (Columns A, B, C, D, and E) measured on a SpectraMax® spectrophotometer. PMPs were eluted in fractions 4-6.

FIG. 9B is a scatter plot showing purified grapefruit PMPs (combined and pelleted PMP SEC fractions), as measured by nano flow cytometry (NanoFCM). The final grapefruit PMP concentration (6.34×10¹² PMPs/ml) was determined using a bead standard according to NanoFCM's instructions.

FIG. 9C is a graph showing size distribution (nm) of purified grapefruit PMPs. The median PMP size was determined using Exo bead standards according to NanoFCM's instructions. The median grapefruit PMPs size was 63.7 nm+/−11.5 nm (SD).

FIG. 9D is a graph showing the absorbance at 280 nm (A.U.) in eluted lemon SEC fractions of different SEC columns used, measured on a SpectraMax® spectrophotometer. PMPs were eluted in fractions 4-6.

FIG. 9E is a scatter plot showing purified lemon PMPs (combined and pelleted PMP SEC fractions), as measured by nano flow cytometry (NanoFCM). The final lemon PMP concentration (7.42×10¹² PMPs/ml) was determined using a bead standard according to NanoFCM's instructions.

FIG. 9F is a graph showing size distribution (nm) of purified lemon PMPs. The median PMP size was determined using Exo bead standards according to NanoFCM's instructions. The median lemon PMPs size was 68 nm+/−17.5 nm (SD).

FIG. 9G is a bar graph showing the DOX loading capacity (μg DOX per 1000 PMPs) of lemon (LM) and grapefruit (GF) PMPs that were actively (sonication/extrusion) or passively (incubation) loaded with doxorubicin. The loading capacity was calculated by dividing the total concentration of DOX (μg/mL) in the PMP-DOX sample (assessed by fluorescence intensity measurement (Ex/Em=485/550 nm) using a SpectraMax® spectrophotometer) by the total PMP concentration (PMPs/mL) in the sample.

FIG. 9H is a graph showing the stability of grapefruit and lemon DOX-loaded PMP at 4° Celsius, as determined by the PMP concentration (PMP particles/ml) at different time points (days after loading), as measured by NanoFCM.

FIG. 10A is a schematic diagram showing a protocol production of PMPs from 4 liters of grapefruit juice treated with pectinase and EDTA, concentrated 5× using a 300 kDa TFF, washed by 6 volume exchanges of PBS, and concentrated to a final concentration of 20×. Size exclusion chromatography was used to elute the PMP-containing fractions.

FIG. 10B is a graph showing the absorbance at 280 nm (A.U.) of eluted SEC fractions across 9 different SEC columns used (SEC column A-J). PMPs are eluted in SEC fractions 3-7.

FIG. 10C is a graph showing the protein concentration (μg/ml) of eluted SEC fractions across 9 different SEC columns used (SEC column A-J). PMPs are eluted in SEC fractions 3-7. An arrow indicates a fraction containing contaminants.

FIG. 10D is a scatter plot showing purified grapefruit PMPs (combined and pelleted PMP SEC fractions), as measured by nano flow cytometry (NanoFCM). The final grapefruit PMP concentration (7.56×10¹² PMPs/ml) was determined using a bead standard according to NanoFCM's instructions.

FIG. 10E is a graph showing size distribution (nm) of purified grapefruit PMPs. The median PMP size was determined using Exo bead standards according to NanoFCM's instructions. The median grapefruit PMPs size was 70.3 nm+/−12.4 nm (SD).

FIG. 10F is a graph showing the cytotoxic effect of doxorubicin (DOX)-loaded grapefruit PMP treatment of P. aeruginosa. Bacteria were treated in duplicate with PMP-DOX to an effective DOX concentration of 0 (negative control), 5 μM, 1 μM, 25 μM, 50 μM and 100 μM. A kinetic Absorbance measurement at 600 nm was performed (SpectraMax® spectrophotometer) to monitor the OD of the cultures at the indicated time points. All OD values per treatment dose were first normalized to the OD of the first time point at that dose, to normalize for DOX fluorescence bleed-through at 600 nm at high concentration. To determine the cytotoxic effect of PMP-DOX on bacteria, the relative OD was determined within each treatment group as compared to the untreated control (set to 100%).

FIG. 10G is a graph showing the cytotoxic effect of doxorubicin (DOX)-loaded grapefruit PMP treatment of E. coli. Bacteria were treated in duplicate with PMP-DOX to an effective DOX concentration of 0 (negative control), 5 μM, 10 μM, 25 μM, 50 μM and 100 μM. A kinetic Absorbance measurement at 600 nm was performed (SpectraMax® spectrophotometer) to monitor the OD of the cultures at the indicated time points. All OD values per treatment dose were first normalized to the OD of the first time point at that dose, to normalize for DOX fluorescence bleed-through at 600 nm at high concentration. To determine the cytotoxic effect of PMP-DOX on bacteria, the relative OD was determined within each treatment group as compared to the untreated control (set to 100%).

FIG. 10H is a graph showing the cytotoxic effect of doxorubicin (DOX)-loaded grapefruit PMP treatment of S. cerevisiae. Yeast cells were treated in duplicate with PMP-DOX to an effective DOX concentration of 0 (negative control), 5 μM, 10 μM, 25 μM, 50 μM and 100 μM. A kinetic Absorbance measurement at 600 nm was performed (SpectraMax® spectrophotometer) to monitor the OD of the cultures at the indicated time points. All OD values per treatment dose were first normalized to the OD of the first time point at that dose, to normalize for DOX fluorescence bleed-through at 600 nm at high concentration. To determine the cytotoxic effect of PMP-DOX on yeast, the relative OD was determined within each treatment group as compared to the untreated control (set to 100%).

FIG. 10I is a graph showing the cytotoxic effect of doxorubicin (DOX)-loaded grapefruit PMP treatment of P. syringae. Bacteria were treated in duplicate with PMP-DOX to an effective DOX concentration of 0 (negative control), 5 μM, 10 μM, 25 μM, 50 μM and 100 μM. A kinetic Absorbance measurement at 600 nm was performed (SpectraMax® spectrophotometer) to monitor the OD of the cultures at the indicated time points. All OD values per treatment dose were first normalized to the OD of the first time point at that dose, to normalize for DOX fluorescence bleed-through at 600 nm at high concentration. To determine the cytotoxic effect of PMP-DOX on bacteria, the relative OD was determined within each treatment group as compared to the untreated control (set to 100%).

FIG. 11 is a graph showing the luminescence (R.L.U., relative luminescence unit) of Pseudomonas aeruginosa bacteria that were treated with Ultrapure water (negative control), 3 ng free luciferase protein (protein only control) or with an effective luciferase protein dose of 3 ng by luciferase protein-loaded PMPs (PMP-Luc) in duplicate samples for 2 hrs at RT. Luciferase protein in the supernatant and pelleted bacteria was measured by luminescence using the ONE-Glo™ luciferase assay kit (Promega) and measured on a SpectraMax® spectrophotometer.

FIG. 12A is a scatter plot and a graph showing particle size in AF488-labeled lemon PMPs as measured by nanoflow cytometry (NanoFCM). The top panel is a scatter plot showing AF488-labeled lemon PMPs. Particles were gated on the FITC fluorescence signal, relative to unlabeled particles and background signal. The labeling efficiency was 89.4% as determined by the number of fluorescent particles relative to the total number of particles detected. The final AF488-PMP concentration (2.91×10¹² PMPs/ml) was determined from the number of fluorescent particles and using a bead standard with a known concentration according to NanoFCM's instructions. The bottom panel is a size (nm) distribution graph of 488-labeled lemon PMPs. The median PMP size was determined using Exo bead standards according to NanoFCM's instructions. The median lemon AF488-PMPs size was 79.4 nm+/−14.7 nm (SD).

FIG. 12B is a set of photomicrographs showing uptake of lemon (LM) PMPs labeled with Alexa Fluor® 488 (AF488) by the plant cell lines Glycine max (soy bean), Tritium aestivum (wheat), and maize BMS cell culture. Brightfield panels show the position of cells; panels labeled “GFP” show fluorescence of AF488. Uptake of PMPs by a cell is indicated by the presence of the AF488 signal in the cell. Free AF488 (“Free dye”) is shown as a control.

FIG. 13 is a pair of diagrams and a set of photomicrographs showing uptake of lemon (LM) and grapefruit (GF) PMPs labeled with DL800 by Arabidopsis thaliana seedlings and alfalfa sprouts. Intensity of fluorescence of DL800 dye is displayed. Intensity of fluorescence was measured at 22 hpt (hours post-treatment) for Arabidopsis thaliana seedlings and at 24 hpt for alfalfa sprouts. Seedlings incubated with no dye (“negative control”) and with free DL800 dye (“DL800 dye only”) are shown as controls.

### DETAILED DESCRIPTION

Featured herein are compositions and related methods for controlling plant pests based on pest control, e.g., bio-repellants or biopesticide compositions that include plant messenger packs (PMPs), lipid assemblies produced wholly or in part from plant extracellular vesicles (EVs), or segments, portions, or extracts thereof. The PMPs can have pesticidal or insect repellant activity without the inclusion of additional agents (e.g., heterologous functional agents, e.g., pesticidal agents or repellent agents), but may be optionally modified to include additional pesticidal or pest repellent agents. Also included are formulations in which the PMPs are provided in substantially pure form or concentrated forms. The pest control (e.g., biopesticide or biorepellent) compositions and formulations described herein can be delivered directly to a plant to treat or prevent pest infestations and thereby increase the fitness of the plant, such as an agricultural crop. Additionally, or alternatively, the pest control (e.g., biopesticide or biorepellent) compositions can be delivered to a variety of plant pests, such as those that are harmful to plants important for agriculture or commerce, to decrease the fitness of the plant pests.

### I. Pest Control Compositions

The pest control (e.g., biopesticide or biorepellent) compositions described herein include a plurality of plant messenger packs (PMPs). A PMP is a lipid (e.g., lipid bilayer, unilamellar, or multilamellar structure) structure that includes a plant EV, or segment, portion, or extract (e.g., lipid extract) thereof. Plant EVs refer to an enclosed lipid-bilayer structure that naturally occurs in a plant. Plant EVs may be about 5-2000 nm in diameter. Plant EVs can originate from a variety of plant biogenesis pathways. In nature, plant EVs can be found in the intracellular and extracellular compartments of plants, such as the plant apoplast, the compartment located outside the plasma membrane and formed by a continuum of cell walls and the extracellular space. Alternatively, PMPs can be enriched plant EVs found in cell culture media upon secretion from plant cells. Plant EVs can be separated from plants (e.g., from the apoplastic fluid), thereby providing PMPs, by a variety of methods further described herein.

The pest control (e.g., biopesticide or biorepellent) compositions can include PMPs that have pesticidal activity or repellent activity against plant pests, without the further inclusion of additional pesticidal or repellent agents. However, PMPs can additionally include a heterologous pest control agent, e.g., a pesticidal agent or repellent agent, which can be introduced in vivo or in vitro. As such, the PMPs can include a substance with pesticidal activity or repellent activity that is loaded into or onto the PMP by the plant from which the PMP is produced. For example, the pesticidal agent loaded in to the PMP in vivo may be a factor endogenous to the plant or a factor exogenous to the plant (e.g., as expressed by a heterologous genetic construct in a genetically engineered plant). Alternatively, the PMPs may be loaded with a heterologous functional agent in vitro (e.g., following production by a variety of methods further described herein).

PMPs can include plant EVs, or segments, portions, or extracts, thereof, in which the plant EVs are about 5-2000 nm in diameter. For example, the PMP can include a plant EV, or segment, portion, or extract thereof, that has a mean diameter of about 5-50 nm, about 50-100 nm, about 100-150 nm, about 150-200 nm, about 200-250 nm, about 250-300 nm, about 300-350 nm, about 350-400 nm, about 400-450 nm, about 450-500 nm, about 500-550 nm, about 550-600 nm, about 600-650 nm, about 650-700 nm, about 700-750 nm, about 750-800 nm, about 800-850 nm, about 850-900 nm, about 900-950 nm, about 950-1000 nm, about 1000-1250 nm, about 1250-1500 nm, about 1500-1750 nm, or about 1750-2000 nm. In some instances, the PMP includes a plant EV, or segment, portion, or extract thereof, that has a mean diameter of about 5-950 nm, about 5-900 nm, about 5-850 nm, about 5-800 nm, about 5-750 nm, about 5-700 nm, about 5-650 nm, about 5-600 nm, about 5-550 nm, about 5-500 nm, about 5-450 nm, about 5-400 nm, about 5-350 nm, about 5-300 nm, about 5-250 nm, about 5-200 nm, about 5-150 nm, about 5-100 nm, about 5-50 nm, or about 5-25 nm. In certain instances, the plant EV, or segment, portion, or extract thereof, has a mean diameter of about 50-200 nm. In certain instances, the plant EV, or segment, portion, or extract thereof, has a mean diameter of about 50-300 nm. In certain instances, the plant EV, or segment, portion, or extract thereof, has a mean diameter of about 200-500 nm. In certain instances, the plant EV, or segment, portion, or extract thereof, has a mean diameter of about 30-150 nm.

In some instances, the PMP may include a plant EV, or segment, portion, or extract thereof, that has a mean diameter of at least 5 nm, at least 50 nm, at least 100 nm, at least 150 nm, at least 200 nm, at least 250 nm, at least 300 nm, at least 350 nm, at least 400 nm, at least 450 nm, at least 500 nm, at least 550 nm, at least 600 nm, at least 650 nm, at least 700 nm, at least 750 nm, at least 800 nm, at least 850 nm, at least 900 nm, at least 950 nm, or at least 1000 nm. In some instances, the PMP includes a plant EV, or segment, portion, or extract thereof, that has a mean diameter less than 1000 nm, less than 950 nm, less than 900 nm, less than 850 nm, less than 800 nm, less than 750 nm, less than 700 nm, less than 650 nm, less than 600 nm, less than 550 nm, less than 500 nm, less than 450 nm, less than 400 nm, less than 350 nm, less than 300 nm, less than 250 nm, less than 200 nm, less than 150 nm, less than 100 nm, or less than 50 nm. A variety of methods (e.g., a dynamic light scattering method) standard in the art can be used to measure the particle diameter of the plant EV, or segment, portion, or extract thereof.

In some instances, the PMP may include a plant EV, or segment, portion, or extract thereof, that has a mean surface area of 77 nm² to 3.2×10⁶ nm² (e.g., 77-100 nm², 100-1000 nm², 1000-1×10⁴ nm², 1×10⁴-1×10⁵ nm², 1×10⁵-1×10⁶ nm², or 1×10⁶-3.2×10⁶ nm²). In some instances, the PMP may include a plant EV, or segment, portion, or extract thereof, that has a mean volume of 65 nm³ to 5.3×10⁸ nm³ (e.g., 65-100 nm³, 100-1000 nm³, 1000-1×10⁴ nm³, 1×10⁴-1×10⁵ nm³, 1×10⁵-1×10⁶ nm³, 1×10⁶-1×10⁷ nm³, 1×10⁷-1×10⁸ nm³, 1×10⁸-5.3×10⁸ nm³). In some instances, the PMP may include a plant EV, or segment, portion, or extract thereof, that has a mean surface area of at least 77 nm², (e.g., at least 77 nm², at least 100 nm², at least 1000 nm², at least 1×10⁴ nm², at least 1×10⁵ nm², at least 1×10⁶ nm², or at least 2×10⁶ nm²). In some instances, the PMP may include a plant EV, or segment, portion, or extract thereof, that has a mean volume of at least 65 nm³ (e.g., at least 65 nm³, at least 100 nm³, at least 1000 nm³, at least 1×10⁴ nm³, at least 1×10⁵ nm³, at least 1×10⁶ nm³, at least 1×10⁷ nm³, at least 1×10⁸ nm³, at least 2×10⁸ nm³, at least 3×10⁸ nm³, at least 4×10⁸ nm³, or at least 5×10⁸ nm³.

In some instances, the PMP can have the same size as the plant EV or segment, extract, or portion thereof. Alternatively, the PMP may have a different size than the initial plant EV from which the PMP is produced. For example, the PMP may have a diameter of about 5-2000 nm in diameter. For example, the PMP can have a mean diameter of about 5-50 nm, about 50-100 nm, about 100-150 nm, about 150-200 nm, about 200-250 nm, about 250-300 nm, about 300-350 nm, about 350-400 nm, about 400-450 nm, about 450-500 nm, about 500-550 nm, about 550-600 nm, about 600-650 nm, about 650-700 nm, about 700-750 nm, about 750-800 nm, about 800-850 nm, about 850-900 nm, about 900-950 nm, about 950-1000 nm, about 1000-1200 nm, about 1200-1400 nm, about 1400-1600 nm, about 1600-1800 nm, or about 1800-2000 nm. In some instances, the PMP may have a mean diameter of at least 5 nm, at least 50 nm, at least 100 nm, at least 150 nm, at least 200 nm, at least 250 nm, at least 300 nm, at least 350 nm, at least 400 nm, at least 450 nm, at least 500 nm, at least 550 nm, at least 600 nm, at least 650 nm, at least 700 nm, at least 750 nm, at least 800 nm, at least 850 nm, at least 900 nm, at least 950 nm, at least 1000 nm, at least 1200 nm, at least 1400 nm, at least 1600 nm, at least 1800 nm, or about 2000 nm. A variety of methods (e.g., a dynamic light scattering method) standard in the art can be used to measure the particle diameter of the PMPs. In some instances, the size of the PMP is determined following loading of heterologous functional agents, or following other modifications to the PMPs.

In some instances, the PMP may have a mean surface area of 77 nm² to 1.3×10⁷ nm² (e.g., 77-100 nm², 100-1000 nm², 1000-1×10⁴ nm², 1×10⁴-1×10⁵ nm², 1×10⁵-1×10⁶ nm², or 1×10⁶-1.3×10⁷ nm²). In some instances, the PMP may have a mean volume of 65 nm³ to 4.2×10⁹ nm³ (e.g., 65-100 nm³, 100-1000 nm³, 1000-1×10⁴ nm³, 1×10⁴-1×10⁵ nm³, 1×10⁵-1×10⁶ nm³, 1×10⁶-1×10⁷ nm³, 1×10⁷-1×10⁸ nm³, 1×10⁸-1×10⁹ nm³, or 1×10⁹-4.2×10⁹ nm³). In some instances, the PMP has a mean surface area of at least 77 nm², (e.g., at least 77 nm², at least 100 nm², at least 1000 nm², at least 1×10⁴ nm², at least 1×10⁵ nm², at least 1×10⁶ nm², or at least 1×10⁷ nm²). In some instances, the PMP has a mean volume of at least 65 nm³ (e.g., at least 65 nm³, at least 100 nm³, at least 1000 nm³, at least 1×10⁴ nm³, at least 1×10⁵ nm³, at least 1×10⁶ nm³, at least 1×10⁷ nm³, at least 1×10⁸ nm³, at least 1×10⁹ nm³, at least 2×10⁹ nm³, at least 3×10⁹ nm³, or at least 4×10⁹ nm³).

In some instances, the PMP may include an intact plant EV. Alternatively, the PMP may include a segment, portion, or extract of the full surface area of the vesicle (e.g., a segment, portion, or extract including less than 100% (e.g., less than 90%, less than 80%, less than 70%, less than 60%, less than 50%, less than 40%, less than 30%, less than 20%, less than 10%, less than 10%, less than 5%, or less than 1%) of the full surface area of the vesicle) of a plant EV. The segment, portion, or extract may be any shape, such as a circumferential segment, spherical segment (e.g., hemisphere), curvilinear segment, linear segment, or flat segment. In instances where the segment is a spherical segment of the vesicle, the spherical segment may represent one that arises from the splitting of a spherical vesicle along a pair of parallel lines, or one that arises from the splitting of a spherical vesicle along a pair of non-parallel lines. Accordingly, the plurality of PMPs can include a plurality of intact plant EVs, a plurality of plant EV segments, portions, or extracts, or a mixture of intact and segments of plant EVs. One skilled in the art will appreciate that the ratio of intact to segmented plant EVs will depend on the particular isolation method used. For example, grinding or blending a plant, or part thereof, may produce PMPs that contain a higher percentage of plant EV segments, portions, or extracts than a non-destructive extraction method, such as vacuum-infiltration.

In instances where, the PMP includes a segment, portion, or extract of a plant EV, the EV segment, portion, or extract may have a mean surface area less than that of an intact vesicle, e.g., a mean surface area less than 77 nm², 100 nm², 1000 nm², 1×10⁴ nm², 1×10⁵ nm², 1×10⁶ nm², or 3.2×10⁶ nm²). In some instances, the EV segment, portion, or extract has a surface area of less than 70 nm², 60 nm², 50 nm², 40 nm², 30 nm², 20 nm², or 10 nm²). In some instances, the PMP may include a plant EV, or segment, portion, or extract thereof, that has a mean volume less than that of an intact vesicle, e.g., a mean volume of less than 65 nm³, 100 nm³, 1000 nm³, 1×10⁴ nm³, 1×10⁵ nm³, 1×10⁶ nm³, 1×10⁷ nm³, 1×10⁸ nm³, or 5.3×10⁸ nm³).

In instances where the PMP includes an extract of a plant EV, e.g., in instances where the PMP includes lipids extracted (e.g., with chloroform) from a plant EV, the PMP may include at least 1%, 2%, 5%, 10%, 20%, 30%, 40%, 50%, 60% or more, of lipids extracted (e.g., with chloroform) from a plant EV. The PMPs in the plurality may include plant EV segments and/or plant EV-extracted lipids or a mixture thereof.

Further outlined herein are details regarding methods of producing PMPs, plant EV markers that can be associated with PMPs, and formulations for compositions including PMPs.

A. Production Methods

PMPs may be produced from plant EVs, or a segment, portion or extract (e.g., lipid extract) thereof, that occur naturally in plants, or parts thereof, including plant tissues or plant cells. An exemplary method for producing PMPs includes (a) providing an initial sample from a plant, or a part thereof, wherein the plant or part thereof comprises EVs; and (b) isolating a crude PMP fraction from the initial sample, wherein the crude PMP fraction has a decreased level of at least one contaminant or undesired component from the plant or part thereof relative to the level in the initial sample. The method can further include an additional step (c) comprising purifying the crude PMP fraction, thereby producing a plurality of pure PMPs, wherein the plurality of pure PMPs have a decreased level of at least one contaminant or undesired component from the plant or part thereof relative to the level in the crude EV fraction. Each production step is discussed in further detail, below. Exemplary methods regarding the isolation and purification of PMPs is found, for example, in Rutter and Innes, Plant Physiol. 173(1): 728-741, 2017; Rutter et al, Bio. Protoc. 7(17): e2533, 2017; Regente et al, J of Exp. Biol. 68(20): 5485-5496, 2017; Mu et al, Mol. Nutr. Food Res., 58, 1561-1573, 2014, and Regente et al, FEBS Letters. 583: 3363-3366, 2009, each of which is herein incorporated by reference.

For example, a plurality of PMPs may be isolated from a plant by a process which includes the steps of: (a) providing an initial sample from a plant, or a part thereof, wherein the plant or part thereof comprises EVs; (b) isolating a crude PMP fraction from the initial sample, wherein the crude PMP fraction has a decreased level of at least one contaminant or undesired component from the plant or part thereof relative to the level in the initial sample (e.g., a level that is decreased by at least 1%, 2%, 5%, 10%, 15%, 20%, 25%, 30%, 40%, 45%, 50%, 55%, 60%, 70%, 80%, 90%, 95%, 96%, 98%, 99%, or 100%); and (c) purifying the crude PMP fraction, thereby producing a plurality of pure PMPs, wherein the plurality of pure PMPs have a decreased level of at least one contaminant or undesired component from the plant or part thereof relative to the level in the crude EV fraction (e.g., a level that is decreased by at least 1%, 2%, 5%, 10%, 15%, 20%, 25%, 30%, 40%, 45%, 50%, 55%, 60%, 70%, 80%, 90%, 95%, 96%, 98%, 99%, or 100%).

The PMPs provided herein can include a plant EV, or segment, portion, or extract thereof, isolated from a variety of plants. PMPs may be isolated from any genera of plants (vascular or nonvascular), including but not limited to angiosperms (monocotyledonous and dicotyledonous plants), gymnosperms, ferns, selaginellas, horsetails, psilophytes, lycophytes, algae (e.g., unicellular or multicellular, e.g., archaeplastida), or bryophytes. In certain instances, PMPs can be produced from a vascular plant, for example monocotyledons or dicotyledons or gymnosperms. For example, PMPs can be produced from alfalfa, apple, Arabidopsis, banana, barley, canola, castor bean, chicory, chrysanthemum, clover, cocoa, coffee, cotton, cottonseed, corn, crambe, cranberry, cucumber, dendrobium, dioscorea, eucalyptus, fescue, flax, gladiolus, liliacea, linseed, millet, muskmelon, mustard, oat, oil palm, oilseed rape, papaya, peanut, pineapple, ornamental plants, Phaseolus, potato, rapeseed, rice, rye, ryegrass, safflower, sesame, sorghum, soybean, sugarbeet, sugarcane, sunflower, strawberry, tobacco, tomato, turfgrass, wheat or vegetable crops such as lettuce, celery, broccoli, cauliflower, cucurbits; fruit and nut trees, such as apple, pear, peach, orange, grapefruit, lemon, lime, almond, pecan, walnut, hazel; vines, such as grapes, kiwi, hops; fruit shrubs and brambles, such as raspberry, blackberry, gooseberry; forest trees, such as ash, pine, fir, maple, oak, chestnut, popular; with alfalfa, canola, castor bean, corn, cotton, crambe, flax, linseed, mustard, oil palm, oilseed rape, peanut, potato, rice, safflower, sesame, soybean, sugarbeet, sunflower, tobacco, tomato, or wheat.

PMPs may be produced from a whole plant (e.g., a whole rosettes or seedlings) or alternatively from one or more plant parts (e.g., leaf, seed, root, fruit, vegetable, pollen, phloem sap, or xylem sap). For example, PMPs can be produced from shoot vegetative organs/structures (e.g., leaves, stems, or tubers), roots, flowers and floral organs/structures (e.g., pollen, bracts, sepals, petals, stamens, carpels, anthers, or ovules), seed (including embryo, endosperm, or seed coat), fruit (the mature ovary), sap (e.g., phloem or xylem sap), plant tissue (e.g., vascular tissue, ground tissue, tumor tissue, or the like), and cells (e.g., single cells, protoplasts, embryos, callus tissue, guard cells, egg cells, or the like), or progeny of same. For instance, the isolation step may involve (a) providing a plant, or a part thereof. In some examples, the plant part is an Arabidopsis leaf. The plant may be at any stage of development. For example, the PMP can be produced from seedlings, e.g., 1 week, 2 week, 3 week, 4 week, 5 week, 6 week, 7 week, or 8 week old seedlings (e.g., Arabidopsis seedlings). Other exemplary PMPs can include PMPs produced from roots (e.g., ginger roots), fruit juice (e.g., grapefruit juice), vegetables (e.g., broccoli), pollen (e.g., olive pollen), phloem sap (e.g., Arabidopsis phloem sap), or xylem sap (e.g., tomato plant xylem sap).

PMPs can be produced from a plant, or part thereof, by a variety of methods. Any method that allows release of the EV-containing apoplastic fraction of a plant, or an otherwise extracellular fraction that contains PMPs comprising secreted EVs (e.g., cell culture media) is suitable in the present methods. EVs can be separated from the plant or plant part by either destructive (e.g., grinding or blending of a plant, or any plant part) or non-destructive (washing or vacuum infiltration of a plant or any plant part) methods. For instance, the plant, or part thereof, can be vacuum-infiltrated, ground, blended, or a combination thereof to isolate EVs from the plant or plant part, thereby producing PMPs. For instance, the isolating step may involve (b) isolating a crude PMP fraction from the initial sample (e.g., a plant, a plant part, or a sample derived from a plant or plant part), wherein the isolating step involves vacuum infiltrating the plant (e.g., with a vesicle isolation buffer) to release and collect the apoplastic fraction. Alternatively, the isolating step may involve grinding or blending the plant to release the EVs, thereby producing PMPs.

Upon isolating the plant EVs, thereby producing PMPs, the PMPs can be separated or collected into a crude PMP fraction (e.g., an apoplastic fraction). For instance, the separating step may involve separating the plurality of PMPs into a crude PMP fraction using centrifugation (e.g., differential centrifugation or ultracentrifugation) and/or filtration to separate the PMP-containing fraction from large contaminants, including plant tissue debris, plant cells, or plant cell organelles (e.g., nuclei or chloroplasts). As such, the crude PMP fraction will have a decreased number of large contaminants, including plant tissue debris, plant cells, or plant cell organelles (e.g., nuclei, mitochondria or chloroplasts), as compared to the initial sample from the source plant or plant part.

In some instances, the isolating step may involve separating the plurality of PMPs into a crude PMP fraction using centrifugation (e.g., differential centrifugation or ultracentrifugation) and/or filtration to separate the PMP-containing fraction from plant cells or cellular debris. In such instances, the crude PMP fraction will have a decreased number of plant cells or cellular debris, as compared to the initial sample from the source plant or plant part.

The crude PMP fraction can be further purified by additional purification methods to produce a plurality of pure PMPs. For example, the crude PMP fraction can be separated from other plant components by ultracentrifugation, e.g., using a density gradient (iodixanol or sucrose) and/or use of other approaches to remove aggregated components (e.g., precipitation or size-exclusion chromatography). The resulting pure PMPs may have a decreased level of contaminants or undesired components from the source plant (e.g., one or more non-PMP components, such as protein aggregates, nucleic acid aggregates, protein-nucleic acid aggregates, free lipoproteins, lipido-proteic structures), nuclei, cell wall components, cell organelles, or a combination thereof) relative to one or more fractions generated during the earlier separation steps, or relative to a pre-established threshold level, e.g., a commercial release specification. For example, the pure PMPs may have a decreased level (e.g., by about 5%, 10%, 15%, 20%, 30%, 40%, 50%, 60%, 70%, 80%, 90%, 100%, or more than 100%; or by about 2× fold, 4× fold, 5× fold, 10× fold, 20× fold, 25× fold, 50× fold, 75× fold, 100× fold, or more than 100× fold) of plant organelles or cell wall components relative to the level in the initial sample. In some instances, the pure PMPs are substantially free (e.g., have undetectable levels) of one or more non-PMP components, such as protein aggregates, nucleic acid aggregates, protein-nucleic acid aggregates, free lipoproteins, lipido-proteic structures), nuclei, cell wall components, cell organelles, or a combination thereof. Further examples of the releasing and separation steps can be found in Example 1. The PMPs may be at a concentration of, e.g., 1×10⁹, 5×10⁹, 1×10¹⁰, 5×10¹⁰, 5×10¹⁰, 1×10¹¹, 2×10¹¹, 3×10¹¹, 4×10¹¹, 5×10¹¹, b6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, or more than 1×10¹³ PMPs/mL.

For example, protein aggregates may be removed from isolated PMPs. For example, the isolated PMP solution can be taken through a range of pHs (e.g., as measured using a pH probe) to precipitate out protein aggregates in solution. The pH can be adjusted to, e.g., pH 3, pH 5, pH 7, pH 9, or pH 11 with the addition of, e.g., sodium hydroxide or hydrochloric acid. Once the solution is at the specified pH, it can be filtered to remove particulates. Alternatively, the isolated PMP solution can be flocculated using the addition of charged polymers, such as Polymin-P or Praestol 2640. Briefly, Polymin-P or Praestol 2640 is added to the solution and mixed with an impeller. The solution can then be filtered to remove particulates. Alternatively, aggregates can be solubilized by increasing salt concentration. For example NaCl can be added to the isolated PMP solution until it is at, e.g., 1 mol/L. The solution can then be filtered to isolate the PMPs. Alternatively, aggregates are solubilized by increasing the temperature. For example, the isolated PMPs can be heated under mixing until the solution has reached a uniform temperature of, e.g., 50° C. for 5 minutes. The PMP mixture can then be filtered to isolate the PMPs. Alternatively, soluble contaminants from PMP solutions can be separated by size-exclusion chromatography column according to standard procedures, where PMPs elute in the first fractions, whereas proteins and ribonucleoproteins and some lipoproteins are eluted later. The efficiency of protein aggregate removal can be determined by measuring and comparing the protein concentration before and after removal of protein aggregates via BCA/Bradford protein quantification.

Any of the production methods described herein can be supplemented with any quantitative or qualitative methods known in the art to characterize or identify the PMPs at any step of the production process. PMPs may be characterized by a variety of analysis methods to estimate PMP yield, PMP concentration, PMP purity, PMP composition, or PMP sizes. PMPs can be evaluated by a number of methods known in the art that enable visualization, quantitation, or qualitative characterization (e.g., identification of the composition) of the PMPs, such as microscopy (e.g., transmission electron microscopy), dynamic light scattering, nanoparticle tracking, spectroscopy (e.g., Fourier transform infrared analysis), or mass spectrometry (protein and lipid analysis). In certain instances, methods (e.g., mass spectroscopy) may be used to identify plant EV markers present on the PMP, such as markers disclosed in the Appendix. To aid in analysis and characterization, of the PMP fraction, the PMPs can additionally be labelled or stained. For example, the PMPs can be stained with 3,3′-dihexyloxacarbocyanine iodide (DIOC₆), a fluorescent lipophilic dye, PKH67 (Sigma Aldrich); Alexa Fluor® 488 (Thermo Fisher Scientific), or DyLight™ 800 (Thermo Fisher). In the absence of sophisticated forms of nanoparticle tracking, this relatively simple approach quantifies the total membrane content and can be used to indirectly measure the concentration of PMPs (Rutter and Innes, Plant Physiol. 173(1): 728-741, 2017; Rutter et al, Bio. Protoc. 7(17): e2533, 2017). For more precise measurements, and to assess the size distributions of PMPs, nanoparticle tracking can be used.

During the production process, the PMPs can optionally be prepared such that the PMPs are at an increased concentration (e.g., by about 5%, 10%, 15%, 20%, 30%, 40%, 50%, 60%, 70%, 80%, 90%, 100%, or more than 100%; or by about 2× fold, 4× fold, 5× fold, 10× fold, 20× fold, 25× fold, 50× fold, 75× fold, 100× fold, or more than 100× fold) relative to the EV level in a control or initial sample. The isolated PMPs may make up about 0.1% to about 100% of the pest control (e.g., biopesticide or biorepellent) composition, such as any one of about 0.01% to about 100%, about 1% to about 99.9%, about 0.1% to about 10%, about 1% to about 25%, about 10% to about 50%, about 50% to about 99%, or about 75% to about 100%. In some instances, the composition includes at least any of 0.1%, 0.5%, 1%, 5%, 10%, 20%, 30%, 40%, 50%, 60%, 70%, 80%, 90%, 95%, or more PMPs, e.g., as measured by wt/vol, percent PMP protein composition, and/or percent lipid composition (e.g., by measuring fluorescently labelled lipids); See, e.g., Example 3). In some instances, the concentrated agents are used as commercial products, e.g., the final user may use diluted agents, which have a substantially lower concentration of active ingredient. In some embodiments, the composition is formulated as a pest control concentrate formulation, e.g., an ultra-low-volume concentrate formulation.

As illustrated by Example 1, PMPs can be produced from a variety of plants, or parts thereof (e.g., the leaf apoplast, seed apoplast, root, fruit, vegetable, pollen, phloem, or xylem sap). For example, PMPs can be isolated from the apoplastic fraction of a plant, such as the apoplast of a leaf (e.g., apoplast Arabidopsis thaliana leaves) or the apoplast of seeds (e.g., apoplast of sunflower seeds). Other exemplary PMPs are produced from roots (e.g., ginger roots), fruit juice (e.g., grapefruit juice), vegetables (e.g., broccoli), pollen (e.g., olive pollen), phloem sap (e.g., Arabidopsis phloem sap), xylem sap (e.g., tomato plant xylem sap), or cell culture supernatant (e.g. BY2 tobacco cell culture supernatant). This example further demonstrates the production of PMPs from these various plant sources.

As illustrated by Example 2, PMPs can be purified by a variety of methods, for example, by using a density gradient (iodixanol or sucrose) in conjunction with ultracentrifugation and/or methods to remove aggregated contaminants, e.g., precipitation or size-exclusion chromatography. For instance, Example 2 illustrates purification of PMPs that have been obtained via the separation steps outlined in Example 1. Further, PMPs can be characterized in accordance with the methods illustrated in Example 3.

In some instances, the PMPs of the present compositions and methods can be isolated from a plant, or part thereof, and used without further modification to the PMP. In other instances, the PMP can be modified prior to use, as outlined further herein.

B. Plant EV-Markers

The PMPs of the present compositions and methods may have a range of markers that identify the PMP as being produced from a plant EV, and/or including a segment, portion, or extract thereof. As used herein, the term “plant EV-marker” refers to a component that is naturally associated with a plant and incorporated into or onto the plant EV in planta, such as a plant protein, a plant nucleic acid, a plant small molecule, a plant lipid, or a combination thereof. Examples of plant EV-markers can be found, for example, in Rutter and Innes, Plant Physiol. 173(1): 728-741, 2017; Raimondo et al., Oncotarget. 6(23): 19514, 2015; Ju et al., Mol. Therapy. 21(7):1345-1357, 2013; Wang et al., Molecular Therapy. 22(3): 522-534, 2014; and Regente et al, J of Exp. Biol. 68(20): 5485-5496, 2017; each of which is incorporated herein by reference. Additional examples of plant EV-markers are listed in the Appendix, and are further outlined herein.

The plant EV marker can include a plant lipid. Examples of plant lipid markers that may be found in the PMP include phytosterol, campesterol, β-sitosterol, stigmasterol, avenasterol, glycosyl inositol phosphoryl ceramides (GIPCs), glycolipids (e.g., monogalactosyldiacylglycerol (MGDG) or digalactosyldiacylglycerol (DGDG)), or a combination thereof. For instance, the PMP may include GIPCs, which represent the main sphingolipid class in plants and are one of the most abundant membrane lipids in plants. Other plant EV markers may include lipids that accumulate in plants in response to abiotic or biotic stressors (e.g., bacterial or fungal infection), such as phosphatidic acid (PA) or phosphatidylinositol-4-phosphate (PI4P).

Alternatively, the plant EV marker may include a plant protein. In some instances, the protein plant EV marker may be an antimicrobial protein naturally produced by plants, including defense proteins that plants secrete in response to abiotic or biotic stressors (e.g., bacterial or fungal infection). Plant pathogen defense proteins include soluble N-ethylmalemide-sensitive factor association protein receptor protein (SNARE) proteins (e.g., Syntaxin-121 (SYP121; GenBank Accession No.: NP_187788.1 or NP_974288.1), Penetration1 (PEN1; GenBank Accession No: NP_567462.1)) or ABC transporter Penetration3 (PEN3; GenBank Accession No: NP_191283.2). Other examples of plant EV markers includes proteins that facilitate the long-distance transport of RNA in plants, including phloem proteins (e.g., Phloem protein2-A1 (PP2-A1), GenBank Accession No: NP_193719.1), calcium-dependent lipid-binding proteins, or lectins (e.g., Jacalin-related lectins, e.g., Helianthus annuus jacalin (Helja; GenBank: AHZ86978.1). For example, the RNA binding protein may be Glycine-Rich RNA Binding Protein-7 (GRP7; GenBank Accession Number: NP_179760.1). Additionally, proteins that regulate plasmodesmata function can in some instances be found in plant EVs, including proteins such as Synap-Totgamin A A (GenBank Accession No: NP_565495.1). In some instances, the plant EV marker can include a protein involved in lipid metabolism, such as phospholipase C or phospholipase D. In some instances, the plant protein EV marker is a cellular trafficking protein in plants. In certain instances where the plant EV marker is a protein, the protein marker may lack a signal peptide that is typically associated with secreted proteins. Unconventional secretory proteins seem to share several common features like (i) lack of a leader sequence, (ii) absence of PTMs specific for ER or Golgi apparatus, and/or (iii) secretion not affected by brefeldin A which blocks the classical ER/Golgi-dependent secretion pathway. One skilled in the art can use a variety of tools freely accessible to the public (e.g., SecretomeP Database; SUBA3 (SUBcellular localization database for Arabidopsis proteins)) to evaluate a protein for a signal sequence, or lack thereof.

In instances where the plant EV marker is a protein, the protein may have an amino acid sequence having at least 35%, 40%, 45%, 50%, 55%, 60%, 65%, 70%, 75%, 80%, 85%, 90%, 95%, 98%, 99%, or 100% sequence identity to a plant EV marker, such as any of the plant EV markers listed in the Appendix. For example, the protein may have an amino acid sequence having at least 35%, 40%, 45%, 50%, 55%, 60%, 65%, 70%, 75%, 80%, 85%, 90%, 95%, 98%, 99%, or 100% sequence identity to PEN1 from Arabidopsis thaliana (GenBank Accession Number: NP_567462.1).

In some instances, the plant EV marker includes a nucleic acid encoded in plants, e.g., a plant RNA, a plant DNA, or a plant PNA. For example, the PMP may include dsRNA, mRNA, a viral RNA, a microRNA (miRNA), or a small interfering RNA (siRNA) encoded by a plant. In some instances, the nucleic acid may be one that is associated with a protein that facilitates the long-distance transport of RNA in plants, as discussed herein. In some instances, the nucleic acid plant EV marker may be one involved in host-induced gene silencing (HIGS), which is the process by which plants silence foreign transcripts of plant pests (e.g., pathogens such as fungi). For example, the nucleic acid may be one that silences bacterial or fungal genes. In some instances, the nucleic acid may be a microRNA, such as miR159 or miR166, which target genes in a fungal pathogen (e.g., Verticillium dahliae). In some instances, the protein may be one involved in carrying plant defense compounds, such as proteins involved in glucosinolate (GSL) transport and metabolism, including Glucosinolate Transporter-1-1 (GTR1; GenBank Accension No: NP_566896.2), Glucosinolate Transporter-2 (GTR2; NP_201074.1), or Epithiospecific Modifier 1 (ESM1; NP_188037.1).

In instances where the plant EV marker is a nucleic acid, the nucleic acid may have a nucleotide sequence having at least 35%, 40%, 45%, 50%, 55%, 60%, 65%, 70%, 75%, 80%, 85%, 90%, 95%, 98%, 99%, or 100% sequence identity to a plant EV marker, e.g., such as those encoding the plant EV markers listed in the Appendix. For example, the nucleic acid may have a polynucleotide sequence having at least 35%, 40%, 45%, 50%, 55%, 60%, 65%, 70%, 75%, 80%, 85%, 90%, 95%, 98%, 99%, or 100% sequence identity to miR159 or miR166.

In some instances, the plant EV marker includes a compound produced by plants. For example, the compound may be a defense compound produced in response to abiotic or biotic stressors, such as secondary metabolites. One such secondary metabolite that be found in PMPs are glucosinolates (GSLs), which are nitrogen and sulfur-containing secondary metabolites found mainly in Brassicaceae plants. Other secondary metabolites may include allelochemicals.

In some instances, the PMP may also be identified as being produced from a plant EV based on the lack of certain markers (e.g., lipids, polypeptides, or polynucleotides) that are not typically produced by plants, but are generally associated with other organisms (e.g., markers of animal EVs, bacterial EVs, or fungal EVs). For example, in some instances, the PMP lacks lipids typically found in animal EVs, bacterial EVs, or fungal EVs. In some instances, the PMP lacks lipids typical of animal EVs (e.g., sphingomyelin). In some instances, the PMP does not contain lipids typical of bacterial EVs or bacterial membranes (e.g., LPS). In some instances, the PMP lacks lipids typical of fungal membranes (e.g., ergosterol).

Plant EV markers can be identified using any approaches known in the art that enable identification of small molecules (e.g., mass spectroscopy, mass spectrometry), lipds (e.g., mass spectroscopy, mass spectrometry), proteins (e.g., mass spectroscopy, immunoblotting), or nucleic acids (e.g., PCR analysis). In some instances, a PMP composition described herein includes a detectable amount, e.g., a pre-determined threshold amount, of a plant EV marker described herein.

C. Loading of Agents

The PMP can be modified to include a heterologous functional agent, e.g., a pesticidal agent or repellent agent, such as those described herein. The PMP can carry or associate with such agents by a variety of means to enable delivery of the agent to a target plant or plant pest, e.g., by encapsulating the agent, incorporation of the component in the lipid bilayer structure, or association of the component (e.g., by conjugation) with the surface of the lipid bilayer structure of the PMP.

The heterologous functional agent can be incorporated or loaded into or onto the PMP by any methods known in the art that allow association, directly or indirectly, between the PMP and agent. Heterologous functional agent agents can be incorporated into the PMP by an in vivo method (e.g., in planta, e.g., through production of PMPs from a transgenic plant that comprises the heterologous agent), or in vitro (e.g., in tissue culture, or in cell culture), or both in vivo and in vitro methods.

In instances where the PMPs are loaded with a heterologous functional agent (e.g., a pesticidal agent or repellent) in vivo, the PMP may be produced from an EV, or segment, portion, or extract thereof, that has been loaded in planta, in tissue culture, or in cell culture. In planta methods include expression of the heterologous functional agent (e.g., pesticidal agent or repellent agent) in a plant that has been genetically modified to express the heterologous functional agent. In some instances, the heterologous functional agent is exogenous to the plant. Alternatively, the heterologous functional agent may be naturally found in the plant, but expressed at an elevated level relative to level of that found in a non-genetically modified plant.

In some instances, the PMP can be loaded in vitro. The substance may be loaded onto or into (e.g., may be encapsulated by) the PMPs using, but not limited to, physical, chemical, and/or biological methods. For example, the heterologous functional agent may be introduced into PMP by one or more of electroporation, sonication, passive diffusion, stirring, lipid extraction, or extrusion. Loaded PMPs can be assessed to confirm the presence or level of the loaded agent using a variety methods, such as HPLC (e.g., to assess small molecules); immunoblotting (e.g., to assess proteins); and quantitative PCR (e.g., to assess nucleotides). However, it should be appreciated by those skilled in the art that the loading of a substance of interest into PMPs is not limited to the above-illustrated methods.

In some instances, the heterologous functional agent can be conjugated to the PMP, in which the heterologous functional agent is connected or joined, indirectly or directly, to the PMP. For instance, one or more pesticidal agents can be chemically-linked to a PMP, such that the one or more pesticidal agents are joined (e.g., by covalent or ionic bonds) directly to the lipid bilayer of the PMP. In some instances, the conjugation of various pesticidal agents to the PMPs can be achieved by first mixing the one or more heterologous functional agents with an appropriate cross-linking agent (e.g., N-ethylcarbo-diimide (“EDC”), which is generally utilized as a carboxyl activating agent for amide bonding with primary amines and also reacts with phosphate groups) in a suitable solvent. After a period of incubation sufficient to allow the heterologous functional agent to attach to the cross-linking agent, the cross-linking agent/heterologous functional agent mixture can then be combined with the PMPs and, after another period of incubation, subjected to a sucrose gradient (e.g., and 8, 30, 45, and 60% sucrose gradient) to separate the free heterologous functional agent and free PMPs from the pesticidal agents conjugated to the PMPs. As part of combining the mixture with a sucrose gradient, and an accompanying centrifugation step, the PMPs conjugated to the pesticidal agents are then seen as a band in the sucrose gradient, such that the conjugated PMPs can then be collected, washed, and dissolved in a suitable solution for use as described herein.

In some instances, the PMP is stably associated with the heterologous functional agent prior to and following delivery of the PMP, e.g., to a plant or to a pest. In other instances, the PMP is associated with the heterologous functional agent such that the heterologous functional agent becomes dissociated from the PMP following delivery of the PMP, e.g., to a plant or to a pest.

The PMP can be further modified with other components (e.g., lipids, e.g., sterols, e.g., cholesterol; or small molecules) to further alter the functional and structural characteristics of the PMP. For example, the PMPs can be further modified with stabilizing molecules that increase the stability of the PMP (e.g., for at least one day at room temperature, and/or stable for at least one week at 4° C.).

The PMPs can be loaded with various concentrations of the heterologous functional agent, depending on the particular agent or use. For example, in some instances, the PMPs are loaded such that the pest control (e.g., biopesticide or biorepellent) composition disclosed herein includes about 0.001, 0.01, 0.1, 1.0, 2, 3, 4, 5, 6, 7, 8, 9, 10, 15, 20, 30, 40, 50, 60, 70, 80, 90, or 95 (or any range between about 0.001 and 95) or more wt % of a pesticidal agent and/or a repellent agent. In some instances, the PMPs are loaded such that the pest control (e.g., biopesticide or biorepellent) composition includes about 95, 90, 80, 70, 60, 50, 40, 30, 20, 15, 10, 9, 8, 7, 6, 5, 4, 3, 2, 1.0, 0.1, 0.01, 0.001 (or any range between about 95 and 0.001) or less wt % of a pesticidal agent and/or a repellent agent. For example, the pest control (e.g., biopesticide or biorepellent) composition can include about 0.001 to about 0.01 wt %, about 0.01 to about 0.1 wt %, about 0.1 to about 1 wt %, about 1 to about 5 wt %, or about 5 to about 10 wt %, about 10 to about 20 wt % of the pesticidal agent and/or a repellent agent. In some instances, the PMP can be loaded with about 1, 5, 10, 50, 100, 200, or 500, 1,000, 2,000 (or any range between about 1 and 2,000) or more μg/ml of a pesticidal agent and/or a repellent agent. A liposome of the invention can be loaded with about 2,000, 1,000, 500, 200, 100, 50, 10, 5, 1 (or any range between about 2,000 and 1) or less μg/ml of a pesticidal agent and/or a repellent agent.

In some instances, the PMPs are loaded such that the pest control (e.g., biopesticide or biorepellent) composition disclosed herein includes at least 0.001 wt %, at least 0.01 wt %, at least 0.1 wt %, at least 1.0 wt %, at least 2 wt %, at least 3 wt %, at least 4 wt %, at least 5 wt %, at least 6 wt %, at least 7 wt %, at least 8 wt %, at least 9 wt %, at least 10 wt %, at least 15 wt %, at least 20 wt %, at least 30 wt %, at least 40 wt %, at least 50 wt %, at least 60 wt %, at least 70 wt %, at least 80 wt %, at least 90 wt %, or at least 95 wt % of a pesticidal agent and/or a repellent agent. In some instances, the PMP can be loaded with at least 1 μg/ml, at least 5 μg/ml, at least 10 μg/ml, at least 50 μg/ml, at least 100 μg/ml, at least 200 μg/ml, at least 500 μg/ml, at least 1,000 μg/ml, at least 2,000 μg/ml of a pesticidal agent and/or a repellent agent.

Examples of particular pesticidal agents or repellent agents that can be loaded into the PMP are further outlined in the section entitled “Heterologous Functional Agents.”

D. Formulations

To allow ease of application, handling, transportation, storage, and activity, the active agent, here PMPs, can be formulated with other substances. PMPs can be formulated into, for example, baits, concentrated emulsions, dusts, emulsifiable concentrates, fumigants, gels, granules, microencapsulations, seed treatments, suspension concentrates, suspoemulsions, tablets, water soluble liquids, water dispersible granules or dry flowables, wettable powders, and ultra-low volume solutions. For further information on formulation types see “Catalogue of Pesticide Formulation Types and International Coding System” Technical Monograph n° 2, 5th Edition by CropLife International (2002).

Active agents (e.g., PMPs, additional pesticides) can be applied as aqueous suspensions or emulsions prepared from concentrated formulations of such agents. Such water-soluble, water-suspendable, or emulsifiable formulations are either solids, usually known as wettable powders, or water dispersible granules, or liquids usually known as emulsifiable concentrates, or aqueous suspensions. Wettable powders, which may be compacted to form water dispersible granules, comprise an intimate mixture of the pesticide, a carrier, and surfactants. The carrier is usually selected from among the attapulgite clays, the montmorillonite clays, the diatomaceous earths, or the purified silicates. Effective surfactants, including from about 0.5% to about 10% of the wettable powder, are found among sulfonated lignins, condensed naphthalenesulfonates, naphthalenesulfonates, alkylbenzenesulfonates, alkyl sulfates, and non-ionic surfactants such as ethylene oxide adducts of alkyl phenols.

Emulsifiable concentrates can comprise a suitable concentration of PMPs, such as from about 50 to about 500 grams per liter of liquid dissolved in a carrier that is either a water miscible solvent or a mixture of water-immiscible organic solvent and emulsifiers. Useful organic solvents include aromatics, especially xylenes and petroleum fractions, especially the high-boiling naphthalenic and olefinic portions of petroleum such as heavy aromatic naphtha. Other organic solvents may also be used, such as the terpenic solvents including rosin derivatives, aliphatic ketones such as cyclohexanone, and complex alcohols such as 2-ethoxyethanol. Suitable emulsifiers for emulsifiable concentrates are selected from conventional anionic and non-ionic surfactants.

Aqueous suspensions comprise suspensions of water-insoluble pesticides dispersed in an aqueous carrier at a concentration in the range from about 5% to about 50% by weight. Suspensions are prepared by finely grinding the pesticide and vigorously mixing it into a carrier comprised of water and surfactants. Ingredients, such as inorganic salts and synthetic or natural gums may also be added, to increase the density and viscosity of the aqueous carrier.

PMPs may also be applied as granular compositions that are particularly useful for applications to the soil. Granular compositions usually contain from about 0.5% to about 10% by weight of the pesticide, dispersed in a carrier that comprises clay or a similar substance. Such compositions are usually prepared by dissolving the formulation in a suitable solvent and applying it to a granular carrier which has been pre-formed to the appropriate particle size, in the range of from about 0.5 to about 3 mm. Such compositions may also be formulated by making a dough or paste of the carrier and compound and crushing and drying to obtain the desired granular particle size.

Dusts containing the present PMP formulation are prepared by intimately mixing PMPs in powdered form with a suitable dusty agricultural carrier, such as kaolin clay, ground volcanic rock, and the like. Dusts can suitably contain from about 1% to about 10% of the packets. They can be applied as a seed dressing or as a foliage application with a dust blower machine.

It is equally practical to apply the present formulation in the form of a solution in an appropriate organic solvent, usually petroleum oil, such as the spray oils, which are widely used in agricultural chemistry.

PMPs can also be applied in the form of an aerosol composition. In such compositions the packets are dissolved or dispersed in a carrier, which is a pressure-generating propellant mixture. The aerosol composition is packaged in a container from which the mixture is dispensed through an atomizing valve.

Another embodiment is an oil-in-water emulsion, wherein the emulsion comprises oily globules which are each provided with a lamellar liquid crystal coating and are dispersed in an aqueous phase, wherein each oily globule comprises at least one compound which is agriculturally active, and is individually coated with a monolamellar or oligolamellar layer including: (1) at least one non-ionic lipophilic surface-active agent, (2) at least one non-ionic hydrophilic surface-active agent and (3) at least one ionic surface-active agent, wherein the globules having a mean particle diameter of less than 800 nanometers. Further information on the embodiment is disclosed in U.S. patent publication 20070027034 published Feb. 1, 2007. For ease of use, this embodiment will be referred to as “OIWE.” Additionally, generally, when the molecules disclosed above are used in a formulation, such formulation can also contain other components. These components include, but are not limited to, (this is a non-exhaustive and non-mutually exclusive list) wetters, spreaders, stickers, penetrants, buffers, sequestering agents, drift reduction agents, compatibility agents, anti-foam agents, cleaning agents, and emulsifiers. A few components are described forthwith.

A wetting agent is a substance that when added to a liquid increases the spreading or penetration power of the liquid by reducing the interfacial tension between the liquid and the surface on which it is spreading. Wetting agents are used for two main functions in agrochemical formulations: during processing and manufacture to increase the rate of wetting of powders in water to make concentrates for soluble liquids or suspension concentrates; and during mixing of a product with water in a spray tank to reduce the wetting time of wettable powders and to improve the penetration of water into water-dispersible granules. Examples of wetting agents used in wettable powder, suspension concentrate, and water-dispersible granule formulations are: sodium lauryl sulfate; sodium dioctyl sulfosuccinate; alkyl phenol ethoxylates; and aliphatic alcohol ethoxylates.

A dispersing agent is a substance which adsorbs onto the surface of particles and helps to preserve the state of dispersion of the particles and prevents them from reaggregating. Dispersing agents are added to agrochemical formulations to facilitate dispersion and suspension during manufacture, and to ensure the particles redisperse into water in a spray tank. They are widely used in wettable powders, suspension concentrates and water-dispersible granules. Surfactants that are used as dispersing agents have the ability to adsorb strongly onto a particle surface and provide a charged or steric barrier to reaggregation of particles. The most commonly used surfactants are anionic, non-ionic, or mixtures of the two types. For wettable powder formulations, the most common dispersing agents are sodium lignosulfonates. For suspension concentrates, very good adsorption and stabilization are obtained using polyelectrolytes, such as sodium naphthalene sulfonate formaldehyde condensates.

Tristyrylphenol ethoxylate phosphate esters are also used. Non-ionics such as alkylarylethylene oxide condensates and EO-PO block copolymers are sometimes combined with anionics as dispersing agents for suspension concentrates. In recent years, new types of very high molecular weight polymeric surfactants have been developed as dispersing agents. These have very long hydrophobic ‘backbones’ and a large number of ethylene oxide chains forming the ‘teeth’ of a ‘comb’ surfactant. These high molecular weight polymers can give very good long-term stability to suspension concentrates because the hydrophobic backbones have many anchoring points onto the particle surfaces. Examples of dispersing agents used in agrochemical formulations are: sodium lignosulfonates; sodium naphthalene sulfonate formaldehyde condensates; tristyrylphenol ethoxylate phosphate esters; aliphatic alcohol ethoxylates; alkyl ethoxylates; EO-PO (ethylene oxide-propylene oxide) block copolymers; and graft copolymers.

An emulsifying agent is a substance which stabilizes a suspension of droplets of one liquid phase in another liquid phase. Without the emulsifying agent the two liquids would separate into two immiscible liquid phases. The most commonly used emulsifier blends contain alkylphenol or aliphatic alcohol with twelve or more ethylene oxide units and the oil-soluble calcium salt of dodecylbenzenesulfonic acid. A range of hydrophile-lipophile balance (“HLB”) values from 8 to 18 will normally provide good stable emulsions. Emulsion stability can sometimes be improved by the addition of a small amount of an EO-PO block copolymer surfactant.

A solubilizing agent is a surfactant which will form micelles in water at concentrations above the critical micelle concentration. The micelles are then able to dissolve or solubilize water-insoluble materials inside the hydrophobic part of the micelle. The types of surfactants usually used for solubilization are non-ionics, sorbitan monooleates, sorbitan monooleate ethoxylates, and methyl oleate esters.

Surfactants are sometimes used, either alone or with other additives such as mineral or vegetable oils as adjuvants to spray-tank mixes to improve the biological performance of the pesticide on the target. The types of surfactants used for bioenhancement depend generally on the nature and mode of action of the pesticide. However, they are often non-ionics such as: alkyl ethoxylates; linear aliphatic alcohol ethoxylates; aliphatic amine ethoxylates.

A carrier or diluent in an agricultural formulation is a material added to the pesticide to give a product of the required strength. Carriers are usually materials with high absorptive capacities, while diluents are usually materials with low absorptive capacities. Carriers and diluents are used in the formulation of dusts, wettable powders, granules, and water-dispersible granules.

Organic solvents are used mainly in the formulation of emulsifiable concentrates, oil-in-water emulsions, suspoemulsions, and ultra low volume formulations, and to a lesser extent, granular formulations. Sometimes mixtures of solvents are used. The first main groups of solvents are aliphatic paraffinic oils such as kerosene or refined paraffins. The second main group (and the most common) comprises the aromatic solvents such as xylene and higher molecular weight fractions of C9 and C10 aromatic solvents. Chlorinated hydrocarbons are useful as cosolvents to prevent crystallization of pesticides when the formulation is emulsified into water. Alcohols are sometimes used as cosolvents to increase solvent power. Other solvents may include vegetable oils, seed oils, and esters of vegetable and seed oils.

Thickeners or gelling agents are used mainly in the formulation of suspension concentrates, emulsions, and suspoemulsions to modify the rheology or flow properties of the liquid and to prevent separation and settling of the dispersed particles or droplets. Thickening, gelling, and anti-settling agents generally fall into two categories, namely water-insoluble particulates and water-soluble polymers. It is possible to produce suspension concentrate formulations using clays and silicas. Examples of these types of materials, include, but are not limited to, montmorillonite, bentonite, magnesium aluminum silicate, and attapulgite. Water-soluble polysaccharides have been used as thickening-gelling agents for many years. The types of polysaccharides most commonly used are natural extracts of seeds and seaweeds or are synthetic derivatives of cellulose. Examples of these types of materials include, but are not limited to, guar gum; locust bean gum; carrageenam; alginates; methyl cellulose; sodium carboxymethyl cellulose (SCMC); hydroxyethyl cellulose (HEC). Other types of anti-settling agents are based on modified starches, polyacrylates, polyvinyl alcohol, and polyethylene oxide. Another good anti-settling agent is xanthan gum.

Microorganisms can cause spoilage of formulated products. Therefore preservation agents are used to eliminate or reduce their effect. Examples of such agents include, but are not limited to: propionic acid and its sodium salt; sorbic acid and its sodium or potassium salts; benzoic acid and its sodium salt; p-hydroxybenzoic acid sodium salt; methyl p-hydroxybenzoate; and 1,2-benzisothiazolin-3-one (BIT).

The presence of surfactants often causes water-based formulations to foam during mixing operations in production and in application through a spray tank. In order to reduce the tendency to foam, anti-foam agents are often added either during the production stage or before filling into bottles. Generally, there are two types of anti-foam agents, namely silicones and non-silicones. Silicones are usually aqueous emulsions of dimethyl polysiloxane, while the non-silicone anti-foam agents are water-insoluble oils, such as octanol and nonanol, or silica. In both cases, the function of the anti-foam agent is to displace the surfactant from the air-water interface.

“Green” agents (e.g., adjuvants, surfactants, solvents) can reduce the overall environmental footprint of crop protection formulations. Green agents are biodegradable and generally derived from natural and/or sustainable sources, e.g., plant and animal sources. Specific examples are: vegetable oils, seed oils, and esters thereof, also alkoxylated alkyl polyglucosides.

In some instances, PMPs can be freeze-dried or lyophilized. See U.S. Pat. No. 4,311,712. The PMPs can later be reconstituted on contact with water or another liquid. Other components can be added to the lyophilized or reconstituted liposomes, for example, other pesticidal agents, agriculturally acceptable carriers, or other materials in accordance with the formulations described herein.

Other optional features of the composition include carriers or delivery vehicles that protect the pest control (e.g., biopesticide or biorepellent) composition against UV and/or acidic conditions. In some instances, the delivery vehicle contains a pH buffer. In some instances, the composition is formulated to have a pH in the range of about 4.5 to about 9.0, including for example pH ranges of about any one of 5.0 to about 8.0, about 6.5 to about 7.5, or about 6.5 to about 7.0.

The composition may additionally be formulated with an attractant (e.g., a chemoattractant) that attracts a pest to the vicinity of the composition. Attractants include pheromones, a chemical that is secreted by an animal, especially a pest, or chemoattractants which influences the behavior or development of others of the same species. Other attractants include sugar and protein hydrolysate syrups, yeasts, and rotting meat. Attractants also can be combined with an active ingredient and sprayed onto foliage or other items in the treatment area. Various attractants are known which influence a pest's behavior as a pest's search for food, oviposition, or mating sites, or mates. Attractants useful in the methods and compositions described herein include, for example, eugenol, phenethyl propionate, ethyl dimethylisobutyl-cyclopropane carboxylate, propyl benszodioxancarboxylate, cis-7,8-epoxy-2-methyloctadecane, trans-8, trans-0-dodecadienol, cis-9-tetradecenal (with cis-11-hexadecenal), trans-11-tetradecenal, cis-11-hexadecenal, (Z)-11,12-hexadecadienal, cis-7-dodecenyl acetate, cis-8-dodecenyul acetate, cis-9-dodecenyl acetate, cis-9-tetradecenyl acetate, cis-11-tetradecenyl acetate, trans-11-tetradecenyl acetate (with cis-11), cis-9,trans-11-tetradecadienyl acetate (with cis-9,trans-12), cis-9,trans-1 2-tetradecadienyl acetate, cis-7,cis-11-hexadecadienyl acetate (with cis-7,trans-11), cis-3,cis-13-octadecadienyl acetate, trans-3,cis-13-octadecadienyl acetate, anethole and isoamyl salicylate.

For further information on agricultural formulations, see “Chemistry and Technology of Agrochemical Formulations” edited by D. A. Knowles, copyright 1998 by Kluwer Academic Publishers. Also see “Insecticides in Agriculture and Environment-Retrospects and Prospects” by A. S. Perry, I. Yamamoto, I. Ishaaya, and R. Perry, copyright 1998 by Springer-Verlag.

### II. Agricultural Methods

The pest control (e.g., biopesticide or biorepellent) compositions described herein are useful in a variety of agricultural methods, particularly for the prevention or reduction of infestations by plants pests.

The present methods involve delivering the pest control (e.g., biopesticide or biorepellent) compositions described herein to a plant or a plant pest, such as those described included herein. The compositions and related methods can be used to prevent infestation by or reduce the numbers of plant pests on plants, plant parts (e.g., roots, fruits and seeds), in or on soil, or on another plant medium. Accordingly, the compositions and methods can reduce the damaging effect of plant pests on a plant by, for example, killing, injuring, or slowing the activity of the pest, and can thereby increase the fitness of a plant. Plant pests include, for example, insects, nematodes, mollusks, bacteria, fungi, oomycetes, protozoa, and weeds (see section on “Plant Pests”). Compositions of the invention can be used to control, kill, injure, paralyze, or reduce the activity of one or more of any of these pests in any developmental stage, e.g., their egg, nymph, instar, larvae, adult, juvenile, or desiccated forms. The methods may further be useful in controlling weeds. The details of each of these methods are described further below.

A. Delivery to a Plant

Provided herein are methods of delivering to a plant a pest control (e.g., biopesticide or biorepellent) composition disclosed herein. Included are methods for delivering a pest control (e.g., biopesticide or biorepellent) composition to a plant by contacting the plant, or part thereof, with a pest control (e.g., biopesticide or biorepellent) composition. The methods can be useful for increasing the fitness of a plant, e.g., by treating or preventing a plant pest infestation.

As such, the methods can be used to increase the fitness of a plant. In one aspect, provided herein is a method of increasing the fitness of a plant, the method including delivering to the plant the pest control (e.g., biopesticide or biorepellent) composition described herein (e.g., in an effective amount and duration) to increase the fitness of the plant relative to an untreated plant (e.g., a plant that has not been delivered the pest control (e.g., biopesticide or biorepellent) composition).

An increase in the fitness of the plant as a consequence of delivery of a pest control (e.g., biopesticide or biorepellent) composition can manifest in a number of ways, e.g., thereby resulting in a better production of the plant, for example, an improved yield, improved vigor of the plant or quality of the harvested product from the plant. An improved yield of a plant relates to an increase in the yield of a product (e.g., as measured by plant biomass, grain, seed or fruit yield, protein content, carbohydrate or oil content or leaf area) of the plant by a measurable amount over the yield of the same product of the plant produced under the same conditions, but without the application of the instant compositions or compared with application of conventional pesticides. For example, yield can be increased by at least about 0.5%, about 1%, about 2%, about 3%, about 4%, about 5%, about 10%, about 20%, about 30%, about 40%, about 50%, about 60%, about 70%, about 80%, about 90%, about 100%, or more than 100%. Yield can be expressed in terms of an amount by weight or volume of the plant or a product of the plant on some basis. The basis can be expressed in terms of time, growing area, weight of plants produced, or amount of a raw material used. For example, such methods may increase the yield of plant tissues including, but not limited to: seeds, fruits, kernels, bolls, tubers, roots, and leaves.

An increase in the fitness of a plant as a consequence of delivery of a pest control (e.g., biopesticide or biorepellent) composition can also be measured by other means, such as an increase or improvement of the vigor rating, the stand (the number of plants per unit of area), plant height, stalk circumference, stalk length, leaf number, leaf size, plant canopy, visual appearance (such as greener leaf color), root rating, emergence, protein content, increased tillering, bigger leaves, more leaves, less dead basal leaves, stronger tillers, less fertilizer needed, less seeds needed, more productive tillers, earlier flowering, early grain or seed maturity, less plant verse (lodging), increased shoot growth, earlier germination, or any combination of these factors, by a measurable or noticeable amount over the same factor of the plant produced under the same conditions, but without the administration of the instant compositions or with application of conventional pesticides.

i. Pest Treatment

Included herein is a method of decreasing a pest infestation in a plant having an infestation, wherein the method includes delivering the pest control (e.g., biopesticide or biorepellent) composition to the plant (e.g., in an effective amount and for an effective duration) to decrease the infestation relative to the infestation in an untreated plant. For example, the method may be effective to decrease the infestation by about 1%, 2%, 5%, 10%, 20%, 30%, 40%, 50%, 60%, 70%, 80%, 90%, 95%, 99%, 100%, or more than 100% relative to an untreated plant. In some instances, the method is effective to decrease the infestation by about 2×-fold, 5×-fold, 10×-fold, 25×-fold, 50×-fold, 75×-fold, 100×-fold, or more than 100×-fold relative to an untreated plant. In some instances, the method substantially eliminates the infestation relative to the infestation in an untreated plant. Alternatively, the method may slow progression of a plant infestation or decrease the severity of symptoms associated with a plant infestation. The composition may be sufficient to reduce (e.g., kill or repel) the pest, e.g., at least 5%, 10%, 20%, 30%, 40%, 50%, 60%, 70%, 80% or more, compared to a control.

The pest control (e.g., biopesticide or biorepellent) compositions described herein may be useful to promote the growth of plants. For example, by reducing the fitness of harmful pests, the pest control (e.g., biopesticide or biorepellent) compositions provided herein may be effective to promote the growth of plants that are typically harmed by a pest. This may or may not involve direct application of the pest control (e.g., biopesticide or biorepellent) composition to the plant. For example, in instances where the primary pest habitat is different than the region of plant growth, the pest control (e.g., biopesticide or biorepellent) composition may be applied to either the primary pest habitat, the plants of interest, or a combination of both.

In some instances, the plant may be an agricultural food crop, such as a cereal, grain, legume, fruit, or vegetable crop, or a non-food crop, e.g., grasses, flowering plants, cotton, hay, hemp. The compositions described herein may be delivered to the crop any time prior to or after harvesting the cereal, grain, legume, fruit, vegetable, or other crop. Crop yield is a measurement often used for crop plants and is normally measured in metric tons per hectare (or kilograms per hectare). Crop yield can also refer to the actual seed generation from the plant. In some instances, the pest control (e.g., biopesticide or biorepellent) composition may be effective to increase crop yield (e.g., increase metric tons of cereal, grain, legume, fruit, or vegetable per hectare and/or increase seed generation) by about 2%, 5%, 10%, 20%, 30%, 40%, 50%, 60%, 70%, 80%, 90%, 100%, or more in comparison to a reference level (e.g., a crop to which the pest control (e.g., biopesticide or biorepellent) composition has not been administered).

A decrease in infestation refers to a decrease in the number of pests on or around the plant or a decrease in symptoms or signs in the plant that are directly or indirectly caused by the pest. The degree of infestation may be measured in the plant at any time after treatment and compared to symptoms at or before the time of treatment. The plant may or may not be showing symptoms of the infestation. For example, the plant may be infested with a pest yet not showing signs of the infestation, e.g., a hypersensitive response (HR). An infested plant can be identified through observation of disease symptoms on the plant. The disease symptoms expressed will depend on the disease, but in general the symptoms include lesions, pustules, necrosis, hypersensitive responses, wilt, chlorosis, induction of defense related genes (e.g. SAR genes) and the like.

The skilled artisan will recognize that methods for determining plant infestation and disease by a plant pest depends on the pest and plant being tested. Infestation or associated symptoms can be identified by any means of identifying infestation or related symptoms. Various methods are available to identify infested plants and the associated symptoms. In one aspect, the methods may involve macroscopic or microscopic screening for infection and/or symptoms, quantitative PCR, or the use of microarrays for detection of infection related genes (e.g. Systemic Acquired Resistance genes, defensin genes, and the like). Macroscopic and microscopic methods for determining infestation in a plant are known in the art and include the identification of damage on plant tissue caused by infestation or by the presence of lesions, necrosis, spores, hyphae, growth of fungal mycelium, wilts, blights, spots on fruits, rots, galls and stunts, or the like. Such symptoms can be compared to non-infested plants, photos, or illustrations of infected plants or combinations thereof to determine the presence of an infection or the identity of the pathogen or both. Photos and illustrations of the symptoms of pathogen infection are widely available in the art and are available for example, from the American Phytopathological Society, St. Paul, Minn. 55121-2097. In some instances, the symptoms are visible to the naked eye or by a specified magnification (e.g., 2×, 3×, 4×, 5×, 10×, or 50×).

In some instances, the infestation or associated symptom can be identified using commercially available test kits to identify pests in plants. Such test kits are available, for example, from local agricultural extensions or cooperatives. In some instances, identifying a crop plant in need of treatment is by prediction of weather and environmental conditions conducive for disease development. In some instances, persons skilled in scouting fields of crop plants for plant disease identify a crop in need of treatment.

In some instance, an infection or associated symptom can be identified using Polymerase chain reaction (PCR)-based diagnostic assays. PCR-based assays can be used to perform PCR amplification of DNA or RNA sequences specific to the pest, including chromosomal DNA, mitochondrial DNA, or ribosomal RNA. The specific methods of identification will depend on the pathogen.

The plant can be pre-determined to have a pest infestation. Alternatively, the method may also include identifying plants having an infestation. As such, also provided are methods of treating a plant pest infestation by identifying a plant infested by a plant pest (i.e. post-infestation) and contacting the infected plant with an effective amount of a pest control (e.g., biopesticide or biorepellent) composition such that the infestation is treated. Infestation can be measured by any reproducible means of measurement. For example, infestation can be measured by counting the number of lesions on the plant visible to the naked eye, or at a specified magnification (e.g., 2×, 3×, 4×, 5×, 10×, or 50×). In other instances, infestation can be measured by measuring the concentration of pests over a provided area of the plant or an area surrounding the plant.

ii. Pest Prevention

Included herein is a method of preventing a plant infestation in a plant (e.g., a plant at risk of infestation), wherein the method includes delivering the pest control (e.g., biopesticide or biorepellent) composition to the plant (e.g., in an effective amount and duration) to decrease the likelihood of infestation relative to the likelihood of infestation in an untreated plant. For example, the method can decrease the likelihood of infestation by about 1%, 2%, 5%, 10%, 20%, 30%, 40%, 50%, 60%, 70%, 80%, 90%, 95%, 99%, 100%, or more than 100% relative to an untreated plant. In some instances, the method can decrease the likelihood of infestation by about 2×-fold, 5×-fold, 10×-fold, 25×-fold, 50×-fold, 75×-fold, 100×-fold, or more than 100×-fold relative to an untreated plant. The pests may be prevented or reduced from causing disease, the associated disease symptoms, or both.

The methods and compositions described herein may be used to reduce or prevent pest infestation in plants at risk of developing an infestation by reducing the fitness of pests that infest the plants. In some instances, the pest control (e.g., biopesticide or biorepellent) composition may be effective to reduce infestation (e.g., reduce the number of plants infested, reduce the pest population size, reduce damage to plants) by about 2%, 5%, 10%, 20%, 30%, 40%, 50%, 60%, 70%, 80%, 90%, 100%, or more in comparison to a reference level (e.g., a crop to which the pest control (e.g., biopesticide or biorepellent) composition has not been administered). In other instances, the pest control (e.g., biopesticide or biorepellent) composition may be effective to prevent or reduce the likelihood of crop infestation by about 2%, 5%, 10%, 20%, 30%, 40%, 50%, 60%, 70%, 80%, 90%, 100%, or more in comparison to a reference level (e.g., a crop to which the pest control (e.g., biopesticide or biorepellent) composition has not been administered).

These preventive methods can be useful to prevent infestation in a plant at risk of being infested by a plant pest. For example, the plant may be one that has not been exposed to a plant pest, but the plant may be at risk of infection in circumstances where pests are more likely to infest the plant, for example, in pest optimal climate conditions. Plant risk may be further increased in instances where the plant is located in a habitat where weeds in the habitat have been treated with an herbicide and disease crossover from the dying plant to the standing plant is possible. In some instances, identifying a crop plant in need of treatment is by prediction of weather and environmental conditions conducive for disease development.

The methods may prevent infestation for a period of time after treatment with the pest control (e.g., biopesticide or biorepellent) composition. For example, the method may prevent infestation of the plant for several weeks after application of the pest control (e.g., biopesticide or biorepellent) composition. For instance, the disease may be prevented for at least about 14, 15, 16, 17, 18, 19, 20, 21, 22, 23, 24, 25, 26, 27, 28, 29, 30, 35 days after treatment with a pest control (e.g., biopesticide or biorepellent) composition. In some instances, the disease is prevented for at least about 40 days after delivery of a pest control (e.g., biopesticide or biorepellent) composition to the plant. Prevention of disease may be measured by any reproducible means of measurement. In certain instances, infestation is assessed 7, 8, 9, 10, 11, 12, 13, 14, 15, 16, 17, 18, 19, 20, 25, 30 days after delivery of the pest control (e.g., biopesticide or biorepellent) composition.

B. Delivery to a Plant Pest

Provided herein are methods of delivering to a plant pest a pest control (e.g., biopesticide or biorepellent) composition disclosed herein. Included are methods for delivering a pest control (e.g., biopesticide or biorepellent) composition to a pest by contacting the pest with a pest control (e.g., biopesticide or biorepellent) composition. The methods can be useful for decreasing the fitness of a pest, e.g., to prevent or treat a pest infestation as a consequence of delivery of a pest control (e.g., biopesticide or biorepellent) composition.

As such, the methods can be used to decrease the fitness of a pest. In one aspect, provided herein is a method of decreasing the fitness of a pest, the method including delivering to the pest the pest control (e.g., biopesticide or biorepellent) composition described herein (e.g., in an effective amount and for an effective duration) to decrease the fitness of the pest relative to an untreated pest (e.g., a pest that has not been delivered the pest control (e.g., biopesticide or biorepellent) composition).

In one aspect, provided herein is a method of decreasing a fungal infection in (e.g., treating) a plant having a fungal infection, wherein the method includes delivering to the plant a pest control (e.g., biopesticide or biorepellent) composition including a plurality of PMPs (e.g., any of the pest control (e.g., biopesticide or biorepellent) compositions described herein).

In another aspect, provided herein is a method of decreasing a fungal infection in (e.g., treating) a plant having a fungal infection, wherein the method includes delivering to the plant a pest control (e.g., biopesticide or biorepellent) composition including a plurality of PMPs (e.g., any of the pest control (e.g., biopesticide or biorepellent) compositions described herein), and wherein the plurality of PMPs include an antifungal agent. In some instances, the antifungal agent is a nucleic acid that inhibits expression of a gene (e.g., dcl1 and dcl2 (i.e., dcl12) in a fungus that causes the fungal infection. In some instances, the fungal infection is caused be a fungus belonging to a Sclerotinia spp. (e.g., Sclerotinia sclerotiorum), a Botrytis spp. (e.g., Botrytis cinerea), an Aspergillus spp., a Fusarium spp., or a Penicillium spp. In some instances, the composition includes a PMP produced from an Arabidopsis apoplast EV. In some instances, the method decreases or substantially eliminates the fungal infection.

In another aspect, provided herein is a method of decreasing a bacterial infection in (e.g., treating) a plant having a bacterial infection, wherein the method includes delivering to the plant a pest control (e.g., biopesticide or biorepellent) composition including a plurality of PMPs (e.g., any of the pest control (e.g., biopesticide or biorepellent) compositions described herein).

In another aspect, provided herein is a method of decreasing a bacterial infection in (e.g., treating) a plant having a bacterial infection, wherein the method includes delivering to the plant a pest control (e.g., biopesticide or biorepellent) composition including a plurality of PMPs, and wherein the plurality of PMPs include an antibacterial agent. In some instances, the antibacterial agent is streptomycin. In some instances, the bacterial infection is caused by a bacterium belonging to a Pseudomonas spp (e.g., Pseudomonas syringae or Pseudomonas aeruginosa). In some instances, the composition includes a PMP produced from an Arabidopsis apoplast EV. In some instances, the method decreases or substantially eliminates the bacterial infection. In some instances, the antibacterial agent is doxorubicin or vancomycin.

In another aspect, provided herein is a method of decreasing the fitness of an insect plant pest, wherein the method includes delivering to the insect plant pest a pest control (e.g., biopesticide or biorepellent) composition including a plurality of PMPs (e.g., any of the pest control (e.g., biopesticide or biorepellent) compositions described herein).

In another aspect, provided herein is a method of decreasing the fitness of an insect plant pest, wherein the method includes delivering to the insect plant pest a pest control (e.g., biopesticide or biorepellent) composition including a plurality of PMPs (e.g., any of the pest control (e.g., biopesticide or biorepellent) compositions described herein), and wherein the plurality of PMPs includes an insecticidal agent. In some instances, the insecticidal agent is a peptide nucleic acid. In some instances, the insect plant pest is an aphid. In some instances, the insect plant pest is a lepidopteran (e.g., Spodoptera frugiperda). In some instances, the method decreases the fitness of the insect plant pest relative to an untreated insect plant pest. In another aspect, provided herein is a method of decreasing the fitness of a nematode plant pest, wherein the method includes delivering to the nematode plant pest a pest control (e.g., biopesticide or biorepellent) composition including a plurality of PMPs (e.g., any of the pest control (e.g., biopesticide or biorepellent) compositions described herein).

In another aspect, provided herein is a method of decreasing the fitness of a nematode plant pest, wherein the method includes delivering to the nematode plant pest a pest control (e.g., biopesticide or biorepellent) composition including a plurality of PMPs (e.g., any of the pest control (e.g., biopesticide or biorepellent) compositions described herein), and wherein the plurality of PMPs include a nematicidal agent. In some instances, the nematicidal agent is a neuropeptide (e.g., Mi-NLP-15b). In some instances, the nematode plant pest is a corn root-knot nematode. In some instances, the method decreases the fitness of the nematode plant pest relative to an untreated nematode plant pest.

In another aspect, provided herein is a method of decreasing the fitness of a weed, wherein the method includes delivering to the weed a pest control (e.g., biopesticide or biorepellent) composition including a plurality of PMPs (e.g., any of the pest control (e.g., biopesticide or biorepellent) compositions described herein).

In another aspect, provided herein is a method of decreasing the fitness of a weed, wherein the method includes delivering to the weed a pest control (e.g., biopesticide or biorepellent) composition including a plurality of PMPs (e.g., any of the pest control (e.g., biopesticide or biorepellent) compositions described herein), and wherein the plurality of PMPs include an herbicidal agent (e.g. doxorubicin or glufosinate). In some instances, the weed is an Indian goosegrass (Eleusine indica). In some instances, the method decreases the fitness of the weed relative to an untreated weed.

A decrease in the fitness of the pest as a consequence of delivery of a pest control (e.g., biopesticide or biorepellent) composition can manifest in a number of ways. In some instances, the decrease in fitness of the pest may manifest as a deterioration or decline in the physiology of the pest (e.g., reduced health or survival) as a consequence of delivery of the pest control (e.g., biopesticide or biorepellent) composition. In some instances, the fitness of an organism may be measured by one or more parameters, including, but not limited to, reproductive rate, fertility, lifespan, viability, mobility, fecundity, pest development, body weight, metabolic rate or activity, or survival in comparison to a pest to which the pest control (e.g., biopesticide or biorepellent) composition has not been administered. For example, the methods or compositions provided herein may be effective to decrease the overall health of the pest or to decrease the overall survival of the pest. In some instances, the decreased survival of the pest is about 2%, 5%, 10%, 20%, 30%, 40%, 50%, 60%, 70%, 80%, 90%, 100%, or greater than 100% greater relative to a reference level (e.g., a level found in a pest that does not receive a pest control (e.g., biopesticide or biorepellent) composition). In some instances, the methods and compositions are effective to decrease pest reproduction (e.g., reproductive rate, fertility) in comparison to a pest to which the pest control (e.g., biopesticide or biorepellent) composition has not been administered. In some instances, the methods and compositions are effective to decrease other physiological parameters, such as mobility, body weight, life span, fecundity, or metabolic rate, by about 2%, 5%, 10%, 20%, 30%, 40%, 50%, 60%, 70%, 80%, 90%, 100%, or greater than 100% relative to a reference level (e.g., a level found in a pest that does not receive a pest control (e.g., biopesticide or biorepellent) composition).

In some instances, the decrease in pest fitness may manifest as a decrease in the production of one or more nutrients in the pest (e.g., vitamins, carbohydrates, amino acids, or polypeptides) in comparison to a pest to which the pest control (e.g., biopesticide or biorepellent) composition has not been administered. In some instances, the methods or compositions provided herein may be effective to decrease the production of nutrients in the pest (e.g., vitamins, carbohydrates, amino acids, or polypeptides) by about 2%, 5%, 10%, 20%, 30%, 40%, 50%, 60%, 70%, 80%, 90%, 100%, or greater than 100% relative to a reference level (e.g., a level found in a pest that does not receive a pest control (e.g., biopesticide or biorepellent) composition).

In some instances, the decrease in pest fitness may manifest as an increase in the pest's sensitivity to a pesticidal agent and/or a decrease in the pest's resistance to a pesticidal agent in comparison to a pest to which the pest control (e.g., biopesticide or biorepellent) composition has not been administered. In some instances, the methods or compositions provided herein may be effective to increase the pest's sensitivity to a pesticidal agent by about 2%, 5%, 10%, 20%, 30%, 40%, 50%, 60%, 70%, 80%, 90%, 100%, or greater than 100% relative to a reference level (e.g., a level found in a pest that does not receive a pest control (e.g., biopesticide or biorepellent) composition). The pesticidal agent may be any pesticidal agent known in the art, including insecticidal agents. In some instances, the methods or compositions provided herein may increase the pest's sensitivity to a pesticidal agent by decreasing the pest's ability to metabolize or degrade the pesticidal agent into usable substrates in comparison to a pest to which the pest control (e.g., biopesticide or biorepellent) composition has not been administered.

In some instances, the decrease in pest fitness may manifest as an increase in the pest's sensitivity to an allelochemical agent and/or a decrease in the pest's resistance to an allelochemical agent in comparison to a pest to which the pest control (e.g., biopesticide or biorepellent) composition has not been administered. In some instances, the methods or compositions provided herein may be effective to decrease the pest's resistance to an allelochemical agent by about 2%, 5%, 10%, 20%, 30%, 40%, 50%, 60%, 70%, 80%, 90%, 100%, or greater than 100% relative to a reference level (e.g., a level found in a pest that does not receive a pest control (e.g., biopesticide or biorepellent) composition). In some instances, the allelochemical agent is caffeine, soyacystatin, fenitrothion, monoterpenes, diterpene acids, or phenolic compounds (e.g., tannins, flavonoids). In some instances, the methods or compositions provided herein may increase the pest's sensitivity to an allelochemical agent by decreasing the pest's ability to metabolize or degrade the allelochemical agent into usable substrates in comparison to a pest to which the pest control (e.g., biopesticide or biorepellent) composition has not been administered.

In some instances, the methods or compositions provided herein may be effective to decease the pest's resistance to parasites or pathogens (e.g., fungal, bacterial, or viral pathogens or parasites) in comparison to a pest to which the pest control (e.g., biopesticide or biorepellent) composition has not been administered. In some instances, the methods or compositions provided herein may be effective to decrease the pest's resistance to a pathogen or parasite (e.g., fungal, bacterial, or viral pathogens; or parasitic mites) by about 2%, 5%, 10%, 20%, 30%, 40%, 50%, 60%, 70%, 80%, 90%, 100%, or greater than 100% relative to a reference level (e.g., a level found in a pest that does not receive a pest control (e.g., biopesticide or biorepellent) composition).

In some instances, the methods or compositions provided herein may be effective to decrease the pest's ability to carry or transmit a plant pathogen (e.g., plant virus (e.g., TYLCV) or a plant bacterium (e.g., Agrobacterium spp)) in comparison to a pest to which the pest control (e.g., biopesticide or biorepellent) composition has not been administered. For example, the methods or compositions provided herein may be effective to decrease the pest's ability to carry or transmit a plant pathogen (e.g., a plant virus (e.g., TYLCV) or plant bacterium (e.g., Agrobacterium spp)) by about 2%, 5%, 10%, 20%, 30%, 40%, 50%, 60%, 70%, 80%, 90%, 100%, or greater than 100% relative to a reference level (e.g., a level found in a pest that does not receive a pest control (e.g., biopesticide or biorepellent) composition).

Additionally or alternatively, in cases where an herbicide is included in the PMP, or compositions thereof, the methods may be further used to decrease the fitness of or kill weeds. In such instances, the method may be effective to decrease the fitness of the weed by about 2%, 5%, 10%, 20%, 30%, 40%, 50%, 60%, 70%, 80%, 90%, 100%, or more in comparison to an untreated weed (e.g., a weed to which the pest control (e.g., biopesticide or biorepellent) composition has not been administered). For example, the method may be effective to kill the weed, thereby decreasing a population of the weed by about 2%, 5%, 10%, 20%, 30%, 40%, 50%, 60%, 70%, 80%, 90%, 100%, or more in comparison to an untreated weed. In some instances, the method substantially eliminates the weed. Examples of weeds that can be treated in accordance with the present methods are further described herein.

In some instances, the decrease in pest fitness may manifest as other fitness disadvantages, such as a decreased tolerance to certain environmental factors (e.g., a high or low temperature tolerance), a decreased ability to survive in certain habitats, or a decreased ability to sustain a certain diet in comparison to a pest to which the pest control (e.g., biopesticide or biorepellent) composition has not been administered. In some instances, the methods or compositions provided herein may be effective to decrease pest fitness in any plurality of ways described herein. Further, the pest control (e.g., biopesticide or biorepellent) composition may decrease pest fitness in any number of pest classes, orders, families, genera, or species (e.g., 1 pest species, 2, 3, 4, 5, 6, 7, 8, 9, 10, 15, 20, 30, 40, 50, 60, 70, 80, 90, 100, 150, 200, 200, 250, 500, or more pest species). In some instances, the pest control (e.g., biopesticide or biorepellent) composition acts on a single pest class, order, family, genus, or species.

Pest fitness may be evaluated using any standard methods in the art. In some instances, pest fitness may be evaluated by assessing an individual pest. Alternatively, pest fitness may be evaluated by assessing a pest population. For example, a decrease in pest fitness may manifest as a decrease in successful competition against other insects, thereby leading to a decrease in the size of the pest population.

C. Application Methods

A pest described herein can be exposed to any of the compositions described herein in any suitable manner that permits delivering or administering the composition to the pest. The pest control (e.g., biopesticide or biorepellent) composition may be delivered either alone or in combination with other active (e.g., pesticidal agents) or inactive substances and may be applied by, for example, spraying, injection (e.g, microinjection), through plants, pouring, dipping, in the form of concentrated liquids, gels, solutions, suspensions, sprays, powders, pellets, briquettes, bricks and the like, formulated to deliver an effective concentration of the pest control (e.g., biopesticide or biorepellent) composition. Amounts and locations for application of the compositions described herein are generally determined by the habits of the pest, the lifecycle stage at which the pest can be targeted by the pest control (e.g., biopesticide or biorepellent) composition, the site where the application is to be made, and the physical and functional characteristics of the pest control (e.g., biopesticide or biorepellent) composition. The pest control (e.g., biopesticide or biorepellent) compositions described herein may be administered to the pest by oral ingestion, but may also be administered by means which permit penetration through the cuticle or penetration of the pest respiratory system.

In some instances, the pest can be simply “soaked” or “sprayed” with a solution including the pest control (e.g., biopesticide or biorepellent) composition. Alternatively, the pest control (e.g., biopesticide or biorepellent) composition can be linked to a food component (e.g., comestible) of the pest for ease of delivery and/or in order to increase uptake of the pest control (e.g., biopesticide or biorepellent) composition by the pest. Methods for oral introduction include, for example, directly mixing a pest control (e.g., biopesticide or biorepellent) composition with the pest's food, spraying the pest control (e.g., biopesticide or biorepellent) composition in the pest's habitat or field, as well as engineered approaches in which a species that is used as food is engineered to express a pest control (e.g., biopesticide or biorepellent) composition, then fed to the pest to be affected. In some instances, for example, the pest control (e.g., biopesticide or biorepellent) composition can be incorporated into, or overlaid on the top of, the pest's diet. For example, the pest control (e.g., biopesticide or biorepellent) composition can be sprayed onto a field of crops which a pest inhabits.

In some instances, the composition is sprayed directly onto a plant e.g., crops, by e.g., backpack spraying, aerial spraying, crop spraying/dusting etc. In instances where the pest control (e.g., biopesticide or biorepellent) composition is delivered to a plant, the plant receiving the pest control (e.g., biopesticide or biorepellent) composition may be at any stage of plant growth. For example, formulated pest control (e.g., biopesticide or biorepellent) compositions can be applied as a seed-coating or root treatment in early stages of plant growth or as a total plant treatment at later stages of the crop cycle. In some instances, the pest control (e.g., biopesticide or biorepellent) composition may be applied as a topical agent to a plant, such that the pest ingests or otherwise comes in contact with the plant upon interacting with the plant.

Further, the pest control (e.g., biopesticide or biorepellent) composition may be applied (e.g., in the soil in which a plant grows, or in the water that is used to water the plant) as a systemic agent that is absorbed and distributed through the tissues of a plant or animal pest, such that a pest feeding thereon will obtain an effective dose of the pest control (e.g., biopesticide or biorepellent) composition. In some instances, plants or food organisms may be genetically transformed to express the pest control (e.g., biopesticide or biorepellent) composition such that a pest feeding upon the plant or food organism will ingest the pest control (e.g., biopesticide or biorepellent) composition.

Delayed or continuous release can also be accomplished by coating the pest control (e.g., biopesticide or biorepellent) composition or a composition with the pest control (e.g., biopesticide or biorepellent) composition(s) with a dissolvable or bioerodable coating layer, such as gelatin, which coating dissolves or erodes in the environment of use, to then make the pest control (e.g., biopesticide or biorepellent) composition available, or by dispersing the agent in a dissolvable or erodable matrix. Such continuous release and/or dispensing means devices may be advantageously employed to consistently maintain an effective concentration of one or more of the pest control (e.g., biopesticide or biorepellent) compositions described herein in a specific pest habitat.

The pest control (e.g., biopesticide or biorepellent) composition can also be incorporated into the medium in which the pest grows, lives, reproduces, feeds, or infests. For example, a pest control (e.g., biopesticide or biorepellent) composition can be incorporated into a food container, feeding station, protective wrapping, or a hive. For some applications the pest control (e.g., biopesticide or biorepellent) composition may be bound to a solid support for application in powder form or in a trap or feeding station. As an example, for applications where the composition is to be used in a trap or as bait for a particular pest, the compositions may also be bound to a solid support or encapsulated in a time-release material. For example, the compositions described herein can be administered by delivering the composition to at least one habitat where an agricultural pest (e.g., aphid) grows, lives, reproduces, or feeds.

Pesticides are often recommended for field application as an amount of pesticide per hectare (g/ha or kg/ha) or the amount of active ingredient or acid equivalent per hectare (kg a.i./ha or g a.i./ha). In some instances, a lower amount of pesticide in the present compositions may be required to be applied to soil, plant media, seeds plant tissue, or plants to achieve the same results as where the pesticide is applied in a composition lacking PMPs. For example, the amount of pesticidal agent may be applied at levels about 2, 3, 4, 5, 6, 7, 8, 9, 10, 15, 20, 30, 50, or 100-fold (or any range between about 2 and about 100-fold, for example about 2- to 10-fold; about 5- to 15-fold, about 10- to 20-fold; about 10- to 50-fold) less than the same pesticidal agent applied in a non-PMP composition, e.g., direct application of the same pesticidal agent. Pest control (e.g., biopesticide or biorepellent) compositions of the invention can be applied at a variety of amounts per hectare, for example at about 0.0001, 0.001, 0.005, 0.01, 0.1, 1, 2, 10, 100, 1,000, 2,000, 5,000 (or any range between about 0.0001 and 5,000) kg/ha. For example, about 0.0001 to about 0.01, about 0.01 to about 10, about 10 to about 1,000, about 1,000 to about 5,000 kg/ha.

### III. Plants

A variety of plants can be delivered to or treated with a pest control (e.g., biopesticide or biorepellent) composition described herein. Plants that can be delivered a pest control (e.g., biopesticide or biorepellent) composition (i.e., “treated”) in accordance with the present methods include whole plants and parts thereof, including, but not limited to, shoot vegetative organs/structures (e.g., leaves, stems and tubers), roots, flowers and floral organs/structures (e.g., bracts, sepals, petals, stamens, carpels, anthers and ovules), seed (including embryo, endosperm, cotyledons, and seed coat) and fruit (the mature ovary), plant tissue (e.g., vascular tissue, ground tissue, and the like) and cells (e.g., guard cells, egg cells, and the like), and progeny of same. Plant parts can further refer parts of the plant such as the shoot, root, stem, seeds, stipules, leaves, petals, flowers, ovules, bracts, branches, petioles, internodes, bark, pubescence, tillers, rhizomes, fronds, blades, pollen, stamen, and the like.

The class of plants that can be treated in a method disclosed herein includes the class of higher and lower plants, including angiosperms (monocotyledonous and dicotyledonous plants), gymnosperms, ferns, horsetails, psilophytes, lycophytes, bryophytes, and algae (e.g., multicellular or unicellular algae). Plants that can be treated in accordance with the present methods further include any vascular plant, for example monocotyledons or dicotyledons or gymnosperms, including, but not limited to alfalfa, apple, Arabidopsis, banana, barley, canola, castor bean, chrysanthemum, clover, cocoa, coffee, cotton, cottonseed, corn, crambe, cranberry, cucumber, dendrobium, dioscorea, eucalyptus, fescue, flax, gladiolus, liliacea, linseed, millet, muskmelon, mustard, oat, oil palm, oilseed rape, papaya, peanut, pineapple, ornamental plants, Phaseolus, potato, rapeseed, rice, rye, ryegrass, safflower, sesame, sorghum, soybean, sugarbeet, sugarcane, sunflower, strawberry, tobacco, tomato, turfgrass, wheat and vegetable crops such as lettuce, celery, broccoli, cauliflower, cucurbits; fruit and nut trees, such as apple, pear, peach, orange, grapefruit, lemon, lime, almond, pecan, walnut, hazel; vines, such as grapes (e.g., a vineyard), kiwi, hops; fruit shrubs and brambles, such as raspberry, blackberry, gooseberry; forest trees, such as ash, pine, fir, maple, oak, chestnut, popular; with alfalfa, canola, castor bean, corn, cotton, crambe, flax, linseed, mustard, oil palm, oilseed rape, peanut, potato, rice, safflower, sesame, soybean, sugarbeet, sunflower, tobacco, tomato, and wheat. Plants that can be treated in accordance with the methods of the present invention include any crop plant, for example, forage crop, oilseed crop, grain crop, fruit crop, vegetable crop, fiber crop, spice crop, nut crop, turf crop, sugar crop, beverage crop, and forest crop. In certain instances, the crop plant that is treated in the method is a soybean plant. In other certain instances, the crop plant is wheat. In certain instances, the crop plant is corn. In certain instances, the crop plant is cotton. In certain instances, the crop plant is alfalfa. In certain instances, the crop plant is sugarbeet. In certain instances, the crop plant is rice. In certain instances, the crop plant is potato. In certain instances, the crop plant is tomato.

In certain instances, the plant is a crop. Examples of such crop plants include, but are not limited to, monocotyledonous and dicotyledonous plants including, but not limited to, fodder or forage legumes, ornamental plants, food crops, trees, or shrubs selected from Acer spp., Allium spp., Amaranthus spp., Ananas comosus, Apium graveolens, Arachis spp, Asparagus officinalis, Beta vulgaris, Brassica spp. (e.g., Brassica napus, Brassica rapa ssp. (canola, oilseed rape, turnip rape), Camellia sinensis, Canna indica, Cannabis saliva, Capsicum spp., Castanea spp., Cichorium endivia, Citrullus lanatus, Citrus spp., Cocos spp., Coffea spp., Coriandrum sativum, Corylus spp., Crataegus spp., Cucurbita spp., Cucumis spp., Daucus carota, Fagus spp., Ficus carica, Fragaria spp., Ginkgo biloba, Glycine spp. (e.g., Glycine max, Soja hispida or Soja max), Gossypium hirsutum, Helianthus spp. (e.g., Helianthus annuus), Hibiscus spp., Hordeum spp. (e.g., Hordeum vulgare), Ipomoea batatas, Juglans spp., Lactuca sativa, Linum usitatissimum, Litchi chinensis, Lotus spp., Luffa acutangula, Lupinus spp., Lycopersicon spp. (e.g., Lycopersicon esculenturn, Lycopersicon lycopersicum, Lycopersicon pyriforme), Malus spp., Medicago sativa, Mentha spp., Miscanthus sinensis, Morus nigra, Musa spp., Nicotiana spp., Olea spp., Oryza spp. (e.g., Oryza sativa, Oryza latifolia), Panicum miliaceum, Panicum virgatum, Passiflora edulis, Petroselinum crispum, Phaseolus spp., Pinus spp., Pistacia vera, Pisum spp., Poa spp., Populus spp., Prunus spp., Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis spp., Solanum spp. (e.g., Solanum tuberosum, Solanum integrifolium or Solanum lycopersicum), Sorghum bicolor, Sorghum halepense, Spinacia spp., Tamarindus indica, Theobroma cacao, Trifolium spp., Triticosecale rimpaui, Triticum spp. (e.g., Triticum aestivum, Triticum durum, Triticum turgidum, Triticum hybernum, Triticum macha, Triticum sativum or Triticum vulgare), Vaccinium spp., Vicia spp., Vigna spp., Viola odorata, Vitis spp., and Zea mays. In certain embodiments, the crop plant is rice, oilseed rape, canola, soybean, corn (maize), cotton, sugarcane, alfalfa, sorghum, or wheat.

In certain instance, the compositions and methods can be used to treat post-harvest plants or plant parts, food, or feed products. In some instances, the food or feed product is a non-plant food or feed product (e.g., a product edible for humans, veterinary animals, or livestock (e.g., mushrooms)).

The plant or plant part for use in the present invention include plants of any stage of plant development. In certain instances, the delivery can occur during the stages of germination, seedling growth, vegetative growth, and reproductive growth. In certain instances, delivery to the plant occurs during vegetative and reproductive growth stages. Alternatively, the delivery can occur to a seed. The stages of vegetative and reproductive growth are also referred to herein as “adult” or “mature” plants.

### IV. Pests

The pest control (e.g., biopesticide or biorepellent) compositions and related methods described herein are useful to decrease the fitness of plant pests and thereby treat or prevent pest infestations in plants. “Pests” refer to invertebrates, e.g., insects, nematodes, or mollusks; microorganisms (e.g., phytopathogens, endophytes, obligate parasites, facultative parasites, or facultative saprophytes), such as bacteria, fungi, or viruses, or weeds. Such pests cause damage to plants or other organisms, are present where they are not wanted, or otherwise are detrimental to humans, for example, by impacting human agricultural methods or products.

Examples of plant pests that can be treated with the present compositions or related methods are further described herein.

A. Fungi

The pest control (e.g., biopesticide or biorepellent) compositions and related methods can be useful for decreasing the fitness of a fungus, e.g., to prevent or treat a fungal infection in a plant. Included are methods for delivering a pest control (e.g., biopesticide or biorepellent) composition to a fungus by contacting the fungus with the pest control (e.g., biopesticide or biorepellent) composition. Additionally or alternatively, the methods include delivering the pest control (e.g., biopesticide or biorepellent) composition to a plant at risk of or having a fungal infection, by contacting the plant with the pest control (e.g., biopesticide or biorepellent) composition.

The pest control (e.g., biopesticide or biorepellent) compositions and related methods are suitable for delivery to fungi that cause fungal diseases in plants, including diseases caused by powdery mildew pathogens, for example Blumeria species, for example Blumeria graminis; Podosphaera species, for example Podosphaera leucotricha; Sphaerotheca species, for example Sphaerotheca fuliginea; Uncinula species, for example Uncinula necator; diseases caused by rust disease pathogens, for example Gymnosporangium species, for example Gymnosporangium sabinae; Hemileia species, for example Hemileia vastatrix; Phakopsora species, for example Phakopsora pachyrhizi and Phakopsora meibomiae; Puccinia species, for example Puccinia recondite, P. triticina, P. graminis or P. striiformis or P. hordei; Uromyces species, for example Uromyces appendiculatus; diseases caused by pathogens from the group of the Oomycetes, for example Albugo species, for example Algubo candida; Bremia species, for example Bremia lactucae; Peronospora species, for example Peronospora pisi, P. parasitica or P. brassicae; Phytophthora species, for example Phytophthora infestans; Plasmopara species, for example Plasmopara viticola; Pseudoperonospora species, for example Pseudoperonospora humuli or Pseudoperonospora cubensis; Pythium species, for example Pythium ultimum; leaf blotch diseases and leaf wilt diseases caused, for example, by Alternaria species, for example Alternaria solani; Cercospora species, for example Cercospora beticola; Cladiosporium species, for example Cladiosporium cucumerinum; Cochliobolus species, for example Cochliobolus sativus (conidia form: Drechslera, Syn: Helminthosporium), Cochliobolus miyabeanus; Colletotrichum species, for example Colletotrichum lindemuthanium; Cycloconium species, for example Cycloconium oleaginum; Diaporthe species, for example Diaporthe citri; Elsinoe species, for example Elsinoe fawcettii; Gloeosporium species, for example Gloeosporium laeticolor; Glomerella species, for example Glomerella cingulata; Guignardia species, for example Guignardia bidwelli; Leptosphaeria species, for example Leptosphaeria maculans, Leptosphaeria nodorum; Magnaporthe species, for example Magnaporthe grisea; Microdochium species, for example Microdochium nivale; Mycosphaerella species, for example Mycosphaerella graminicola, M. arachidicola and M. fifiensis; Phaeosphaeria species, for example Phaeosphaeria nodorum; Pyrenophora species, for example Pyrenophora teres, Pyrenophora tritici repentis; Ramularia species, for example Ramularia collo-cygni, Ramularia areola; Rhynchosporium species, for example Rhynchosporium secalis; Septoria species, for example Septoria apii, Septoria lycopersii; Typhula species, for example Typhula incarnata; Venturia species, for example Venturia inaequalis; root and stem diseases caused, for example, by Corticium species, for example Corticium graminearum; Fusarium species, for example Fusarium oxysporum; Gaeumannomyces species, for example Gaeumannomyces graminis; Rhizoctonia species, such as, for example Rhizoctonia solani; Sarocladium diseases caused for example by Sarocladium oryzae; Sclerotium diseases caused for example by Sclerotium oryzae; Tapesia species, for example Tapesia acuformis; Thielaviopsis species, for example Thielaviopsis basicola; ear and panicle diseases (including corn cobs) caused, for example, by Alternaria species, for example Alternaria spp.; Aspergillus species, for example Aspergillus flavus; Cladosporium species, for example Cladosporium cladosporioides; Claviceps species, for example Claviceps purpurea; Fusarium species, for example Fusarium culmorum; Gibberella species, for example Gibberella zeae; Monographella species, for example Monographella nivalis; Septoria species, for example Septoria nodorum; diseases caused by smut fungi, for example Sphacelotheca species, for example Sphacelotheca reiliana; Tilletia species, for example Tilletia caries, T. controversa; Urocystis species, for example Urocystis occulta; Ustilago species, for example Ustilago nuda, U. nuda tritici; fruit rot caused, for example, by Aspergillus species, for example Aspergillus flavus; Botrytis species, for example Botrytis cinerea; Penicillium species, for example Penicillium expansum and P. purpurogenum; Sclerotinia species, for example Sclerotinia sclerotiorum; Verticilium species, for example Verticilium alboatrum; seed and soilborne decay, mould, wilt, rot and damping-off diseases caused, for example, by Alternaria species, caused for example by Alternaria brassicicola; Aphanomyces species, caused for example by Aphanomyces euteiches; Ascochyta species, caused for example by Ascochyta lentis; Aspergillus species, caused for example by Aspergillus flavus; Cladosporium species, caused for example by Cladosporium herbarum; Cochliobolus species, caused for example by Cochlliobolus sativus; (Conidiaform: Drechslera, Bipolaris Syn: Helminthosporium); Colletotrichum species, caused for example by Colletotrichum coccodes; Fusarium species, caused for example by Fusarium culmorum; Gibberella species, caused for example by Gibberella zeae; Macrophomina species, caused for example by Macrophomina phaseolina; Monographella species, caused for example by Monographella nivalis; Penicillium species, caused for example by Penicillium expansum; Phoma species, caused for example by Phoma lingam; Phomopsis species, caused for example by Phomopsis sojae; Phytophthora species, caused for example by Phytophthora cactorum; Pyrenophora species, caused for example by Pyrenophora graminea; Pyricularia species, caused for example by Pyricularia oryzae; Pythium species, caused for example by Pythium ultimum; Rhizoctonia species, caused for example by Rhizoctonia solani; Rhizopus species, caused for example by Rhizopus oryzae; Sclerotium species, caused for example by Sclerotium rolfsii; Septoria species, caused for example by Septoria nodorum; Typhula species, caused for example by Typhula incarnata; Verticillium species, caused for example by Verticillium dahliae; cancers, galls and witches' broom caused, for example, by Nectria species, for example Nectria galligena; wilt diseases caused, for example, by Monilinia species, for example Monilinia laxa; leaf blister or leaf curl diseases caused, for example, by Exobasidium species, for example Exobasidium vexans; Taphrina species, for example Taphrina deformans; decline diseases of wooden plants caused, for example, by Esca disease, caused for example by Phaemoniella clamydospora, Phaeoacremonium aleophilum and Fomitiporia mediterranea; Eutypa dyeback, caused for example by Eutypa lata; Ganoderma diseases caused for example by Ganoderma boninense; Rigidoporus diseases caused for example by Rigidoporus lignosus; diseases of flowers and seeds caused, for example, by Botrytis species, for example Botrytis cinerea; diseases of plant tubers caused, for example, by Rhizoctonia species, for example Rhizoctonia solani; Helminthosporium species, for example Helminthosporium solani; Club root caused, for example, by Plasmodiophora species, for example Plamodiophora brassicae; diseases caused by bacterial pathogens, for example Xanthomonas species, for example Xanthomonas campestris pv. oryzae; Pseudomonas species, for example Pseudomonas syringae pv. lachrymans; Erwinia species, for example Erwinia amylovora.

Fungal diseases on leaves, stems, pods and seeds caused, for example, by Alternaria leaf spot (Alternaria spec. atrans tenuissima), Anthracnose (Colletotrichum gloeosporoides dematium var. truncatum), brown spot (Septoria glycines), cercospora leaf spot and blight (Cercospora kikuchi), choanephora leaf blight (Choanephora infundibulifera trispora (Syn.)), dactuliophora leaf spot (Dactuliophora glycines), downy mildew (Peronospora manshurica), drechslera blight (Drechslera glycini), frogeye leaf spot (Cercospora sojina), leptosphaerulina leaf spot (Leptosphaerulina trifoli), phyllostica leaf spot (Phyllosticta sojaecola), pod and stem blight (Phomopsis sojae), powdery mildew (Microsphaera diffusa), pyrenochaeta leaf spot (Pyrenochaeta glycines), rhizoctonia aerial, foliage, and web blight (Rhizoctonia solani), rust (Phakopsora pachyrhizi, Phakopsora meibomiae), scab (Sphaceloma glycines), stemphylium leaf blight (Stemphylium botryosum), target spot (Corynespora cassiicola).

Fungal diseases on roots and the stem base caused, for example, by black root rot (Calonectria crotalariae), charcoal rot (Macrophomina phaseolina), fusarium blight or wilt, root rot, and pod and collar rot (Fusarium oxysporum, Fusarium orthoceras, Fusarium semitectum, Fusarium equiseti), mycoleptodiscus root rot (Mycoleptodiscus terrestris), neocosmospora (Neocosmospora vasinfecta), pod and stem blight (Diaporthe phaseolorum), stem canker (Diaporthe phaseolorum var. caulivora), phytophthora rot (Phytophthora megasperma), brown stem rot (Phialophora gregata), pythium rot (Pythium aphanidermatum, Pythium irregulare, Pythium debaryanum, Pythium myriotylum, Pythium ultimum), rhizoctonia root rot, stem decay, and damping-off (Rhizoctonia solant), sclerotinia stem decay (Sclerotinia sclerotiorum), sclerotinia southern blight (Sclerotinia rolfsi), thielaviopsis root rot (Thielaviopsis basicola).

In certain instances, the fungus is a Sclerotinia spp (Scelrotinia sclerotiorum). In certain instances, the fungus is a Botrytis spp (e.g., Botrytis cinerea). In certain instances, the fungus is an Aspergillus spp. In certain instances, the fungus is a Fusarium spp. In certain instances, the fungus is a Penicillium spp.

Compositions of the present invention are useful in various fungal control applications. The above-described compositions may be used to control fungal phytopathogens prior to harvest or post-harvest fungal pathogens. In one embodiment, any of the above-described compositions are used to control target pathogens such as Fusarium species, Botrytis species, Verticillium species, Rhizoctonia species, Trichoderma species, or Pythium species by applying the composition to plants, the area surrounding plants, or edible cultivated mushrooms, mushroom spawn, or mushroom compost. In another embodiment, compositions of the present invention are used to control post-harvest pathogens such as Penicillium, Geotrichum, Aspergillus niger, and Colletotrichum species.

Table 1 provides further examples of fungi, and plant diseases associated therewith, that can be treated or prevented using the pest control (e.g., biopesticide or biorepellent) composition and related methods described herein.

TABLE 1
Fungal pests

| Disease | Causative Agent |
|---|---|
| Alternaria leaf blight of wheat | Alternaria triticina |
| Alternaria leaf spot of cole crops | Alternaria japonica |
| American soybean rust | Phakopsora meibomiae |
| Ampelopsis rust | Phakopsora ampelopsidis |
| Anemone | Ochropsora ariae |
| Angular leaf spot of Citrus | Pseudocercospora angolensis |
| Arctic Rubus rust | Phragmidium arcticum |
| Ascochyta blight of broad beans | Didymella fabae |
| Ash dieback | Chalara fraxinea |
| Asia mountain Rosa rust | Phragmidium butleri |
| Asian filbert rust | Pucciniastrum coryli |
| Asian Kuehneola rose rust | Kuehneola japonica |
| Asian Mountain Rubus rust | Phragmidium assamense |
| Asian Phragmidium Rubus rust | Phragmidium arisanense |
| Asian pistacio rust | Pileolaria pistaciae |
| Asian rose rust | Gerwasia rosae |
| Asian Rubus rust | Hamaspora hashiokai |
| Asian soybean rust | Phakopsora pachyrhizi |
| Asian sugarcane smut | Sporisorium sacchari |
| Asian Wart bark, blister canker, | Botryosphaeria berengeriana |
| ring rot, Physalospora canker of | f. sp. pyricola |
| pear and apple | |
| Asian/European brown rot of | Monilinia fructigena |
| rosaceae | |
| Asiatic brown fruit rot | Monilia polystroma |
| Barclay's Asian Rubus rust | Phragmidium barclayi |
| Black leaf blight of soybean | Arkoola nigra |
| Blister blight of tea | Exobasidium vexans |
| Blue stain of Mongolian oak | Ophiostoma longicollum |
| Box Rust or Boxwood Rust | Puccinia buxi |
| Brown rust of sugarcane | Puccinia melanocephala |
| Cherry leaf scorch | Apiognomonia erythrostoma |
| Chocolate spot of Ya Li pears | Alternaria yaliinficiens |
| Chrysanthemum White Rust | Puccinia horiana |
| Coffee Leaf Rust | Hemileia vastatrix |
| Common Asian Rubus Rust | Hamaspora acutissima |
| Common larch | Melampsora capraearum |
| Common potato and tomato rust | Puccinia pittieriana |
| Crumenulopsis pine dieback | Crumenulopsis sororia |
| Daylily Rust | Puccinia hemerocallidis |
| Digitalis Downy Mildew | Peronospora digitalis |
| Downy mildew (Plasmopara) of | Plasmopara obducens |
| Impatiens | |
| Eggplant | Puccinia substriata var. substriata |
| Ergot of pearl millet | Claviceps fusiformis |
| European Larch canker | Lachnellula willkommii |
| Few-loculed Asian Rubus rust | Phragmidium pauciloculare |
| Flag smut of wheat | Urocystis agropyri |
| Gladiolus Rust | Uromyces transversalis |
| Goplana dioscoreae | Goplana dioscoreae |
| Grape leaf rust | Phakopsora euvitis |
| Gray Rubus rust | Phragmidium griseum |
| Himalayan rhododendron | Chrysomyxa himalensis |
| spruce rust | |
| Hiratsuka Rubus rust | Phragmidium hiratsukanum |
| Horse's tooth or ergot of maize | Claviceps gigantea |
| Japanese apple rust | Gymnosporangium yamadae |
| Japanese Chamaecyparis | Gymnosporangium miyabei |
| Japanese ergot of sorghum | Claviceps sorghicola |
| Kamtschatka rose rust | Phragmidium kamtschatkae |
| Late wilt of maize | Harpophora maydis |
| Long-Spored Asian Rubus rust | Hamaspora longissima |
| Mal secco disease of Citrus | Phoma tracheiphila |
| Miscanthus | Puccinia miscanthi |
| Mulberry rust | Aecidium mori |
| Nambu Rubus rust | Phragmidium nambuanum |
| Neck rot of onion | Ciborinia allii |
| New Zealand Rubus Rust | Hamaspora australis |
| Northern blue stain of pine | Leptographium wingfieldii |
| Northern spruce | Chrysomyxa rhododendri |
| Oak Wilt | Ceratocystis fagacearum |
| Orange rust of sugarcane | Puccinia kuehnii |
| Peronospora radii | Peronospora radii |
| Pistachio Rust | Pileolaria terebinthi |
| Poinsettia scab | Sphaceloma poinsettiae |
| Potato smut | Thecaphora solani |
| Puccinia gladioli on Gladiolus | Puccinia gladioli |
| Puccinia glyceriae (anam. | Puccinia glyceriae |
| Aecidium hydrangea | |
| Puccinia mccleanii on Gladiolus | Puccinia mccleanii |
| Puccinia psidii | Puccinia psidii |
| Pucciniastrum actinidiae on | Pucciniastrum actinidiae |
| Actinidia spp. | |
| Red Miscanthus rust | Puccinia erythropus |
| Rust of European blackberry | Phragmidium bulbosum |
| Rust of Rubus saxitilis | Phragmidium acuminatum |
| Rust on Asian Rubus | Gerwasia rubi |
| Rust on South American Rubus | Gerwasia imperialis |
| Scots stem pine rust | Cronartium flaccidum |
| Shoot blight of boxwood | Calonectria pseudonaviculata |
| Sirex wasp fungus | Amylostereum areolatum |
| Solanum | Puccinia agrophila |
| South American Rubus rust | Gerwasia mayorii |
| Sporisorium smut of wild | Sporisorium pulverulentum |
| Saccharum | |
| Spruce needle rust | Chrysomyxa abietis |
| Stackburn, seedling blight, leaf | Alternaria padwickii |
| spot of rice | |
| Sudden needle drop of Spruce | Setomelanomma holmii |
| (SNEED) | |
| Sugary disease or Asian ergot | Claviceps sorghi |
| of sorghum | |
| Sweet potato rust | Endophyllum kaembachii |
| Taiwan Rubus rust | Phragmidium formosanum |
| Tar spot of corn | Phyllachora maydis |
| Teak Rust | Olivea tectonae |
| Thekopsora areolate | Thekopsora areolata |
| Tip over disease of egglant | Diaporthe vexans |
| Tropical American Kuehneola | Kuehneola loeseneriana |
| rust of Rubus | |
| Tropical American Mainsia | Mainsia rubi |
| Rubus rust | |
| Tropical Soybean Rust | Aecidium glycines |
| Uromyces gladioli on Gladiolus | Uromyces gladioli |
| Uromyces nyikensis on | Uromyces nyikensis |
| Gladiolus | |
| Uromycladium tepperianum on | Uromycladium tepperianum |
| Acacia spp. | |
| Variable Rubus | Gerwasia variabilis |
| Wineberry Rubus rust | Hamaspora sinica var. sinica |
| Yamada Rubusrust | Phragmidium yamadanum |
| Anthracnose leaf blight and stalk | Colletotrichum graminicola anthracnose (teleomorph: Glomerella |
| rot | graminicola), Glomerella tucumanensis (anamorph: Glomerella |
| | falcatum) |
| Aspergillus ear and kernel rot | Aspergillus flavus |
| Banded leaf and sheath spot | Rhizoctonia solani = Rhizoctonia microsclerotia (teleomorph: |
| | Thanatephorus cucumeris) |
| Bean rust | Uromyces appendiculatus |
| Black bundle disease | Acremonium strictum = Cephalosporium acremonium |
| Black kernel rot | Lasiodiplodia theobromae = Botryodiplodia theobromae |
| Borde blanco | Marasmiellus sp. |
| Brown spot (black spot, stalk rot) | Physoderma maydis |
| Brown stripe downy mildew | Sclerophthora rayssiae var. zeae |
| Cephalosporium kernel rot | Acremonium strictum = Cephalosporium acremonium |
| Charcoal rot | Macrophomina phaseolina |
| Corn common rust | Puccinia sorghi |
| Corn southern rust | Puccinia polysora |
| Corn tropical rust | Physopella pallescens, P. zeae = Angiospora zeae |
| Corticium ear rot | Thanatephorus cucumeris = Corticium sasakii |
| Cotton rust | Puccinia schedonnardi |
| Cotton southwestern rust | Puccinia cacabata |
| Cotton tropical rust | Phakopsora gossypii |
| Crazy top downy mildew | Sclerophthora macrospora = S. macrospora |
| Curvularia leaf spot | Curvularia clavata, C. eragrostidis, = C. maculans (teleomorph: |
| | Cochliobolus eragrostidis), Curvularia inaequalis, C. intermedia |
| | (teleomorph: Cochliobolus intermedius), Curvularia lunata |
| | (teleomorph: Cochliobolus lunatus), Curvularia pallescens |
| | (teleomorph: Cochliobolus pallescens), Curvularia senegalensis, C. |
| | tuberculata (teleomorph: Cochliobolus tuberculatus) |
| Didymella leaf spot | Didymella exitialis |
| Diplodia ear rot and stalk rot | Diplodia frumenti (teleomorph: Botryosphaeria festucae) |
| Diplodia ear rot, stalk rot, seed | Diplodia maydis = Stenocarpella maydis |
| rot and seedling blight | |
| Diplodia leaf spot or leaf streak | Stenocarpella macrospora = Diplodia macrospore |
| Grape leaf Downey mildew | Plasmopara viticola |
| Dry ear rot (cob, kernel and stalk | Nigrospora oryzae (teleomorph: Khuskia oryzae) |
| rot) | |
| Ear rots, minor | Aspergillus glaucus, A. niger, Aspergillus spp., Cunninghamella |
| | sp., Curvularia pallescens, Doratomyces stemonitis = |
| | Cephalotrichum stemonitis, Fusarium culmorum, Gonatobotrys |
| | simplex, Pithomyces maydicus, Rhizopus microsporus, R. |
| | stolonifer = R. nigricans, Scopulariopsis brumptii |
| epitea | Melampsora larici |
| Ergot (horse's tooth, diente del | Claviceps gigantea (anamorph: Sphacelia sp.) |
| caballo) | |
| Eyespot | Aureobasidium zeae = Kabatiella zeae |
| Fusarium ear and stalk rot | Fusarium subglutinans = F. moniliforme var. subglutinans |
| Fusarium kernel, root and stalk | Fusarium moniliforme (teleomorph: Gibberella fujikuroi) |
| rot, seed rot and seedling blight | |
| Fusarium stalk rot, seedling root | Fusarium avenaceum (teleomorph: Gibberella avenacea) |
| rot | |
| Gibberella ear and stalk rot | Gibberella zeae (anamorph: Fusarium graminearum) |
| Gray ear rot | Botryosphaeria zeae = Physalospora zeae (anamorph: |
| | Macrophoma zeae) |
| Gray leaf spot (Cercospora | Cercospora sorghi = C. sorghi var. maydis, C. zeae-maydis leaf |
| | spot) |
| Green ear downy mildew | Sclerospora graminicola |
| Helminthosporium ear rot (race | Bipolaris zeicola = Helminthosporium carbonum |
| 1) | |
| Helminthosporium root rot | Exserohilum pedicellatum = Helminthosporium pedicellatum |
| | (teleomorph: Setosphaeria) |
| Hormodendrum ear rot | Cladosporium cladosporioides = Hormodendrum cladosporioides, |
| (Cladosporium rot) | C. herbarum (teleomorph: Mycosphaerella tassiana) |
| Hyalothyridium leaf spot | Hyalothyridium maydis |
| Java downy mildew | Peronosclerospora maydis = Sclerospora maydis |
| Late wilt | Cephalosporium maydis |
| Leaf (brown) rust | Puccinia recondita (anamorph: Aecidium clematitis) |
| Leaf spots, minor | Alternaria altemata, Ascochyta maydis, A. tritici, A. zeicola, |
| | Bipolaris victoriae = Helminthosporium victoriae (teleomorph: |
| | Cochliobolus victoriae), C. sativus (anamorph: Bipolaris |
| | sorokiniana = H. Exserohilum maydis, Leptothyrium |
| | zeae, Ophiosphaerella herpotricha, Setosphaeria prolata) |
| | Graphium penicillioides, Leptosphaeria prolatum = Drechslera |
| | prolata (teleomorph: sorokinianum = H. sativum), Epicoccum |
| | nigrum, (anamorph: Scolecosporiella sp.), Paraphaeosphaeria |
| | michotii, Phoma sp., Septoria zeae, S. zeicola, S. zeina |
| Rust fungi | Puccinia veronicae-longifoliae |
| Musk rose rust | Phragmidium rosae-moschatae |
| Multiflora rose rust | Phragmidium rosae-multiflorae |
| Northern corn leaf blight | Exaerohilum turcicum = Helminthosporium turcicum, Setosphaeria |
| | turcica |
| Northern corn leaf spot | Cochliobolus carbonum |
| Oat crown rust | Puccinia coronata |
| Oat stern Rust | Puccinia graminis |
| Peanut rust | Puccinia arachidis |
| Penicillium ear rot (blue eye, | Penicillium spp., P. chrysogenum, P. expansum, P. oxalicum |
| blue mold) | |
| Bay willow-larch rust | Melampsora larici-pentandrae |
| Phaeocytostroma stalk rot and | Phaeocytostroma ambiguum, Phaeocytosporella zeae |
| root rot | |
| Phaeosphaeria leaf spot | Phaeosphaeria maydis, Sphaerulina maydis |
| Philippine downy mildew | Peronosclerospora philippinensis = Sclerospora philippinensis |
| Physalospora ear rot | Botryosphaeria Botryosphaeria festucae = Physalospora zeicola, |
| | (anamorph: Diplodia frumenti) |
| Potato common rust | Puccinia pittierianap |
| Potato deforming rust | Aecidium cantensis |
| Cereals and grasses Powdery | Erysiphe graminis |
| mildew | |
| Rose Powdery mildew | Sphaerotheca pannosa |
| Wheat Powdery mildew | Blumeria graminis f. sp. tritici, |
| Barley Powdery mildew | Blumeria graminis f. sp. hordei |
| Grape Powdery mildew | Microsphaera diffusa |
| Legume Powdery mildew | Erysiphe necator (or Uncinula necator) |
| Grape Powdery mildew | Leveillula taurica, or Oidiopsis taurica |
| Onion Powdery mildew | Podosphaera leucotricha |
| Apple Powdery mildew | Podosphaera xanthii, Erysiphe cichoracearum, Podosphaera fusca, |
| | Leveillula taurica |
| Cucurbits Powdery mildew | Microsphaera syringae |
| Lilacs Powdery mildew | Podosphaera aphanis, Geum rivale |
| Strawberry Powdery mildew | Erysiphe berberidis |
| Hawthorn Powdery mildew | Podosphaera oxyacanthae |
| Gooseberry Powdery mildew | Sphaerotheca mors-uvae |
| Purple leaf sheath | Hemiparasitic bacteria and fungi |
| Pyrenochaeta stalk rot and root | Phoma terrestris, Pyrenochaeta terrestris |
| rot | |
| Pythium root rot | Pythium spp., P. arrhenomanes, P. graminicola |
| Pythium stalk rot | Pythium aphanidermatum = P. butleri L. |
| Red kernel disease (ear mold, | Epicoccum nigrum |
| leaf and seed rot) | |
| Rhizoctonia ear rot | Rhizoctonia zeae (teleomorph: Waitea circinata) |
| Rhizoctonia root rot and stalk rot | Rhizoctonia solani, Rhizoctonia zeae |
| Root rots, minor | Alternaria altemata, Cercospora sorghi, Dictochaeta fertilis, |
| | Fusarium acuminatum (teleomorph: Gibberella acuminate), F. |
| | equiseti (teleomorph: G. intricans), F. oxysporum, F. |
| | paffidoroseum, F. poae, F. roseum, F. cyanogena, (anamorph: F. |
| | sulphureum), Microdochium bolleyi, Mucor sp., Periconia |
| | circinata, Phytophthora cactorum, P. drechsleri, P. nicotianae var. |
| | parasitica, Rhizopus arrhizus |
| Rostratum leaf spot (leaf | Setosphaeria rostrata, Helminthosporium (anamorph: Exserohilum |
| disease, ear and, stalk rot) | rostratum = Helminthosporium rostratum) |
| rugosae | Phragmidium rosae |
| Rust, common corn | Puccinia sorghi |
| Rust, southern corn | Puccinia polysora |
| Rust, tropical corn | Physopella pallescens, P. zeae = Angiospora zeae |
| sativae | Balansia oryzae |
| Sclerotium ear rot (southern | Sclerotium rolfsii (teleomorph: Athelia rolfsii) |
| blight) | |
| Seed rot-seedling blight | Bipolaris sorokiniana, B. zeicola = Helminthosporium carbonum, |
| | Diplodia maydis, Exserohilum pedicellatum, Exserohilum |
| | turcicum = Helminthosporium turcicum, Fusarium avenaceum, F. |
| | culmorum, F. moniliforme, Gibberella zeae (anamorph: F. |
| | graminearum), Macrophomina |
| | phaseolina, Penicillium spp., Phomopsis sp., |
| | Pythium spp., Rhizoctonia solani, R. zeae, Sclerotium rolfsii, |
| | Spicaria sp. |
| Selenophoma leaf spot | Selenophoma sp. |
| Sheath rot | Gaeumannomyces graminis |
| Shuck rot | Myrothecium gramineum |
| sieboldii | Hamaspora rubi |
| Silage mold | Monascus purpureus, M. rubber |
| Smut, common | Ustilago zeae = U. maydis |
| Smut, false | Ustilaginoidea virens |
| Smut, head | Sphacelotheca reiliana = Sporisorium holci-sorghi |
| Sorghum downy mildew | Peronosclerospora sorghi = Sclerospora sorghi |
| Southern corn leaf blight and | Cochliobolus heterostrophus (anamorph: Bipolaris maydis = |
| stalk rot | Helminthosporium maydis) |
| Southern leaf spot | Stenocarpella macrospora = Diplodia macrospora |
| Soybean rust | Phakopsora pachyrhizi |
| Spontaneum downy mildew | Peronosclerospora spontanea = Sclerospora spontanea |
| Stalk rots, minor | Cercospora sorghi, Fusarium episphaeria, F. merismoides, F. |
| | oxysportum, F. poae, F. roseum, F. solani (teleomorph: Nectria |
| | haematococca), F. tricinctum, Mariannaea elegans, Mucor sp., |
| | Rhopographus zeae, Spicaria sp. |
| Stem rust | Puccinia graminis = P. graminis f. sp. secalis |
| Storage rots | Aspergillus spp., Penicillium spp. and other fungi |
| Sugarcane common rust | Puccinia melanocephala = P. eriantha |
| Sugarcane downy mildew | Peronosclerospora sacchari = Sclerospora sacchari |
| Tar spot | Phyllachora maydis |
| thunbergii | Phragmidium rubi |
| Trichoderma ear rot and root rot | Trichoderma viride = T. lignorum (teleomorph: Hypocrea sp.) |
| Wheat leaf (brown) rust | Puccinia triticina = P. Recondita f. Sp. tritici = P. tritici-duri |
| Wheat stem (black) rust | Puccinia graminis = P. graminis f. sp. tritici |
| Wheat stripe (yellow) rust | Puccinia striiformis (anamorph: P. uredoglumarum) |
| White ear rot, root and stalk rot | Stenocarpella maydis = Diplodia zeae |
| Yellow leaf blight | Ascochyta ischaemi, Phyllosticta maydis (teleomorph: |
| | Mycosphaerella zeae-maydis) |
| Zonate leaf spot | Gloeocercospora sorghi |

B. Bacteria

The pest control (e.g., biopesticide or biorepellent) compositions and related methods can be useful for decreasing the fitness of a bacterium, e.g., to prevent or treat a bacterial infection in a plant. Included are methods for delivering a pest control (e.g., biopesticide or biorepellent) composition to a bacterium by contacting the bacteria with the pest control (e.g., biopesticide or biorepellent) composition. Additionally or alternatively, the methods include delivering the biopesticide to a plant at risk of or having a bacterial infection, by contacting the plant with the pest control (e.g., biopesticide or biorepellent) composition.

The pest control (e.g., biopesticide or biorepellent) compositions and related methods are suitable for delivery to bacteria, or a plant infected therewith, including any bacteria described further below. For example, the bacteria may be one belonging to Actinobacteria or Proteobacteria, such as bacteria in the families of the Burkholderiaceae, Xanthomonadaceae, Pseudomonadaceae, Enterobacteriaceae, Microbacteriaceae, and Rhizobiaceae.

In some instances, the bacteria is a Acidovorax avenae subsp., including e.g., Acidovorax avenae subsp. avenae (=Pseudomonas avenae subsp. avenae), Acidovorax avenae subsp. cattleyae (=Pseudomonas cattleyae), or Acidovorax avenae subsp. citrulli (=Pseudomonas pseudoalcaligenes subsp. citrulli, Pseudomonas avenae subsp. citrulli)).

In some instances, the bacteria is a Burkholderia spp., including e.g., Burkholderia andropogonis (=Pseudomonas andropogonis, Pseudomonas woodsil), Burkholderia caryophylli (=Pseudomonas caryophylli), Burkholderia cepacia (=Pseudomonas cepacia), Burkholderia gladioli (=Pseudomonas gladioli), Burkholderia gladioli pv. agaricicola (=Pseudomnas gladioli pv. agaricicola), Burkholderia gladioli pv. alliicola (i.e., Pseudomonas gladioli pv. alliicola), Burkholderia gladioli pv. gladioli (i.e., Pseudomonas gladioli, Pseudomonas gladioli pv. gladioli), Burkholderia glumae (i.e., Pseudomonas glumae), Burkholderia plantarii (i.e., Pseudomonas plantaril), Burkholderia solanacearum (i.e., Ralstonia solanacearum), or Ralstonia spp.

In some instances, the bacteria is a Liberibacter spp., including Candidatus Liberibacter spec., including e.g., Candidatus Liberibacter asiaticus, Liberibacter africanus (Laf), Liberibacter americanus (Lam), Liberibacter asiaticus (Las), Liberibacter europaeus (Leu), Liberibacter psyllaurous, or Liberibacter solanacearum (Lso).

In some instances, the bacteria is a Corynebacterium spp. including e.g., Corynebacterium fascians, Corynebacterium flaccumfaciens pv. flaccumfaciens, Corynebacterium michiganensis, Corynebacterium michiganense pv. tritici, Corynebacterium michiganense pv. nebraskense, or Corynebacterium sepedonicum.

In some instances, the bacteria is a Erwinia spp. including e.g., Erwinia amylovora, Erwinia ananas, Erwinia carotovora (i.e., Pectobacterium carotovorum), Erwinia carotovora subsp. atroseptica, Erwinia carotovora subsp. carotovora, Erwinia chrysanthemi, Erwinia chrysanthemi pv. zeae, Erwinia dissolvens, Erwinia herbicola, Erwinia rhapontic, Erwinia stewartiii, Erwinia tracheiphila, or Erwinia uredovora.

In some instances, the bacteria is a Pseudomonas syringae subsp., including e.g., Pseudomonas syringae pv. actinidiae (Psa), Pseudomonas syringae pv. atrofaciens, Pseudomonas syringae pv. coronafaciens, Pseudomonas syringae pv. glycinea, Pseudomonas syringae pv. lachrymans, Pseudomonas syringae pv. maculicola Pseudomonas syringae pv. papulans, Pseudomonas syringae pv. striafaciens, Pseudomonas syringae pv. syringae, Pseudomonas syringae pv. tomato, or Pseudomonas syringae pv. tabaci.

In some instances, the bacteria is Pseudomonas aeruginosa.

In some instances, the bacteria is a Streptomyces spp., including e.g., Streptomyces acidiscabies, Streptomyces albidoflavus, Streptomyces candidus (i.e., Actinomyces candidus), Streptomyces caviscabies, Streptomyces collinus, Streptomyces europaeiscabiei, Streptomyces intermedius, Streptomyces ipomoeae, Streptomyces luridiscabiei, Streptomyces niveiscabiei, Streptomyces puniciscabiei, Streptomyces retuculiscabiei, Streptomyces scabiei, Streptomyces scabies, Streptomyces setonii, Streptomyces steliscabiei, Streptomyces turgidiscabies, or Streptomyces wedmorensis.

In some instances, the bacteria is a Xanthomonas axonopodis subsp., including e.g., Xanthomonas axonopodis pv. alfalfae (=Xanthomonas alfalfae), Xanthomonas axonopodis pv. aurantifolii (=Xanthomonas fuscans subsp. aurantifolil), Xanthomonas axonopodis pv. allii (=Xanthomonas campestris pv. alli), Xanthomonas axonopodis pv. axonopodis, Xanthomonas axonopodis pv. bauhiniae (=Xanthomonas campestris pv. bauhiniae), Xanthomonas axonopodis pv. begoniae (=Xanthomonas campestris pv. begoniae), Xanthomonas axonopodis pv. betlicola (=Xanthomonas campestris pv. betlicola), Xanthomonas axonopodis pv. biophyti (=Xanthomonas campestris pv. biophyti), Xanthomonas axonopodis pv. cajani (=Xanthomonas campestris pv. cajanl), Xanthomonas axonopodis pv. cassavae (=Xanthomonas cassavae, Xanthomonas campestris pv. cassavae), Xanthomonas axonopodis pv. cassiae (=Xanthomonas campestris pv. cassiae), Xanthomonas axonopodis pv. citri (=Xanthomonas citri), Xanthomonas axonopodis pv. citrumelo (=Xanthomonas alfalfae subsp. citrumelonis), Xanthomonas axonopodis pv. clitoriae (=Xanthomonas campestris pv. clitoriae), Xanthomonas axonopodis pv. coracanae (=Xanthomonas campestris pv. coracanae), Xanthomonas axonopodis pv. cyamopsidis (=Xanthomonas campestris pv. cyamopsidis), Xanthomonas axonopodis pv. desmodii (=Xanthomonas campestris pv. desmodi), Xanthomonas axonopodis pv. desmodiigangetici (=Xanthomonas campestris pv. desmodiigangeticl), Xanthomonas axonopodis pv. desmodiilaxiflori (=Xanthomonas campestris pv. desmodiilaxiflon), Xanthomonas axonopodis pv. desmodiirotundifolii (=Xanthomonas campestris pv. desmodiirotundifoli), Xanthomonas axonopodis pv. dieffenbachiae (=Xanthomonas campestris pv. dieffenbachiae), Xanthomonas axonopodis pv. erythrinae (=Xanthomonas campestris pv. erythrinae), Xanthomonas axonopodis pv. fascicularis (=Xanthomonas campestris pv. fasciculan), Xanthomonas axonopodis pv. glycines (=Xanthomonas campestris pv. glycines), Xanthomonas axonopodis pv. khayae (=Xanthomonas campestris pv. khayae), Xanthomonas axonopodis pv. lespedezae (=Xanthomonas campestris pv. lespedezae), Xanthomonas axonopodis pv. maculifoliigardeniae (=Xanthomonas campestris pv. maculifoliigardeniae), Xanthomonas axonopodis pv. malvacearum (=Xanthomonas citri subsp. malvacearum), Xanthomonas axonopodis pv. manihotis (=Xanthomonas campestris pv. manihotis), Xanthomonas axonopodis pv. martyniicola (=Xanthomonas campestris pv. martyniicola), Xanthomonas axonopodis pv. melhusii (=Xanthomonas campestris pv. melhusu), Xanthomonas axonopodis pv. nakataecorchori (=Xanthomonas campestris pv. nakataecorchon), Xanthomonas axonopodis pv. passiflorae (=Xanthomonas campestris pv. passiflorae), Xanthomonas axonopodis pv. patelii (=Xanthomonas campestris pv. patelil), Xanthomonas axonopodis pv. pedalii (=Xanthomonas campestris pv. pedalii), Xanthomonas axonopodis pv. phaseoli (=Xanthomonas campestris pv. phaseoli, Xanthomonas phaseoli), Xanthomonas axonopodis pv. phaseolivar. fuscans (=Xanthomonas fuscans), Xanthomonas axonopodis pv. phyllanthi (=Xanthomonas campestris pv. phyllanth), Xanthomonas axonopodis pv. physalidicola (=Xanthomonas campestris pv. physalidicola), Xanthomonas axonopodis pv. poinsettiicola (=Xanthomonas campestris pv. poinsettiicola), Xanthomonas axonopodis pv. punicae (=Xanthomonas campestris pv. punicae), Xanthomonas axonopodis pv. rhynchosiae (=Xanthomonas campestris pv. rhynchosiae), Xanthomonas axonopodis pv. ricini (=Xanthomonas campestris pv. ricini), Xanthomonas axonopodis pv. sesbaniae (=Xanthomonas campestris pv. sesbaniae), Xanthomonas axonopodis pv. tamarindi (=Xanthomonas campestris pv. tamarind), Xanthomonas axonopodis pv. vasculorum (=Xanthomonas campestris pv. vasculorum), Xanthomonas axonopodis pv. vesicatoria (=Xanthomonas campestris pv. vesicatoria, Xanthomonas vesicatoria), Xanthomonas axonopodis pv. vignaeradiatae (=Xanthomonas campestris pv. vignaeradiatae), Xanthomonas axonopodis pv. vignicola (=Xanthomonas campestris pv. vignicola), or Xanthomonas axonopodis pv. vitians (=Xanthomonas campestris pv. vitians).

In some instances, the bacteria is Xanthomonas campestris pv. musacearum, Xanthomonas campestris pv. pruni (=Xanthomonas arboricola pv. pruni), or Xanthomonas fragariae.

In some instances, the bacteria is a Xanthomonas translucens supsp. (=Xanthomonas campestris pv. horde) including e.g., Xanthomonas translucens pv. arrhenatheri (=Xanthomonas campestris pv. arrhenathen), Xanthomonas translucens pv. cerealis (=Xanthomonas campestris pv. cerealis), Xanthomonas translucens pv. graminis (=Xanthomonas campestris pv. graminis), Xanthomonas translucens pv. phlei (=Xanthomonas campestris pv. phlet), Xanthomonas translucens pv. phleipratensis (=Xanthomonas campestris pv. phleipratensis), Xanthomonas translucens pv. poae (=Xanthomonas campestris pv. poae), Xanthomonas translucens pv. secalis (=Xanthomonas campestris pv. secalis), Xanthomonas translucens pv. translucens (=Xanthomonas campestris pv. translucens), or Xanthomonas translucens pv. undulosa (=Xanthomonas campestris pv. undulosa).

In some instances, the bacteria is a Xanthomonas oryzae supsp., Xanthomonas oryzae pv. oryzae (=Xanthomonas campestris pv. oryzae), or Xanthomonas oryzae pv. oryzicola (=Xanthomonas campestris pv. oryzicola).

In some instances, the bacteria is a Xylella fastidiosa from the family of Xanthomonadaceae.

Table 2 shows further examples of bacteria, and diseases associated therewith, that can be treated or prevented using the pest control (e.g., biopesticide or biorepellent) composition and related methods described herein.

TABLE 2
Bacterial pests

| Disease | Causative Agent |
|---|---|
| Bacterial leaf blight and stalk rot | Pseudomonas avenae subsp. avenae |
| Bacterial leaf spot | Xanthomonas campestris pv. holcicola |
| Bacterial stalk rot | Enterobacter dissolvens = Erwinia dissolvens |
| Bacterial stalk and top rot | Erwinia carotovora subsp. carotovora, Erwinia |
| | chrysanthemi pv. Zeae |
| Bacterial stripe | Pseudomonas andropogonis |
| Chocolate spot | Pseudomonas syringae pv. Coronafaciens |
| Goss's bacterial wilt blight (leaf | Clavibacter michiganensis subsp. |
| freckles and wilt) | nebraskensis = Cornebacterium michiganense |
| | pv. Nebraskense |
| Holcus spot | Pseudomonas syringae pv. Syringae |
| Purple leaf sheath | Hemiparasitic bacteria |
| Seed rot-seedling blight | Bacillus subtilis |
| Stewart's disease (bacterial wilt) | Pantoea stewartii = Erwinia stewartii |
| Corn stunt (Mesa Central or Rio | Achapparramiento, stunt, Spiroplasma kunkelii |
| Grande stunt) | |
| Soft rot | Dickeya dianthicola |
| Soft rot | Dickeya solani |
| Fire blight | Erwinia amylovora |
| Soft rot | P. atrosepticum |
| Soft rot | Pectobacterium carotovorum ssp. carotovorum |
| Soft rot | Pectobacterium wasabiae |
| Bacterial blight | Pseudomonas syringae pv. Porri and pv. Tomato |
| Brown blotch Disease | Pseudomonas tolaasii |
| Bacterial wilt | Ralstonia solanacearum |
| Bacteria wilt | Ralstonia solanacearum |
| Common scab | Streptomyces scabies |
| Common scab | Streptomyces scabies |
| Xanthomonasleaf blight of onion | Xanthomonas axonopodis pv. allii |
| Asiatic citrus canker | Xanthomonas axonopodis pv. citri |
| Citrus bacterial spot | Xanthomonas axonopodis pv. citrumelo |
| Bacterial spot | Xanthomonas campestris pv. vesicatoria |
| Pierce's Disease | Xylella fastidiosa |

C. Insects

The pest control (e.g., biopesticide or biorepellent) compositions and related methods can be useful for decreasing the fitness of an insect, e.g., to prevent or treat an insect infestation in a plant. The term “insect” includes any organism belonging to the phylum Arthropoda and to the class Insecta or the class Arachnida, in any stage of development, i.e., immature and adult insects. Included are methods for delivering a pest control (e.g., biopesticide or biorepellent) composition to an insect by contacting the insect with the pest control (e.g., biopesticide or biorepellent) composition. Additionally or alternatively, the methods include delivering the biopesticide to a plant at risk of or having an insect infestation, by contacting the plant with the pest control (e.g., biopesticide or biorepellent) composition.

The pest control (e.g., biopesticide or biorepellent) compositions and related methods are suitable for preventing or treating infestation by an insect, or a plant infested therewith, including insects belonging to the following orders: Acari, Araneae, Anoplura, Coleoptera, Collembola, Dermaptera, Dictyoptera, Diplura, Diptera (e.g., spotted-wing Drosophila), Embioptera, Ephemeroptera, Grylloblatodea, Hemiptera (e.g., aphids, Greenhouse whitefly), Homoptera, Hymenoptera, Isoptera, Lepidoptera, Mallophaga, Mecoptera, Neuroptera, Odonata, Orthoptera, Phasmida, Plecoptera, Protura, Psocoptera, Siphonaptera, Siphunculata, Thysanura, Strepsiptera, Thysanoptera, Trichoptera, or Zoraptera.

In some instances, the insect is from the class Arachnida, for example, Acarus spp., Aceria sheldoni, Aculops spp., Aculus spp., Amblyomma spp., Amphitetranychus viennensis, Argas spp., Boophilus spp., Brevipalpus spp., Bryobia graminum, Bryobia praetiosa, Centruroides spp., Chorioptes spp., Dermanyssus gallinae, Dermatophagoides pteronyssinus, Dermatophagoides farinae, Dermacentor spp., Eotetranychus spp., Epitrimerus pyri, Eutetranychus spp., Eriophyes spp., Glycyphagus domesticus, Halotydeus destructor, Hemitarsonemus spp., Hyalomma spp., Ixodes spp., Latrodectus spp., Loxosceles spp., Metatetranychus spp., Neutrombicula autumnalis, Nuphersa spp., Oligonychus spp., Ornithodorus spp., Ornithonyssus spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Steneotarsonemus spp., Steneotarsonemus spinki, Tarsonemus spp., Tetranychus spp., Trombicula alfreddugesi, Vaejovis spp., or Vasates lycopersici.

In some instances, the insect is from the class Chilopoda, for example, Geophilus spp. or Scutigera spp.

In some instances, the insect is from the order Collembola, for example, Onychiurus armatus.

In some instances, the insect is from the class Diplopoda, for example, Blaniulus guttulatus; from the class Insecta, e.g. from the order Blattodea, for example, Blattella asahinai, Blattella germanica, Blatta orientalis, Leucophaea maderae, Panchlora spp., Parcoblatta spp., Periplaneta spp., or Supella longipalpa.

In some instances, the insect is from the order Coleoptera, for example, Acalymma vittatum, Acanthoscelides obtectus, Adoretus spp., Agelastica alni, Agriotes spp., Alphitobius diaperinus, Amphimallon solstitialis, Anobium punctatum, Anoplophora spp., Anthonomus spp., Anthrenus spp., Apion spp., Apogonia spp., Atomaria spp., Attagenus spp., Bruchidius obtectus, Bruchus spp., Cassida spp., Cerotoma trifurcata, Ceutorrhynchus spp., Chaetocnema spp., Cleonus mendicus, Conoderus spp., Cosmopolites spp., Costelytra zealandica, Ctenicera spp., Curculio spp., Cryptolestes ferrugineus, Cryptorhynchus lapathi, Cylindrocopturus spp., Dermestes spp., Diabrotica spp. (e.g., corn rootworm), Dichocrocis spp., Dicladispa armigera, Diloboderus spp., Epilachna spp., Epitrix spp., Faustinus spp., Gibbium psylloides, Gnathocerus cornutus, Hellula undalis, Heteronychus arator, Heteronyx spp., Hylamorpha elegans, Hylotrupes bajulus, Hypera postica, Hypomeces squamosus, Hypothenemus spp., Lachnosterna consanguinea, Lasioderma serricorne, Latheticus oryzae, Lathridius spp., Lema spp., Leptinotarsa decemlineata, Leucoptera spp., Lissorhoptrus oryzophilus, Lixus spp., Luperodes spp., Lyctus spp., Megascelis spp., Melanotus spp., Meligethes aeneus, Melolontha spp., Migdolus spp., Monochamus spp., Naupactus xanthographus, Necrobia spp., Niptus hololeucus, Oryctes rhinoceros, Oryzaephilus surinamensis, Oryzaphagus oryzae, Otiorrhynchus spp., Oxycetonia jucunda, Phaedon cochleariae, Phyllophaga spp., Phyllophaga helleri, Phyllotreta spp., Popillia japonica, Premnotrypes spp., Prostephanus truncatus, Psylliodes spp., Ptinus spp., Rhizobius ventralis, Rhizopertha dominica, Sitophilus spp., Sitophilus oryzae, Sphenophorus spp., Stegobium paniceum, Sternechus spp., Symphyletes spp., Tanymecus spp., Tenebrio molitor, Tenebrioides mauretanicus, Tribolium spp., Trogoderma spp., Tychius spp., Xylotrechus spp., or Zabrus spp.

In some instances, the insect is from the order Diptera, for example, Aedes spp., Agromyza spp., Anastrepha spp., Anopheles spp., Asphondylia spp., Bactrocera spp., Bibio hortulanus, Calliphora erythrocephala, Calliphora vicina, Ceratitis capitata, Chironomus spp., Chrysomyia spp., Chrysops spp., Chrysozona pluvialis, Cochliomyia spp., Contarinia spp., Cordylobia anthropophaga, Cricotopus sylvestris, Culex spp., Culicoides spp., Culiseta spp., Cuterebra spp., Dacus oleae, Dasyneura spp., Delia spp., Dermatobia hominis, Drosophila spp., Echinocnemus spp., Fannia spp., Gasterophilus spp., Glossina spp., Haematopota spp., Hydrellia spp., Hydrellia griseola, Hylemya spp., Hippobosca spp., Hypoderma spp., Liriomyza spp., Lucilia spp., Lutzomyia spp., Mansonia spp., Musca spp. (e.g., Musca domestica), Oestrus spp., Oscinella frit, Paratanytarsus spp., Paralauterborniella subcincta, Pegomyia spp., Phlebotomus spp., Phorbia spp., Phormia spp., Piophila casei, Prodiplosis spp., Psila rosae, Rhagoletis spp., Sarcophaga spp., Simullium spp., Stomoxys spp., Tabanus spp., Tetanops spp., or Tipula spp.

In some instances, the insect is from the order Heteroptera, for example, Anasa tristis, Antestiopsis spp., Boisea spp., Blissus spp., Calocoris spp., Campylomma livida, Cavelerius spp., Cimex spp., Collaria spp., Creontiades dilutus, Dasynus piperis, Dichelops furcatus, Diconocoris hewetti, Dysdercus spp., Euschistus spp., Eurygaster spp., Heliopeltis spp., Horcias nobilellus, Leptocorisa spp., Leptocorisa varicornis, Leptoglossus phyllopus, Lygus spp., Macropes excavatus, Miridae, Monalonion atratum, Nezara spp., Oebalus spp., Pentatomidae, Piesma quadrata, Piezodorus spp., Psallus spp., Pseudacysta persea, Rhodnius spp., Sahlbergella singularis, Scaptocoris castanea, Scotinophora spp., Stephanitis nashi, Tibraca spp., or Triatoma spp.

In some instances, the insect is from the order Homiptera, for example, Acizzia acaciaebaileyanae, Acizzia dodonaeae, Acizzia uncatoides, Acrida turrita, Acyrthosipon spp., Acrogonia spp., Aeneolamia spp., Agonoscena spp., Aleyrodes proletella, Aleurolobus barodensis, Aleurothrixus floccosus, Allocaridara malayensis, Amrasca spp., Anuraphis cardui, Aonidiella spp., Aphanostigma pini, Aphis spp. (e.g., Apis gossypii), Arboridia apicalis, Arytainilla spp., Aspidiella spp., Aspidiotus spp., Atanus spp., Aulacorthum solani, Bemisia tabaci, Blastopsylla occidentalis, Boreioglycaspis melaleucae, Brachycaudus helichrysi, Brachycolus spp., Brevicoryne brassicae, Cacopsylla spp., Calligypona marginata, Carneocephala fulgida, Ceratovacuna lanigera, Cercopidae, Ceroplastes spp., Chaetosiphon fragaefolii, Chionaspis tegalensis, Chlorita onukii, Chondracris rosea, Chromaphis juglandicola, Chrysomphalus ficus, Cicadulina mbila, Coccomytilus halli, Coccus spp., Cryptomyzus ribis, Cryptoneossa spp., Ctenarytaina spp., Dalbulus spp., Dialeurodes citri, Diaphorina citri, Diaspis spp., Drosicha spp., Dysaphis spp., Dysmicoccus spp., Empoasca spp., Eriosoma spp., Erythroneura spp., Eucalyptolyma spp., Euphyllura spp., Euscelis bilobatus, Ferrisia spp., Geococcus coffeae, Glycaspis spp., Heteropsylla cubana, Heteropsylla spinulosa, Homalodisca coagulata, Homalodisca vitripennis, Hyalopterus arundinis, Icerya spp., Idiocerus spp., Idioscopus spp., Laodelphax striatellus, Lecanium spp., Lepidosaphes spp., Lipaphis erysimi, Macrosiphum spp., Macrosteles facifrons, Mahanarva spp., Melanaphis sacchari, Metcalfiella spp., Metopolophium dirhodum, Monellia costalis, Monelliopsis pecanis, Myzus spp., Nasonovia ribisnigri, Nephotettix spp., Nettigoniclla spectra, Nilaparvata lugens, Oncometopia spp., Orthezia praelonga, Oxya chinensis, Pachypsylla spp., Parabemisia myricae, Paratrioza spp., Parlatoria spp., Pemphigus spp., Pentatomidae spp. (e.g., Halyomorpha halys), Peregrinus maidis, Phenacoccus spp., Phloeomyzus passerinii, Phorodon humuli, Phylloxera spp., Pinnaspis aspidistrae, Planococcus spp., Prosopidopsylla flava, Protopulvinaria pyriformis, Pseudaulacaspis pentagona, Pseudococcus spp., Psyllopsis spp., Psylla spp., Pteromalus spp., Pyrilla spp., Quadraspidiotus spp., Quesada gigas, Rastrococcus spp., Rhopalosiphum spp., Saissetia spp., Scaphoideus titanus, Schizaphis graminum, Selenaspidus articulatus, Sogata spp., Sogatella furcifera, Sogatodes spp., Stictocephala festina, Siphoninus phillyreae, Tenalaphara malayensis, Tetragonocephela spp., Tinocallis caryaefoliae, Tomaspis spp., Toxoptera spp., Trialeurodes vaporariorum, Trioza spp., Typhlocyba spp., Unaspis spp., Viteus vitifolii, Zygina spp.; from the order Hymenoptera, for example, Acromyrmex spp., Athalia spp., Atta spp., Diprion spp., Hoplocampa spp., Lasius spp., Monomorium pharaonis, Sirex spp., Solenopsis invicta, Tapinoma spp., Urocerus spp., Vespa spp., or Xeris spp.

In some instances, the insect is from the order Isopoda, for example, Armadillidium vulgare, Oniscus asellus, or Porcellio scaber.

In some instances, the insect is from the order Isoptera, for example, Coptotermes spp., Cornitermes cumulans, Cryptotermes spp., Incisitermes spp., Microtermes obesi, Odontotermes spp., or Reticulitermes spp.

In some instances, the insect is from the order Lepidoptera, for example, Achroia grisella, Acronicta major, Adoxophyes spp., Aedia leucomelas, Agrotis spp., Alabama spp., Amyelois transitella, Anarsia spp., Anticarsia spp., Argyroploce spp., Barathra brassicae, Borbo cinnara, Bucculatrix thurberiella, Bupalus piniarius, Busseola spp., Cacoecia spp., Caloptilia theivora, Capua reticulana, Carpocapsa pomonella, Carposina niponensis, Cheimatobia brumata, Chilo spp., Choristoneura spp., Clysia ambiguella, Cnaphalocerus spp., Cnaphalocrocis medinalis, Cnephasia spp., Conopomorpha spp., Conotrachelus spp., Copitarsia spp., Cydia spp., Dalaca noctuides, Diaphania spp., Diatraea saccharalis, Earias spp., Ecdytolopha aurantium, Elasmopalpus lignosellus, Eldana saccharina, Ephestia spp., Epinotia spp., Epiphyas postvittana, Etiella spp., Eulia spp., Eupoecilia ambiguella, Euproctis spp., Euxoa spp., Feltia spp., Galleria mellonella, Gracillaria spp., Grapholitha spp., Hedylepta spp., Helicoverpa spp., Heliothis spp., Hofmannophila pseudospretella, Homoeosoma spp., Homona spp., Hyponomeuta padella, Kakivoria flavofasciata, Laphygma spp., Laspeyresia molesta, Leucinodes orbonalis, Leucoptera spp., Lithocolletis spp., Lithophane antennata, Lobesia spp., Loxagrotis albicosta, Lymantria spp., Lyonetia spp., Malacosoma neustria, Maruca testulalis, Mamstra brassicae, Melanitis leda, Mocis spp., Monopis obviella, Mythimna separata, Nemapogon cloacellus, Nymphula spp., Oiketicus spp., Oria spp., Orthaga spp., Ostrinia spp., Oulema oryzae, Panolis flammea, Parnara spp., Pectinophora spp., Perileucoptera spp., Phthorimaea spp., Phyllocnistis citrella, Phyllonorycter spp., Pieris spp., Platynota stultana, Plodia interpunctella, Plusia spp., Plutella xylostella, Prays spp., Prodenia spp., Protoparce spp., Pseudaletia spp., Pseudaletia unipuncta, Pseudoplusia includens, Pyrausta nubilalis, Rachiplusia nu, Schoenobius spp., Scirpophaga spp., Scirpophaga innotata, Scotia segetum, Sesamia spp., Sesamia inferens, Sparganothis spp., Spodoptera spp., Spodoptera praefica, Stathmopoda spp., Stomopteryx subsecivella, Synanthedon spp., Tecia solanivora, Thermesia gemmatalis, Tinea cloacella, Tinea pellionella, Tineola bisselliella, Tortrix spp., Trichophaga tapetzella, Trichoplusia spp., Tryporyza incertulas, Tuta absoluta, or Virachola spp.

In some instances, the insect is from the order Orthoptera or Saltatoria, for example, Acheta domesticus, Dichroplus spp., Gryllotalpa spp., Hieroglyphus spp., Locusta spp., Melanoplus spp., or Schistocerca gregaria.

In some instances, the insect is from the order Phthiraptera, for example, Damalinia spp., Haematopinus spp., Linognathus spp., Pediculus spp., Ptirus pubis, Trichodectes spp.

In some instances, the insect is from the order Psocoptera for example Lepinatus spp., or Liposcelis spp.

In some instances, the insect is from the order Siphonaptera, for example, Ceratophyllus spp., Ctenocephalides spp., Pulex irritans, Tunga penetrans, or Xenopsylla cheopsis.

In some instances, the insect is from the order Thysanoptera, for example, Anaphothrips obscurus, Baliothrips biformis, Drepanothrips reuteri, Enneothrips flavens, Frankliniella spp., Heliothrips spp., Hercinothrips femoralis, Rhipiphorothrips cruentatus, Scirtothrips spp., Taeniothrips cardamomi, or Thrips spp.

In some instances, the insect is from the order Zygentoma (=Thysanura), for example, Ctenolepisma spp., Lepisma saccharina, Lepismodes inquilinus, or Thermobia domestica.

In some instances, the insect is from the class Symphyla, for example, Scutigerella spp.

In some instances, the insect is a mite, including but not limited to, Tarsonemid mites, such as Phytonemus pallidus, Polyphagotarsonemus latus, Tarsonemus bilobatus, or the like; Eupodid mites, such as Penthaleus erythrocephalus, Penthaleus major, or the like; Spider mites, such as Oligonychus shinkajii, Panonychus citri, Panonychus mori, Panonychus ulmi, Tetranychus kanzawai, Tetranychus urticae, or the like; Eriophyid mites, such as Acaphylla theavagrans, Aceria tulipae, Aculops lycopersici, Aculops pelekassi, Aculus schlechtendali, Eriophyes chibaensis, Phyllocoptruta oleivora, or the like; Acarid mites, such as Rhizoglyphus robini, Tyrophagus putrescentiae, Tyrophagus similis, or the like; Bee brood mites, such as Varroa jacobsoni, Varroa destructoror the like; Ixodides, such as Boophilus microplus, Rhipicephalus sanguineus, Haemaphysalis longicornis, Haemophysalis flava, Haemophysalis campanulata, Ixodes ovatus, Ixodes persulcatus, Amblyomma spp., Dermacentor spp., or the like; Cheyletidae, such as Cheyletiella yasguri, Cheyletiella blakei, or the like; Demodicidae, such as Demodex canis, Demodex cati, or the like; Psoroptidae, such as Psoroptes ovis, or the like; Scarcoptidae, such as Sarcoptes scabiei, Notoedres cati, Knemidocoptes spp., or the like.

Table 3 shows further examples of insects that cause infestations that can be treated or prevented using the pest control (e.g., biopesticide or biorepellent) compositions and related methods described herein.

TABLE 3
Insect pests

| Common Name | Latin name |
|---|---|
| European corn borer | Ostrinia nubilalis |
| Corn earworm | Helicoverpa zea |
| Beet armyworm | Spodoptera exigua |
| Fall armyworm | Spodoptera frugiperda |
| Southwestern corn borer | Diatraea grandiosella |
| Lesser cornstalk borer | Elasmopalpus lignosellus |
| Stalk borer | Papaipema nebris |
| Common armyworm | Pseudaletia unipuncta |
| Black cutworm | Agrotis ipsilon |
| Western bean cutworm | Striacosta albicosta |
| Yellowstriped armyworm | Spodoptera ornithogalli |
| Western yellowstriped | Spodoptera praefica |
| armyworm | |
| Southern armyworm | Spodoptera eridania |
| Southern armyworm | Spodoptera eridania |
| Variegated cutworm | Peridroma saucia |
| Stalk borer | Papaipema nebris |
| Cabbage looper | Trichoplusia ni |
| Tomato pinworm | Keiferia lycopersicella |
| Tobacco hornworm | Manduca sexta |
| Tomato hornworm | Manduca quinquemaculata |
| Imported cabbageworm | Artogeia rapae |
| Cabbage butterfly | Pieris brassicae |
| Cabbage looper | Trichoplusia ni |
| Diamondback moth | Plutella xylostella |
| Beet armyworm | Spodoptera exigua |
| Common cutworm | Agrotis segetum |
| Potato tuberworm | Phthorimaea operculella |
| Diamondback moth | Plutella xylostella |
| Sugarcane borer | Diatraea saccharalis |
| Glassy cutworm | Crymodes devastator |
| Dingy cutworm | Feltia ducens |
| Claybacked cutworm | Agrotis gladiaria |
| Green cloverworm | Plathypena scabra |
| Soybean looper | Pseudoplusia includes |
| Velvetbean caterpillar | Anticarsia gemmatalis |
| Northern corn rootworm | Coleoptera Diabrotica barberi |
| Southern corn rootworm | Diabrotica undecimpunctata |
| Western corn rootworm | Diabrotica virgifera |
| Maize weevil | Sitophilus zeamais |
| Colorado potato beetle | Leptinotarsa decemlineata |
| Tobacco flea beetle | Epitrix hirtipennis |
| Crucifer flea beetle | Phyllotreta Cruciferae |
| Western black flea beetle | Phyllotreta pusilia |
| Pepper weevil | Anthonomus eugenii |
| Colorado potato beetle | Leptinotarsa decemlineata |
| Potato flea beetle | Epitrix cucumeris |
| Wireworms Melanpotus spp. | Hemicrepidus memnonius |
| Wireworms | Ceutorhychus assimilis |
| Cabbage seedpod weevil | Phyllotreta Cruciferae |
| Crucifer flea beetle | Melanolus spp. |
| Wireworm | Aeolus mellillus |
| Wheat wireworm | Aeolus mancus |
| Sand wireworm | Horistonotus uhlerii |
| Maize billbug | Sphenophorus maidis |
| Timothy bilibug | Sphenophorus zeae |
| Bluegrass billbug | Sphenophorus parvulus |
| Southern corn billbug | Sphenophorus callosus |
| White grubs | Phyllophaga spp. |
| Corn flea beetle | Chaetocnema pulicaria |
| Japanese beetle | Popillia japonica |
| Mexican bean beetle | Epilachna varivestis |
| Bean leaf beetle | Cerotoma trifurcate |
| Blister beetles | Epicauta pestifera Epicauta lemniscata |
| Corn leaf aphid | Homoptera Rhopalosiphum maidis |
| Corn root aphid | Anuraphis maidiradicis |
| Green peach aphid | Myzus persicae |
| Potato aphid | Macrosiphum euphorbiae |
| Greenhouse whitefly | Trileurodes vaporariorum |
| Sweetpotato whitefly | Bemisia tabaci |
| Silverleaf whitefly | Bemisia argentifolii |
| Cabbage aphid | Brevicoryne brassicae |
| Green peach aphid | Myzus persicae |
| Potato leafhopper | Empoasca fabae |
| Potato psyllid | Paratrioza cockerelli |
| Silverleaf whitefly | Bemisia argentifolii |
| Sweetpotato whitefly | Bemisia tabaci |
| Carrot aphid | Cavariella aegopodii |
| Cabbage aphid | Brevicoryne brassicae |
| West Indian canefly | Saccharosydne saccharivora |
| Yellow sugarcane aphid | Sipha flava |
| Threecornered alfalfa hopper | Spissistilus festinus |
| Lygus Hesperus | Hemiptera Lygus lineolaris |
| Lygus bug | Lygus rugulipennis |
| Green stink bug | Acrosternum hilare |
| Brown stick bug | Euschistus servus |
| Chinch bug | Blissus leucopterus leucopterus |
| Leafminer | Diptera Liriomyza trifolii |
| Vegetable leafminer | Liriomyza sativae |
| Tomato leafminer | Scrobipalpula absoluta |
| Seedcorn maggot | Delia platura |
| Cabbage maggot | Delia brassicae |
| Cabbage root fly | Delia radicum |
| Carrot rust fly | Psilia rosae |
| Sugarbeet root maggot | Tetanops myopaeformis |
| Differential grasshopper | Orthoptera Melanoplus differentialis |
| Redlegged grasshopper | Melanoplus femurrubrum |
| Twostriped grasshopper | Melanoplus bivittatus |

D. Mollusks

The pest control (e.g., biopesticide or biorepellent) compositions and related methods can be useful for decreasing the fitness of a mollusk, e.g., to prevent or treat a mollusk infestation in a plant. The term “mollusk” includes any organism belonging to the phylum Mollusca. Included are methods for delivering a pest control (e.g., biopesticide or biorepellent) composition to a mollusk by contacting the mollusk with the pest control (e.g., biopesticide or biorepellent) composition. Additionally or alternatively, the methods include delivering the biopesticide to a plant at risk of or having a mollusk infestation, by contacting the plant with the pest control (e.g., biopesticide or biorepellent) composition.

The pest control (e.g., biopesticide or biorepellent) compositions and related methods are suitable for preventing or treating infestation by terrestrial Gastropods (e.g., slugs and snails) in agriculture and horticulture. They include all terrestrial slugs and snails which mostly occur as polyphagous pests on agricultural and horticultural crops. For example, the mollusk may belong to the family Achatinidae, Agriolimacidae, Ampullariidae, Arionidae, Bradybaenidae, Helicidae, Hydromiidae, Lymnaeidae, Milacidae, Urocyclidae, or Veronicellidae.

For example, in some instances, the mollusk is Achatina spp., Archachatina spp. (e.g., Archachatina marginata), Agriolimax spp., Arion spp. (e.g., A. ater, A. circumscriptus, A. distinctus, A. fasciatus, A. hortensis, A. intermedius, A. rufus, A. subfuscus, A. silvaticus, A. lusitanicus), Arliomax spp. (e.g., Ariolimax columbianus), Biomphalaria spp., Bradybaena spp. (e.g., B. fruticum), Bulinus spp., Cantareus spp. (e.g., C. asperses), Cepaea spp. (e.g., C. hortensis, C. nemoralis, C. hortensis), Cernuella spp., Cochlicella spp., Cochlodina spp. (e.g., C. laminata), Deroceras spp. (e.g., D. agrestis, D. empiricorum, D. laeve, D. panornimatum, D. reticulatum), Discus spp. (e.g., D. rotundatus), Euomphalia spp., Galba spp. (e.g., G. trunculata), Helicella spp. (e.g., H. itala, H. obvia), Helicigona spp. (e.g., H. arbustorum), Helicodiscus spp., Helix spp. (e.g., H. aperta, H. aspersa, H. pomatia), Limax spp. (e.g., L. cinereoniger, L. flavus, L. marginatus, L. maximus, L. tenellus), Limicolaria spp. (e.g., Limicolaria aurora), Lymnaea spp. (e.g., L. stagnalis), Mesodon spp. (e.g., Meson thyroidus), Monadenia spp. (e.g., Monadenia fidelis), Milax spp. (e.g., M. gagates, M. marginatus, M. sowerbyi, M. budapestensis), Oncomelania spp., Neohelix spp. (e.g., Neohelix albolabris), Opeas spp., Otala spp. (e.g., Otala lacteal), Oxyloma spp. (e.g., O. pfeifferi), Pomacea spp. (e.g., P. canaliculata), Succinea spp., Tandonia spp. (e.g., T. budapestensis, T. sowerbyl), Theba spp., Vallonia spp., or Zonitoides spp. (e.g., Z. nitidus).

E. Nematodes

The pest control (e.g., biopesticide or biorepellent) compositions and related methods can be useful for decreasing the fitness of a nematode, e.g., to prevent or treat a nematode infestation in a plant. The term “nematode” includes any organism belonging to the phylum Nematoda. Included are methods for delivering a pest control (e.g., biopesticide or biorepellent) composition to a nematode by contacting the nematode with the pest control (e.g., biopesticide or biorepellent) composition. Additionally or alternatively, the methods include delivering the biopesticide to a plant at risk of or having a nematode infestation, by contacting the plant with the pest control (e.g., biopesticide or biorepellent) composition.

The pest control (e.g., biopesticide or biorepellent) compositions and related methods are suitable for preventing or treating infestation by nematodes that cause damage plants including, for example, Meloidogyne spp. (root-knot), Heterodera spp., Globodera spp., Pratylenchus spp., Helicotylenchus spp., Radopholus similis, Ditylenchus dipsaci, Rotylenchulus reniformis, Xiphinema spp., Aphelenchoides spp. and Belonolaimus longicaudatus. In some instances, the nematode is a plant parasitic nematodes or a nematode living in the soil. Plant parasitic nematodes include, but are not limited to, ectoparasites such as Xiphinema spp., Longidorus spp., and Trichodorus spp.; semiparasites such as Tylenchulus spp.; migratory endoparasites such as Pratylenchus spp., Radopholus spp., and Scutellonema spp.; sedentary parasites such as Heterodera spp., Globodera spp., and Meloidogyne spp., and stem and leaf endoparasites such as Ditylenchus spp., Aphelenchoides spp., and Hirshmaniella spp. Especially harmful root parasitic soil nematodes are such as cystforming nematodes of the genera Heterodera or Globodera, and/or root knot nematodes of the genus Meloidogyne. Harmful species of these genera are for example Meloidogyne incognita, Heterodera glycines (soybean cyst nematode), Globodera pallida and Globodera rostochiensis (potato cyst nematode), which species are effectively controlled with the pest control (e.g., biopesticide or biorepellent) compositions described herein. However, the use of the pest control (e.g., biopesticide or biorepellent) compositions described herein is in no way restricted to these genera or species, but also extends in the same manner to other nematodes.

Other examples of nematodes that can be targeted by the methods and compositions described herein include but are not limited to e.g. Aglenchus agricola, Anguina tritici, Aphelenchoides arachidis, Aphelenchoides fragaria and the stem and leaf endoparasites Aphelenchoides spp. in general, Belonolaimus gracilis, Belonolaimus longicaudatus, Belonolaimus nortoni, Bursaphelenchus cocophilus, Bursaphelenchus eremus, Bursaphelenchus xylophilus, Bursaphelenchus mucronatus, and Bursaphelenchus spp. in general, Cacopaurus pestis, Criconemella curvata, Criconemella onoensis, Criconemella ornata, Criconemella rusium, Criconemella xenoplax (=Mesocriconema xenoplax) and Criconemella spp. in general, Criconemoides femiae, Criconemoides onoense, Criconemoides ornatum and Criconemoides spp. in general, Ditylenchus destructor, Ditylenchus dipsaci, Ditylenchus myceliophagus and the stem and leaf endoparasites Ditylenchus spp. in general, Dolichodorus heterocephalus, Globodera pallida (=Heterodera pallida), Globodera rostochiensis (potato cyst nematode), Globodera solanacearum, Globodera tabacum, Globodera virginia and the sedentary, cyst forming parasites Globodera spp. in general, Helicotylenchus digonicus, Helicotylenchus dihystera, Helicotylenchus erythrine, Helicotylenchus multicinctus, Helicotylenchus nannus, Helicotylenchus pseudorobustus and Helicotylenchus spp. in general, Hemicriconemoides, Hemicycliophora arenaria, Hemicycliophora nudata, Hemicycliophora parvana, Heterodera avenae, Heterodera cruciferae, Heterodera glycines (soybean cyst nematode), Heterodera oryzae, Heterodera schachtii, Heterodera zeae and the sedentary, cyst forming parasites Heterodera spp. in general, Hirschmaniella gracilis, Hirschmaniella oryzae Hirschmaniella spinicaudata and the stem and leaf endoparasites Hirschmaniella spp. in general, Hoplolaimus aegyptii, Hoplolaimus califomicus, Hoplolaimus columbus, Hoplolaimus galeatus, Hoplolaimus indicus, Hoplolaimus magnistylus, Hoplolaimus pararobustus, Longidorus africanus, Longidorus breviannulatus, Longidorus elongatus, Longidorus laevicapitatus, Longidorus vineacola and the ectoparasites Longidorus spp. in general, Meloidogyne acronea, Meloidogyne africana, Meloidogyne arenaria, Meloidogyne arenaria thamesi, Meloidogyne artiella, Meloidogyne chitwoodi, Meloidogyne coffeicola, Meloidogyne ethiopica, Meloidogyne exigua, Meloidogyne fallax, Meloidogyne graminicola, Meloidogyne graminis, Meloidogyne hapla, Meloidogyne incognita, Meloidogyne incognita acrita, Meloidogyne javanica, Meloidogyne kikuyensis, Meloidogyne minor, Meloidogyne naasi, Meloidogyne paranaensis, Meloidogyne thamesi and the sedentary parasites Meloidogyne spp. in general, Meloinema spp., Nacobbus aberrans, Neotylenchus vigissi, Paraphelenchus pseudoparietinus, Paratrichodorus allius, Paratrichodorus lobatus, Paratrichodorus minor, Paratrichodorus nanus, Paratrichodorus porosus, Paratrichodorus teres and Paratrichodorus spp. in general, Paratylenchus hamatus, Paratylenchus minutus, Paratylenchus projectus and Paratylenchus spp. in general, Pratylenchus agilis, Pratylenchus alleni, Pratylenchus andinus, Pratylenchus brachyurus, Pratylenchus cerealis, Pratylenchus coffeae, Pratylenchus crenatus, Pratylenchus delattrei, Pratylenchus giibbicaudatus, Pratylenchus goodeyi, Pratylenchus hamatus, Pratylenchus hexincisus, Pratylenchus loosi, Pratylenchus neglectus, Pratylenchus penetrans, Pratylenchus pratensis, Pratylenchus scribneri, Pratylenchus teres, Pratylenchus thornei, Pratylenchus vulnus, Pratylenchus zeae and the migratory endoparasites Pratylenchus spp. in general, Pseudohalenchus minutus, Psilenchus magnidens, Psilenchus tumidus, Punctodera chalcoensis, Quinisulcius acutus, Radopholus citrophilus, Radopholus similis, the migratory endoparasites Radopholus spp. in general, Rotylenchulus borealis, Rotylenchulus parvus, Rotylenchulus reniformis and Rotylenchulus spp. in general, Rotylenchus laurentinus, Rotylenchus macrodoratus, Rotylenchus robustus, Rotylenchus uniformis and Rotylenchus spp. in general, Scutellonema brachyurum, Scutellonema bradys, Scutellonema clathricaudatum and the migratory endoparasites Scutellonema spp. in general, Subanguina radiciola, Tetylenchus nicotianae, Trichodorus cylindricus, Trichodorus minor, Trichodorus primitivus, Trichodorus proximus, Trichodorus similis, Trichodorus sparsus and the ectoparasites Trichodorus spp. in general, Tylenchorhynchus agri, Tylenchorhynchus brassicae, Tylenchorhynchus clarus, Tylenchorhynchus claytoni, Tylenchorhynchus digitatus, Tylenchorhynchus ebriensis, Tylenchorhynchus maximus, Tylenchorhynchus nudus, Tylenchorhynchus vulgaris and Tylenchorhynchus spp. in general, Tylenchulus semipenetrans and the semiparasites Tylenchulus spp. in general, Xiphinema americanum, Xiphinema brevicolle, Xiphinema dimorphicaudatum, Xiphinema index and the ectoparasites Xiphinema spp. in general.

Other examples of nematode pests include species belonging to the family Criconematidae, Belonolaimidae, Hoploaimidae, Heteroderidae, Longidoridae, Pratylenchidae, Trichodoridae, or Anguinidae.

Table 4 shows further examples of nematodes, and diseases associated therewith, that can be treated or prevented using the pest control (e.g., biopesticide or biorepellent) compositions and related methods described herein.

TABLE 4
Nematode Pests

| Disease | Causative Agent |
|---|---|
| Awl | Dolichoderus spp., D. heterocephalus |
| Bulb and stem (Europe) | Ditylenchus dipsaci |
| Burrowing | Radopholus similes R. similis |
| Cyst | Heterodera avenae, H. zeae, |
| | H. schachti; Globodera |
| | rostochiensis, G. pallida, and |
| | G. tabacum; Heterodera trifolii, |
| | H. medicaginis, H. ciceri, |
| | H. mediterranea, H. cyperi, |
| | H. salixophila, H. zeae, H. goettingiana, |
| | H. riparia, H. humuli, H. latipons, |
| | H. sorghi, H. fici, H. litoralis, and |
| | H. turcomanica; Punctodera chalcoensis |
| Dagger | Xiphinema spp., X. americanum, |
| | X. Mediterraneum |
| False root-knot | Nacobbus dorsalis |
| Lance | Hoplolaimus spp., H. galeatus |
| Lance, Columbia | Hoplolaimus Columbus |
| Lesion | Pratylenchus spp., P. brachyurus, |
| | P. coffeae P. crenatus, P. hexincisus, |
| | P. neglectus, P. penetrans, P. scribneri, |
| | P. magnica, P. neglectus, P. thornei, |
| | P. vulnus, P. zeae |
| Needle | Longidorus spp., L. breviannulatus |
| Others | Hirschmanniella species, Pratylenchoid |
| | magnicauda |
| Ring | Criconemella spp., C. ornata |
| Root-knot | Meloidogyne spp., M. arenaria, M. chitwoodi, |
| | M. artiellia, M. fallax, M. hapla, |
| | M. javanica, M. incognita, M. microtyla, |
| | M. partityla, M. panyuensis, |
| | M, paranaensis |
| Spiral | Helicotylenchus spp. |
| Sting | Belonolaimus spp., B. longicaudatus |
| Stubby-root | Paratrichodorus spp., P. christiei, |
| | P. minor, Quinisulcius acutus, |
| | Trichodorus spp. |
| Stunt | Tylenchorhynchus dubius |

F. Viruses

The pest control (e.g., biopesticide or biorepellent) compositions and related methods can be useful for decreasing the fitness of a virus, e.g., to prevent or treat a viral infection in a plant. Included are methods for delivering a pest control (e.g., biopesticide or biorepellent) composition to a virus by contacting the virus with the pest control (e.g., biopesticide or biorepellent) composition. Additionally or alternatively, the methods include delivering the pest control (e.g., biopesticide or biorepellent) composition to a plant at risk of or having a viral infection, by contacting the plant with the pest control (e.g., biopesticide or biorepellent) composition.

The pest control (e.g., biopesticide or biorepellent) compositions and related methods are suitable for delivery to a virus that causes viral diseases in plants, including the viruses and diseases listed in Table 5.

TABLE 5
Viral Plant Pathogens

| Disease | Causative Agent |
|---|---|
| Alfamoviruses: | Alfalfa mosaic alfamovirus |
| Bromoviridae | |
| Alphacryptoviruses: | Alfalfa 1 alphacryptovirus, Beet 1 alphacryptovirus, Beet 2 |
| Partitiviridae | alphacryptovirus, Beet 3 alphacryptovirus, Carnation 1 |
| | alphacryptovirus, Carrot temperate 1 alphacryptovirus, Carrot |
| | temperate 3 alphacryptovirus, Carrot temperate 4 alphacryptovirus, |
| | Cocksfoot alphacryptovirus, Hop trefoil 1 alphacryptovirus, Hop |
| | trefoil 3 alphacryptovirus, Radish yellow edge alphacryptovirus, |
| | Ryegrass alphacryptovirus, Spinach temperate alphacryptovirus, |
| | Vicia alphacryptovirus, White clover 1 alphacryptovirus, White |
| | clover 3 alphacryptovirus |
| Badnaviruses | Banana streak badnavirus, Cacao swollen shoot badnavirus, Canna |
| | yellow mottle badnavirus, Commelina yellow mottle badnavirus, |
| | Dioscorea bacilliform badnavirus, Kalanchoe top-spotting |
| | badnavirus, Rice tungro bacilliform badnavirus, Schefflera ringspot |
| | badnavirus, Sugarcane bacilliform badnavirus |
| Betacryptoviruses: | Carrot temperate 2 betacryptovirus, Hop trefoil 2 betacryptovirus, |
| Partitiviridae | Red clover 2 betacryptovirus, White clover 2 betacryptovirus |
| Bigeminiviruses: | Abutilon mosaic bigeminivirus, Ageratum yellow vein |
| Geminiviridae | bigeminivirus, Bean calico mosaic bigeminivirus, Bean golden |
| | mosaic bigeminivirus, Bhendi yellow vein mosaic bigeminivirus, |
| | Cassava African mosaic bigeminivirus, Cassava Indian mosaic |
| | bigeminivirus, Chino del tomate bigeminivirus, Cotton leaf crumple |
| | bigeminivirus, Cotton leaf curl bigeminivirus, Croton yellow vein |
| | mosaic bigeminivirus, Dolichos yellow mosaic bigeminivirus, |
| | Euphorbia mosaic bigeminivirus, Horsegram yellow mosaic |
| | bigeminivirus, Jatropha mosaic bigeminivirus, Lima bean golden |
| | mosaic bigeminivirus, Melon leaf curl bigeminivirus, Mung bean |
| | yellow mosaic bigeminivirus, Okra leaf-curl bigeminivirus, Pepper |
| | hausteco bigeminivirus, Pepper Texas bigeminivirus, Potato yellow |
| | mosaic bigeminivirus, Rhynchosia mosaic bigeminivirus, Serrano |
| | golden mosaic bigeminivirus, Squash leaf curl bigeminivirus, |
| | Tobacco leaf curl bigeminivirus, Tomato Australian leafcurl |
| | bigeminivirus, Tomato golden mosaic bigeminivirus, Tomato |
| | Indian leafcurl bigeminivirus, Tomato leaf crumple bigeminivirus, |
| | Tomato mottle bigeminivirus, Tomato yellow leaf curl |
| | bigeminivirus, Tomato yellow mosaic bigeminivirus, Watermelon |
| | chlorotic stunt bigeminivirus, Watermelon curly mottle |
| | Bigeminivirus |
| Bromoviruses: | Broad bean mottle bromovirus, Brome mosaic bromovirus, Cassia |
| Bromoviridae | yellow blotch bromovirus, Cowpea chlorotic mottle bromovirus, |
| | Melandrium yellow fleck bromovirus, Spring beauty latent |
| | Bromovirus |
| Bymoviruses: | Barley mild mosaic bymovirus, Barley yellow mosaic bymovirus, |
| Potyviridae | Oat mosaic bymovirus, Rice necrosis mosaic bymovirus, Wheat |
| | spindle streak mosaic bymovirus, Wheat yellow mosaic bymovirus |
| Capilloviruses | Apple stem grooving capillovirus, Cherry A capillovirus, Citrus |
| | tatter leaf capillovirus, Lilac chlorotic leafspot capillovirus |
| Carlaviruses | Blueberry scorch carlavirus, Cactus 2 carlavirus, Caper latent |
| | carlavirus, Carnation latent carlavirus, Chrysanthemum B |
| | carlavirus, Dandelion latent carlavirus, Elderberry carlavirus, Fig S |
| | carlavirus, Helenium S carlavirus, Honeysuckle latent carlavirus, |
| | Hop American latent carlavirus, Hop latent carlavirus, Hop mosaic |
| | carlavirus, Kalanchoe latent carlavirus, Lilac mottle carlavirus, Lily |
| | symptomless carlavirus, Mulberry latent carlavirus, Muskmelon |
| | vein necrosis carlavirus, Nerine latent carlavirus, Passiflora latent |
| | carlavirus, Pea streak carlavirus, Poplar mosaic carlavirus, Potato M |
| | carlavirus, Potato S carlavirus, Red clover vein mosaic carlavirus, |
| | Shallot latent carlavirus, Strawberry pseudo mild yellow edge |
| | Carlavirus |
| Carmoviruses: | Bean mild mosaic carmovirus, Cardamine chlorotic fleck |
| Tombusviridae | carmovirus, Carnation mottle carmovirus, Cucumber leaf spot |
| | carmovirus, Cucumber soil-borne carmovirus, Galinsoga mosaic |
| | carmovirus, Hibiscus chlorotic ringspot carmovirus, Melon necrotic |
| | spot carmovirus, Pelargonium flower break carmovirus, Turnip |
| | crinkle carmovirus |
| Caulimoviruses | Blueberry red ringspot caulimovirus, Carnation etched ring |
| | caulimovirus, Cauliflower mosaic caulimovirus, Dahlia mosaic |
| | caulimovirus, Figwort mosaic caulimovirus, Horseradish latent |
| | caulimovirus, Mirabilis mosaic caulimovirus, Peanut chlorotic |
| | streak caulimovirus, Soybean chlorotic mottle caulimovirus, Sweet |
| | potato caulimovirus, Thistle mottle caulimovirus |
| Closteroviruses | Beet yellow stunt closterovirus, Beet yellows closterovirus, Broad |
| | bean severe chlorosis closterovirus, Burdock yellows closterovirus, |
| | Carnation necrotic fleck closterovirus, Citrus tristeza closterovirus, |
| | Clover yellows closterovirus, Grapevine stem pitting associated |
| | closterovirus, Wheat yellow leaf closterovirus |
| Comoviruses: | Bean pod mottle comovirus, Bean rugose mosaic comovirus, Broad |
| Comoviridae | bean stain comovirus, Broad bean true mosaic comovirus, Cowpea |
| | mosaic comovirus, Cowpea severe mosaic comovirus, Glycine |
| | mosaic comovirus, Pea mild mosaic comovirus, Potato Andean |
| | mottle comovirus, Quail pea mosaic comovirus, Radish mosaic |
| | comovirus, Red clover mottle comovirus, Squash mosaic |
| | comovirus, Ullucus C comovirus |
| Cucumoviruses: | Cucumber mosaic cucuamovirus, Peanut stunt cucumovirus, Tomato |
| Bromoviridae | aspermy cucumovirus |
| Cytorhabdoviruses: | Barley yellow striate mosaic cytorhabdovirus, Broad bean yellow |
| Rhabdoviridae | vein cytorhabdovirus, Broccoli necrotic yellows cytorhabdovirus, |
| | Cereal northern mosaic cytorhabdovirus, Festuca leaf streak |
| | cytorhabdovirus, Lettuce necrotic yellows cytorhabdovirus, |
| | Sonchus cytorhabdovirus, Strawberry crinkle cytorhabdovirus |
| Dianthoviruses | Carnation ringspot dianthovirus, Red clover necrotic mosaic |
| | dianthovirus, Sweet clover necrotic mosaic dianthovirus |
| Enamoviruses | Pea enation mosaic enamovirus |
| Fijiviruses: | Maize rough dwarf fijivirus, Oat sterile dwarf fijivirus, Pangola |
| Reoviridae | stunt fijivirus, Rice black-streaked dwarf fijivirus, Sugarcane Fiji |
| | disease fijivirus |
| Furoviruses | Beet necrotic yellow vein furovirus, Beet soil-borne furovirus, |
| | Broad bean necrosis furovirus, Oat golden stripe furovirus, Peanut |
| | clump furovirus, Potato mop-top furovirus, Sorghum chlorotic spot |
| | furovirus, Wheat soil-borne mosaic furovirus |
| Hordeiviruses | Anthoxanthum latent blanching hordeivirus, Barley stripe mosaic |
| | hordeivirus, Lychnis ringspot hordeivirus, Poa semilatent |
| | Hordeivirus |
| Hybrigeminiviruses: | Beet curly top hybrigeminivirus, Tomato pseudo curly top |
| Geminiviridae | Hybrigeminivirus |
| Idaeoviruses | Raspberry bushy dwarf idaeovirus |
| llarviruses: | Apple mosaic ilarvirus, Asparagus 2 ilarvirus, Blueberry necrotic |
| Bromoviridae | shock ilarvirus, Citrus leaf rugose ilarvirus, Citrus variegation |
| | ilarvirus, Elm mottle ilarvirus, Humulus japonicus ilarvirus, |
| | Hydrangea mosaic ilarvirus, Lilac ring mottle ilarvirus, Parietaria |
| | mottle ilarvirus, Plum American line pattern ilarvirus, Prune dwarf |
| | ilarvirus, Prunus necrotic ringspot ilarvirus, Spinach latent ilarvirus, |
| | Tobacco streak ilarvirus, Tulare apple mosaic ilarvirus |
| Ipomoviruses: | Sweet potato mild mottle ipomovirus, Sweet potato yellow dwarf |
| Potyviridae | Ipomovirus |
| Luteoviruses | Barley yellow dwarf luteovirus, Bean leaf roll luteovirus, Beet mild |
| | yellowing luteovirus, Beet western yellows luteovirus, Carrot red |
| | leaf luteovirus, Groundnut rosette assistor luteovirus, Potato leafroll |
| | luteovirus, Solanum yellows luteovirus, Soybean dwarf luteovirus, |
| | Soybean Indonesian dwarf luteovirus, Strawberry mild yellow edge |
| | luteovirus, Subterranean clover red leaf luteovirus, Tobacco |
| | necrotic dwarf luteovirus |
| Machlomoviruses | Maize chlorotic mottle machlomovirus |
| Macluraviruses | Madura mosaic macluravirus, Narcissus latent macluravirus |
| Marafiviruses | Bermuda grass etched-line marafivirus, Maize rayado fino |
| | marafivirus, Oat blue dwarf marafivirus |
| Monogeminiviruses: | Chloris striate mosaic monogeminivirus, Digitaria striate mosaic |
| Geminiviridae | monogeminivirus, Digitaria streak monogeminivirus, Maize streak |
| | monogeminivirus, Miscanthus streak monogeminivirus, Panicum |
| | streak monogeminivirus, Paspalum striate mosaic |
| | monogeminivirus, Sugarcane streak monogeminivirus, Tobacco |
| | yellow dwarf monogeminivirus, Wheat dwarf monogeminivirus |
| Nanaviruses | Banana bunchy top nanavirus, Coconut foliar decay nanavirus, |
| | Faba bean necrotic yellows nanavirus, Milk vetch dwarf nanavirus, |
| | Subterranean clover stunt nanavirus |
| Necroviruses | Tobacco necrosis necrovirus, Carnation yellow stripe necrovirus, |
| | Lisianthus necrosis necrovirus |
| Nepoviruses: | Arabis mosaic nepovirus, Arracacha A nepovirus, Artichoke Italian |
| Comoviridae | latent nepovirus, Artichoke yellow ringspot nepovirus, Blueberry |
| | leaf mottle nepovirus, Cacao necrosis nepovirus, Cassava green |
| | mottle nepovirus, Cherry leaf roll nepovirus, Cherry rasp leaf |
| | nepovirus, Chicory yellow mottle nepovirus, Crimson clover latent |
| | nepovirus, Cycas necrotic stunt nepovirus, Grapevine Bulgarian |
| | latent nepovirus, Grapevine chrome mosaic nepovirus, Grapevine |
| | fanleaf nepovirus, Hibiscus latent ringspot nepovirus, Lucerne |
| | Australian latent nepovirus, Mulberry ringspot nepovirus, |
| | Myrobalan latent ringspot nepovirus, Olive latent ringspot |
| | nepovirus, Peach rosette mosaic nepovirus, Potato black ringspot |
| | nepovirus, Potato U nepovirus, Raspberry ringspot nepovirus, |
| | Tobacco ringspot nepovirus, Tomato black ring nepovirus, Tomato |
| | ringspot nepovirus |
| Nucleorhabdoviruses: | Carrot latent nucleorhabdovirus, Coriander feathery red vein |
| Rhabdoviridae | nucleorhabdovirus, Cow parsnip mosaic nucleorhabdovirus, |
| | Cynodon chlorotic streak nucleorhabdovirus, Datura yellow vein |
| | nucleorhabdovirus, Eggplant mottled dwarf nucleorhabdovirus, |
| | Maize mosaic nucleorhabdovirus, Pittosporum vein yellowing |
| | nucleorhabdovirus, Potato yellow dwarf nucleorhabdovirus, |
| | Sonchus yellow net nucleorhabdovirus, Sowthistle yellow vein |
| | nucleorhabdovirus, Tomato vein clearing nucleorhabdovirus, Wheat |
| | American striate mosaic nucleorhabdovirus |
| Oryzaviruses: | Echinochloa ragged stunt oryzavirus, Rice ragged stunt oryzavirus |
| Reoviridae | |
| Ourmiaviruses | Cassava Ivorian bacilliform ourmiavirus, Epirus cherry |
| | ourmiavirus, Melon Ourmia ourmiavirus, Pelargonium zonate spot |
| | Ourmiavirus |
| Phytoreoviruses: | Clover wound tumor phytoreovirus, Rice dwarf phytoreovirus, Rice |
| Reoviridae | gall dwarf phytoreovirus, Rice bunchy stunt phytoreovirus, Sweet |
| | potato phytoreovirus |
| Potexviruses | Asparagus 3 potexvirus, Cactus × potexvirus, Cassava × |
| | potexvirus, Chicory × potexvirus, Clover yellow mosaic |
| | potexvirus, Commelina × potexvirus, Cymbidium mosaic |
| | potexvirus, Daphne × potexvirus, Foxtail mosaic potexvirus, |
| | Hydrangea ringspot potexvirus, Lily × potexvirus, Narcissus |
| | mosaic potexvirus, Nerine × potexvirus, Papaya mosaic potexvirus, |
| | Pepino mosaic potexvirus, Plantago asiatica mosaic potexvirus, |
| | Plantain × potexvirus, Potato aucuba mosaic potexvirus, Potato × |
| | potexvirus, Tulip × potexvirus, Viola mottle potexvirus, White |
| | clover mosaic potexvirus |
| Potyviruses: | Alstroemeria mosaic potyvirus, Amaranthus leaf mottle potyvirus, |
| Potyviridae | Araujia mosaic potyvirus, Arracacha Y potyvirus, Artichoke latent |
| | potyvirus, Asparagus 1 potyvirus, Banana bract mosaic potyvirus, |
| | Bean common mosaic necrosis potyvirus, Bean common mosaic |
| | potyvirus, Bean yellow mosaic potyvirus, Beet mosaic potyvirus, |
| | Bidens mosaic potyvirus, Bidens mottle potyvirus, Cardamom |
| | mosaic potyvirus, Carnation vein mottle potyvirus, Carrot thin leaf |
| | potyyirus, Cassava brown streak potyvirus, Cassia yellow spot |
| | potyvirus, Celery mosaic potyvirus, Chickpea bushy dwarf |
| | potyvirus, Chickpea distortion mosaic potyvirus, Clover yellow |
| | vein potyvirus, Commelina diffusa potyvirus, Commelina mosaic |
| | potyvirus, Cowpea green vein-banding potyvirus, Cowpea |
| | Moroccan aphid-borne mosaic potyvirus, Cowpea rugose mosaic |
| | potyvirus, Crinum mosaic potyvirus, Daphne Y potyvirus, Dasheen |
| | mosaic potyvirus, Datura Colombian potyvirus, Datura distortion |
| | mosaic potyvirus, Datura necrosis potyvirus, Datura shoestring |
| | potyvirus, Dendrobium mosaic potyvirus, Desmodium mosaic |
| | potyvirus, Dioscorea alata potyvirus, Dioscorea green banding |
| | mosaic potyvirus, Eggplant green mosaic potyvirus, Euphorbia |
| | ringspot potyvirus, Freesia mosaic potyvirus, Groundnut eyespot |
| | potyvirus, Guar symptomless potyvirus, Guinea grass mosaic |
| | potyvirus, Helenium Y potyvirus, Henbane mosaic potyvirus, |
| | Hippeastrum mosaic potyvirus, Hyacinth mosaic potyvirus, Iris |
| | fulva mosaic potyvirus, Iris mild mosaic potyvirus, Iris severe |
| | mosaic potyvirus, Johnsongrass mosaic potyvirus, Kennedya Y |
| | potyvirus, Leek yellow stripe potyvirus, Lettuce mosaic potyvirus, |
| | Lily mottle potyvirus, Maize dwarf mosaic potyvirus, Malva vein |
| | clearing potyvirus, Marigold mottle potyvirus, Narcissus yellow |
| | stripe potyvirus, Nerine potyvirus, Onion yellow dwarf potyvirus, |
| | Ornithogalum mosaic potyvirus, Papaya ringspot potyvirus, Parsnip |
| | mosaic potyvirus, Passiflora ringspot potyvirus, Passiflora South |
| | African potyvirus, Passionfruit woodiness potyvirus, Patchouli |
| | mosaic potyvirus, Pea mosaic potyvirus, Pea seed-borne mosaic |
| | potyvirus, Peanut green mosaic potyvirus, Peanut mottle potyvirus, |
| | Pepper Indian mottle potyvirus, Pepper mottle potyvirus, Pepper |
| | severe mosaic potyvirus, Pepper veinal mottle potyvirus, Plum pox |
| | potyvirus, Pokeweed mosaic potyvirus, Potato A potyvirus, Potato |
| | V potyvirus, Potato Y potyvirus, Primula mosaic potyvirus, |
| | Ranunculus mottle potyvirus, Sorghum mosaic potyvirus, Soybean |
| | mosaic potyvirus, Statice Y potyvirus, Sugarcane mosaic potyvirus, |
| | Sweet potato feathery mottle potyvirus, Sweet potato G potyvirus, |
| | Swordbean distortion mosaic potyvirus, Tamarillo mosaic |
| | potyvirus, Telfairia mosaic potyvirus, Tobacco etch potyvirus, |
| | Tobacco vein-banding mosaic potyvirus, Tobacco vein mottling |
| | potyvirus, Tobacco wilt potyvirus, Tomato Peru potyvirus, |
| | Tradescantia-Zebrina potyvirus, Tropaeolum 1 potyvirus, |
| | Tropaeolum 2 potyvirus, Tuberose potyvirus, Tulip band-breaking |
| | potyvirus, Tulip breaking potyvirus, Tulip chlorotic blotch |
| | potyvirus, Turnip mosaic potyvirus, Ullucus mosaic potyvirus, |
| | Vallota mosaic potyvirus, Vanilla mosaic potyvirus, Vanilla |
| | necrosis potyvirus, Voandzeia distortion mosaic potyvirus, |
| | Watermelon mosaic 1 potyvirus, Watermelon mosaic 2 potyvirus, |
| | Wild potato mosaic potyvirus, Wisteria vein mosaic potyvirus, Yam |
| | mosaic potyvirus, Zucchini yellow fleck potyvirus, Zucchini yellow |
| | mosaic potyvirus |
| Rymoviruses: | Hordeum mosaic rymovirus, Oat necrotic mottle |
| Potyviridae | |
| Agropyron mosaic | |
| rymovirus | rymovirus, Ryegrass mosaic rymovirus, Wheat streak mosaic |
| | Rymovirus |
| Satellite RNAs | Arabis mosaic satellite RNA, Chicory yellow mottle satellite RNA, |
| | Cucumber mosaic satellite RNA, Grapevine fanleaf satellite RNA, |
| | Strawberry latent ringspot satellite RNA, Tobacco ringspot satellite |
| | RNA, Tomato black ring satellite RNA, Velvet tobacco mottle |
| | satellite RNA |
| Satelliviruses | Maize white line mosaic satellivirus, Panicum mosaic satellivirus, |
| | Tobacco mosaic satellivirus, Tobacco necrosis satellivirus |
| Sequiviruses: | Dandelion yellow mosaic sequivirus, Parsnip yellow fleck |
| Sequiviridae | Sequivirus |
| Sobemoviruses | Bean southern mosaic sobemovirus, Blueberry shoestring |
| | sobemovirus, Cocksfoot mottle sobemovirus, Lucerne transient |
| | streak sobemovirus, Rice yellow mottle sobemovirus, Rottboellia |
| | yellow mottle sobemovirus, Solanum nodiflorum mottle |
| | sobemovirus, Sowbane mosaic sobemovirus, Subterranean clover |
| | mottle sobemovirus, Turnip rosette sobemovirus, Velvet tobacco |
| | mottle, sobemovirus |
| Tenuiviruses | Maize stripe tenuivirus, Rice grassy stunt tenuivirus, Rice hoja |
| | blanca tenuivirus, Rice stripe tenuivirus |
| Tobamoviruses | Cucumber green mottle mosaic tobamovirus, Frangipani mosaic |
| | tobamovirus, Kyuri green mottle mosaic tobamovirus, |
| | Odontoglossum ringspot tobamovirus, Paprika mild mottle |
| | tobamovirus, Pepper mild mottle tobamovirus, Ribgrass mosaic |
| | tobamovirus, Opuntia Sammons' tobamovirus, Sunn-hemp mosaic |
| | tobamovirus, Tobacco mild green mosaic tobamovirus, Tobacco |
| | mosaic tobamovirus, Tomato mosaic tobamovirus, Ullucus mild |
| | mottle tobamovirus |
| Tobraviruses | Pea early browning tobravirus, Pepper ringspot tobravirus, Tobacco |
| | rattle tobravirus |
| Tombusviruses: | Artichoke mottled crinkle tombusvirus, Carnation Italian ringspot |
| Tombusviridae | tombusvirus, Cucumber necrosis tombusvirus, Cymbidium ringspot |
| | tombusvirus, Eggplant mottled crinkle tombusvirus, Grapevine |
| | Algerian latent tombusvirus, Lato River tombusvirus, Neckar River |
| | tombusvirus, Pelargonium leaf curl tombusvirus, Pepper Moroccan |
| | tombusvirus, Petunia asteroid mosaic tombusvirus, Tomato bushy |
| | stunt tombusvirus |
| Tospoviruses: | Impatiens necrotic spot tospovirus, Peanut yellow spot tospovirus, |
| Bunyaviridae | Tomato spotted wilt tospovirus |
| Trichoviruses | Apple chlorotic leaf spot trichovirus, Heracleum latent trichovirus, |
| | Potato T trichovirus |
| Tymoviruses | Abelia latent tymovirus, Belladonna mottle tymovirus, Cacao |
| | yellow mosaic tymovirus, Clitoria yellow vein tymovirus, |
| | Desmodium yellow mottle tymovirus, Dulcamara mottle tymovirus, |
| | Eggplant mosaic tymovirus, Erysimum latent tymovirus, Kennedya |
| | yellow mosaic tymovirus, Melon rugose mosaic tymovirus, Okra |
| | mosaic tymovirus, Ononis yellow mosaic tymovirus, Passionfruit |
| | yellow mosaic tymovirus, Physalis mosaic tymovirus, Plantago |
| | mottle tymovirus, Potato Andean latent tymovirus, Scrophularia |
| | mottle tymovirus, Turnip yellow mosaic, tymovirus, Voandzeia |
| | necrotic mosaic tymovirus, Wild cucumber mosaic tymovirus |
| Umbraviruses | Bean yellow vein banding umbravirus, Carrot mottle mimic |
| | umbravirus, Carrot mottle umbravirus, Carrot mottle mimic |
| | umbravirus, Groundnut rosette umbravirus, Lettuce speckles mottle |
| | umbravirus, Tobacco mottle umbravirus |
| Varicosaviruses | Freesia leaf necrosis varicosavirus, Lettuce big-vein varicosavirus, |
| | Tobacco stunt varicosavirus |
| Waikaviruses: | Anthriscus yellows waikavirus, Maize chlorotic dwarf waikavirus, |
| Sequiviridae | Rice tungro spherical waikavirus |
| Putative | Alsike clover vein mosaic virus, Alstroemeria streak potyvirus, |
| Ungrouped | Amaranthus mosaic potyvirus, Amazon lily mosaic potyvirus, |
| Viruses | Anthoxanthum mosaic potyvirus, Apple stem pitting virus, |
| | Aquilegia potyvirus, Asclepias rhabdovirus, Atropa belladonna |
| | rhabdovirus, Barley mosaic virus, Barley yellow streak mosaic |
| | virus, Beet distortion mosaic virus, Beet leaf curl rhabdovirus, Beet |
| | western yellows ST9-associated RNA virus, Black raspberry |
| | necrosis virus, Bramble yellow mosaic potyvirus, Brinjal mild |
| | mosaic potyvirus, Broad bean B virus, Broad bean V potyvirus, |
| | Broad bean yellow ringspot virus, Bryonia mottle potyvirus, |
| | Burdock mosaic virus, Burdock mottle virus, Callistephus chinensis |
| | chlorosis rhabdovirus, Canary reed mosaic potyvirus, Canavalia |
| | maritima mosaic potyvirus, Carnation rhabdovirus, Carrot mosaic |
| | potyvirus, Cassava symptomless rhabdovirus, Cassia mosaic virus, |
| | Cassia ringspot virus, Celery yellow mosaic potyvirus, Celery |
| | yellow net virus, Cereal flame chlorosis virus, Chickpea filiform |
| | potyvirus, Chilli veinal mottle potyvirus, Chrysanthemum spot |
| | potyvirus, Chrysanthemum vein chlorosis rhabdovirus, Citrus |
| | leprosis rhabdovirus, Citrus ringspot virus, Clover mild mosaic |
| | virus, Cocksfoot streak potyvirus, Colocasia bobone disease |
| | rhabdovirus, Cucumber toad-skin rhabdovirus, Cucumber vein |
| | yellowing virus, Cypripedium calceolus potyvirus, Datura innoxia |
| | Hungarian mosaic potyvirus, Dioscorea trifida potyvirus, Dock |
| | mottling mosaic potyvirus, Dodonaea yellows-associated virus, |
| | Eggplant severe mottle potyvirus, Euonymus fasciation |
| | rhabdovirus, Euonymus rhabdovirus, Fern potyvirus, Fig potyvirus, |
| | Gerbera symptomless rhabdovirus, Grapevine fleck virus, |
| | Grapevine stunt virus, Guar top necrosis virus, Habenaria mosaic |
| | potyvirus, Holcus lanatus yellowing rhabdovirus, Holcus streak |
| | potyvirus, Iris germanica leaf stripe rhabdovirus, Iris Japanese |
| | necrotic ring virus, Isachne mosaic potyvirus, Kalanchoe isometric |
| | virus, Kenaf vein-clearing rhabdovirus, Launaea mosaic potyvirus, |
| | Lupin yellow vein rhabdovirus, Maize eyespot virus, Maize line |
| | virus, Maize mottle/chlorotic stunt virus, Maize white line mosaic |
| | virus, Malvastrum mottle virus, Melilotus mosaic potyvirus, Melon |
| | vein-banding mosaic potyvirus, Melothria mottle potyvirus, |
| | Mimosa mosaic virus, Mung bean mottle potyvirus, Narcissus |
| | degeneration potyvirus, Narcissus late season yellows potyvirus, |
| | Nerine Y potyvirus, Nothoscordum mosaic potyvirus, Oak ringspot |
| | virus, Orchid fleck rhabdovirus, Palm mosaic potyvirus, Parsley |
| | green mottle potyvirus, Parsley rhabdovirus, Parsnip leafcurl virus, |
| | Passionfruit Sri Lankan mottle potyvirus, Passionfruit vein-clearing |
| | rhabdovirus, Patchouli mottle rhabdovirus, Pea stem necrosis virus, |
| | Peanut top paralysis potyvirus, Peanut veinal chlorosis rhabdovirus, |
| | Pecteilis mosaic potyvirus, Pepper mild mosaic potyvirus, Perilla |
| | mottle potyvirus, Pigeonpea proliferation rhabdovirus, Pigeonpea |
| | sterility mosaic virus, Plantain 7 potyvirus, Plantain mottle |
| | rhabdovirus, Pleioblastus chino potyvirus, Poplar decline potyvirus, |
| | Primula mottle potyvirus, Purple granadilla mosaic virus, |
| | Ranunculus repens symptomless rhabdovirus, Rice yellow stunt |
| | virus, Saintpaulia leaf necrosis rhabdovirus, Sambucus vein |
| | clearing rhabdovirus, Sarracenia purpurea rhabdovirus, Shamrock |
| | chlorotic ringspot potyvirus, Soybean mild mosaic virus, Soybean |
| | rhabdovirus, Soybean spherical virus, Soybean yellow vein virus, |
| | Soybean Z potyvirus, Strawberry latent C rhabdovirus, Strawberry |
| | mottle virus, Strawberry pallidosis virus, Sunflower mosaic |
| | potyvirus, Sweet potato latent potyvirus, Teasel mosaic potyvirus, |
| | Thimbleberry ringspot virus, Tomato mild mottle potyvirus, |
| | Trichosanthes mottle potyvirus, Tulip halo necrosis virus, Tulip |
| | mosaic virus, Turnip vein-clearing virus, Urd bean leaf crinkle |
| | virus, Vigna sinensis mosaic rhabdovirus, Watercress yellow spot |
| | virus, Watermelon Moroccan mosaic potyvirus, Wheat chlorotic |
| | spot rhabdovirus, White bryony potyvirus, Wineberry latent virus, |
| | Zinnia mild mottle potyvirus, Zoysia mosaic potyvirus |

G. Weeds

As used herein, the term “weed” refers to a plant that grows where it is not wanted. Such plants are typically invasive and, at times, harmful, or have the risk of becoming so. Weeds may be treated with the present pest control (e.g., biopesticide or biorepellent) compositions to reduce or eliminate the presence, viability, or reproduction of the plant. For example, and without being limited thereto, the methods can be used to target weeds known to damage plants. For example, and without being limited thereto, the weeds can be any member of the following group of families: Gramineae, Umbelliferae, Papilionaceae, Cruciferae, Malvaceae, Eufhorbiaceae, Compositae, Chenopodiaceae, Fumariaceae, Charyophyllaceae, Primulaceae, Geraniaceae, Polygonaceae, Juncaceae, Cyperaceae, Aizoaceae, Asteraceae, Convolvulaceae, Cucurbitaceae, Euphorbiaceae, Polygonaceae, Portulaceae, Solanaceae, Rosaceae, Simaroubaceae, Lardizabalaceae, Liliaceae, Amaranthaceae, Vitaceae, Fabaceae, Primulaceae, Apocynaceae, Araliaceae, Caryophyllaceae, Asclepiadaceae, Celastraceae, Papaveraceae, Onagraceae, Ranunculaceae, Lamiaceae, Commelinaceae, Scrophulariaceae, Dipsacaceae, Boraginaceae, Equisetaceae, Geraniaceae, Rubiaceae, Cannabaceae, Hyperiacaceae, Balsaminaceae, Lobeliaceae, Caprifoliaceae, Nyctaginaceae, Oxalidaceae, Vitaceae, Urticaceae, Polypodiaceae, Anacardiaceae, Smilacaceae, Araceae, Campanulaceae, Typhaceae, Valerianaceae, Verbenaceae, Violaceae. For example, and without being limited thereto, the weeds can be any member of the group consisting of Lolium Rigidum, Amaramthus palmeri, Abutilon theopratsi, Sorghum halepense, Conyza Canadensis, Setaria verticillata, Capsella pastoris, and Cyperus rotundas. Additional weeds include, for example, Mimosapigra, salvinia, hyptis, senna, noogoora, burr, Jatropha gossypifolia, Parkinsonia aculeate, Chromolaena odorata, Cryptoslegia grandiflora, or Andropogon gayanus. Weeds can include monocotyledonous plants (e.g., Agrostis, Alopecurus, Avena, Bromus, Cyperus, Digitaria, Echinochloa, Lolium, Monochoria, Rottboellia, Sagittaria, Scirpus, Setaria, Sida or Sorghum) or dicotyledonous plants (Abutilon, Amaranthus, Chenopodium, Chrysanthemum, Conyza, Galium, Ipomoea, Nasturtium, Sinapis, Solanum, Stellaria, Veronica, Viola or Xanthium).

### V. Heterologous Functional Agents

The pest control (e.g., biopesticide or biorepellent) compositions described herein can further include a heterologous functional agent, such as a heterologous effective agent (e.g., a pesticidal agent or a repellent agent). In some instances, the pest control (e.g., biopesticide or biorepellent) compositions include two or more (e.g., 2, 3, 4, 5, 6, 7, 8, 9, 10, or more than 10) different pesticidal and/or repellent agents. In some instances, the heterologous functional agent (e.g., pesticidal agent and/or repellent agent) is included in the PMP. For example, the PMP may encapsulate the heterologous functional agent (e.g., pesticidal agent and/or repellent agent). Alternatively, the heterologous functional agent (e.g., pesticidal agent and/or repellent agent) can be embedded on or conjugated to the surface of the PMP.

In other instances, the pest control (e.g., biopesticide or biorepellent) composition can be formulated to include the heterologous functional agent (e.g., pesticidal agent and/or repellent agent), without it necessarily being associated with the PMP. In formulations and in the use forms prepared from these formulations, the pest control (e.g., biopesticide or biorepellent) composition may include additional active compounds, such as pesticidal agents (e.g., insecticides, sterilants, acaricides, nematicides, molluscicides, bactericides, fungicides, virucides, or herbicides), attractants, or repellents.

The pesticidal agent can be an antifungal agent, an antibacterial agent, an insecticidal agent, a molluscicidal agent, a nematicidal agent, a virucidal agent, or a combination thereof. The pesticidal agent can be a chemical agent, such as those well known in the art. Alternatively or additionally, the pesticidal agent can be a peptide, a polypeptide, a nucleic acid, a polynucleotide, or a small molecule. The pesticidal agent may be an agent that can decrease the fitness of a variety of plant pests or can be one that targets one or more specific target plant pests (e.g., a specific species or genus of plant pests).

In some instances, the heterologous functional agent (e.g., chemical, nucleic acid molecule, peptide, polypeptide, or small molecule) can be modified. For example, the modification can be a chemical modification, e.g., conjugation to a marker, e.g., fluorescent marker or a radioactive marker. In other examples, the modification can include conjugation or operational linkage to a moiety that enhances the stability, delivery, targeting, bioavailability, or half-life of the agent, e.g., a lipid, a glycan, a polymer (e.g., PEG), a cation moiety.

Examples of heterologous functional agents (e.g., pesticidal or repellent agent) that can be used in the presently disclosed pest control (e.g., biopesticide or biorepellent) compositions and methods are outlined below.

A. Antibacterial Agents

The pest control (e.g., biopesticide or biorepellent) compositions described herein can further include an antibacterial agent. In some instances, the pest control (e.g., biopesticide or biorepellent) compositions include two or more (e.g., 2, 3, 4, 5, 6, 7, 8, 9, 10, or more than 10) different antibacterial agents. For example, the antibacterial agent can decrease the fitness of (e.g., decrease growth or kill) a bacterial plant pest (e.g., a bacterial plant pathogen). A pest control (e.g., biopesticide or biorepellent) composition including an antibiotic as described herein can be contacted with a target pest, or plant infested thereof, in an amount and for a time sufficient to: (a) reach a target level (e.g., a predetermined or threshold level) of antibiotic concentration inside or on the target pest; and (b) decrease fitness of the target pest. The antibacterials described herein may be formulated in a pest control (e.g., biopesticide or biorepellent) composition for any of the methods described herein, and in certain instances, may be associated with the PMP thereof.

As used herein, the term “antibacterial agent” refers to a material that kills or inhibits the growth, proliferation, division, reproduction, or spread of bacteria, such as phytopathogenic bacteria, and includes bactericidal (e.g., disinfectant compounds, antiseptic compounds, or antibiotics) or bacteriostatic agents (e.g., compounds or antibiotics). Bactericidal antibiotics kill bacteria, while bacteriostatic antibiotics only slow their growth or reproduction.

Bactericides can include disinfectants, antiseptics, or antibiotics. The most used disinfectants can comprise: active chlorine (i.e., hypochlorites (e.g., sodium hypochlorite), chloramines, dichloroisocyanurate and trichloroisocyanurate, wet chlorine, chlorine dioxide etc.), active oxygen (peroxides, such as peracetic acid, potassium persulfate, sodium perborate, sodium percarbonate and urea perhydrate), iodine (iodpovidone (povidone-iodine, Betadine), Lugol's solution, iodine tincture, iodinated nonionic surfactants), concentrated alcohols (mainly ethanol, 1-propanol, called also n-propanol and 2-propanol, called isopropanol and mixtures thereof; further, 2-phenoxyethanol and 1- and 2-phenoxypropanols are used), phenolic substances (such as phenol (also called carbolic acid), cresols (called Lysole in combination with liquid potassium soaps), halogenated (chlorinated, brominated) phenols, such as hexachlorophene, triclosan, trichlorophenol, tribromophenol, pentachlorophenol, Dibromol and salts thereof), cationic surfactants, such as some quaternary ammonium cations (such as benzalkonium chloride, cetyl trimethylammonium bromide or chloride, didecyldimethylammonium chloride, cetylpyridinium chloride, benzethonium chloride) and others, non-quaternary compounds, such as chlorhexidine, glucoprotamine, octenidine dihydrochloride etc.), strong oxidizers, such as ozone and permanganate solutions; heavy metals and their salts, such as colloidal silver, silver nitrate, mercury chloride, phenylmercury salts, copper sulfate, copper oxide-chloride, copper hydroxide, copper octanoate, copper oxychloride sulfate, copper sulfate, copper sulfate pentahydrate, etc. Heavy metals and their salts are the most toxic, and environment-hazardous bactericides and therefore, their use is strongly oppressed or canceled; further, also properly concentrated strong acids (phosphoric, nitric, sulfuric, amidosulfuric, toluenesulfonic acids) and alkalis (sodium, potassium, calcium hydroxides).

As antiseptics (i.e., germicide agents that can be used on human or animal body, skin, mucoses, wounds and the like), few of the above mentioned disinfectants can be used, under proper conditions (mainly concentration, pH, temperature and toxicity toward man/animal). Among them, important are: properly diluted chlorine preparations (i.e. Daquin's solution, 0.5% sodium or potassium hypochlorite solution, pH-adjusted to pH 7-8, or 0.5-1% solution of sodium benzenesulfochloramide (chloramine B)), some iodine preparations, such as iodopovidone in various galenics (ointment, solutions, wound plasters), in the past also Lugol's solution, peroxides as urea perhydrate solutions and pH-buffered 0.1-0.25% peracetic acid solutions, alcohols with or without antiseptic additives, used mainly for skin antisepsis, weak organic acids such as sorbic acid, benzoic acid, lactic acid and salicylic acid some phenolic compounds, such as hexachlorophene, triclosan and Dibromol, and cation-active compounds, such as 0.05-0.5% benzalkonium, 0.5-4% chlorhexidine, 0.1-2% octenidine solutions.

The pest control (e.g., biopesticide or biorepellent) composition described herein may include an antibiotic. Any antibiotic known in the art may be used. Antibiotics are commonly classified based on their mechanism of action, chemical structure, or spectrum of activity.

The antibiotic described herein may target any bacterial function or growth processes and may be either bacteriostatic (e.g., slow or prevent bacterial growth) or bactericidal (e.g., kill bacteria). In some instances, the antibiotic is a bactericidal antibiotic. In some instances, the bactericidal antibiotic is one that targets the bacterial cell wall (e.g., penicillins and cephalosporins); one that targets the cell membrane (e.g., polymyxins); or one that inhibits essential bacterial enzymes (e.g., rifamycins, lipiarmycins, quinolones, and sulfonamides). In some instances, the bactericidal antibiotic is an aminoglycoside (e.g., kasugamycin). In some instances, the antibiotic is a bacteriostatic antibiotic. In some instances the bacteriostatic antibiotic targets protein synthesis (e.g., macrolides, lincosamides, and tetracyclines). Additional classes of antibiotics that may be used herein include cyclic lipopeptides (such as daptomycin), glycylcyclines (such as tigecycline), oxazolidinones (such as linezolid), or lipiarmycins (such as fidaxomicin). Examples of antibiotics include rifampicin, ciprofloxacin, doxycycline, ampicillin, and polymyxin B. The antibiotic described herein may have any level of target specificity (e.g., narrow- or broad-spectrum). In some instances, the antibiotic is a narrow-spectrum antibiotic, and thus targets specific types of bacteria, such as gram-negative or gram-positive bacteria. Alternatively, the antibiotic may be a broad-spectrum antibiotic that targets a wide range of bacteria. In some instances, the antibiotic is doxorubicin or vancomycin.

Other non-limiting examples of antibiotics are found in Table 6. One skilled in the art will appreciate that a suitable concentration of each antibiotic in the composition depends on factors such as efficacy, stability of the antibiotic, number of distinct antibiotics, the formulation, and methods of application of the composition.

TABLE 6
Examples of Antibiotics

| Antibiotics | Action |
|---|---|
| Penicillins, cephalosporins, vancomycin | Cell wall synthesis |
| Polymixin, gramicidin | Membrane active agent, |
| | disrupt cell membrane |
| Tetracyclines, macrolides, chloramphenicol, | Inhibit protein synthesis |
| clindamycin, spectinomycin | |
| Sulfonamides | Inhibit folate-dependent |
| | pathways |
| Ciprofloxacin | Inhibit DNA-gyrase |
| Isoniazid, rifampicin, pyrazinamide, | Antimycobacterial agents |
| ethambutol, (myambutol), streptomycin | |

B. Antifungal Agents

The pest control (e.g., biopesticide or biorepellent) compositions described herein can further include an antifungal agent. In some instances, the pest control (e.g., biopesticide or biorepellent) compositions include two or more (e.g., 2, 3, 4, 5, 6, 7, 8, 9, 10, or more than 10) different antifungal agents. For example, the antifungal agent can decrease the fitness of (e.g., decrease growth or kill) a fungal plant pest. A pest control (e.g., biopesticide or biorepellent) composition including an antifungal as described herein can be contacted with a target fungal pest, or plant infested therewith, in an amount and for a time sufficient to: (a) reach a target level (e.g., a predetermined or threshold level) of antibiotic concentration inside or on the target fungus; and (b) decrease fitness of the target fungus. The antifungals described herein may be formulated in a pest control (e.g., biopesticide or biorepellent) composition for any of the methods described herein, and in certain instances, may be associated with the PMP thereof.

As used herein, the term “fungicide” or “antifungal agent” refers to a substance that kills or inhibits the growth, proliferation, division, reproduction, or spread of fungi, such as phytopathogenic fungi. Many different types of antifungal agent have been produced commercially. Non limiting examples of antifungal agents include: azoxystrobin, mancozeb, prothioconazole, folpet, tebuconazole, difenoconazole, captan, bupirimate, or fosetyl-Al. Further exemplary fungicides include, but are not limited to, strobilurins, azoxystrobin, dimoxystrobin, enestroburin, fluoxastrobin, kresoxim-methyl, metominostrobin, picoxystrobin, pyraclostrobin, trifloxystrobin, orysastrobin, carboxamides, carboxanilides, benalaxyl, benalaxyl-M, benodanil, carboxin, mebenil, mepronil, fenfuram, fenhexamid, flutolanil, furalaxyl, furcarbanil, furametpyr, metalaxyl, metalaxyl-M (mefenoxam), methfuroxam, metsulfovax, ofurace, oxadixyl, oxycarboxin, penthiopyrad, pyracarbolid, salicylanilide, tecloftalam, thifluzamide, tiadinil, N-biphenylamides, bixafen, boscalid, carboxylic acid morpholides, dimethomorph, flumorph, benzamides, flumetover, fluopicolid (picobenzamid), zoxamid, carboxamides, carpropamid, diclocymet, mandipropamid, silthiofam, azoles, triazoles, bitertanol, bromuconazole, cyproconazole, difenoconazole, diniconazole, enilconazole, epoxiconazole, fenbuconazole, flusilazol, fluquinconazole, flutriafol, hexaconazole, imibenconazole, ipconazole, metconazole, myclobutanil, penconazole, propiconazole, prothioconazole, simeconazole, tebuconazole, tetraconazole, triadimenol, triadimefon, triticonazole, Imidazoles, cyazofamid, imazalil, pefurazoate, prochloraz, triflumizole, benzimidazoles, benomyl, carbendazim, fuberidazole, thiabendazole, ethaboxam, etridiazole, hymexazol, nitrogen-containing heterocyclyl compounds, pyridines, fuazinam, pyrifenox, pyrimidines, bupirimate, cyprodinil, ferimzone, fenarimol, mepanipyrim, nuarimol, pyrimethanil, piperazines, triforine, pyrroles, fludioxonil, fenpiclonil, morpholines, aldimorph, dodemorph, fenpropimorph, tridemorph, dicarboximides, iprodione, procymidone, vinclozolin, acibenzolar-S-methyl, anilazine, captan, captafol, dazomet, diclomezin, fenoxanil, folpet, fenpropidin, famoxadon, fenamidon, octhilinone, probenazole, proquinazid, pyroquilon, quinoxyfen, tricyclazole, carbamates, dithiocarbamates, ferbam, mancozeb, maneb, metiram, metam, propineb, thiram, zineb, ziram, diethofencarb, flubenthiavalicarb, iprovalicarb, propamocarb, guanidines, dodine, iminoctadine, guazatine, kasugamycin, polyoxins, streptomycin, validamycin A, organometallic compounds, fentin salts, sulfur-containing heterocyclyl compounds, isoprothiolane, dithianone, organophosphorous compounds, edifenphos, fosetyl, fosetyl-aluminum, iprobenfos, pyrazophos, tolclofos-methyl, Organochlorine compounds, thiophanate-methyl, chlorothalonil, dichlofluanid, tolylfluanid, flusulfamide, phthalide, hexachlorobenzene, pencycuron, quintozene, nitrophenyl derivatives, binapacryl, dinocap, dinobuton, spiroxamine, cyflufenamid, cymoxanil, metrafenon, N-2-cyanophenyl-3,4-dichloroisothiazol-5-carboxamide (isotianil), N-(3′,4′,5′-trifluorobiphenyl-2-yl)-3-difluoromethyl-1-methylpyrazole-4-carboxamide, 3-[5-(4-chlorophenyl)-2,3-dimethylisoxazolidin-3-yl]-pyridine, N-(3′,4′-dichloro-4-fluorobiphenyl-2-yl)-3-difluoromethyl-1-methylpyrazol-e-4-carboxamide, 5-chloro-7-(4-methylpiperidin-1-yl)-6-(2,4,6-trifluorophenyl)-[1,2,4]tria-zolo[1,5-a]pyrimidine, 2-butoxy-6-iodo-3-propylchromen-4-one, N,N-dimethyl-3-(3-bromo-6-fluoro-2-methylindole-1-sulfonyl)-[1,2,4]triazo-le-1-sulfonamide, methyl-(2-chloro-5-[1-(3-methylbenzyloxyimino)-ethyl]benzyl)carbamate, methyl-(2-chloro-5-[1-(6-methylpyridin-2-ylmethoxy-imino)ethyl]benzyl)carbamate, methyl 3-(4-chlorophenyl)-3-(2-isopropoxycarbonylamino-3-methylbutyryl-amino)propionate, 4-fluorophenyl N-(1-(1-(4-cyanophenyl)ethanesulfonyl)but-2-yl)carbamate, N-(2-(4-[3-(4-chlorophenyl)prop-2-ynyloxy]-3-methoxyphenyl)ethyl)-2-metha-nesulfonylamino-3-methylbutyramide, N-(2-(4-[3-(4-chlorophenyl)prop-2-ynyloxy]-3-methoxyphenyl)ethyl)-2-ethan-esulfonylamino-3-methylbutyramide, N-(4′-bromobiphenyl-2-yl)-4-difluoromethyl-2-methylthiazol-5-carboxamide, N-(4′-trifluoromethylbiphenyl-2-yl)-4-difluoromethyl-2-methylthiazol-5-carboxamide, N-(4′-chloro-3′-fluorobiphenyl-2-yl)-4-difluoromethyl-2-methylt-hiazol-5-carboxamide, or methyl 2-(ortho-((2,5-dimethylphenyloxy-methylene)phenyl)-3-methoxyacrylate. One skilled in the art will appreciate that a suitable concentration of each antifungal in the composition depends on factors such as efficacy, stability of the antifungal, number of distinct antifungals, the formulation, and methods of application of the composition.

C. Insecticides

The pest control (e.g., biopesticide or biorepellent) compositions described herein can further include an insecticide. In some instances, the pest control (e.g., biopesticide or biorepellent) compositions include two or more (e.g., 2, 3, 4, 5, 6, 7, 8, 9, 10, or more than 10) different insecticide agents. For example, the insecticide can decrease the fitness of (e.g., decrease growth or kill) an insect plant pest. A pest control (e.g., biopesticide or biorepellent) composition including an insecticide as described herein can be contacted with a target insect pest, or plant infested therewith, in an amount and for a time sufficient to: (a) reach a target level (e.g., a predetermined or threshold level) of insecticide concentration inside or on the target insect; and (b) decrease fitness of the target insect. The insecticides described herein may be formulated in a pest control (e.g., biopesticide or biorepellent) composition for any of the methods described herein, and in certain instances, may be associated with the PMP thereof.

As used herein, the term “insecticide” or “insecticidal agent” refers to a substance that kills or inhibits the growth, proliferation, reproduction, or spread of insects, such as agricultural insect pests. Non limiting examples of insecticides are shown in Table 7. Additional non-limiting examples of suitable insecticides include biologics, hormones or pheromones such as azadirachtin, Bacillus species, Beauveria species, codlemone, Metarrhizium species, Paecilomyces species, thuringiensis, and Verticillium species, and active compounds having unknown or non-specified mechanisms of action such as fumigants (such as aluminium phosphide, methyl bromide and sulphuryl fluoride) and selective feeding inhibitors (such as cryolite, flonicamid and pymetrozine). One skilled in the art will appreciate that a suitable concentration of each insecticide in the composition depends on factors such as efficacy, stability of the insecticide, number of distinct insecticides, the formulation, and methods of application of the composition.

TABLE 7
Examples of insecticides

| Class | Compounds |
|---|---|
| chloronicotinyls/ | acetamiprid, clothianidin, dinotefuran, imidacloprid, nitenpyram, |
| neonicotinoids | nithiazine, thiacloprid, thiamethoxam, imidaclothiz, (2E)-1-[(2- |
| | chloro-1,3-thiazol-5-yl)methyl]-3,5-dimethyl-N-nitro-1,3,5-tri-azinan- |
| | 2-imine, acetylcholinesterase (AChE) inhibitors (such as |
| | carbamates and organophosphates) |
| carbamates | alanycarb, aldicarb, aldoxycarb, allyxycarb, aminocarb, bendiocarb, |
| | benfuracarb, bufencarb, butacarb, butocarboxim, butoxycarboxim, |
| | carbaryl, carbofuran, carbosulfan, chloethocarb, dimetilan, |
| | ethiofencarb, fenobucarb, fenothiocarb, formetanate, furathiocarb, |
| | isoprocarb, metam-sodium, methiocarb, methomyl, metolcarb, |
| | oxamyl, phosphocarb, pirimicarb, promecarb, propoxur, thiodicarb, |
| | thiofanox, triazamate, trimethacarb, XMC, xylylcarb |
| organophosphates | acephate, azamethiphos, azinphos (-methyl, -ethyl), bromophos-ethyl, |
| | bromfenvinfos (-methyl), butathiofos, cadusafos, |
| | carbophenothion, chlorethoxyfos, chlorfenvinphos, chlormephos, |
| | chlorpyrifos (-methyl/-ethyl), coumaphos, cyanofenphos, |
| | cyanophos, demeton-S-methyl, demeton-S-methylsulphon, dialifos, |
| | diazinon, dichlofenthion, dichlorvos/DDVP, dicrotophos, |
| | dimethoate, dimethylvinphos, dioxabenzofos, disulfoton, EPN, |
| | ethion, ethoprophos, etrimfos, famphur, fenamiphos, fenitrothion, |
| | fensulfothion, fenthion, flupyrazofos, fonofos, formothion, |
| | fosmethilan, fosthiazate, heptenophos, iodofenphos, iprobenfos, |
| | isazofos, isofenphos, isopropyl O-salicylate, isoxathion, malathion, |
| | mecarbam, methacrifos, methamidophos, methidathion, |
| | mevinphos, monocrotophos, naled, omethoate, oxydemeton-methyl, |
| | parathion (-methyl/-ethyl), phenthoate, phorate, phosalone, |
| | phosmet, phosphamidon, phosphocarb, phoxim, pirimiphos |
| | (-methyl/-ethyl), profenofos, propaphos, propetamphos, prothiofos, |
| | prothoate, pyraclofos, pyridaphenthion, pyridathion, quinalphos, |
| | sebufos, sulfotep, sulprofos, tebupirimfos, temephos, terbufos, |
| | tetrachlorvinphos, thiometon, triazophos, triclorfon, vamidothion |
| pyrethroids | acrinathrin, allethrin (d-cis-trans, d-trans), cypermethrin (alpha-, |
| | beta-, theta-, zeta-), permethrin (cis-, trans-), beta-cyfluthrin, |
| | bifenthrin, bioallethrin, bioallethrin-S-cyclopentyl-isomer, |
| | bioethanomethrin, biopermethrin, bioresmethrin, chlovaporthrin, |
| | cis-cypermethrin, cis-resmethrin, cis-permethrin, clocythrin, |
| | cycloprothrin, cyfluthrin, cyhalothrin, cyphenothrin, DDT, |
| | deltamethrin, empenthrin (1R-isomer), esfenvalerate, etofenprox, |
| | fenfluthrin, fenpropathrin, fenpyrithrin, fenvalerate, flubrocythrinate, |
| | flucythrinate, flufenprox, flumethrin, fluvalinate, fubfenprox, |
| | gamma-cyhalothrin, imiprothrin, kadethrin, lambda, cyhalothrin, |
| | metofluthrin, phenothrin (1R-trans isomer), prallethrin, profluthrin, |
| | protrifenbute, pyresmethrin, resmethrin, RU 15525, silafluofen, |
| | tau-fluvalinate, tefluthrin, terallethrin, tetramethrin (1R-isomer), |
| | tralocythrin, tralomethrin, transfluthrin, ZXI 8901, pyrethrins |
| | (pyrethrum) |
| oxadiazines | indoxacarb, acetylcholine receptor modulators (such as spinosyns) |
| spinosyns | Spinosad |
| cyclodiene | camphechlor, chlordane, endosulfan, gamma-HCH, HCH, |
| | heptachlor, |
| organochlorines | lindane, methoxychlor |
| fiproles | acetoprole, ethiprole, vaniliprole, fipronil |
| mectins | abamectin, avermectin, emamectin, emamectin-benzoate, |
| | fenoxycarb, hydroprene, kinoprene, methoprene, ivermectin, |
| | lepimectin, epofenonane, pyriproxifen, milbemectin, milbemycin, |
| | triprene |
| diacylhydrazines | chromafenozide, halofenozide, methoxyfenozide, tebufenozide |
| benzoylureas | bistrifluoron, chlorfluazuron, diflubenzuron, fluazuron, flucycloxuron, |
| | flufenoxuron, hexaflumuron, lufenuron, novaluron, noviflumuron, |
| | penfluoron, teflubenzuron, triflumuron |
| organotins | azocyclotin, cyhexatin, fenbutatin oxide |
| pyrroles | chlorfenapyr |
| dinitrophenols | binapacyrl, dinobuton, dinocap, DNOC |
| METIs | fenazaquin, fenpyroximate, pyrimidifen, pyridaben, tebufenpyrad, |
| | tolfenpyrad, rotenone, acequinocyl, fluacrypyrim, microbial |
| | disrupters of the intestinal membrane of insects (such as Bacillus |
| | thuringiensis strains), inhibitors of lipid synthesis (such as tetronic |
| | acids and tetramic acids) |
| tetronic acids | spirodiclofen, spiromesifen, spirotetramat |
| tetramic acids | cis-3-(2,5-dimethylphenyl)-8-methoxy-2-oxo-1-azaspiro[4.5]dec-3- |
| | en-4-yl ethyl carbonate (alias: carbonic acid, 3-(2,5- |
| | dimethylphenyl)-8-methoxy-2-oxo-1-azaspiro[4.5]dec-3-en-4-yl |
| | ethyl ester; CAS Reg. No.: 382608-10-8), carboxamides (such as |
| | flonicamid), octopaminergic agonists (such as amitraz), inhibitors of |
| | the magnesium-stimulated ATPase (such as propargite), ryanodin |
| | receptor agonists (such as phthalamides or rynaxapyr) |
| phthalamides | N2-[1,1-dimethyl-2-(methylsulphonyl)ethyl]-3-iodo-N1-[2-methyl--4- |
| | [1,2,2,2-tetrafluoro-1-(trifluoromethyl)ethyl]phenyl]-1,2-benzenedi- |
| | carboxamide (i.e., flubendiamide; CAS reg. No.: 272451-65-7) |

D. Nematicides

The pest control (e.g., biopesticide or biorepellent) compositions described herein can further include a nematicide. In some instances, the pest control (e.g., biopesticide or biorepellent) compositions include two or more (e.g., 2, 3, 4, 5, 6, 7, 8, 9, 10, or more than 10) different nematicides. For example, the nematicide can decrease the fitness of (e.g., decrease growth or kill) a nematode plant pest. A pest control (e.g., biopesticide or biorepellent) composition including a nematicide as described herein can be contacted with a target nematode pest, or plant infested therewith, in an amount and for a time sufficient to: (a) reach a target level (e.g., a predetermined or threshold level) of nematicide concentration inside or on the target nematode; and (b) decrease fitness of the target nematode. The nematicides described herein may be formulated in a pest control (e.g., biopesticide or biorepellent) composition for any of the methods described herein, and in certain instances, may be associated with the PMP thereof.

As used herein, the term “nematicide” or “nematicidal agent” refers to a substance that kills or inhibits the growth, proliferation, reproduction, or spread of nematodes, such as agricultural nematode pests. Non limiting examples of nematicides are shown in Table 8. One skilled in the art will appreciate that a suitable concentration of each nematicide in the composition depends on factors such as efficacy, stability of the nematicide, number of distinct nematicides, the formulation, and methods of application of the composition.

TABLE 8
Examples of Nematicides

| | |
|---|---|
| FUMIGANTS | D-D, 1,3-Dichloropropene, Ethylene Dibromide, 1,2-Dibromo-3- |
| | Chloropropane, Methyl Bromide, Chloropicrin, Metam Sodium, Dazomet, |
| | Methyl Isothiocyanate (MITC), Sodium Tetrathiocarbonate, Chloropicrin, |
| CARBAMATES | Aldicarb, Aldoxycarb, Carbofuran, Oxamyl, Cleothocarb |
| ORGANOPHOSPHATES | Ethoprophos, Fenamiphos, Cadusafos, Fosthiazate, Fensulfothion, |
| | Thionazin, Isazofos, |
| BIOCHEMICALS | DITERA ®, CLANDOSAN ®, SINCOCIN ® |

E. Molluscicides

The pest control (e.g., biopesticide or biorepellent) compositions described herein can further include a molluscicide. In some instances, the pest control (e.g., biopesticide or biorepellent) compositions include two or more (e.g., 2, 3, 4, 5, 6, 7, 8, 9, 10, or more than 10) different molluscicides. For example, the molluscicide can decrease the fitness of (e.g., decrease growth or kill) a mollusk plant pest. A pest control (e.g., biopesticide or biorepellent) composition including a molluscicide as described herein can be contacted with a target mollusk pest, or plant infested therewith, in an amount and for a time sufficient to: (a) reach a target level (e.g., a predetermined or threshold level) of molluscicide concentration inside or on the target mollusk; and (b) decrease fitness of the target mollusk. The molluscicides described herein may be formulated in a pest control (e.g., biopesticide or biorepellent) composition for any of the methods described herein, and in certain instances, may be associated with the PMP thereof.

As used herein, the term “molluscicide” or “molluscicidal agent” refers to a substance that kills or inhibits the growth, proliferation, reproduction, or spread of mollusks, such as agricultural mollusk pests. A number of chemicals can be employed as a molluscicide, including metal salts such as iron(III) phosphate, aluminium sulfate, and ferric sodium EDTA, [3][4], metaldehyde, methiocarb, or acetylcholinesterase inhibitors. One skilled in the art will appreciate that a suitable concentration of each molluscicide in the composition depends on factors such as efficacy, stability of the molluscicide, number of distinct molluscicides, the formulation, and methods of application of the composition.

F. Virucides

The pest control (e.g., biopesticide or biorepellent) compositions described herein can further include a virucide. In some instances, the pest control (e.g., biopesticide or biorepellent) compositions include two or more (e.g., 2, 3, 4, 5, 6, 7, 8, 9, 10, or more than 10) different virucides. For example, the virucide can decrease the fitness of (e.g., decrease or eliminate) a viral plant pathogen. A pest control (e.g., biopesticide or biorepellent) composition including a virucide as described herein can be contacted with a target virus, or plant infested therewith, in an amount and for a time sufficient to: (a) reach a target level (e.g., a predetermined or threshold level) of virucide concentration; and (b) decrease or eliminate the target virus. The virucides described herein may be formulated in a pest control (e.g., biopesticide or biorepellent) composition for any of the methods described herein, and in certain instances, may be associated with the PMP thereof.

As used herein, the term “virucide” or “antiviral” refers to a substance that kills or inhibits the growth, proliferation, reproduction, development, or spread of viruses, such as agricultural virus pathogens. A number of agents can be employed as a virucide, including chemicals or biological agents (e.g., nucleic acids, e.g., dsRNA). One skilled in the art will appreciate that a suitable concentration of each virucide in the composition depends on factors such as efficacy, stability of the virucide, number of distinct virucides, the formulation, and methods of application of the composition.

G. Herbicides

The pest control (e.g., biopesticide or biorepellent) compositions described herein can further include one or more (e.g., 1, 2, 3, 4, 5, 6, 7, 8, 9, 10, or more than 10) herbicide. For example, the herbicide can decrease the fitness of (e.g., decrease or eliminate) a weed. A pest control (e.g., biopesticide or biorepellent) composition including an herbicide as described herein can be contacted with a target weed in an amount and for a time sufficient to: (a) reach a target level (e.g., a predetermined or threshold level) of herbicide concentration on the plant and (b) decrease the fitness of the weed. The herbicides described herein may be formulated in a pest control (e.g., biopesticide or biorepellent) composition for any of the methods described herein, and in certain instances, may be associated with the PMP thereof.

As used herein, the term “herbicide” refers to a substance that kills or inhibits the growth, proliferation, reproduction, or spread of weeds. A number of chemicals can be employed as a herbicides, including Glufosinate, Propaquizafop, Metamitron, Metazachlor, Pendimethalin, Flufenacet, Diflufenican, Clomazone, Nicosulfuron, Mesotrione, Pinoxaden, Sulcotrione, Prosulfocarb, Sulfentrazone, Bifenox, Quinmerac, Triallate, Terbuthylazine, Atrazine, Oxyfluorfen, Diuron, Trifluralin, or Chlorotoluron. Further examples of herbicides include, but are not limited to, benzoic acid herbicides, such as dicamba esters, phenoxyalkanoic acid herbicides, such as 2,4-D, MCPA and 2,4-DB esters, aryloxyphenoxypropionic acid herbicides, such as clodinafop, cyhalofop, fenoxaprop, fluazifop, haloxyfop, and quizalofop esters, pyridinecarboxylic acid herbicides, such as aminopyralid, picloram, and clopyralid esters, pyrimidinecarboxylic acid herbicides, such as aminocyclopyrachlor esters, pyridyloxyalkanoic acid herbicides, such as fluoroxypyr and triclopyr esters, and hydroxybenzonitrile herbicides, such as bromoxynil and ioxynil esters, esters of the arylpyridine carboxylic acids, and arylpyrimidine carboxylic acids of the generic structures disclosed in U.S. Pat. Nos. 7,314,849, 7,300,907, and 7,642,220, each of which is incorporated by reference herein in its entirety. In certain embodiments, the herbicide can be selected from the group consisting of 2,4-D, 2,4-DB, acetochlor, acifluorfen, alachlor, ametryn, amitrole, asulam, atrazine, azafenidin, benefin, bensulfuron, bensulide, bentazon, bromacil, bromoxynil, butylate, carfentrazone, chloramben, chlorimuron, chlorproham, chlorsulfuron, clethodim, clomazone, clopyralid, cloransulam, cyanazine, cycloate, DCPA, desmedipham, dichlobenil, diclofop, diclosulam, diethatyl, difenzoquat, diflufenzopyr, dimethenamid-p, diquat, diuron, DSMA, endothall, EPTC, ethalfluralin, ethametsulfuron, ethofumesate, fenoxaprop, fluazifop-P, flucarbazone, flufenacet, flumetsulam, flumiclorac, flumioxazin, fluometuron, fluroxypyr, fluthiacet, fomesafen, foramsulfuron, glufosinate, glyphosate, halosulfuron, haloxyfop, hexazinone, imazamethabenz, imazamox, imazapic, imazaquin, imazethapyr, isoxaben, isoxaflutole, lactofen, linuron, MCPA, MCPB, mesotrione, methazole, metolachlor-s, metribuzin, metsulfuron, molinate, MSMA, napropamide, naptalam, nicosulfuron, norflurazon, oryzalin, oxadiazon, oxasulfuron, oxyfluorfen, paraquat, pebulate, pelargonic acid, pendimethalin, phenmedipham, picloram, primisulfuron, prodiamine, prometryn, pronamide, propachlor, propanil, prosulfuron, pyrazon, pyridate, pyrithiobac, quinclorac, quizalofop, rimsulfuron, sethoxydim, siduron, simazine, sulfentrazone, sulfometuron, sulfosulfuron, tebuthiuron, terbacil, thiazopyr, thifensulfuron, thiobencarb, tralkoxydim, triallate, triasulfuron, tribenuron, triclopyr, trifluralin, triflusulfuron, vernolate. In some examples, the herbicide is doxorubicin. One skilled in the art will appreciate that a suitable concentration of each herbicide in the composition depends on factors such as efficacy, stability of the herbicide, number of distinct herbicides, the formulation, and methods of application of the composition.

H. Repellents

The pest control (e.g., biopesticide or biorepellent) compositions described herein can further include a repellent. In some instances, the pest control (e.g., biopesticide or biorepellent) compositions include two or more (e.g., 2, 3, 4, 5, 6, 7, 8, 9, 10, or more than 10) different repellents. For example, the repellent can repel any of the pests described herein (e.g., insects, nematodes, or mollusks); microorganisms (e.g., phytopathogens or endophytes, such as bacteria, fungi, or viruses); or weeds. A pest control (e.g., biopesticide or biorepellent) composition including a repellent as described herein can be contacted with a target plant, or plant infested therewith, in an amount and for a time sufficient to: (a) reach a target level (e.g., a predetermined or threshold level) of repellent concentration; and (b) decrease the levels of the pest on the plant relative to an untreated plant. The repellent described herein may be formulated in a pest control composition for any of the methods described herein, and in certain instances, may be associated with the PMP thereof.

In some instances, the repellent is an insect repellent. Some examples of well-known insect repellents include: benzil; benzyl benzoate; 2,3,4,5-bis(butyl-2-ene)tetrahydrofurfural (MGK Repellent 11); butoxypolypropylene glycol; N-butylacetanilide; normal-butyl-6,6-dimethyl-5,6-dihydro-1,4-pyrone-2-carboxylate (Indalone); dibutyl adipate; dibutyl phthalate; di-normal-butyl succinate (Tabatrex); N,N-diethyl-meta-toluamide (DEET); dimethyl carbate (endo,endo)-dimethyl bicyclo[2.2.1] hept-5-ene-2,3-dicarboxylate); dimethyl phthalate; 2-ethyl-2-butyl-1,3-propanediol; 2-ethyl-1,3-hexanediol (Rutgers 612); di-normal-propyl isocinchomeronate (MGK Repellent 326); 2-phenylcyclohexanol; p-methane-3,8-diol, and normal-propyl N,N-diethylsuccinamate. Other repellents include citronella oil, dimethyl phthalate, normal-butylmesityl oxide oxalate and 2-ethyl hexanediol-1,3 (See, Kirk-Othmer Encyclopedia of Chemical Technology, 2nd Ed., Vol. 11: 724-728; and The Condensed Chemical Dictionary, 8th Ed., p 756).

An insect repellent may be a synthetic or nonsynthetic insect repellent. Examples of synthetic insect repellents include methyl anthranilate and other anthranilate-based insect repellents, benzaldehyde, DEET (N,N-diethyl-m-toluamide), dimethyl carbate, dimethyl phthalate, icaridin (i.e., picaridin, Bayrepel, and KBR 3023), indalone (e.g., as used in a “6-2-2” mixture (60% Dimethyl phthalate, 20% Indalone, 20% Ethylhexanediol), IR3535 (3-[N-Butyl-N-acetyl]-aminopropionic acid, ethyl ester), metofluthrin, permethrin, SS220, or tricyclodecenyl allyl ether. Examples of natural insect repellents include beautyberry (Callicarpa) leaves, birch tree bark, bog myrtle (Myrica Gale), catnip oil (e.g., nepetalactone), citronella oil, essential oil of the lemon eucalyptus (Corymbia citriodora; e.g., p-menthane-3,8-diol (PMD)), neem oil, lemongrass, tea tree oil from the leaves of Melaleuca alternifolia, tobacco, or extracts thereof.

I. Biological Agents

i. Polypeptides

The pest control (e.g., biopesticide or biorepellent) composition (e.g., PMPs) described herein may include a polypeptide, e.g., a polypeptide that is an antibacterial, antifungal, insecticidal, nematicidal, molluscicidal, virucidal, or herbicidal agent. In some instances, the pest control (e.g., biopesticide or biorepellent) composition described herein includes a polypeptide or functional fragments or derivative thereof, which target pathways in the pest. For example, the polypeptide can decrease the fitness of a plant pest. A pest control (e.g., biopesticide or biorepellent) composition including a polypeptide as described herein can be contacted with a target pest, or plant infested therewith, in an amount and for a time sufficient to: (a) reach a target level (e.g., a predetermined or threshold level) of polypeptide concentration; and (b) decrease or eliminate the target pest. The polypeptides described herein may be formulated in a pest control (e.g., biopesticide or biorepellent) composition for any of the methods described herein, and in certain instances, may be associated with the PMP thereof.

Examples of polypeptides that can be used herein can include an enzyme (e.g., a metabolic recombinase, a helicase, an integrase, a RNAse, a DNAse, or an ubiquitination protein), a pore-forming protein, a signaling ligand, a cell penetrating peptide, a transcription factor, a receptor, an antibody, a nanobody, a gene editing protein (e.g., CRISPR-Cas system, TALEN, or zinc finger), riboprotein, a protein aptamer, or a chaperone.

Polypeptides included herein may include naturally occurring polypeptides or recombinantly produced variants. In some instances, the polypeptide may be a functional fragments or variants thereof (e.g., an enzymatically active fragment or variant thereof). For example, the polypeptide may be a functionally active variant of any of the polypeptides described herein with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire sequence, to a sequence of a polypeptide described herein or a naturally occurring polypeptide. In some instances, the polypeptide may have at least 50% (e.g., at least 50%, 60%, 70%, 80%, 90%, 95%, 97%, 99%, or greater) identity to a protein of interest.

The polypeptides described herein may be formulated in a composition for any of the uses described herein. The compositions disclosed herein may include any number or type (e.g., classes) of polypeptides, such as at least about any one of 1 polypeptide, 2, 3, 4, 5, 10, 15, 20, or more polypeptides. A suitable concentration of each polypeptide in the composition depends on factors such as efficacy, stability of the polypeptide, number of distinct polypeptides in the composition, the formulation, and methods of application of the composition. In some instances, each polypeptide in a liquid composition is from about 0.1 ng/mL to about 100 mg/mL. In some instances, each polypeptide in a solid composition is from about 0.1 ng/g to about 100 mg/g.

Methods of making a polypeptide are routine in the art. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013).

Methods for producing a polypeptide involve expression in plant cells, although recombinant proteins can also be produced using insect cells, yeast, bacteria, mammalian cells, or other cells under the control of appropriate promoters. Mammalian expression vectors may comprise nontranscribed elements such as an origin of replication, a suitable promoter and enhancer, and other 5′ or 3′ flanking nontranscribed sequences, and 5′ or 3′ nontranslated sequences such as necessary ribosome binding sites, a polyadenylation site, splice donor and acceptor sites, and termination sequences. DNA sequences derived from the SV40 viral genome, for example, SV40 origin, early promoter, enhancer, splice, and polyadenylation sites may be used to provide the other genetic elements required for expression of a heterologous DNA sequence. Appropriate cloning and expression vectors for use with bacterial, fungal, yeast, and mammalian cellular hosts are described in Green & Sambrook, Molecular Cloning: A Laboratory Manual (Fourth Edition), Cold Spring Harbor Laboratory Press (2012).

Various mammalian cell culture systems can be employed to express and manufacture a recombinant polypeptide agent. Examples of mammalian expression systems include CHO cells, COS cells, HeLA and BHK cell lines. Processes of host cell culture for production of protein therapeutics are described in, e.g., Zhou and Kantardjieff (Eds.), Mammalian Cell Cultures for Biologics Manufacturing (Advances in Biochemical Engineering/Biotechnology), Springer (2014). Purification of proteins is described in Franks, Protein Biotechnology: Isolation, Characterization, and Stabilization, Humana Press (2013); and in Cutler, Protein Purification Protocols (Methods in Molecular Biology), Humana Press (2010). Formulation of protein therapeutics is described in Meyer (Ed.), Therapeutic Protein Drug Products: Practical Approaches to formulation in the Laboratory, Manufacturing, and the Clinic, Woodhead Publishing Series (2012).

In some instances, the pest control (e.g., biopesticide or biorepellent) composition includes an antibody or antigen binding fragment thereof. For example, an agent described herein may be an antibody that blocks or potentiates activity and/or function of a component of the pest. The antibody may act as an antagonist or agonist of a polypeptide (e.g., enzyme or cell receptor) in the pest. The making and use of antibodies against a target antigen in a pest is known in the art. See, for example, Zhiqiang An (Ed.), Therapeutic Monoclonal Antibodies: From Bench to Clinic, 1st Edition, Wiley, 2009 and also Greenfield (Ed.), Antibodies: A Laboratory Manual, 2nd Edition, Cold Spring Harbor Laboratory Press, 2013, for methods of making recombinant antibodies, including antibody engineering, use of degenerate oligonucleotides, 5′-RACE, phage display, and mutagenesis; antibody testing and characterization; antibody pharmacokinetics and pharmacodynamics; antibody purification and storage; and screening and labeling techniques.

The pest control (e.g., biopesticide or biorepellent) composition described herein may include a bacteriocin. In some instances, the bacteriocin is naturally produced by Gram-positive bacteria, such as Pseudomonas, Streptomyces, Bacillus, Staphylococcus, or lactic acid bacteria (LAB, such as Lactococcus lactis). In some instances, the bacteriocin is naturally produced by Gram-negative bacteria, such as Hafnia alvei, Citrobacter freundii, Klebsiella oxytoca, Klebsiella pneumonia, Enterobacter cloacae, Serratia plymithicum, Xanthomonas campestris, Erwinia carotovora, Ralstonia solanacearum, or Escherichia coli. Exemplary bacteriocins include, but are not limited to, Class I-IV LAB antibiotics (such as lantibiotics), colicins, microcins, and pyocins.

The pest control (e.g., biopesticide or biorepellent) composition described herein may include an antimicrobial peptide (AMP). Any AMP suitable for inhibiting a microorganism may be used. AMPs are a diverse group of molecules, which are divided into subgroups on the basis of their amino acid composition and structure. The AMP may be derived or produced from any organism that naturally produces AMPs, including AMPs derived from plants (e.g., copsin), insects (e.g., mastoparan, poneratoxin, cecropin, moricin, melittin), frogs (e.g., magainin, dermaseptin, aurein), and mammals (e.g., cathelicidins, defensins and protegrins).

ii. Nucleic Acids

Numerous nucleic acids are useful in the compositions and methods described herein. The compositions disclosed herein may include any number or type (e.g., classes) of nucleic acids (e.g., DNA molecule or RNA molecule, e.g., mRNA, guide RNA (gRNA), or inhibitory RNA molecule (e.g., siRNA, shRNA, or miRNA), or a hybrid DNA-RNA molecule), such as at least about 1 class or variant of a nucleic acid, 2, 3, 4, 5, 10, 15, 20, or more classes or variants of nucleic acids. A suitable concentration of each nucleic acid in the composition depends on factors such as efficacy, stability of the nucleic acid, number of distinct nucleic acids, the formulation, and methods of application of the composition. Examples of nucleic acids useful herein include a Dicer substrate small interfering RNA (dsiRNA), an antisense RNA, a short interfering RNA (siRNA), a short hairpin (shRNA), a microRNA (miRNA), an (asymmetric interfering RNA) aiRNA, a peptide nucleic acid (PNA), a morpholino, a locked nucleic acid (LNA), a piwi-interacting RNA (piRNA), a ribozyme, a deoxyribozymes (DNAzyme), an aptamer (DNA, RNA), a circular RNA (circRNA), a guide RNA (gRNA), or a DNA molecule.

A pest control (e.g., biopesticide or biorepellent) composition including a nucleic acid as described herein can be contacted with a target pest, or plant infested therewith, in an amount and for a time sufficient to: (a) reach a target level (e.g., a predetermined or threshold level) of nucleic acid concentration; and (b) decrease or eliminate the target pest. The nucleic acids described herein may be formulated in a pest control (e.g., biopesticide or biorepellent) composition for any of the methods described herein, and in certain instances, may be associated with the PMP thereof.

(a) Nucleic Acid Encoding Peptides

In some instances, the pest control (e.g., biopesticide or biorepellent) composition includes a nucleic acid encoding a polypeptide. Nucleic acids encoding a polypeptide may have a length from about 10 to about 50,000 nucleotides (nts), about 25 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, about 5000 to about 6000 nts, about 6000 to about 7000 nts, about 7000 to about 8000 nts, about 8000 to about 9000 nts, about 9000 to about 10,000 nts, about 10,000 to about 15,000 nts, about 10,000 to about 20,000 nts, about 10,000 to about 25,000 nts, about 10,000 to about 30,000 nts, about 10,000 to about 40,000 nts, about 10,000 to about 45,000 nts, about 10,000 to about 50,000 nts, or any range therebetween.

The pest control (e.g., biopesticide or biorepellent) composition may also include functionally active variants of a nucleic acid sequence of interest. In some instances, the variant of the nucleic acids has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire sequence, to a sequence of a nucleic acid of interest. In some instances, the invention includes a functionally active polypeptide encoded by a nucleic acid variant as described herein. In some instances, the functionally active polypeptide encoded by the nucleic acid variant has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire amino acid sequence, to a sequence of a polypeptide of interest or the naturally derived polypeptide sequence.

Some methods for expressing a nucleic acid encoding a protein may involve expression in cells, including insect, yeast, bacteria, or other cells under the control of appropriate promoters. Expression vectors may include nontranscribed elements, such as an origin of replication, a suitable promoter and enhancer, and other 5′ or 3′ flanking nontranscribed sequences, and 5′ or 3′ nontranslated sequences such as necessary ribosome binding sites, a polyadenylation site, splice donor and acceptor sites, and termination sequences. DNA sequences derived from the SV40 viral genome, for example, SV40 origin, early promoter, enhancer, splice, and polyadenylation sites may be used to provide the other genetic elements required for expression of a heterologous DNA sequence. Appropriate cloning and expression vectors for use with bacterial, fungal, yeast, and mammalian cellular hosts are described in Green et al., Molecular Cloning: A Laboratory Manual, Fourth Edition, Cold Spring Harbor Laboratory Press, 2012.

Genetic modification using recombinant methods is generally known in the art. A nucleic acid sequence coding for a desired gene can be obtained using recombinant methods known in the art, such as, for example by screening libraries from cells expressing the gene, by deriving the gene from a vector known to include the same, or by isolating directly from cells and tissues containing the same, using standard techniques. Alternatively, a gene of interest can be produced synthetically, rather than cloned.

Expression of natural or synthetic nucleic acids is typically achieved by operably linking a nucleic acid encoding the gene of interest to a promoter, and incorporating the construct into an expression vector. Expression vectors can be suitable for replication and expression in bacteria. Expression vectors can also be suitable for replication and integration in eukaryotes. Typical cloning vectors contain transcription and translation terminators, initiation sequences, and promoters useful for expression of the desired nucleic acid sequence.

Additional promoter elements, e.g., enhancers, regulate the frequency of transcriptional initiation. Typically, these are located in the region 30-110 basepairs (bp) upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

Alternatively, the promoter may be an inducible promoter. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

The expression vector to be introduced can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes may be used for identifying potentially transformed cells and for evaluating the functionality of regulatory sequences. In general, a reporter gene is a gene that is not present in or expressed by the recipient source and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., FEBS Letters 479:79-82, 2000). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some instances, an organism may be genetically modified to alter expression of one or more proteins. Expression of the one or more proteins may be modified for a specific time, e.g., development or differentiation state of the organism. In one instances, the invention includes a composition to alter expression of one or more proteins, e.g., proteins that affect activity, structure, or function. Expression of the one or more proteins may be restricted to a specific location(s) or widespread throughout the organism.

(b) Synthetic mRNA

The pest control (e.g., biopesticide or biorepellent) composition may include a synthetic mRNA molecule, e.g., a synthetic mRNA molecule encoding a polypeptide. The synthetic mRNA molecule can be modified, e.g., chemically. The mRNA molecule can be chemically synthesized or transcribed in vitro. The mRNA molecule can be disposed on a plasmid, e.g., a viral vector, bacterial vector, or eukaryotic expression vector. In some examples, the mRNA molecule can be delivered to cells by transfection, electroporation, or transduction (e.g., adenoviral or lentiviral transduction).

In some instances, the modified RNA agent of interest described herein has modified nucleosides or nucleotides. Such modifications are known and are described, e.g., in WO 2012/019168. Additional modifications are described, e.g., in WO 2015/038892; WO 2015/038892; WO 2015/089511; WO 2015/196130; WO 2015/196118 and WO 2015/196128 A2.

In some instances, the modified RNA encoding a polypeptide of interest has one or more terminal modification, e.g., a 5′ cap structure and/or a poly-A tail (e.g., of between 100-200 nucleotides in length). The 5′ cap structure may be selected from the group consisting of CapO, Capl, ARCA, inosine, NI-methyl-guanosine, 2′fluoro-guanosine, 7-deaza-guanosine, 8-oxo-guanosine, 2-amino-guanosine, LNA-guanosine, and 2-azido-guanosine. In some cases, the modified RNAs also contain a 5′ UTR including at least one Kozak sequence, and a 3′ UTR. Such modifications are known and are described, e.g., in WO 2012/135805 and WO 2013/052523. Additional terminal modifications are described, e.g., in WO 2014/164253 and WO 2016/011306, WO 2012/045075, and WO 2014/093924. Chimeric enzymes for synthesizing capped RNA molecules (e.g., modified mRNA) which may include at least one chemical modification are described in WO 2014/028429.

In some instances, a modified mRNA may be cyclized, or concatemerized, to generate a translation competent molecule to assist interactions between poly-A binding proteins and 5′-end binding proteins. The mechanism of cyclization or concatemerization may occur through at least 3 different routes: 1) chemical, 2) enzymatic, and 3) ribozyme catalyzed. The newly formed 5′-/3′-linkage may be intramolecular or intermolecular. Such modifications are described, e.g., in WO 2013/151736.

Methods of making and purifying modified RNAs are known and disclosed in the art. For example, modified RNAs are made using only in vitro transcription (IVT) enzymatic synthesis. Methods of making IVT polynucleotides are known in the art and are described in WO 2013/151666, WO 2013/151668, WO 2013/151663, WO 2013/151669, WO 2013/151670, WO 2013/151664, WO 2013/151665, WO 2013/151671, WO 2013/151672, WO 2013/151667 and WO 2013/151736.S Methods of purification include purifying an RNA transcript including a polyA tail by contacting the sample with a surface linked to a plurality of thymidines or derivatives thereof and/or a plurality of uracils or derivatives thereof (polyT/U) under conditions such that the RNA transcript binds to the surface and eluting the purified RNA transcript from the surface (WO 2014/152031); using ion (e.g., anion) exchange chromatography that allows for separation of longer RNAs up to 10,000 nucleotides in length via a scalable method (WO 2014/144767); and subjecting a modified mRNA sample to DNAse treatment (WO 2014/152030).

Formulations of modified RNAs are known and are described, e.g., in WO 2013/090648. For example, the formulation may be, but is not limited to, nanoparticles, poly(lactic-co-glycolic acid)(PLGA) microspheres, lipidoids, lipoplex, liposome, polymers, carbohydrates (including simple sugars), cationic lipids, fibrin gel, fibrin hydrogel, fibrin glue, fibrin sealant, fibrinogen, thrombin, rapidly eliminated lipid nanoparticles (reLNPs) and combinations thereof.

Modified RNAs encoding polypeptides in the fields of human disease, antibodies, viruses, and a variety of in vivo settings are known and are disclosed in for example, Table 6 of International Publication Nos. WO 2013/151666, WO 2013/151668, WO 2013/151663, WO 2013/151669, WO 2013/151670, WO 2013/151664, WO 2013/151665, WO 2013/151736; Tables 6 and 7 International Publication No. WO 2013/151672; Tables 6, 178 and 179 of International Publication No. WO 2013/151671; Tables 6, 185 and 186 of International Publication No WO 2013/151667. Any of the foregoing may be synthesized as an IVT polynucleotide, chimeric polynucleotide or a circular polynucleotide, and each may include one or more modified nucleotides or terminal modifications.

(c) Inhibitory RNA

In some instances, the pest control (e.g., biopesticide or biorepellent) composition includes an inhibitory RNA molecule, e.g., that acts via the RNA interference (RNAi) pathway. In some instances, the inhibitory RNA molecule decreases the level of gene expression in a pest and/or decreases the level of a protein in the pest. In some instances, the inhibitory RNA molecule inhibits expression of a pest gene. For example, an inhibitory RNA molecule may include a short interfering RNA, short hairpin RNA, and/or a microRNA that targets a gene in the pest. Certain RNA molecules can inhibit gene expression through the biological process of RNA interference (RNAi). RNAi molecules include RNA or RNA-like structures typically containing 15-50 base pairs (such as about 18-25 base pairs) and having a nucleobase sequence identical (complementary) or nearly identical (substantially complementary) to a coding sequence in an expressed target gene within the cell. RNAi molecules include, but are not limited to: Dicer substrate small interfering RNAs (dsiRNA), short interfering RNAs (siRNAs), double-strand RNAs (dsRNA), short hairpin RNAs (shRNA), meroduplexes, dicer substrates, and multivalent RNA interference (U.S. Pat. Nos. 8,084,599 8,349,809, 8,513,207 and 9,200,276). A shRNA is a RNA molecule including a hairpin turn that decreases expression of target genes via RNAi. shRNAs can be delivered to cells in the form of plasmids, e.g., viral or bacterial vectors, e.g., by transfection, electroporation, or transduction). A microRNA is a non-coding RNA molecule that typically has a length of about 22 nucleotides. MiRNAs bind to target sites on mRNA molecules and silence the mRNA, e.g., by causing cleavage of the mRNA, destabilization of the mRNA, or inhibition of translation of the mRNA. In some instances, the inhibitory RNA molecule decreases the level and/or activity of a negative regulator of function. In other instances, the inhibitor RNA molecule decreases the level and/or activity of an inhibitor of a positive regulator of function. The inhibitory RNA molecule can be chemically synthesized or transcribed in vitro.

In some instances, the nucleic acid is a DNA, a RNA, or a PNA. In some instances, the RNA is an inhibitory RNA. In some instances, the inhibitory RNA inhibits gene expression in a plant pest. In some instances, the nucleic acid is an mRNA, a modified mRNA, or a DNA molecule that increases expression in the pest of an enzyme (e.g., a metabolic recombinase, a helicase, an integrase, a RNAse, a DNAse, or an ubiquitination protein), a pore-forming protein, a signaling ligand, a cell penetrating peptide, a transcription factor, a receptor, an antibody, a nanobody, a gene editing protein (e.g., CRISPR-Cas system, TALEN, or zinc finger), riboprotein, a protein aptamer, or a chaperone. In some instances, the nucleic acid is an mRNA, a modified mRNA, or a DNA molecule that increases the expression of an enzyme (e.g., a metabolic enzyme, a recombinase enzyme, a helicase enzyme, an integrase enzyme, a RNAse enzyme, a DNAse enzyme, or an ubiquitination protein), a pore-forming protein, a signaling ligand, a cell penetrating peptide, a transcription factor, a receptor, an antibody, a nanobody, a gene editing protein (e.g., a CRISPR-Cas system, a TALEN, or a zinc finger), a riboprotein, a protein aptamer, or a chaperone. In some instances, the increase in expression in the pest is an increase in expression of about 5%, 10%, 15%, 20%, 30%, 40%, 50%, 60%, 70%, 80%, 90%, 100%, or more than 100% relative to a reference level (e.g., the expression in an untreated pest). In some instances, the increase in expression in the pest is an increase in expression of about 2× fold, about 4× fold, about 5× fold, about 10× fold, about 20× fold, about 25× fold, about 50× fold, about 75× fold, or about 100× fold or more, relative to a reference level (e.g., the expression in an untreated pest).

In some instances, the nucleic acid is an antisense RNA, a dsiRNA, a siRNA, a shRNA, a miRNA, an aiRNA, a PNA, a morpholino, a LNA, a piRNA, a ribozyme, a DNAzyme, an aptamer (DNA, RNA), a circRNA, a gRNA, or a DNA molecule (e.g., an antisense polynucleotide) that acts to reduce expression in the pest of, e.g., an enzyme (a metabolic enzyme, a recombinase enzyme, a helicase enzyme, an integrase enzyme, a RNAse enzyme, a DNAse enzyme, a polymerase enzyme, a ubiquitination protein, a superoxide management enzyme, or an energy production enzyme), a transcription factor, a secretory protein, a structural factor (actin, kinesin, or tubulin), a riboprotein, a protein aptamer, a chaperone, a receptor, a signaling ligand, or a transporter. In some instances, the decrease in expression in the pest is a decrease in expression of about 5%, 10%, 15%, 20%, 30%, 40%, 50%, 60%, 70%, 80%, 90%, 100%, or more than 100% relative to a reference level (e.g., the expression in an untreated pest). In some instances, the decrease in expression in the pest is a decrease in expression of about 2× fold, about 4× fold, about 5× fold, about 10× fold, about 20× fold, about 25× fold, about 50× fold, about 75× fold, or about 100× fold or more, relative to a reference level (e.g., the expression in an untreated pest).

RNAi molecules include a sequence substantially complementary, or fully complementary, to all or a fragment of a target gene. RNAi molecules may complement sequences at the boundary between introns and exons to prevent the maturation of newly-generated nuclear RNA transcripts of specific genes into mRNA for transcription. RNAi molecules complementary to specific genes can hybridize with the mRNA for a target gene and prevent its translation. The antisense molecule can be DNA, RNA, or a derivative or hybrid thereof. Examples of such derivative molecules include, but are not limited to, peptide nucleic acid (PNA) and phosphorothioate-based molecules such as deoxyribonucleic guanidine (DNG) or ribonucleic guanidine (RNG).

RNAi molecules can be provided as ready-to-use RNA synthesized in vitro or as an antisense gene transfected into cells which will yield RNAi molecules upon transcription. Hybridization with mRNA results in degradation of the hybridized molecule by RNAse H and/or inhibition of the formation of translation complexes. Both result in a failure to produce the product of the original gene.

The length of the RNAi molecule that hybridizes to the transcript of interest may be around 10 nucleotides, between about 15 or 30 nucleotides, or about 15, 16, 17, 18, 19, 20, 21, 22, 23, 24, 25, 26, 27, 28, 29, 30 or more nucleotides. The degree of identity of the antisense sequence to the targeted transcript may be at least 75%, at least 80%, at least 85%, at least 90%, or at least 95.

RNAi molecules may also include overhangs, i.e., typically unpaired, overhanging nucleotides which are not directly involved in the double helical structure normally formed by the core sequences of the herein defined pair of sense strand and antisense strand. RNAi molecules may contain 3′ and/or 5′ overhangs of about 1-5 bases independently on each of the sense strands and antisense strands. In some instances, both the sense strand and the antisense strand contain 3′ and 5′ overhangs. In some instances, one or more of the 3′ overhang nucleotides of one strand base pairs with one or more 5′ overhang nucleotides of the other strand. In other instances, the one or more of the 3′ overhang nucleotides of one strand base do not pair with the one or more 5′ overhang nucleotides of the other strand. The sense and antisense strands of an RNAi molecule may or may not contain the same number of nucleotide bases. The antisense and sense strands may form a duplex wherein the 5′ end only has a blunt end, the 3′ end only has a blunt end, both the 5′ and 3′ ends are blunt ended, or neither the 5′ end nor the 3′ end are blunt ended. In another instance, one or more of the nucleotides in the overhang contains a thiophosphate, phosphorothioate, deoxynucleotide inverted (3′ to 3′ linked) nucleotide or is a modified ribonucleotide or deoxynucleotide.

Small interfering RNA (siRNA) molecules include a nucleotide sequence that is identical to about 15 to about 25 contiguous nucleotides of the target mRNA. In some instances, the siRNA sequence commences with the dinucleotide AA, includes a GC-content of about 30-70% (about 30-60%, about 40-60%, or about 45%-55%), and does not have a high percentage identity to any nucleotide sequence other than the target in the genome in which it is to be introduced, for example as determined by standard BLAST search.

siRNAs and shRNAs resemble intermediates in the processing pathway of the endogenous microRNA (miRNA) genes (Bartel, Cell 116:281-297, 2004). In some instances, siRNAs can function as miRNAs and vice versa (Zeng et al., Mol. Cell 9:1327-1333, 2002; Doench et al., Genes Dev. 17:438-442, 2003). Exogenous siRNAs downregulate mRNAs with seed complementarity to the siRNA (Birmingham et al., Nat. Methods 3:199-204, 2006). Multiple target sites within a 3′ UTR give stronger downregulation (Doench et al., Genes Dev. 17:438-442, 2003).

Known effective siRNA sequences and cognate binding sites are also well represented in the relevant literature. RNAi molecules are readily designed and produced by technologies known in the art. In addition, there are computational tools that increase the chance of finding effective and specific sequence motifs (Pei et al., Nat. Methods 3(9):670-676, 2006; Reynolds et al., Nat. Biotechnol. 22(3):326-330, 2004; Khvorova et al., Nat. Struct. Biol. 10(9):708-712, 2003; Schwarz et al., Cell 115(2):199-208, 2003; Ui-Tei et al., Nucleic Acids Res. 32(3):936-948, 2004; Heale et al., Nucleic Acids Res. 33(3):e30, 2005; Chalk et al., Biochem. Biophys. Res. Commun. 319(1):264-274, 2004; and Amarzguioui et al., Biochem. Biophys. Res. Commun. 316(4):1050-1058, 2004).

The RNAi molecule modulates expression of RNA encoded by a gene. Because multiple genes can share some degree of sequence homology with each other, in some instances, the RNAi molecule can be designed to target a class of genes with sufficient sequence homology. In some instances, the RNAi molecule can contain a sequence that has complementarity to sequences that are shared amongst different gene targets or are unique for a specific gene target. In some instances, the RNAi molecule can be designed to target conserved regions of an RNA sequence having homology between several genes thereby targeting several genes in a gene family (e.g., different gene isoforms, splice variants, mutant genes, etc.). In some instances, the RNAi molecule can be designed to target a sequence that is unique to a specific RNA sequence of a single gene.

An inhibitory RNA molecule can be modified, e.g., to contain modified nucleotides, e.g., 2′-fluoro, 2′-o-methyl, 2′-deoxy, unlocked nucleic acid, 2′-hydroxy, phosphorothioate, 2′-thiouridine, 4′-thiouridine, 2′-deoxyuridine. Without being bound by theory, it is believed that such modifications can increase nuclease resistance and/or serum stability, or decrease immunogenicity.

In some instances, the RNAi molecule is linked to a delivery polymer via a physiologically labile bond or linker. The physiologically labile linker is selected such that it undergoes a chemical transformation (e.g., cleavage) when present in certain physiological conditions, (e.g., disulfide bond cleaved in the reducing environment of the cell cytoplasm). Release of the molecule from the polymer, by cleavage of the physiologically labile linkage, facilitates interaction of the molecule with the appropriate cellular components for activity.

The RNAi molecule-polymer conjugate may be formed by covalently linking the molecule to the polymer. The polymer is polymerized or modified such that it contains a reactive group A. The RNAi molecule is also polymerized or modified such that it contains a reactive group B. Reactive groups A and B are chosen such that they can be linked via a reversible covalent linkage using methods known in the art.

Conjugation of the RNAi molecule to the polymer can be performed in the presence of an excess of polymer. Because the RNAi molecule and the polymer may be of opposite charge during conjugation, the presence of excess polymer can reduce or eliminate aggregation of the conjugate. Alternatively, an excess of a carrier polymer, such as a polycation, can be used. The excess polymer can be removed from the conjugated polymer prior to administration of the conjugate. Alternatively, the excess polymer can be co-administered with the conjugate.

Injection of double-stranded RNA (dsRNA) into mother insects efficiently suppresses their offspring's gene expression during embryogenesis, see for example, Khila et al., PLoS Genet. 5(7):e1000583, 2009; and Liu et al., Development 131(7):1515-1527, 2004. Matsuura et al. (PNAS 112(30):9376-9381, 2015) has shown that suppression of Ubx eliminates bacteriocytes and the symbiont localization of bacteriocytes.

The making and use of inhibitory agents based on non-coding RNA such as ribozymes, RNAse P, siRNAs, and miRNAs are also known in the art, for example, as described in Sioud, RNA Therapeutics: Function, Design, and Delivery (Methods in Molecular Biology). Humana Press (2010).

(d) Gene Editing

The pest control (e.g., biopesticide or biorepellent) compositions described herein may include a component of a gene editing system. For example, the agent may introduce an alteration (e.g., insertion, deletion (e.g., knockout), translocation, inversion, single point mutation, or other mutation) in a gene in the pest. Exemplary gene editing systems include the zinc finger nucleases (ZFNs), Transcription Activator-Like Effector-based Nucleases (TALEN), and the clustered regulatory interspaced short palindromic repeat (CRISPR) system. ZFNs, TALENs, and CRISPR-based methods are described, e.g., in Gaj et al., Trends Biotechnol. 31(7):397-405, 2013.

In a typical CRISPR/Cas system, an endonuclease is directed to a target nucleotide sequence (e.g., a site in the genome that is to be sequence-edited) by sequence-specific, non-coding guide RNAs that target single- or double-stranded DNA sequences. Three classes (I-III) of CRISPR systems have been identified. The class II CRISPR systems use a single Cas endonuclease (rather than multiple Cas proteins). One class II CRISPR system includes a type II Cas endonuclease such as Cas9, a CRISPR RNA (crRNA), and a trans-activating crRNA (tracrRNA). The crRNA contains a guide RNA, i.e., typically an about 20-nucleotide RNA sequence that corresponds to a target DNA sequence. The crRNA also contains a region that binds to the tracrRNA to form a partially double-stranded structure which is cleaved by RNase III, resulting in a crRNA/tracrRNA hybrid. The RNAs serve as guides to direct Cas proteins to silence specific DNA/RNA sequences, depending on the spacer sequence. See, e.g., Horvath et al., Science 327:167-170, 2010; Makarova et al., Biology Direct 1:7, 2006; Pennisi, Science 341:833-836, 2013. The target DNA sequence must generally be adjacent to a protospacer adjacent motif (PAM) that is specific for a given Cas endonuclease; however, PAM sequences appear throughout a given genome. CRISPR endonucleases identified from various prokaryotic species have unique PAM sequence requirements; examples of PAM sequences include 5′-NGG (SEQ ID NO: 78) (Streptococcus pyogenes), 5′-NNAGAA (SEQ ID NO: 79) (Streptococcus thermophilus CRISPRI), 5′-NGGNG (SEQ ID NO: 80) (Streptococcus thermophilus CRISPR3), and 5′-NNNGATT (SEQ ID NO: 81) (Neisseria meningiditis). Some endonucleases, e.g., Cas9 endonucleases, are associated with G-rich PAM sites, e.g., 5′-NGG (SEQ ID NO: 78), and perform blunt-end cleaving of the target DNA at a location 3 nucleotides upstream from (5′ from) the PAM site. Another class II CRISPR system includes the type V endonuclease Cpf1, which is smaller than Cas9; examples include AsCpf1 (from Acidaminococcus sp.) and LbCpf1 (from Lachnospiraceae sp.). Cpf1-associated CRISPR arrays are processed into mature crRNAs without the requirement of a tracrRNA; in other words, a Cpf1 system requires only the Cpf1 nuclease and a crRNA to cleave the target DNA sequence. Cpf1 endonucleases, are associated with T-rich PAM sites, e.g., 5′-TTN. Cpf1 can also recognize a 5′-CTA PAM motif. Cpf1 cleaves the target DNA by introducing an offset or staggered double-strand break with a 4- or 5-nucleotide 5′ overhang, for example, cleaving a target DNA with a 5-nucleotide offset or staggered cut located 18 nucleotides downstream from (3′ from) from the PAM site on the coding strand and 23 nucleotides downstream from the PAM site on the complimentary strand; the 5-nucleotide overhang that results from such offset cleavage allows more precise genome editing by DNA insertion by homologous recombination than by insertion at blunt-end cleaved DNA. See, e.g., Zetsche et al., Cell 163:759-771, 2015.

For the purposes of gene editing, CRISPR arrays can be designed to contain one or multiple guide RNA sequences corresponding to a desired target DNA sequence; see, for example, Cong et al., Science 339:819-823, 2013; Ran et al., Nature Protocols 8:2281-2308, 2013. At least about 16 or 17 nucleotides of gRNA sequence are required by Cas9 for DNA cleavage to occur; for Cpf1 at least about 16 nucleotides of gRNA sequence is needed to achieve detectable DNA cleavage. In practice, guide RNA sequences are generally designed to have a length of between 17-24 nucleotides (e.g., 19, 20, or 21 nucleotides) and complementarity to the targeted gene or nucleic acid sequence. Custom gRNA generators and algorithms are available commercially for use in the design of effective guide RNAs. Gene editing has also been achieved using a chimeric single guide RNA (sgRNA), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNAs have also been demonstrated to be effective in genome editing; see, for example, Hendel et al., Nature Biotechnol. 985-991, 2015.

Whereas wild-type Cas9 generates double-strand breaks (DSBs) at specific DNA sequences targeted by a gRNA, a number of CRISPR endonucleases having modified functionalities are available, for example: a nickase version of Cas9 generates only a single-strand break; a catalytically inactive Cas9 (dCas9) does not cut the target DNA but interferes with transcription by steric hindrance. dCas9 can further be fused with an effector to repress (CRISPRi) or activate (CRISPRa) expression of a target gene. For example, Cas9 can be fused to a transcriptional repressor (e.g., a KRAB domain) or a transcriptional activator (e.g., a dCas9-VP64 fusion). A catalytically inactive Cas9 (dCas9) fused to Fokl nuclease (dCas9-Fokl) can be used to generate DSBs at target sequences homologous to two gRNAs. See, e.g., the numerous CRISPR/Cas9 plasmids disclosed in and publicly available from the Addgene repository (Addgene, 75 Sidney St., Suite 550A, Cambridge, Mass. 02139; addgene.org/crispr/). A double nickase Cas9 that introduces two separate double-strand breaks, each directed by a separate guide RNA, is described as achieving more accurate genome editing by Ran et al., Cell 154:1380-1389, 2013.

CRISPR technology for editing the genes of eukaryotes is disclosed in US Patent Application Publications US 2016/0138008 A1 and US 2015/0344912 A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1.

In some instances, the desired genome modification involves homologous recombination, wherein one or more double-stranded DNA breaks in the target nucleotide sequence is generated by the RNA-guided nuclease and guide RNA(s), followed by repair of the break(s) using a homologous recombination mechanism (homology-directed repair). In such instances, a donor template that encodes the desired nucleotide sequence to be inserted or knocked-in at the double-stranded break is provided to the cell or subject; examples of suitable templates include single-stranded DNA templates and double-stranded DNA templates (e.g., linked to the polypeptide described herein). In general, a donor template encoding a nucleotide change over a region of less than about 50 nucleotides is provided in the form of single-stranded DNA; larger donor templates (e.g., more than 100 nucleotides) are often provided as double-stranded DNA plasmids. In some instances, the donor template is provided to the cell or subject in a quantity that is sufficient to achieve the desired homology-directed repair but that does not persist in the cell or subject after a given period of time (e.g., after one or more cell division cycles). In some instances, a donor template has a core nucleotide sequence that differs from the target nucleotide sequence (e.g., a homologous endogenous genomic region) by at least 1, at least 5, at least 10, at least 20, at least 30, at least 40, at least 50, or more nucleotides. This core sequence is flanked by homology arms or regions of high sequence identity with the targeted nucleotide sequence; in some instances, the regions of high identity include at least 10, at least 50, at least 100, at least 150, at least 200, at least 300, at least 400, at least 500, at least 600, at least 750, or at least 1000 nucleotides on each side of the core sequence. In some instances where the donor template is in the form of a single-stranded DNA, the core sequence is flanked by homology arms including at least 10, at least 20, at least 30, at least 40, at least 50, at least 60, at least 70, at least 80, or at least 100 nucleotides on each side of the core sequence. In instances, where the donor template is in the form of a double-stranded DNA, the core sequence is flanked by homology arms including at least 500, at least 600, at least 700, at least 800, at least 900, or at least 1000 nucleotides on each side of the core sequence. In one instance, two separate double-strand breaks are introduced into the cell or subject's target nucleotide sequence with a double nickase Cas9 (see Ran et al., Cell 154:1380-1389, 2013), followed by delivery of the donor template.

In some instances, the composition includes a gRNA and a targeted nuclease, e.g., a Cas9, e.g., a wild type Cas9, a nickase Cas9 (e.g., Cas9 D10A), a dead Cas9 (dCas9), eSpCas9, Cpf1, C2C1, or C2C3, or a nucleic acid encoding such a nuclease. The choice of nuclease and gRNA(s) is determined by whether the targeted mutation is a deletion, substitution, or addition of nucleotides, e.g., a deletion, substitution, or addition of nucleotides to a targeted sequence. Fusions of a catalytically inactive endonuclease e.g., a dead Cas9 (dCas9, e.g., D10A; H840A) tethered with all or a portion of (e.g., biologically active portion of) an (one or more) effector domain create chimeric proteins that can be linked to the polypeptide to guide the composition to specific DNA sites by one or more RNA sequences (sgRNA) to modulate activity and/or expression of one or more target nucleic acids sequences.

In instances, the agent includes a guide RNA (gRNA) for use in a CRISPR system for gene editing. In some instances, the agent includes a zinc finger nuclease (ZFN), or a mRNA encoding a ZFN, that targets (e.g., cleaves) a nucleic acid sequence (e.g., DNA sequence) of a gene in the pest. In some instances, the agent includes a TALEN, or an mRNA encoding a TALEN, that targets (e.g., cleaves) a nucleic acid sequence (e.g., DNA sequence) in a gene in the pest.

For example, the gRNA can be used in a CRISPR system to engineer an alteration in a gene in the pest. In other examples, the ZFN and/or TALEN can be used to engineer an alteration in a gene in the pest. Exemplary alterations include insertions, deletions (e.g., knockouts), translocations, inversions, single point mutations, or other mutations. The alteration can be introduced in the gene in a cell, e.g., in vitro, ex vivo, or in vivo. In some examples, the alteration increases the level and/or activity of a gene in the pest. In other examples, the alteration decreases the level and/or activity of (e.g., knocks down or knocks out) a gene in the pest. In yet another example, the alteration corrects a defect (e.g., a mutation causing a defect), in a gene in the pest.

In some instances, the CRISPR system is used to edit (e.g., to add or delete a base pair) a target gene in the pest. In other instances, the CRISPR system is used to introduce a premature stop codon, e.g., thereby decreasing the expression of a target gene. In yet other instances, the CRISPR system is used to turn off a target gene in a reversible manner, e.g., similarly to RNA interference. In some instances, the CRISPR system is used to direct Cas to a promoter of a gene, thereby blocking an RNA polymerase sterically.

In some instances, a CRISPR system can be generated to edit a gene in the pest, using technology described in, e.g., U.S. Publication No. 20140068797, Cong, Science 339: 819-823, 2013; Tsai, Nature Biotechnol. 32:6 569-576, 2014; U.S. Pat. Nos. 8,871,445; 8,865,406; 8,795,965; 8,771,945; and 8,697,359.

In some instances, the CRISPR interference (CRISPRi) technique can be used for transcriptional repression of specific genes in the pest. In CRISPRi, an engineered Cas9 protein (e.g., nuclease-null dCas9, or dCas9 fusion protein, e.g., dCas9-KRAB or dCas9-SID4X fusion) can pair with a sequence specific guide RNA (sgRNA). The Cas9-gRNA complex can block RNA polymerase, thereby interfering with transcription elongation. The complex can also block transcription initiation by interfering with transcription factor binding. The CRISPRi method is specific with minimal off-target effects and is multiplexable, e.g., can simultaneously repress more than one gene (e.g., using multiple gRNAs). Also, the CRISPRi method permits reversible gene repression.

In some instances, CRISPR-mediated gene activation (CRISPRa) can be used for transcriptional activation of a gene in the pest. In the CRISPRa technique, dCas9 fusion proteins recruit transcriptional activators. For example, dCas9 can be fused to polypeptides (e.g., activation domains) such as VP64 or the p65 activation domain (p65D) and used with sgRNA (e.g., a single sgRNA or multiple sgRNAs), to activate a gene or genes in the pest. Multiple activators can be recruited by using multiple sgRNAs—this can increase activation efficiency. A variety of activation domains and single or multiple activation domains can be used. In addition to engineering dCas9 to recruit activators, sgRNAs can also be engineered to recruit activators. For example, RNA aptamers can be incorporated into a sgRNA to recruit proteins (e.g., activation domains) such as VP64. In some examples, the synergistic activation mediator (SAM) system can be used for transcriptional activation. In SAM, MS2 aptamers are added to the sgRNA. MS2 recruits the MS2 coat protein (MCP) fused to p65AD and heat shock factor 1 (HSF1).

The CRISPRi and CRISPRa techniques are described in greater detail, e.g., in Dominguez et al., Nat. Rev. Mol. CellBiol. 17:5-15, 2016, incorporated herein by reference. In addition, dCas9-mediated epigenetic modifications and simultaneous activation and repression using CRISPR systems, as described in Dominguez et al., can be used to modulate a gene in the pest.

iii. Small Molecules

In some instances, the pest control (e.g., biopesticide or biorepellent) composition includes a small molecule, e.g., a biological small molecule. Numerous small molecule agents are useful in the methods and compositions described herein.

Small molecules include, but are not limited to, small peptides, peptidomimetics (e.g., peptoids), amino acids, amino acid analogs, synthetic polynucleotides, polynucleotide analogs, nucleotides, nucleotide analogs, organic and inorganic compounds (including heterorganic and organometallic compounds) generally having a molecular weight less than about 5,000 grams per mole, e.g., organic or inorganic compounds having a molecular weight less than about 2,000 grams per mole, e.g., organic or inorganic compounds having a molecular weight less than about 1,000 grams per mole, e.g., organic or inorganic compounds having a molecular weight less than about 500 grams per mole, and salts, esters, and other pharmaceutically acceptable forms of such compounds.

The small molecule described herein may be formulated in a composition or associated with the PMP for any of the pest control (e.g., biopesticide or biorepellent) compositions or related methods described herein. The compositions disclosed herein may include any number or type (e.g., classes) of small molecules, such as at least about any one of 1 small molecule, 2, 3, 4, 5, 10, 15, 20, or more small molecules. A suitable concentration of each small molecule in the composition depends on factors such as efficacy, stability of the small molecule, number of distinct small molecules, the formulation, and methods of application of the composition. In some instances, wherein the composition includes at least two types of small molecules, the concentration of each type of small molecule may be the same or different.

A pest control (e.g., biopesticide or biorepellent) composition including a small molecule as described herein can be contacted with the target pest in an amount and for a time sufficient to: (a) reach a target level (e.g., a predetermined or threshold level) of small molecule concentration inside or on a target pest, or plant infested therewith, and (b) decrease the fitness of the target pest.

In some instances, the pest control (e.g., biopesticide or biorepellent) composition of the compositions and methods described herein includes a secondary metabolite. Secondary metabolites are derived from organic molecules produced by an organism. Secondary metabolites may act (i) as competitive agents used against bacteria, fungi, amoebae, plants, insects, and large animals; (ii) as metal transporting agents; (iii) as agents of symbiosis between microbes and plants, insects, and higher animals; (iv) as sexual hormones; and (v) as differentiation effectors.

The secondary metabolite used herein may include a metabolite from any known group of secondary metabolites. For example, secondary metabolites can be categorized into the following groups: alkaloids, terpenoids, flavonoids, glycosides, natural phenols (e.g., gossypol acetic acid), enals (e.g., trans-cinnamaldehyde), phenazines, biphenols and dibenzofurans, polyketides, fatty acid synthase peptides, nonribosomal peptides, ribosomally synthesized and post-translationally modified peptides, polyphenols, polysaccharides (e.g., chitosan), and biopolymers. For an in-depth review of secondary metabolites see, for example, Vining, Annu. Rev. Microbiol. 44:395-427, 1990.

A pest control (e.g., biopesticide or biorepellent) composition including a secondary metabolite as described herein can be contacted with the target pest in an amount and for a time sufficient to: (a) reach a target level (e.g., a predetermined or threshold level) of secondary metabolite concentration inside or on a target pest, or plant infested therewith, and (b) decrease the fitness of the target pest.

### VI. Kits

The present invention also provides a kit for the control, prevention, or treatment of plant disease, where the kit includes a container having a pest control (e.g., biopesticide or biorepellent) composition described herein. The kit may further include instructional material for applying or delivering (e.g., to a plant or to a plant pest) the pest control (e.g., biopesticide or biorepellent) composition to control, prevent, or treat a plant pest infestation in accordance with a method of the present invention. The skilled artisan will appreciate that the instructions for applying the pest control (e.g., biopesticide or biorepellent) composition in the methods of the present invention can be any form of instruction. Such instructions include, but are not limited to, written instruction material (such as, a label, a booklet, a pamphlet), oral instructional material (such as on an audio cassette or CD) or video instructions (such as on a video tape or DVD).

### EXAMPLES

The following are examples of the methods of the invention. It is understood that various other embodiments may be practiced, given the general description provided above.

### Example 1: Isolation of Plant Messenger Packs from Plants

This example demonstrates the isolation of crude plant messenger packs (PMPs) from various plant sources, including the leaf apoplast, seed apoplast, root, fruit, vegetable, pollen, phloem, xylem sap, and plant cell culture medium.

### Experimental Design:

a) PMP Isolation from the Apoplast of Arabidopsis thaliana Leaves

Arabidopsis (Arabidopsis thaliana Col-0) seeds are surface sterilized with 50% bleach and plated on 0.53 Murashige and Skoog medium containing 0.8% agar. The seeds are vernalized for 2 d at 4° C. before being moved to short-day conditions (9-h days, 22° C., 150 μEm²). After 1 week, the seedlings are transferred to Pro-Mix PGX. Plants are grown for 4-6 weeks before harvest.

PMPs are isolated from the apoplastic wash of 4-6-week old Arabidopsis rosettes, as described by Rutter and Innes, Plant Physiol. 173(1): 728-741, 2017. Briefly, whole rosettes are harvested at the root and vacuum infiltrated with vesicle isolation buffer (20 mM MES, 2 mM CaCl2), and 0.1 M NaCl, pH6). Infiltrated plants are carefully blotted to remove excess fluid, placed inside 30-mL syringes, and centrifuged in 50 mL conical tubes at 700 g for 20 min at 2° C. to collect the apoplast extracellular fluid containing EVs. Next, the apoplast extracellular fluid is filtered through a 0.85 μm filter to remove large particles, and PMPs are purified as described in Example 2.

b) PMP Isolation from the Apoplast of Sunflower Seeds

Intact sunflower seeds (H. annuus L.), and are imbibed in water for 2 hours, peeled to remove the pericarp, and the apoplastic extracellular fluid is extracted by a modified vacuum infiltration-centrifugation procedure, adapted from Regente et al, FEBS Letters. 583: 3363-3366, 2009. Briefly, seeds are immersed in vesicle isolation buffer (20 mM MES, 2 mM CaCl2), and 0.1 M NaCl, pH6) and subjected to three vacuum pulses of 10 s, separated by 30 s intervals at a pressure of 45 kPa. The infiltrated seeds are recovered, dried on filter paper, placed in fritted glass filters and centrifuged for 20 min at 400 g at 4° C. The apoplast extracellular fluid is recovered, filtered through a 0.85 μm filter to remove large particles, and PMPs are purified as described in Example 2.

c) PMP Isolation from Ginger Roots

Fresh ginger (Zingiber officinale) rhizome roots are purchased from a local supplier and washed 3× with PBS. A total of 200 grams of washed roots is ground in a mixer (Osterizer 12-speed blender) at the highest speed for 10 min (pause 1 min for every 1 min of blending), and PMPs are isolated as described in Zhuang et al., J Extracellular Vesicles. 4(1):28713, 2015. Briefly, ginger juice is sequentially centrifuged at 1,000 g for 10 min, 3,000 g for 20 min and 10,000 g for 40 min to remove large particles from the PMP-containing supernatant. PMPs are purified as described in Example 2.

d) PMP Isolation from Grapefruit Juice

Fresh grapefruits (Citrus x paradisi) are purchased from a local supplier, their skins are removed, and the fruit is manually pressed, or ground in a mixer (Osterizer 12-speed blender) at the highest speed for 10 min (pause 1 min for every minute of blending) to collect the juice, as described by Wang et al., Molecular Therapy. 22(3): 522-534, 2014 with minor modifications. Briefly, juice/juice pulp is sequentially centrifuged at 1,000 g for 10 min, 3,000 g for 20 min, and 10,000 g for 40 min to remove large particles from the PMP-containing supernatant. PMPs are purified as described in Example 2.

e) PMP Isolation from Broccoli Heads

Broccoli (Brassica oleracea var. italica) PMPs are isolated as previously described (Deng et al., Molecular Therapy, 25(7): 1641-1654, 2017). Briefly, fresh broccoli is purchased from a local supplier, washed three times with PBS, and ground in a mixer (Osterizer 12-speed blender) at the highest speed for 10 min (pause 1 min for every minute of blending). Broccoli juice is then sequentially centrifuged at 1,000 g for 10 min, 3,000 g for 20 min, and 10,000 g for 40 min to remove large particles from the PMP-containing supernatant. PMPs are purified as described in Example 2.

f) PMP Isolation from Olive Pollen

Olive (Olea europaea) pollen PMPs are isolated as previously described in Prado et al., Molecular Plant. 7(3):573-577, 2014. Briefly, olive pollen (0.1 g) is hydrated in a humid chamber at room temperature for 30 min before transferring to petri dishes (15 cm in diameter) containing 20 ml germination medium: 10% sucrose, 0.03% Ca(NO₃)₂, 0.01% KNO₃, 0.02% MgSO₄, and 0.03% H₃BO₃. Pollen is germinated at 30° C. in the dark for 16 h. Pollen grains are considered germinated only when the tube is longer than the diameter of the pollen grain. Cultured medium containing PMPs is collected and cleared of pollen debris by two successive filtrations on 0.85 um filters by centrifugation. PMPs are purified as described in Example 2.

a) PMP Isolation from Arabidopsis Phloem Sap

Arabidopsis (Arabidopsis thaliana Col-0) seeds are surface sterilized with 50% bleach and plated on 0.53 Murashige and Skoog medium containing 0.8% agar. The seeds are vernalized for 2 d at 4° C. before being moved to short-day conditions (9-h days, 22° C., 150 μEm⁻²). After 1 week, the seedlings are transferred to Pro-Mix PGX. Plants are grown for 4-6 weeks before harvest.

Phloem sap from 4-6-week old Arabidopsis rosette leaves is collected as described by Tetyuk et al., JoVE. 80, 2013. Briefly, leaves are cut at the base of the petiole, stacked, and placed in a reaction tube containing 20 mM K2-EDTA for one hour in the dark to prevent sealing of the wound. Leaves are gently removed from the container, washed thoroughly with distilled water to remove all EDTA, put in a clean tube, and phloem sap is collected for 5-8 hours in the dark. Leaves are discarded, phloem sap is filtered through a 0.85 μm filter to remove large particles, and PMPs are purified as described in Example 2.

h) PMP Isolation from Tomato Plant Xylem Sap

Tomato (Solanum lycopersicum) seeds are planted in a single pot in an organic-rich soil, such as Sunshine Mix (Sun Gro Horticulture, Agawam, Mass.) and maintained in a greenhouse between 22° C. and 28° C. About two weeks after germination, at the two true-leaf stage, the seedlings are transplanted individually into pots (10 cm diameter and 17 cm deep) filled with sterile sandy soil containing 90% sand and 10% organic mix. Plants are maintained in a greenhouse at 22-28° C. for four weeks.

Xylem sap from 4-week old tomato plants is collected as described by Kohlen et al., Plant Physiology. 155(2):721-734, 2011. Briefly, tomato plants are decapitated above the hypocotyl, and a plastic ring is placed around the stem. The accumulating xylem sap is collected for 90 min after decapitation. Xylem sap is filtered through a 0.85 μm filter to remove large particles, and PMPs are purified as described in Example 2.

i) PMP Isolation from Tobacco BY-2 Cell Culture Medium

Tobacco BY-2 (Nicotiana tabacum L cv. Bright Yellow 2) cells are cultured in the dark at 26° C., on a shaker at 180 rpm in MS (Murashige and Skoog, 1962) BY-2 cultivation medium (pH 5.8) comprised MS salts (Duchefa, Haarlem, Netherlands, at #M0221) supplemented with 30 g/L sucrose, 2.0 mg/L potassium dihydrogen phosphate, 0.1 g/L myo-inositol, 0.2 mg/L 2,4-dichlorophenoxyacetic acid, and 1 mg/L thiamine HCl. The BY-2 cells are subcultured weekly by transferring 5% (v/v) of a 7-day-old cell culture into 100 mL fresh liquid medium. After 72-96 hours, BY-2 cultured medium is collected and centrifuged at 300 g at 4° C. for 10 minutes to remove cells. The supernatant containing PMPs is collected and cleared of debris by filtration on 0.85 um filter. PMPs are purified as described in Example 2.

### Example 2: Production of Purified Plant Messenger Packs (PMPs)

This example demonstrates the production of purified PMPs from crude PMP fractions as described in Example 1, using ultrafiltration combined with size-exclusion chromatography, a density gradient (iodixanol or sucrose), and the removal of aggregates by precipitation or size-exclusion chromatography.

### Experimental Design:

a) Production of Purified Grapefruit PMPs Using Ultrafiltration Combined with Size-Exclusion Chromatography

The crude grapefruit PMP fraction from Example 1a is concentrated using 100-kDA molecular weight cut-off (MWCO) Amicon spin filter (Merck Millipore). Subsequently, the concentrated crude PMP solution is loaded onto a PURE-EV size exclusion chromatography column (HansaBioMed Life Sciences Ltd) and isolated according to the manufacturer's instructions. The purified PMP-containing fractions are pooled after elution. Optionally, PMPs can be further concentrated using a 100-kDa MWCO Amicon spin filter, or by Tangential Flow Filtration (TFF). The purified PMPs are analyzed as described in Example 3.

b) Production of Purified Arabidopsis Apoplast PMPs Using an Iodixanol Gradient

Crude Arabidopsis leaf apoplast PMPs are isolated as described in Example 1a, and purified PMPs are produced by using an iodixanol gradient as described in Rutter and Innes, Plant Physiol. 173(1): 728-741, 2017. To prepare discontinuous iodixanol gradients (OptiPrep; Sigma-Aldrich), solutions of 40% (v/v), 20% (v/v), 10% (v/v), and 5% (v/v) iodixanol are created by diluting an aqueous 60% OptiPrep stock solution in vesicle isolation buffer (VIB; 20 mM MES, 2 mM CaCl2), and 0.1 M NaCl, pH6). The gradient is formed by layering 3 ml of 40% solution, 3 mL of 20% solution, 3 mL of 10% solution, and 2 mL of 5% solution. The crude apoplast PMP solution from Example 1a is centrifuged at 40,000 g for 60 min at 4° C. The pellet is resuspended in 0.5 ml of VIB and layered on top of the gradient. Centrifugation is performed at 100,000 g for 17 h at 4° C. The first 4.5 ml at the top of the gradient is discarded, and subsequently 3 volumes of 0.7 ml that contain the apoplast PMPs are collected, brought up to 3.5 mL with VIB and centrifuged at 100,000 g for 60 min at 4° C. The pellets are washed with 3.5 ml of VIB and repelleted using the same centrifugation conditions. The purified PMP pellets are combined for subsequent analysis, as described in Example 3.

c) Production of Purified Grapefruit PMPs Using a Sucrose Gradient

Crude grapefruit juice PMPs are isolated as described in Example 1d, centrifuged at 150,000 g for 90 min, and the PMP-containing pellet is resuspended in 1 ml PBS as described (Mu et al., Molecular Nutrition & Food Research. 58(7):1561-1573, 2014). The resuspended pellet is transferred to a sucrose step gradient (8%/15%/30%/45%/60%) and centrifuged at 150,000 g for 120 min to produce purified PMPs. Purified grapefruit PMPs are harvested from the 30%/45% interface, and subsequently analyzed, as described in Example 3.

d) Removal of Aggregates from Grapefruit PMPs

In order to remove protein aggregates from produced grapefruit PMPs as described in Example 1d or purified PMPs from Example 2a-c, an additional purification step can be included. The produced PMP solution is taken through a range of pHs to precipitate protein aggregates in solution. The pH is adjusted to 3, 5, 7, 9, or 11 with the addition of sodium hydroxide or hydrochloric acid. pH is measured using a calibrated pH probe. Once the solution is at the specified pH, it is filtered to remove particulates. Alternatively, the isolated PMP solution can be flocculated using the addition of charged polymers, such as Polymin-P or Praestol 2640. Briefly, 2-5 g per L of Polymin-P or Praestol 2640 is added to the solution and mixed with an impeller. The solution is then filtered to remove particulates. Alternatively, aggregates are solubilized by increasing salt concentration. NaCl is added to the PMP solution until it is at 1 mol/L. The solution is then filtered to purify the PMPs. Alternatively, aggregates are solubilized by increasing the temperature. The isolated PMP mixture is heated under mixing until it has reached a uniform temperature of 50° C. for 5 minutes. The PMP mixture is then filtered to isolate the PMPs. Alternatively, soluble contaminants from PMP solutions are separated by size-exclusion chromatography column according to standard procedures, where PMPs elute in the first fractions, whereas proteins and ribonucleoproteins and some lipoproteins are eluted later. The efficiency of protein aggregate removal is determined by measuring and comparing the protein concentration before and after removal of protein aggregates via BCA/Bradford protein quantification. The produced PMPs are analyzed as described in Example 3.

### Example 3: Plant Messenger Pack Characterization

This example demonstrates the characterization of PMPs produced as described in Example 1 or Example 2.

### Experimental Design:

a) Determining PMP Concentration

PMP particle concentration is determined by Nanoparticle Tracking Analysis (NTA) using a Malvern NanoSight, or by Tunable Resistive Pulse Sensing (TRPS) using an iZon qNano, following the manufacturer's instructions. The protein concentration of purified PMPs is determined by using the DC Protein assay (Bio-Rad). The lipid concentration of purified PMPs is determined using a fluorescent lipophilic dye, such as DiOC6 (ICN Biomedicals) as described by Rutter and Innes, Plant Physiol. 173(1): 728-741, 2017. Briefly, purified PMP pellets from Example 2 are resuspended in 100 ml of 10 mM DiOC6 (ICN Biomedicals) diluted with MES buffer (20 mM MES, pH 6) plus 1% plant protease inhibitor cocktail (Sigma-Aldrich) and 2 mM 2,29-dipyridyl disulfide. The resuspended PMPs are incubated at 37° C. for 10 min, washed with 3 mL of MES buffer, repelleted (40,000 g, 60 min, at 4° C.), and resuspended in fresh MES buffer. DiOC6 fluorescence intensity is measured at 485 nm excitation and 535 nm emission.

b) Biophysical and Molecular Characterization of PMPs

PMPs are characterized by electron and cryo-electron microscopy on a JEOL 1010 transmission electron microscope, following the protocol from Wu et al., Analyst. 140(2):386-406, 2015. The size and zeta potential of the PMPs are also measured using a Malvern Zetasizer or iZon qNano, following the manufacturer's instructions. Lipids are isolated from PMPs using chloroform extraction and characterized with LC-MS/MS as demonstrated in Xiao et al. Plant Cell. 22(10): 3193-3205, 2010. Glycosyl inositol phosphorylceramides (GIPCs) lipids are extracted and purified as described by Cacas et al., Plant Physiology. 170: 367-384, 2016, and analyzed by LC-MS/MS as described above. Total RNA, DNA, and protein are characterized using Quant-It kits from Thermo Fisher according to instructions. Proteins on the PMPs are characterized by LC-MS/MS following the protocol in Rutter and Innes, Plant Physiol. 173(1): 728-741, 2017. RNA and DNA are extracted using Trizol, prepared into libraries with the TruSeq Total RNA with Ribo-Zero Plant kit and the Nextera Mate Pair Library Prep Kit from Illumina, and sequenced on an Illumina MiSeq following manufacturer's instructions.

### Example 4: Characterization of Plant Messenger Pack Stability

This example demonstrates measuring the stability of PMPs under a wide variety of storage and physiological conditions.

### Experimental Design:

PMPs produced as described in Examples 1 and 2 are subjected to various conditions. PMPs are suspended in water, 5% sucrose, or PBS and left for 1, 7, 30, and 180 days at −20° C., 4° C., 20° C., and 37° C. PMPs are also suspended in water and dried using a rotary evaporator system and left for 1, 7, and 30, and 180 days at 4° C., 20° C., and 37° C. PMPs are also suspended in water or 5% sucrose solution, flash-frozen in liquid nitrogen and lyophilized. After 1, 7, 30, and 180 days, dried and lyophilized PMPs are then resuspended in water. The previous three experiments with conditions at temperatures above 0° C. are also exposed to an artificial sunlight simulator in order to determine content stability in simulated outdoor UV conditions. PMPs are also subjected to temperatures of 37° C., 40° C., 45° C., 50° C., and 55° C. for 1, 6, and 24 hours in buffered solutions with a pH of 1, 3, 5, 7, and 9 with or without the addition of 1 unit of trypsin or in other simulated gastric fluids.

After each of these treatments, PMPs are bought back to 20° C., neutralized to pH 7.4, and characterized using some or all of the methods described in Example 3.

### Example 5: Treatment of a Fungus with Plant Messenger Packs

This example demonstrates the ability of PMPs produced from a plant, such as Arabidopsis thaliana rosettes, to decrease fitness of a pathogenic fungus, e.g., S. sclerotorium, by treating the fungus directly, or spraying an apoplast PMP solution on Arabidopsis leaves prior to fungal exposure. In this example, Arabidopsis is used as a model plant, and S. sclerotorium as a model pathogenic fungus.

Plant diseases triggered by aggressive eukaryotic pathogens, such as fungi and oomycetes, cause significant crop losses worldwide. For instance, the broad range pathogenic fungi Botrytis cinerea and Sclerotinia sclerotiorum pose a serious threat to almost all vegetables and fruits, as well as many flowers, in their pre- and post-harvest stages by causing grey or white mould disease, respectively. Fungicide treatments are essential for maintaining healthy crops and reliable, high-quality yields.

### Therapeutic Design:

The Arabidopsis apoplast PMP solution was formulated with 0 (negative control), 1, 10, 50, 100, or 250 μg PMP protein/ml from Example 1a, in 10 ml of sterile water or PBS.

### Experimental Design:

a) Labeling Apoplast PMPs with a Lipophilic Membrane Dye

Arabidopsis thaliana apoplast PMPs are isolated and purified as described in Examples 1-2, and are labeled with PKH26 (Sigma), according to the manufacturer's protocol, with some modifications. Briefly, 50 mg apoplast PMPs in 1 mL dilute C of the PKH26 labelling kit are mixed with 2 ml of 1 mM PKH26 and incubated at 37° C. for 5 min. Labelling is stopped by adding 1 mL of 1% BSA. All unlabeled dye is washed away by centrifugation at 150,000 g for 90 min, and labelled PMP pellets are resuspended in sterile water.

b) Apoplast PMP uptake by S. sclerotiorum Ascospores

To determine the PMP uptake by S. sclerotiorum (ATCC, #18687) ascospores, 10,000 ascospores are incubated with 0 (negative control), 1, 10, 50, 100, or 250 μg/ml of PKH26-labeled apoplast-derived PMPs directly on glass slides. In addition to a PBS control, S. sclerotiorum ascospores are incubated in the presence of PKH26 dye (final concentration 5 μg/ml). After incubation of 5 min, 30 min and 1 h at room temperature, images are acquired on a high-resolution fluorescence microscope. Apoplast-derived PMPs are taken up by spores when the cytoplasm of the spore turns red versus exclusive staining of the cell membrane by PKH26 dye. The percentage of PMP treated spores with a red cytoplasm as compared to control treatments with PBS and PKH26 dye only are recorded.

c) Treatment of S. sclerotiorum with an Arabidopsis Apoplast PMP Solution In Vitro

To determine the effect of PMP treatment on the germination of fungal spores, ˜1500 S. sclerotiorum ascospores are incubated with 4% sucrose, and 0 (negative control), 1, 10, 50, 100, or 250 μg/ml PMPs, in a final volume of 20 μl on microslides using standard protocols, as described by Regente et al, J of Exp. Biol. 68(20): 5485-5496, 2017. After 16 h of incubation at 25° C. and 100% relative humidity, the slides are evaluated for the presence and morphology of hyphae using high resolution optical microscopy. Hyphal length is recorded using a scalebar, and the relative growth after PMP treatment is determined relative to the negative control. To determine fungal death, Evans Blue dye is added to a final concentration of 0.05% w/v and incubated for 10 min at room temperature before microscopic observation (a fungus is considered dead when it turns blue). To determine fungal viability, propidium iodide (PI) is added to a final concentration of 50 μg/ml and observed under a fluorescence microscope (a fungus is considered viable when stained positive (red) for PI). The relative viability between PMP-treated versus the non-treated control is determined.

d) Treatment of S. sclerotiorum with an Arabidopsis Apoplast PMP Solution in Planta

To determine the effect in planta of externally applied apoplast PMPs on fungal growth, four-week-old Arabidopsis thaliana Col-0 plants are sprayed with Arabidopsis apoplast PMPs at concentrations ranging from 0 (negative control), 1, 10, 50, 100, or 250 μg/ml of PMPs formulated in 10 mL of sterile water, 2 days, 1 day, and 2 hours prior to fungal infection.

Plant leaves are infected by S. sclerotiorum by applying a single 20 μl droplet or by spray-inoculating the entire plant, using 2×10⁵ spores/ml of S. sclerotiorum, as described in Weiberg et al Science. 342(6154):118-123, 2013.

One, two, three and five days after the initial infection, disease is assessed by measuring lesion size and a DNA-based real-time PCR assay is used for quantification of S sclerotiorum growth relative to Arabidopsis thaliana leaf biomass, as described by Ross and Somssich, Plant Methods. 12(1):48, 2016. DNA from 6 leaves from 6 individual plants are collected, and DNA is extracted using the FastDNA SPIN Kit for soil (MP Biomedicals) according to the manufacturer's instructions. For qPCR analysis 33 ng of DNA is mixed with 0.4 mM gene specific primers: S sclerotiorum fungal biomass (AF342243, Reich et al., Letters in Applied Microbiology. 62(5): 379-385, 2016): sense CCTACATTCTACTTGATCTAGTA, antisense GTTGGTAGTTGTGGGTTA; Arabidopsis plant biomass (At4 g26410, Ross and Somssich, Plant Methods. 12(1):48, 20161, sense GAGCTGAAGTGGCTTCCATGAC, anti-sense GGTCCGACATACCCATGATCC), and qPCR is performed using PowerUp™ SYBR™ Green Master Mix (Thermo Scientific) with three technical replicates according to the following protocol: denaturation at 95° C. for 3 min, 40 repeats of 95° C. for 20 s, 61° C. for 20 s and 72° C. for 15 s.

The abundance of the fungal-derived PCR product is normalized to the abundance of the plant derived PCR product. The in planta effect of Arabidopsis apoplast PMPs on fungal growth is determined by calculating the ΔΔCt value, comparing the normalized fungal growth in the negative PBS control to the normalized fungal growth in the PMP treatment samples.

### Example 6: Treatment of a Bacterium with Plant Messenger Packs

This example demonstrates the ability of purified apoplast PMPs from a plant, such as Arabidopsis thaliana rosettes, to be uptaken by bacteria, and to decrease the fitness of a pathogenic bacterium, e.g., Pseudomonas syringae, by treating the bacterium directly, or by spraying an apoplast PMP solution on Arabidopsis leaves prior to bacterial exposure. In this example, Arabidopsis is used as a model plant, and P syringae as a model bacterial pathogen.

Plant diseases triggered by bacterial pathogens, cause significant crop losses worldwide. For instance, broad range pathogenic bacteria like Pseudomonas syringae and Xanthomonas campestris pose a serious threat to global crop production. Bactericide treatments are essential for maintaining healthy crops and reliable, high-quality yields.

### Therapeutic Design:

The Arabidopsis apoplast PMP solution is formulated with 0 (negative control), 1, 10, 50, 100, or 250 μg PMP protein/ml in 10 ml sterile water.

a) Labeling Apoplast PMPs with a Lipophilic Membrane Dye

Arabidopsis thaliana apoplast PMPs are PMPs produced as described in Examples 1-2, and are labeled with PKH26 (Sigma) according to the manufacturer's protocol with some modifications. Briefly, 50 mg PMPs are diluted in 1 mL dilute C of the PKH26 labelling kit, and are mixed with 2 ml of 1 mM PKH26 and incubated at 37° C. for 5 min. Labelling is stopped by adding 1 mL 1% BSA. All unlabeled dye is washed away by centrifugation at 150,000 g for 90 min, and labelled PMP pellets are resuspended in sterile water, and analyzed as described in Example 3.

b) Apoplast PMP Uptake by P. syringae

Pseudomonas syringae pv. tomato str. DC3000 bacteria are obtained from the ATCC (#BAA-871) and grown on King's Medium B agar with 50 mg/ml rifampicin according to the manufacturer's instructions. To determine the PMP uptake by P. syringae, 10 ul of a 1 ml overnight bacterial suspension is incubated with 0 (negative control), 1, 10, 50, 100, or 250 μg/ml of PKH26-labeled apoplast PMPs directly on a glass slides. In addition to a water control, P. syringae bacteria are incubated in the presence of PKH26 dye (final concentration 5 μg/ml). After incubation of 5 min, 30 min and 1 h at room temperature, images are acquired on a high-resolution fluorescence microscope. Apoplast PMPs are taken up by bacteria when the cytoplasm of the bacteria turns red versus exclusive staining of the cell membrane by PKH26 dye. The percentage of PKH26-PMP treated bacteria with a red cytoplasm compared to control treatments with PBS and PKH26 dye only are recorded to determine PMP uptake.

c) Treatment of P. syringae with an Arabidopsis Apoplast PMP Solution In Vitro

The ability of Arabidopsis apoplast PMPs to affect the growth of P. syringae is determined as described by Hoefler et al. Cell Chem. Bio. 24(10):1238-1249, 2017. Briefly, P. syringae cultures at the stationary phase are concentrated to OD₆₀₀=4 by centrifugation and resuspension in medium. 1.5 mL of concentrated P. syringae culture is mixed with 4.5 mL agar and spread evenly over a 25 mL agar plate. After solidification, 3 uL of 0 (negative control), 1, 10, 50, 100, or 250 μg/ml of PMPs is spotted onto the overlay and allowed to dry. The plates are incubated overnight, photographed, and scanned. The diameter of the lytic zone (area without bacteria) around the spotted area is measured. Control and PMP-treated lytic zones are compared to determine the bactericidal effect of Arabidopsis apoplast PMPs.

d) Treatment of P. syringae with an Arabidopsis Apoplast PMP Solution in Planta

To determine the effect in planta of externally applied apoplast PMPs on bacterial growth, four-week-old Arabidopsis thaliana Col-0 plants are sprayed with Arabidopsis apoplast PMPs at concentrations ranging from 0 (negative control), 1, 10, 50, 100, or 250 μg/ml of PMPs formulated in 10 mL of sterile water, 2 days, 1 day, and 2 hours prior to bacterial infection.

P. syringae is grown as a lawn on King's Medium B agar overnight at 30° C. The bacterial lawn is scraped from the plate and resuspended to an optical density at 600 nm of 0.2 using 10 mM MgCl₂ plus 0.01% Silwet L77. Col-0 Arabidopsis plants are sprayed with the bacterial solution or a control solution lacking bacteria. Plastic domes are placed over the plants overnight to maintain high humidity and are removed the following morning.

One, two, three and five days after the initial infection, a DNA-based real-time PCR assay is used for quantification of Pseudomonas syringae growth relative to Arabidopsis thaliana leaf biomass, as described by Ross and Somssich, Plant Methods. 12(1):48, 2016. DNA from 6 leaves from 6 individual plants are collected, and DNA is extracted using the FastDNA SPIN Kit for soil (MP Biomedicals) according to the manufacturer's instructions. For qPCR analysis 33 ng of DNA is mixed with 0.4 mM gene specific primers (P syringae bacterial biomass: sense AACTGAAAAACACCTTGGGC, anti-sense CCTGGGTTGTTGAAGTGGTA (NC_004578.1); Arabidopsis plant biomass: A. thaliana expressed protein At4 g26410, sense GAGCTGAAGTGGCTTCCATGAC, anti-sense GGTCCGACATACCCATGATCC), and qPCR is performed using PowerUp™ SYBR™ Green Master Mix (Thermo Scientific) with three technical replicates according to the following protocol: denaturation at 95° C. for 3 min, 40 repeats of 95° C. for 20 s, 61° C. for 20 s and 72° C. for 15 s.

The abundance of the bacterial derived PCR product is normalized to the abundance of the plant derived PCR product. The in planta effect of Arabidopsis apoplast PMPs on bacterial growth is determined by calculating the ΔΔCt value, comparing the normalized bacterial growth in the negative control to the normalized bacterial growth in the PMP-treated samples.

### Example 7: Treatment of a Sap-Sucking Insect with Plant Messenger Packs

This example demonstrates the ability to kill or decrease the fitness of aphids by treating them with solutions of apoplast PMPs produced from a plant, such as Arabidopsis thaliana rosettes. The insect can be treated directly or by spraying a solution on a crop leaf prior to infestation by the aphids. In this example, aphids are used as a model organism for sap-sucking insects.

Aphids are one of the most important agricultural insect pests. They cause direct feeding damage to the plant and serve as vectors of plant viruses. In addition, aphid honeydew promotes the growth of sooty mold and attracts nuisance ants. The use of chemical treatments leads to the selection of resistant individuals whose eradication becomes increasingly difficult.

### Therapeutic Design:

The Arabidopsis apoplast PMP solution is formulated with 0 (negative control), 1, 10, 50, 100, or 250 μg PMP protein/ml in 10 ml of sterile water or PBS.

### Experimental Design:

a) Cultivation of Aphids

To prepare for the treatment, aphids are grown in a lab environment and medium. In a climate-controlled room (16 h light photoperiod; 60±5% RH; 20±2° C.), fava bean plants are grown in a mixture of vermiculite and perlite at 24° C. with 16 h of light and 8 h of darkness. To limit maternal effects or health differences between plants, 5-10 adults from different plants are distributed among 10 two-week-old plants and allowed to multiply to high density for 5-7 days. For experiments, second and third instar aphids are collected from healthy plants and divided into treatments so that each treatment receives approximately the same number of individuals from each of the collection plants.

b) Treatment of Third Instar Aphids with an Arabidopsis Apoplast PMP Solution

For each replicate treatment, 30-50 second and third instar aphids are placed individually in wells of a 96-well plate and a feeding sachet plate is inverted above them, allowing the insects to feed through the parafilm while keeping them restricted to individual wells. Experimental aphids are kept under the same environmental conditions as aphid colonies. After the aphids are fed for 24 h, the feeding sachet is replaced with a new one containing sterile artificial diet or sterile artificial diet supplemented with 1, 10, 50, 100, or 250 μg/ml apoplast PMPs and a new sterile sachet is provided every 24 h for four days. At the time that the sachet is replaced, aphids are also checked for mortality. An aphid is counted as dead if it had turned brown or is at the bottom of the well and does not move during the observation. If an aphid is on the parafilm of the feeding sachet but not moving, it is assumed to be feeding and alive. The survival rate of aphids treated with the PMP solution are compared to the aphids treated with the negative control. Developmental stages and sizes of aphids are also recorded daily to observe any delay in development.

c) Treatment of Aphids with an Arabidopsis Apoplast PMP Solution in Planta

To determine the effect in planta of externally applied apoplast PMPs on aphid fitness, leaves are picked from four-week-old fava beans plants and inserted in Eppendorf tubes containing solutions at concentrations ranging from 0 (negative control), 1, 10, 50, 100, or 250 μg/ml of PMPs formulated in 10 mL of sterile water. Alternatively, the leaves are sprayed according to the protocol in Wang et al., Nature Plants. 2(10):16151, 2016, and allowed to dry for 2 hours at RT. Plant leaves are then infected with 100 second and third instar aphids.

The survival rates of aphids treated with the PMP solutions are compared to the aphids treated with the negative control. Developmental stages and sizes of aphids are recorded daily to observe any delay in development.

### Example 8: Treatment of Corn Root-Knot Nematodes with Plant Messenger Packs

This example demonstrates the ability to kill or decrease the fitness of a nematode, e.g., corn root-knot nematodes, Meliodogyne, by treating them with a solution of apoplast PMPs isolated from a plant, such as Arabidopsis thaliana rosettes. In this example, Meliodogyne are used as a model pathogenic nematode.

Nematodes causing root-knots (Meliodogyne), cysts (Heterodera), reniforms (Rotylenchulus), and nematodes infecting citrus roots (Tylenchulus semipenetrans), of the phylum Nematoda, are threats to agricultural production. Plant parasitic nematodes feed on living plant root tissues (a few species will attack the leaves), using an oral stylet to puncture plant cells and suck their content. Nematodes cause symptoms similar to those caused by nutrient or water deficiency, such as yield loss, yellowing, wilting, and malformations of the root caused by direct feeding damage. In addition, invasion by plant-parasitic nematodes often provides an infection route for other organisms, such as bacteria or fungi, since nematode activity creates an entryway into the root that would otherwise not be available. The treatment of this pest typically involves chemical nematicides, such as Aldicarb, that are applied at concentrations that have raised concerns for human health safety and environmental impact due to the widespread deregistration of several chemical nematicides.

### Therapeutic Design:

The Arabidopsis apoplast PMP solution is formulated with 0 (negative control), 1, 10, 50, 100, or 250 μg PMP protein/ml from Example 1a in 10 ml of sterile water.

### Experimental Design:

a) Cultivation of Meliodogyne Nematodes

To prepare for the treatment, tomato seeds are planted in a single pot in an organic-rich soil, such as Sunshine Mix (Sun Gro Horticulture, Agawam, Mass.) and maintained in a greenhouse between 22° C. and 28° C. About two weeks after germination, at the two true-leaf stage, the seedlings are transplanted individually into pots (10 cm diameter and 17 cm deep) filled with sterile sandy soil containing 90% sand and 10% organic mix. Plants are maintained in a greenhouse at 22-28° C. for two weeks.

About 3000 Meliodogyne nematodes at the J2 stage (immediately after they hatch) are used to inoculate a plant. The nematodes are suspended in 6 mL of water. Three holes of about half-pot depth are made in the sand around each tomato root system using a pencil. Each plant is inoculated by delivering the J2s into the three holes using a pipette. Afterwards, the holes are covered. The plants are maintained in a greenhouse at 24-27° C. for six to eight weeks.

b) Treatment of Meliodogyne Eggs with Arabidopsis Apoplast PMPs

To assess the nematicidal activity of the PMP solution on the eggs of Meliodogyne nematodes, an in vitro hatching test is conducted. Eggs masses of Meliodogyne nematodes are obtained from the infected roots. Single egg masses, containing an average of 300-350 eggs, are placed in Syracuse dishes and treated with 2 ml of the PMP solution at concentrations of 0 (negative control), 1, 10, 50, 100, or 250 μg/ml and kept at 28±1° C. for different exposure times. The numbers of juveniles emerged from the eggs are counted after 24, 48, and 72 h. The effect on egg hatching is determined by comparing the percentage of juveniles emerged from the sterile water control with those from the PMP treatments. The hatching rate of nematode eggs treated with a PMP solution is decreased compared to the control.

c) Treatment of Meliodogyne Juveniles with Arabidopsis Apoplast PMPs

To assess the nematicidal activity of the PMP solution on juvenile Meliodogyne nematodes, an in vitro mortality test is conducted. Egg masses of Meliodogyne nematodes are collected from the infected roots and incubated in water for 3 days to allow eggs to hatch. After 3 days, 100 second stage juveniles are added to Syracuse dishes, containing 2 ml of the PMP solutions at concentrations of 0 (negative control), 1, 10, 50, 100, or 250 μg/ml, and incubated at 28±1° C. Observations are recorded on the mortality of juveniles at 24, 48, and 72 h intervals using a stereoscope. Thereafter, juveniles treated with the PMP solution are transferred to distilled water and observed again after 24 h to confirm their mortality. The survival rates of nematodes treated with the PMP solution are compared to the nematodes treated with the negative control. The survival rate of nematodes treated with the PMP solution is decreased compared to the control.

### Example 9: Treatment of an Herbivorous Insect with Plant Messenger Packs

This example demonstrates the ability to kill or decrease the fitness of an herbivorous insect, e.g., Spodoptera litura, by treatment with a solution of apoplast PMPs isolated from a plant, such as Arabidopsis thaliana rosettes. The Lepidoptera can be treated directly or by spraying an Arabidopsis apoplast PMP solution on crop leaf prior to infestation by the pest. In this example, Spodoptera litura is used as a model organism for herbivorous pathogenic insects.

S. litura is a serious polyphagous pest in America, Asia, Oceania, and India. The species parasitize the plants through the larvae's vigorous eating patterns, oftentimes leaving the leaves completely destroyed. The moth's effects are quite disastrous, destroying economically important agricultural crops and decreasing yield in some plants completely. Their impact on many different cultivated crops, and subsequently the local agricultural economy, has led to serious efforts to control the pests.

### Therapeutic Design:

The Arabidopsis apoplast PMP solution is formulated with 0 (negative control), 1, 10, 50, 100, or 250 μg PMP protein/ml in 10 ml of sterile water

### Experimental Design:

a) Cultivation of Spodoptera litura on Tobacco Plants

Spodoptera litura is maintained on tobacco plants for two consecutive generations. The tobacco plants are maintained at 28±1° C. under 16/8 h (light/dark) photoperiod with light supplied by cool white fluorescent lamps at an intensity of about 1600 lux for a period of 15 days for seed germination and sufficient seedling growth for transfer to new soil mixture.

S. litura eggs are supplied by Genralpest. Upon hatching, the 1^{st} instar larvae are reared on artificial diets as described in Shu et al., Chemosphere. 139:441-451, 2015. The rearing is carried out under constant conditions of 27° C., 65% relative humidity, and a 12-hour dark/12-hour light photoperiod in a climatic chamber. Pupae and adults are kept under the same conditions.

b) Treatment of Spodoptera litura Eggs with Arabidopsis Apoplast PMPs

To determine the effect of apoplast PMPs on S. litura development, hatching and mortality tests are performed. For the hatching test, single egg masses are placed in Syracuse dishes and treated with 2 ml of PMP solution at concentrations of 0 (negative control), 1, 10, 50, 100, or 250 μg/ml formulated in sterile water and kept at 26±1° C. for different exposure times. The number of juveniles emerged from the eggs is counted after 24, 48 and 72 h.

For the mortality test, egg masses are collected and incubated in water for 3 days to allow eggs to hatch. After 3 days, 100 second stage juveniles are added to the Syracuse dishes containing 2 ml of PMP solution at concentrations of 0 (negative control), 1, 10, 50, 100, or 250 μg/ml formulated in sterile water and are incubated at 26±1° C. Observations are recorded on the mortality of juveniles at 24, 48, and 72 h intervals using a stereoscope. Thereafter, juveniles treated with the PMP solution are transferred to distilled water and observed again after 24 hours to confirm their mortality. The survival rates, hatching rates, pupation rates of S. litura treated with the PMP solution are compared to the Lepidoptera treated with the negative control. The rates of S. litura treated with a PMP solution are decreased compared to the control, the fitness is negatively affected at each developmental stage.

c) Treatment of Spodoptera litura Larvae with Arabidopsis Apoplast PMPs

To determine the effect of apoplast PMPs on S. litura larval fitness, one hundred fresh S. litura eggs are carefully collected from the egg masses using a wet camel hair brush and are distributed 10 eggs/Petri dish (1.0×5.0 cm). Upon hatching, the larvae are transferred individually into plastic vials containing tobacco leaves that have been spray-treated with a solution of 0 (negative control), 1, 10, 50, 100, or 250 μg/ml of Arabidopsis apoplast PMPs, two hours prior to inoculation. Fresh leaves are provided daily. Observations on larval development, formation of pupae and successful emergence of adults and fecundity are recorded daily for two weeks. Age specific mortality in different developmental stages like, larvae, pupae, and adults are also recorded.

d) Treatment of Spodoptera litura Adults with Arabidopsis Apoplast PMPs in Planta

To determine the effect of apoplast PMPs on S. litura adult fitness, a non-infected 4-6 week old tobacco plant is sprayed with a solution of 0 (negative control), 1, 10, 50, 100, or 250 μg/ml of Arabidopsis apoplast PMPs isolated and purified as described in Examples 1-2. Two hours after spray inoculation, synchronized S. litura pupae collected 48 h after hatching, are transferred to the treated plants and kept at 26±1° C. After 72 h, adults are removed from the plant, counted and their fitness is assessed for their developmental stage—by size and morphological traits. Next, adults are transferred to 30×30×45 cm wooden cages lined with muslin cloth, to assess their fecundity. Five pairs of moths (5 females and 5 males), brought together in the mating cage the previous night, are released at 19.00 h into the cage. The following morning, moths are removed from the cage and eggs laid on leaves and muslin cloth in the cage are counted. Each female is used only once, and each test is replicated 5 times. As the eggs hatch, the weight of the larvae is compared between insects fed on different concentrations of PMPs compared to the negative control.

### Example 10: Treatment of a Fungus with Short Nucleic Acid-Loaded Plant Plant Messenger Packs

This example demonstrates the ability of PMPs to deliver short nucleic acids to a pest, by isolating PMP lipids and synthesizing them into vesicles containing short nucleic acids. In this example, short double-stranded RNAs (dsRNA)-loaded PMPs are used to knock down a virulence factor in a pathogenic fungus, Botrytis cinerea both in plants as in post-harvest produce. It also demonstrates that short nucleic-acid loaded-PMPs are stable and retain their activity over a range of processing and environmental conditions. In this example, dsRNA is used as a model nucleic acid, and Botrytis cinerea is used as a model pathogenic fungus, and grapes are used as model fruit.

### Therapeutic Dose:

PMPs loaded with dsRNA, formulated in water to a concentration that delivers an equivalent of an effective dsRNA dose of 0, 1, 5, 10 and 20 ng/μl in sterile water.

### Experimental Protocol:

a) Lipid Isolation from Grapefruit-Derived PMPs

Lipids are isolated from purified PMPs as described in Example 1-2, adapted from Xiao et al., Plant Cell, 22(5): 1463-1482, 2010. Briefly, 3.75 ml 2:1 (v/v) MeOH:CHCl3 is added to 1 ml of PMPs in PBS and vortexed. CHCl3 (1.25 ml) and ddH2O (1.25 ml) are added sequentially and vortexed. The mixture is then centrifuged at 2,000 r.p.m. for 10 min at 22° C. in glass tubes to separate the mixture into two phases (aqueous phase and organic phase). For collection of the organic phase, a glass pipette is inserted through the aqueous phase with gentle positive pressure, and the bottom phase (organic phase) is aspirated and dispensed into fresh glass tubes. The organic phase samples are aliquoted and dried by heating under nitrogen (2 psi).

b) Synthesis of Grapefruit PMPs Loaded with Dcl1/2 dsRNA

Short nucleic acids are loaded in PMPs according to a modified protocol from Wang et al, Nature Comm., 4:1867, 2013. Briefly, purified PMPs are produced from grapefruit according to Example 1-2, and grapefruit PMP lipids are isolated as described in Example 10a. Short Double stranded RNA (dsRNA) targeting Botrytis cinerea dcl1/2 with sequences as specified in Wang et al., Nature Plants. 2(10):16151, 2016, and a scrambled dsRNA control are obtained from IDT. dsRNA loaded-PMPs are synthesized from both targeted and control dsRNA, by mixing the lipids and short nucleic acids, which are dried to form a thin film. The film is dispersed in PBS and sonicated to form loaded liposomal formulations. PMPs are purified using a sucrose gradient as described in Example 2 and washed via ultracentrifugation before use to remove unbound nucleic acid. A small portion of both samples are characterized using the methods in Example 3, RNA content is measured using the Quant-It RiboGreen RNA assay kit, and their stability is tested as described in Example 4.

c) Treatment of Botrytis cinerea with Dcl12 Targeting dsRNA-Loaded Grapefruit PMPs for Reducing Fungal Fitness in Planta

To determine the efficiency of fungal blockade using dsRNA-loaded PMPs from Example 10b, Arabidopsis thaliana plants are sprayed with a PMP solution with an effective dsRNA dose of 0, 1, 5, 10 and 20 ng/μl in sterile water 2 d, 1 d and 2 h prior to bacterial inoculation.

Botrytis cinerea strain B05 is cultured on Malt extract agar (2% malt extract, 1% Bacto peptone). Spores are diluted in 1% Sabouraud Maltose Broth buffer to a final concentration of 105 spores/ml, and spray inoculated onto 4-6 week old Arabidopsos leaves, modified from Wang et al., Nature Plants. 2(10):16151, 2016. The effect and efficiency of treatment with dc/l/2-loaded PMPs, and 20 ng/μl dcl1/2 shRNA are compared to scrambled and negative controls.

One, three and five days after the initial infection, disease is assessed by quantifying the amount of Bc-DCL1/2 transcript knockdown in isolated Arabidopsis leaves, using the protocol from Wang et al., Nature Plants. 2(10):16151, 2016. The collected samples are subjected to RNA extraction using the Fisher BioReagents™ SurePrep™ Plant/Fungi Total RNA Purification Kit (Fisher scientific, Waltham, Mass.), cDNA synthesis using SuperScript III Reverse Transcriptase (Invitrogen Carlsbad, Calif.), and quantitative RT-PCR quantification. The expression of Bc-DCL1 and Bc-DCL2 in B. cinerea after treatment of synthesized Bc-DCL1/2-dsRNAs is measured using the following primers: Bc-DCL1-fw ACAATCCTATCTTTCGGAAGC, Bc-DCL1-rev AGACTCTTCTTCTTGAAGACAG, Bc-DCL2-fw GATTGTGCAAAGTCTCAACA, and Bc-DCL2-rev ATTGGGTTTGACTATATGTCTTA.

In addition, a DNA-based real-time PCR assay is used to quantify B. cinerea growth relative to Arabidopsis thaliana leaf biomass, as described by Ross and Somssich, Plant Methods. 12(1):48, 2016. DNA from 6 leaves from 6 individual plants are collected, and DNA is extracted using the FastDNA SPIN Kit for soil (MP Biomedicals) according to the manufacturer's instructions. For qPCR analysis 33 ng of DNA is mixed with 0.4 mM gene specific primers (B. cinerea fungal biomass (Bc3F, Suarez et al. Plant Physiol Bioch. 42(11):924-934, 2005): fw-GCTGTAATTT CAATGTGCAGAATCC, rev-GGAGCAA CAATTAATCGCATTTC; Arabidopsis plant biomass (At4 g26410, Ross and Somssich, Plant Methods. 12(1):48, 2016), fw-GAGCTGAAGTGGCTTCCATGAC, rev-GGTCCGACATACCCATGATCC), and qPCR is performed using SsoAdvanced™ Universal SYBR® Green Supermix (BioRad) with three technical replicates according to the following protocol: denaturation at 95° C. for 3 min, 40 repeats of 95° C. for 20 s, 61° C. for 20 s and 72° C. for 15 s.

The abundance of the fungal derived PCR product is normalized to the abundance of the plant derived PCR product. The in planta effect of Arabidopsis apoplast PMPs on fungal growth is determined by calculating the ΔΔCt value, comparing the normalized fungal growth in the negative PBS control to the normalized fungal growth in the PMP treatment samples.

d) Treatment of Botrytis cinerea with Dcl12 Targeting dsRNA-Loaded Grapefruit-Derived PMPs for Reducing Fungal Fitness on Post-Harvest Grapes

To determine the effect of dcl1/2 dsRNA-loaded grapefruit PMPs on fungal growth in post-harvest fruit, grapes were purchased from a local supermarket, and washed extensively before use.

Grapes are sprayed with a dsRNA-loaded PMP solution with an effective dsRNA dose of 0, 1, 5, 10 and 20 ng/μl in sterile water or a 20 ng/μl dcl1/2 or scrambled shRNA, 5 d, 3 d, 1 d, and 2 h prior to Botrytis cinerea fungal infection by drop inoculation of 20 μl of 105 spores/ml according to Wang et al., Nature Plants. 2(10):16151, 2016. Relative lesion sizes of the infected grape samples are measured 5 days post-inoculation, and quantified by Image J. B. cinerea relative DNA content (relative biomass) is measured by quantitative PCR as described in Example 10c. The effect and efficiency of treatment with dc/l/2-loaded PMPs, and dcl1/2 shRNA are compared to scrambled and negative controls.

### Example 11: Treatment of an Insect with Peptide Nucleic Acid (PNA)-Loaded Plant Messenger Packs

This example demonstrates loading of PMPs with a peptide nucleic acid construct for the purpose of reducing insect fitness by knocking down a gene in in a pest, e.g., Ultraspiracle (USP) in fall armyworm (Spodoptera frugiperda), which has been demonstrated in other lepidopterans to reduce larval viability and pupation rate. This example also demonstrates that PNA-loaded PMPs are stable and retain their activity over a range of processing and environmental conditions. In this example, PNA is used as a model protein, and Spodoptera frugiperda is used as a model pathogenic insect.

### Therapeutic Dose:

PMPs loaded with dsRNA, formulated in water to a concentration that delivers an equivalent of an effective PNA dose of 0, 0.1, 1, 5, and 10 μM in sterile water

### Experimental Protocol:

a) Identification of a Peptide Nucleic Construct Against Spodoptera frugiperda

Ten PNAs against Spodoptera frugiperda Ultraspiracle Protein (USP) are designed and synthesized by an appropriate vendor. Sf21 and Sf9 Spodoptera frugiperda cell lines are obtained from ThermoFisher Scientific and maintained as a suspension culture according to manufacturer's culture instruction. PNAs are tested in vitro by electroporation of the cells using a protocol adapted from elc et al, PLoS One. 10(3), e0119283, 2015. USP knockdown is measured by RT-qPCR using probes designed from an appropriate vendor. The best-performing PNA in terms of UPS knockdown efficiency is selected for further experiments.

b) Loading of Grapefruit PMPs with a Peptide Nucleic Acid

PMPs from grapefruit are isolated according to Example 1. PMPs are placed in solution with the PNA in PBS. The solution is then sonicated to induce poration and diffusion into the PMPs according to the protocol from Wang et al, Nature Comm., 4:1867, 2013. Alternatively, the solution can be passed through a lipid extruder according to the protocol from Haney et al, J Contr. Rel., 207:18-30, 2015. Alternatively, they can be electroporated according to the protocol from Wahlgren et al, Nucl. Acids. Res. 40(17):e130, 2012. After 1 hour, the PMPs are purified using a sucrose gradient and washed via ultracentrifugation as described in Example 2 before use to remove unbound nucleic acid.

Size, zeta potential, and particle count are measured using the methods in Example 3, and their stability is tested as described in Example 4. PNAs in the PMPs are quantified using an electrophoretic gel shift assay following the protocol in Nikravesh et al, Mol. Ther., 15(8): 1537-1542, 2007. Briefly, DNA antisense to the PNAs are mixed with PNA-PMPs treated with detergent to release the PNAs. PNA-DNA complexes are run on a gel and visualized with an ssDNA dye. The duplexes are then quantified by fluorescent imaging. Loaded and unloaded PMPs are compared to determine loading efficiency.

c) Treatment of Spodoptera frugiperda with PNA-Loaded Grapefruit PMPs for Reducing Insect Fitness

PMPs loaded with the USP PNA identified above and a scrambled PNA control are loaded into PMPs according to the method described above. Spodoptera frugiperda are obtained from a suitable vendor and maintained according to vendor's instructions. Larvae are fed PNAs against USP and control PNAs in PMPs according to the protocol for feeding adapted from Yang and Han, J. Integ. Ag. 13(1):115-123, 2014. Survival and pupation rate are measured to determine the effects.

### Example 12: Treatment of a Bacterium with Small Molecule-Loaded Plant Messenger Packs

This example demonstrates methods of loading PMPs with small molecules, in this embodiment, streptomycin, for the purpose of reducing the fitness of a bacteria, e.g., Pseudomonas syringae pv tomato. P. syringae represents a class of seedborne phytopathogenic bacteria that act as primary inoculum source for many important vegetable diseases. These bacterial diseases are economically important to their respective hosts and in most cases, infested seeds and seedlings serve as a primary inoculum source for epidemics in the greenhouse and in the field. This example further demonstrates that application of a coating comprising of streptomycin-loaded PMPs on tomato (Solanum lycopersicum) seeds reduces the fitness of P. syringae. It also demonstrates that small molecule loaded-PMPs are stable and retain their activity over a range of processing and environmental conditions. In this example, streptomycin is used as a model small molecule, and Pseudomonas syringae is used as a model pathogenic bacterium.

### Therapeutic Dose:

PMPs loaded with small molecule, formulated in water to a concentration that delivers an equivalent of an effective Streptomycin sulfate dose of 0, 2.5, 10, 50, 100, or 200 mg/ml

a) Loading of Grapefruit PMPs with a Small Molecule

PMPs produced, as described above, are placed in PBS solution with solubilized Streptomycin. The solution is left for 1 hour at 22° C., according to the protocol in Sun et al., Mol Ther. September; 18(9):1606-14, 2010. Alternatively, the solution is sonicated to induce poration and diffusion into the exosomes according to the protocol from Wang et al, Nature Comm., 4:1867, 2013. Alternatively, the solution can be passed through a lipid extruder according to the protocol from Haney et al, J Contr. Rel., 207:18-30, 2015. Alternatively, they can be electroporated according to the protocol from Wahlgren et al, Nucl. Acids. Res., 40(17): e130, 2012. After 1 hour, the loaded PMPs are purified using a sucrose gradient and washed via ultracentrifugation as described in Example 2 before use to remove unbound small molecules. Streptomycin-loaded PMPs are characterized for size and zeta potential using the methods in Example 3. A small amount of the PMPs are Streptomycin content is assessed using UV-Vis at 195 nm using a standard curve. Briefly, stock solutions of streptomycin at various concentrations of interest are made and 100 microliters of the solution are placed in a flat-bottom clear 96 well plate. The absorbance at 195 nm is measured using a UV-V plate reader. Samples are also put on the plate, and a regression is used to determine what the concentration could be according to the standard. For insufficiently high concentrations, the protocol from Kurosawa et al., J. Chromatogr., 343:379-385, 1985 is used to measure the streptomycin content by HPLC. Streptomycin-loaded PMP stability is tested as described in Example 4.

b) Treatment of Pseudomonas syringae with Streptomycin-Loaded Grapefruit PMPs for Reducing Bacterial Fitness

P. syringae pv are acquired from ATCC and grown according to manufacturer's instructions as described in Example 6. Effective concentrations of streptomycin, PMPs, and streptomycin-loaded PMPs are tested for ability to prevent growth of P. syringae according to the protocol adapted from Hoefler et al. Cell Chem. Bio. 24(10):1238-1249, 2017. Briefly, P. syringae cultures at the stationary phase are concentrated to OD₆₀₀=4 by centrifugation and resuspension in medium. 1.5 mL of concentrated P. syringae culture is mixed with 4.5 mL agar and spread evenly over a 25 mL agar plate. After solidification, 3 uL of a 0 (negative control), 2.5, 10, 50, 100, or 200 mg/ml effective dose of Streptomycin-loaded PMPs is spotted onto the overlay and allowed to dry. The plates are incubated overnight, photographed, and scanned. The diameter of the lytic zone (area without bacteria) around the spotted area is measured. Control (PBS), streptomycin, PMP, and streptomycin-loaded PMP-treated lytic zones are compared to determine the bactericidal effect. After solidification, an effective dose (microliters) of the treatment is spotted onto the overlay and allowed to dry. The plates are incubated overnight, photographed, and scanned. The size of the lytic zone (area without bacteria) is measured to determine efficacy.

c) Treatment of Tomato Seeds with Streptomycin-Loaded Grapefruit PMPs for Reducing Bacterial Fitness

Two hundred Micro-Tom Tomato seeds (USDA) per group were soaked in a suspension of Streptomycin alone or loaded in PMPs with an effective dose of 0, 2.5, 10, 50, 100, or 200 mg/ml for 2 hours at room temperature, and sown immediately after soaking. After 1, 2, and 5 days of incubation, seeds are infested by soaking samples in a P. syringae pv. tomato suspension containing approximately 10⁸ colony-forming units (CFU)/ml under vacuum for 30 min. The vacuum is released abruptly to favor entry of the pathogens into the seed cavities. The relative effect of streptomycin-loaded PMP seed treatments compared to Streptomycin or control treatment alone on P. syringae biomass was determined by qPCR and as described in Example 6d. The effect of streptomycin-loaded PMP seed treatments on germination of tomato seeds was assessed by recording the germination time and seedling development rate for 3-4 weeks, compared to streptomycin only or untreated control.

### Example 13: Treatment of a Nematode with Protein/Peptide-Loaded Plant Messenger Packs

This example demonstrates loading of PMPs with a peptide construct for the purpose of reducing fitness in parasitic nematodes. This example demonstrates PMPs loaded with GFP are taken up in the digestive tract of C. elegans, and that PMPs loaded with Mi-NLP-15b neuropeptide reduces Meloidogyne incognita nematode invasion of tomato plants. It also demonstrates that peptide-loaded PMPs are stable and retain their activity over a range of processing and environmental conditions. In this example, GFP and nematicidal peptide Mi-NLP-15b are used as a model peptide, and Meloidogyne incognita and C. elegans are used as model nematodes.

Plant parasitic nematodes (PPN) seriously threaten global food security. Conventionally an integrated approach to PPN management has relied heavily on carbamate, organophosphate, and fumigant nematicides which are now being withdrawn over environmental health and safety concerns. This progressive withdrawal has left a significant shortcoming in our ability to manage these economically important parasites, and highlights the need for novel and robust control methods.

### Therapeutic Dose:

PMPs loaded with peptide, formulated in water to a concentration that delivers an equivalent of an effective peptide dose of 0 (control), 1 nM, 10 nM, 100 nM, 1 μM, 10 μM, 50 μM, and 100 μM in sterile water. PMPs loaded with GFP, formulated in water to a concentration that delivers 0 (unloaded PMP control), 10, 100, 1000 μg/ml GFP-protein loaded in PMPs

### Experimental Protocol:

a) Loading Grapefruit PMPs with a Protein or Peptide

PMPs are placed in solution with the protein or peptide in PBS. If the protein or peptide is insoluble, pH is adjusted until it is soluble. If the protein or peptide is still insoluble, the insoluble protein or peptide is used. The solution is then sonicated to induce poration and diffusion into the exosomes according to the protocol from Wang et al, Nature Comm., 4:1867, 2013. Alternatively, the solution can be passed through a lipid extruder according to the protocol from Haney et al, J Contr. Rel., 207:18-30, 2015. Alternatively, they can be electroporated according to the protocol from Wahlgren et al, Nucl. Acids. Res. 40(17):e130, 2012. After 1 hour, the PMPs are purified using a sucrose gradient and washed via ultracentrifugation as described in Example 1 before use to remove unbound protein. PMP-derived liposomes are characterized as described in Example 3, and their stability is tested as described in Example 4. To measure loading of the protein or peptide, the Pierce Quantitative Peptide Assay is used on a small sample of the loaded and unloaded PMPs.

b) Treatment of Meliodogyne incognita Eggs with Mi-NLP-15b Neuropeptide-Loaded Grapefruit PMPs

PMPs are isolated from grapefruit according to Example 1-2. Nematicidal synthetic neuropeptide Mi-NLP-15b (sequence: SFDSFTGPGFTGLD) identified in Warnock, PLoS Pathogens, 13(2): e1006237, 2017 is synthesized by a commercial vendor. The peptide is then loaded into PMPs according to the methods above. A scrambled peptide is also loaded as a control. M. incognita were maintained in tomato plants, and eggs and juveniles were collected as described in Example 8 To assess the nematicidal activity of a Mi-NLP-15b neuropeptide-loaded grapefruit PMP solution on the eggs of Meliodogyne nematodes, an in vitro hatching test is conducted. Eggs masses of Meliodogyne nematodes are obtained from the infected roots. Single egg masses, containing an average of 300-350 eggs, are placed in Syracuse dishes and treated with 2 ml of the PMP solution at concentrations of 0 (control), 1 nM, 10 nM, 100 nM, 1 μM, 10 μM, 50 μM, or 100 μM of naked Mi-NLP-15b, scrambled peptide, or the effective dosages in Mi-NLP-15b-loaded PMPs, scrambled peptides-loaded in PMPs, or unloaded PMPs. and kept at 28±1° C. for different exposure times. The numbers of juveniles emerged from the eggs are counted after 24, 48 and 72 h. The effect on egg hatching is determined by comparing the percentage of juveniles emerged from the sterile water control with those from the PMP treatments.

c) Treatment of Meloidogyne incognita Juveniles with Mi-NLP-15b Neuropeptide-Loaded Grapefruit PMPs in Planta

M. incognita were maintained in tomato plants, and eggs and juveniles were collected as described in Example 8. Meloidogyne incognita infection is measured to assess the ability of the neuropeptide-loaded PMPs to reduce nematode infection, as shown by Warnock, PLoS Pathogens, 13(2): e1006237, 2017. Briefly, tomato seeds are germinated on 0.5% Murashige and Skoog plates, and two-day old tomato seedlings are spray-treated or soaked with 0 (control), 1 nM, 10 nM, 100 nM, 1 μM, 10 μM, 50 μM and 100 μM of naked Mi-NLP-15b, scrambled peptide, or the effective dosages in Mi-NLP-15b-loaded PMPs, scrambled peptides-loaded in PMPs, or unloaded PMPs, and left to dry for 2 h, 6 h, 1 d, and 2 d prior to infection. Invasion assays are performed by mixing 500 pre-treated M. incognita J2s with agar slurry and a single treated tomato seedling in a 6 well plate. Assays are left for 24 h under a 16 h light and 8 h darkness cycle. Seedlings are stained using acid fuschin and the number of nematodes within the roots counted and neuropeptide-loaded PMP treatments are compared to controls. At least five seedlings per condition are used for infection assays.

d) Delivery of a Model Protein to a Nematode

PMPs are isolated from grapefruit according to Example 1. Green fluorescent protein is synthesized commercially and solubilized in PBS. It is then loaded into PMPs according to the methods described above, and GFP encapsulation of PMPs was measured by Western blot or fluorescence. C. elegans wild-type N2 Bristol strain (C. elegans Genomics Center) are maintained on an Escherichia coli (strain OP50) lawn on nematode growth medium (NGM) agar plates (3 g/l NaCl, 17 g/l agar, 2.5 g/l peptone, 5 mg/l cholesterol, 25 mM KH₂PO₄ (pH 6.0), 1 mM CaCl₂), 1 mM MgSO₄) at 20° C., from L1 until the L4 stage.

One-day old C. elegans are transferred to a new plate and are fed 0 (unloaded PMP control), 10, 100, 1000 ug/ml GFP-loaded PMPs in a liquid solution following the feeding protocol in Conte et al., Curr. Protoc. Mol. Bio., 109:26.3.1-30 2015. They are then examined under a fluorescent microscope for green fluorescence along the digestive tract, compared to a PMP or sterile water control.

### Example 14: Treatment of a Plant with Herbicide-Loaded Plant Messenger Packs

This example demonstrates the loading and delivery of the herbicide Glufosinate in PMPs, to affect the fitness of a plant. This example further demonstrates that small molecule loaded-PMPs are stable and retain their activity over a range of processing and environmental conditions. In this example, Glufosinate is used as a model small molecule herbicide, and Eleusine indica is used as a model weed.

Eleusine indica (L.) (Indian goosegrass), one of the world's worst weeds, is a very competitive and cosmopolitan species. Eleusine indica is fecund, found across a range of soils and temperatures and infests a wide range of crops including cotton, maize, rice, sugarcane and many fruit and vegetable orchards. Effective and safe herbicides are necessary to prevent major crop yield loss due to weeds, while protecting the environment from the toxic side-effect of herbicide over-use.

### Therapeutic Dose:

PMPs loaded with small molecule Glufosinate, formulated in water to a concentration that delivers an equivalent of an effective dose of 0, 0.25, 0.5, 1, 3, or 6 mg/ml Glufosinate.

### Experimental Protocol:

a) Loading of Grapefruit PMPs with Small Molecule Herbicide Glufosinate

PMPs are produced from grapefruit according to Example 1-2. PMPs are placed in PBS solution with solid or solubilized Glufosinate (CAS 77182-82-2, Sigma-Aldrich). The solution is left for 1 hour at 22° C., according to the protocol in Sun et al., Mol Ther. 2010 September; 18(9):1606-14.

Alternatively, the solution is sonicated to induce poration and diffusion into the exosomes according to the protocol from Wang et al, Nature Comm., 4:1867, 2013. Alternatively, the solution can be passed through a lipid extruder according to the protocol from Haney et al, J Contr. Rel., 207:18-30, 2015. Alternatively, they can be electroporated according to the protocol from Wahlgren et al, Nucl. Acids. Res. 40(17):e130, 2012. After 1 hour, the loaded PMPs are purified using a sucrose gradient and washed via ultracentrifugation as described in Example 2 before use to remove unbound small molecules. Glufosinate-loaded PMPs are characterized for size and zeta potential using the methods in Example 3.

To quantify Glufosinate encapsulation, Glufosinate-loaded PMPs are decomposed using Bligh and Dayer method, Glufosinate being dissolved in the upper phase. Glufosinate is determined using High Performance Liquid Chromatography with Diode-Array Detection (HPLC-DAD) according to the method described in Changa et al, Journal of the Chinese Chemical Society, 52(4): 785-792, 2005. Briefly, 9-fluorenylmethyl chloroformate (FMOC-Cl) is used for pre-column derivatization of the non-absorbing Glufosinate. The samples are separated by HPLC-DAD at 12 min with 25 mM borate buffer at pH 9, followed by determination with a UV detector at 260 nm.

b) Treatment of Indian Goosegrass Weed with Glufosinate-Loaded PMPs

The herbicidal effect of Glufosinate treatment is measured in Indian goosegrass plants (Eleusine indica). Eleusine indica seeds are germinated on water-solidified 0.6% agar containing 0.2% potassium nitrate (KNO3) (Ismail et al., Weed Biology and Management, 2(4):177-185, 2002). At the 3-5 leaf stage, seedlings are subjected to different Glufosinate treatments by spraying the whole plant with 0 (negative control), 0.25, 0.5, 1, 3, or 6 mg/ml Glufosinate, or 0 (unloaded PMP control), 0.25, 0.5, 1, 3, or 6 mg/ml Glufosinate-loaded PMPs, 1 ml solution per plant, with 3 plants per group. Glufosinate activity was assessed phenotypically (signs of chlorosis and wilting, necrosis, plant death) on days 22 and 35 after treatment. At day 35, above-ground shoots were harvested and dried in oven (65° C.) for 3 days for dry-weight measurements, and Glufosinate-loaded PMP treatments are compared to PMP-only and Glufosinate controls.

### Example 15: PMP Production from Blended Fruit Juice Using Ultracentrifugation and Sucrose Gradient Purification

This example demonstrates that PMPs can be produced from fruit by blending the fruit and using a combination of sequential centrifugation to remove debris, ultracentrifugation to pellet crude PMPs, and using a sucrose density gradient to purify PMPs. In this example, grapefruit was used as a model fruit.

a) Production of Grapefruit PMPs by Ultracentrifugation and Sucrose Density Gradient Purification

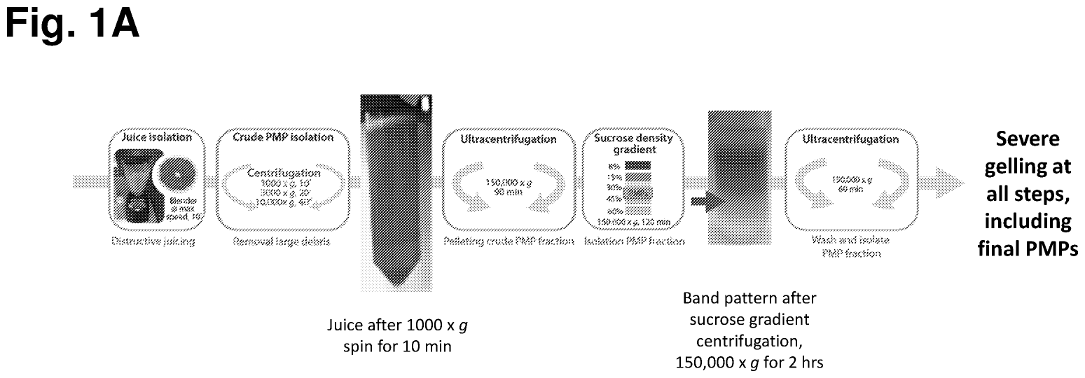

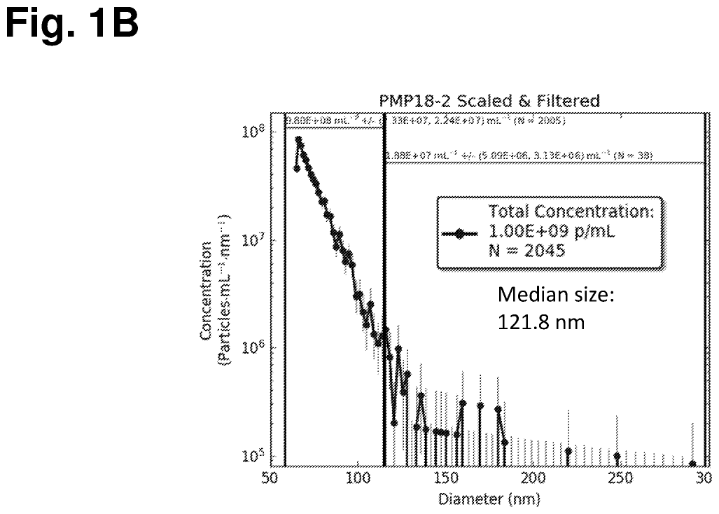

A workflow for grapefruit PMP production using a blender, ultracentrifugation and sucrose gradient purification is shown in FIG. 1A. One red grapefruit was purchased from a local Whole Foods Market®, and the albedo, flavedo, and segment membranes were removed to collect juice sacs, which were homogenized using a blender at maximum speed for 10 minutes. One hundred mL juice was diluted 5× with PBS, followed by subsequent centrifugation at 1000×g for 10 minutes, 3000× g for 20 minutes, and 10,000× g for 40 minutes to remove large debris. 28 mL of cleared juice was ultracentrifuged on a Sorvall™ MX 120 Plus Micro-Ultracentrifuge at 150,000× g for 90 minutes at 4° C. using a S50-ST (4×7 mL) swing bucket rotor to obtain a crude PMP pellet which was resuspended in PBS pH 7.4. Next, a sucrose gradient was prepared in Tris-HCL pH7.2, crude PMPs were layered on top of the sucrose gradient (from top to bottom: 8, 15, 30, 45 and 60% sucrose), and spun down by ultracentrifugation at 150,000×g for 120 minutes at 4° C. using a S50-ST(4×7 mL) swing bucket rotor. One mL fractions were collected and PMPs were isolated at the 30-45% interface. The fractions were washed with PBS by ultracentrifugation at 150,000× g for 120 minutes at 4° C. and pellets were dissolved in a minimal amount of PBS.

PMP concentration (1×10⁹ PMPs/mL) and median PMP size (121.8 nm) were determined using a Spectradyne nCS1™ particle analyzer, using a TS-400 cartridge (FIG. 1B). The zeta potential was determined using a Malvern Zetasizer Ultra and was −11.5+/−0.357 mV.

This example demonstrates that grapefruit PMPs can be isolated using ultracentrifugation combined with sucrose gradient purification methods. However, this method induced severe gelling of the samples at all PMP production steps and in the final PMP solution.

### Example 16: PMP Production from Mesh-Pressed Fruit Juice Using Ultracentrifugation and Sucrose Gradient Purification

This example demonstrates that cell wall and cell membrane contaminants can be reduced during the PMP production process by using a milder juicing process (mesh strainer). In this example, grapefruit was used as a model fruit.

a) Mild Juicing Reduces Gelling During PMP Production from Grapefruit PMPs

Juice sacs were isolated from a red grapefruit as described in Example 15. To reduce gelling during PMP production, instead of using a destructive blending method, juice sacs were gently pressed against a tea strainer mesh to collect the juice and to reduce cell wall and cell membrane contaminants. After differential centrifugation, the juice was clearer than after using a blender, and one clean PMP-containing sucrose band at the 30-45% intersection was observed after sucrose density gradient centrifugation (FIG. 2). There was overall less gelling during and after PMP production.

Our data shows that use of a mild juicing step reduces gelling caused by contaminants during PMP production when compared to a method comprising blending.

### Example 17: PMP Production Using Ultracentrifugation and Size Exclusion Chromatography

This example describes the production of PMPs from fruits by using Ultracentrifugation (UC) and Size Exclusion Chromatography (SEC). In this example, grapefruit is used as a model fruit.

a) Production of Grapefruit PMPs Using UC and SEC

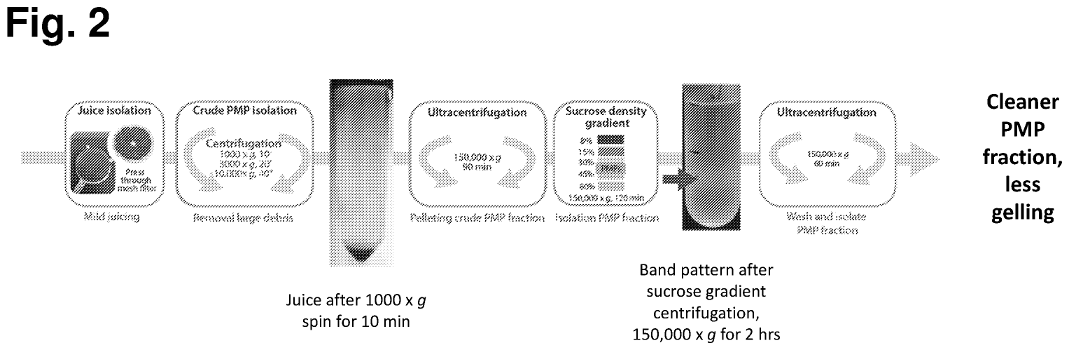

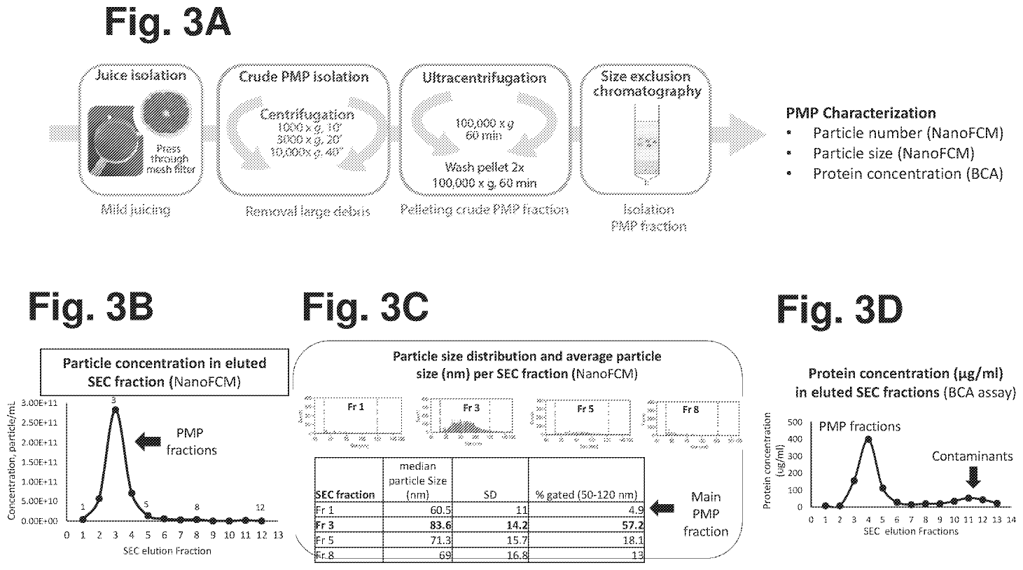

Juice sacs were isolated from a red grapefruit, as described in Example 15a, and were gently pressed against a tea strainer mesh to collect 28 ml juice. The workflow for grapefruit PMP production using UC and SEC is depicted in FIG. 3A. Briefly, juice was subjected to differential centrifugation at 1000×g for 10 minutes, 3000× g for 20 minutes, and 10,000× g for 40 minutes to remove large debris. 28 ml of cleared juice was ultracentrifuged on a Sorvall™ MX 120 Plus Micro-Ultracentrifuge at 100,000× g for 60 minutes at 4° C. using a S50-ST (4×7 mL) swing bucket rotor to obtain a crude PMP pellet which was resuspended in MES buffer (20 mM MES, NaCl, pH 6). After washing the pellets twice with MES buffer, the final pellet was resuspended in 1 ml PBS, pH 7.4. Next, we used size exclusion chromatography to elute the PMP-containing fractions. SEC elution fractions were analyzed by nano-flow cytometry using a NanoFCM to determine PMP size and concentration using concentration and size standards provided by the manufacturer. In addition, absorbance at 280 nm (SpectraMax®) and protein concentration (Pierce™ BCA assay, ThermoFisher) were determined on SEC fractions to identify in which fractions PMPs are eluted (FIGS. 3B-3D). SEC fractions 2-4 were identified as the PMP-containing fractions. Analysis of earlier- and later-eluting fractions indicated that SEC fraction 3 is the main PMP-containing fraction, with a concentration of 2.83×10¹¹ PMPs/mL (57.2% of all particles in the 50-120 nm size range), with a median size of 83.6 nm+/−14.2 nm (SD). While the late elution fractions 8-13 had a very low concentration of particles as shown by NanoFCM, protein contaminants were detected in these fractions by BCA analysis.

Our data shows that TFF and SEC can be used to isolate purified PMPs from late-eluting contaminants, and that a combination of the analysis methods used here can identify PMP fractions from late-eluting contaminants.

### Example 18: Scaled PMP Production Using Tangential Flow Filtration and Size Exclusion Chromatography Combined with EDTA/Dialysis to Reduce Contaminants

This example describes the scaled production of PMPs from fruits by using Tangential Flow Filtration (TFF) and Size Exclusion Chromatography (SEC), combined with an EDTA incubation to reduce the formation of pectin macromolecules, and overnight dialysis to reduce contaminants. In this example, grapefruit is used as a model fruit.

a) Production of Grapefruit PMPs Using TFF and SEC

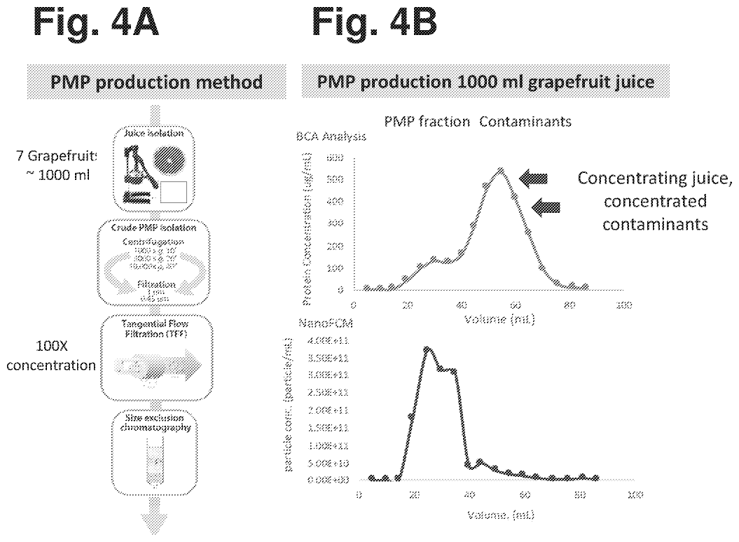

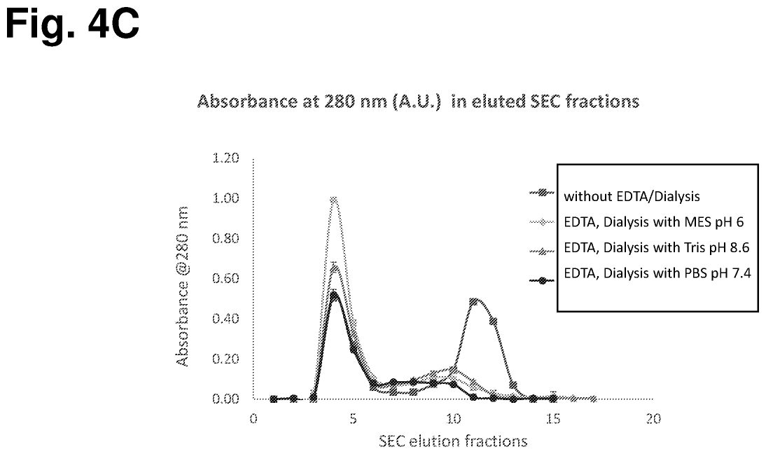

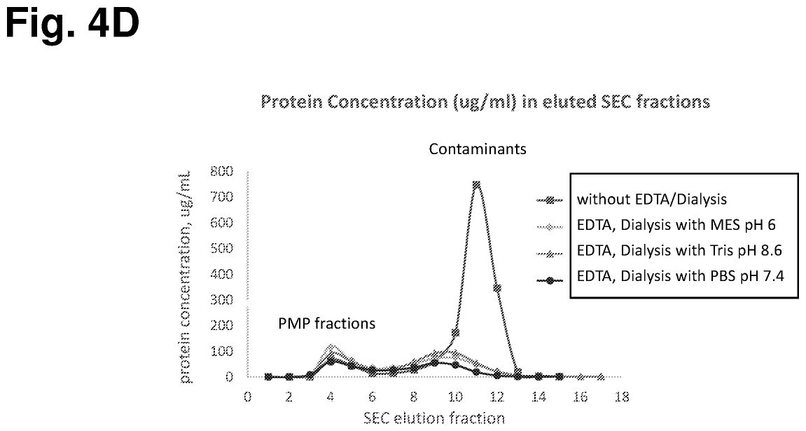

Red grapefruits were obtained from a local Whole Foods Market®, and 1000 ml juice was isolated using a juice press. The workflow for grapefruit PMP production using TFF and SEC is depicted in FIG. 4A. Juice was subjected to differential centrifugation at 1000×g for 10 minutes, 3000× g for 20 minutes, and 10,000× g for 40 minutes to remove large debris. Cleared grapefruit juice was concentrated and washed once using a TFF (5 nm pore size) to 2 mL (100×). Next, we used size exclusion chromatography to elute the PMP-containing fractions. SEC elution fractions were analyzed by nano-flow cytometry using a NanoFCM to determine PMP concentration using concentration and size standards provided by the manufacturer. In addition, protein concentration (Pierce™ BCA assay, ThermoFisher) was determined for SEC fractions to identify the fractions in which PMPs are eluted. The scaled production from 1 liter of juice (100× concentrated) also concentrated a high amount of contaminants in the late SEC fractions as can be detected by BCA assay (FIG. 4B, top panel). The overall total PMP yield (FIG. 4B, bottom panel) was lower in the scaled production when compared to single grapefruit isolations, which may indicate loss of PMPs.

b) Reducing Contaminants by ED TA Incubation and Dialysis

Red grapefruits were obtained from a local Whole Foods Market®, and 800 ml juice was isolated using a juice press. Juice was subjected to differential centrifugation at 1000×g for 10 minutes, 3000× g for 20 minutes, and 10,000× g for 40 minutes to remove large debris, and filtered through a 1 μm and 0.45 μm filter to remove large particles. Cleared grapefruit juice was split into 4 different treatment groups containing 125 ml juice each. Treatment Group 1 was processed as described in Example 18a, concentrated and washed (PBS) to a final concentration of 63×, and subjected to SEC. Prior to TFF, 475 ml juice was incubated with a final concentration of 50 mM EDTA, pH 7.15 for 1.5 hrs at RT to chelate iron and reduce the formation of pectin macromolecules. Afterwards, juice was split in three treatment groups that underwent TFF concentration with either a PBS (without calcium/magnesium) pH 7.4, MES pH 6, or Tris pH 8.6 wash to a final juice concentration of 63×. Next, samples were dialyzed in the same wash buffer overnight at 4° C. using a 300 kDa membrane and subjected to SEC. Compared to the high contaminant peak in the late elution fractions of the TFF only control, EDTA incubation followed by overnight dialysis strongly reduced contaminants, as shown by absorbance at 280 nm (FIG. 4C) and BCA protein analysis (FIG. 4D), which is sensitive to the presence of sugars and pectins. There was no difference in the dialysis buffers used (PBS without calcium/magnesium pH 7.4, MES pH 6, Tris pH 8.6).

Our data indicates that incubation with EDTA followed by dialysis reduces the amount of co-purified contaminants, facilitating scaled PMP production.

### Example 19: PMP Stability

This example demonstrates that PMPs are stable at different environmental conditions. In this example, grapefruit and lemon PMPs are used as model PMPs.

a) Production of Grapefruit PMPs Using TFF Combined with SEC

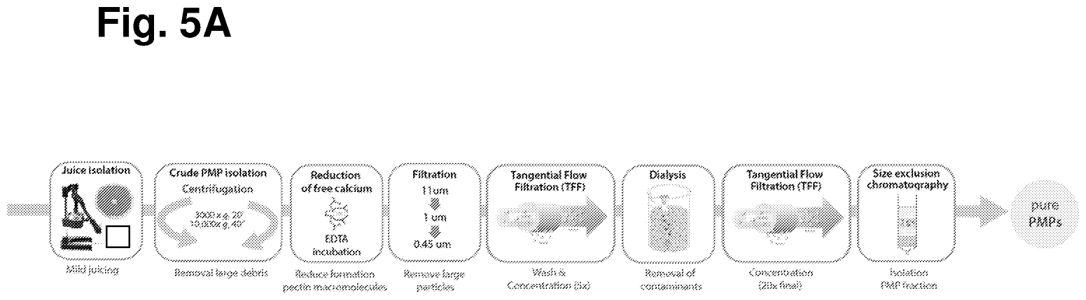

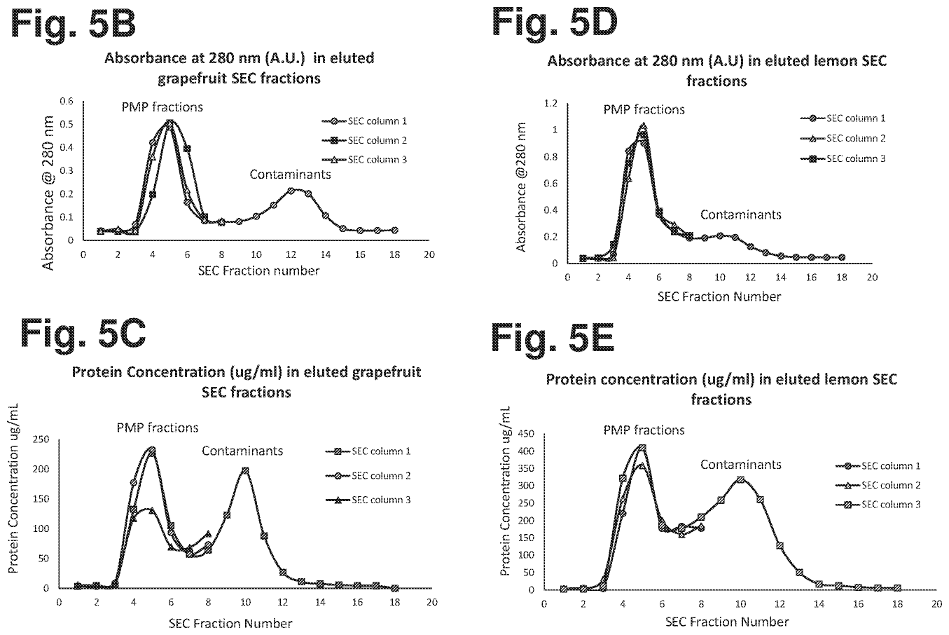

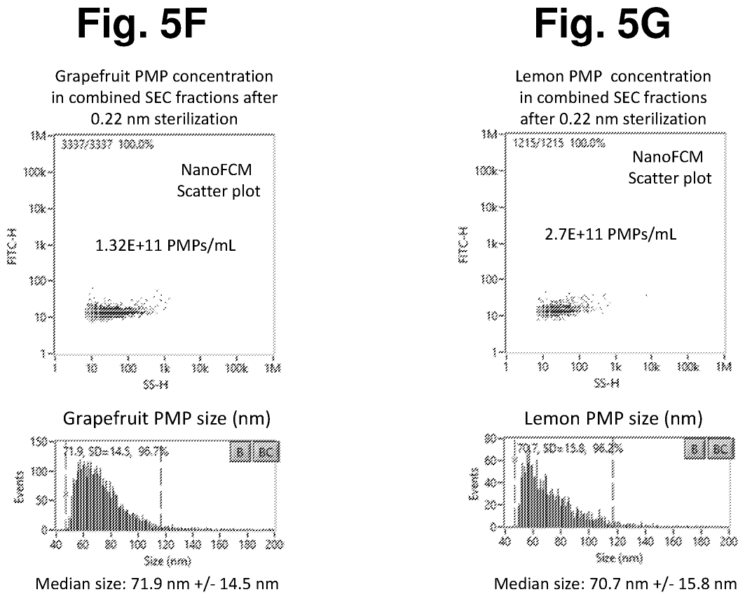

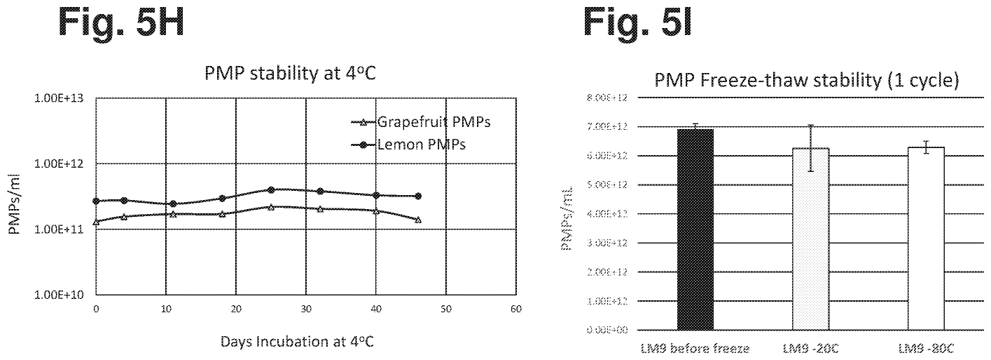

Red organic grapefruits (Florida) were obtained from a local Whole Foods Market®. The PMP production workflow is depicted in FIG. 5A. One liter of grapefruit juice was collected using a juice press, and was subsequently centrifuged at 3000×g for 20 minutes, followed by 10,000× g for 40 minutes to remove large debris. Next, 500 mM EDTA pH 8.6 was added to a final concentration of 50 mM EDTA, pH 7, and the solution was incubated for 30 minutes to chelate calcium and prevent the formation of pectin macromolecules. Subsequently the juice was passaged through 11 μm, 1 μm and 0.45 μm filters to remove large particles. Filtered juice was concentrated and washed (500 ml PBS) by Tangential Flow Filtration (TFF) (pore size 5 nm) to 400 ml (2.5×) and dialyzed overnight in PBS pH 7.4 (with one medium exchange) using a 300 kDa dialysis membrane to remove contaminants. Subsequently, the dialyzed juice was further concentrated by TFF to a final concentration of 50 ml (20×). Next, we used size exclusion chromatography to elute the PMP-containing fractions, which were analyzed by absorbance at 280 nm (SpectraMax®) and a protein concentration assay (Pierce™ BCA assay, ThermoFisher) to verify the PMP-containing fractions and late fractions containing contaminants (FIGS. 5B and 5C). SEC fractions 4-6 contained purified PMPs (fractions 8-14 contained contaminants), were pooled together, and were filter sterilized by sequential filtration using 0.8 m, 0.45 m and 0.22 μm syringe filters. The final PMP concentration (1.32×10¹¹ PMPs/mL) and median PMP size (71.9 nm+/−14.5 nm) in the combined sterilized PMP-containing fractions were determined by NanoFCM using concentration and size standards provided by the manufacturer (FIG. 5F).

b) Production of Lemon PMPs Using TFF Combined with SEC

Lemons were obtained from a local Whole Foods Market®. One liter of lemon juice was collected using a juice press, and was subsequently centrifuged at 3000 g for 20 minutes, followed by 10,000 g for 40 minutes to remove large debris. Next, 500 mM EDTA pH 8.6 was added to a final concentration of 50 mM EDTA, pH 7, and the solution was incubated for 30 minutes to chelate calcium and prevent the formation of pectin macromolecules. Subsequently the juice was passaged through a coffee filter, 1 μm and 0.45 μm filters to remove large particles. Filtered juice was concentrated by Tangential Flow Filtration (TFF) (5 nm pore size) to 400 ml (2.5× concentrated) and dialyzed overnight in PBS pH 7.4 using a 300 kDa dialysis membrane to remove contaminants. Subsequently, the dialyzed juice was further concentrated by TFF to a final concentration of 50 ml (20×). Next, we used size exclusion chromatography to elute the PMP-containing fractions, which were analyzed by absorbance at 280 nm (SpectraMax®) and a protein concentration assay (Pierce™ BCA assay, ThermoFisher) to verify the PMP-containing fractions and late fractions containing contaminants (FIGS. 5D and 5E). SEC fractions 4-6 contained purified PMPs (fractions 8-14 contained contaminants), were pooled together, and were filter sterilized by sequential filtration using 0.8 μm, 0.45 μm and 0.22 μm syringe filters. The final PMP concentration (2.7×10¹¹ PMPs/mL) and median PMP size (70.7 nm+/−15.8 nm) in the combined sterilized PMP-containing fractions were determined by NanoFCM, using concentration and size standards provided by the manufacturer (FIG. 5G).

c) Stability of Grapefruit and Lemon PMPs at 4° C.

Grapefruit and lemon PMPs were produced as described in Examples 19a and 19b. The stability of PMPs was assessed by measurement of concentration of total PMPs (PMP/ml) in the sample over time using NanoFCM. The stability study was carried out at 4° C. for 46 days in the dark. Aliquots of PMPs were stored at 4° C. and analyzed by NanoFCM on predetermined days. The concentrations of total PMPs in the sample were analyzed (FIG. 5H). The relative measured PMP concentration of lemon and grapefruit PMPs between the start and endpoint of the experiment at 46 days was 119% and 107%, respectively. Our data indicate that PMPs are stable for at least 46 days at 4° C.

d) Freeze-Thaw Stability of Lemon PMPs

To determine the freeze-thaw stability of PMPs, lemon PMPs were produced from organic lemons purchased at a local Whole Foods Market®. One liter of lemon juice was collected using a juice press, and was subsequently centrifuged at 3000 g for 20 minutes, followed by 10,000 g for 40 minutes to remove large debris. Next, 500 mM EDTA pH 8.6 was added to final concentration of 50 mM EDTA, pH 7.5 and incubated for 30 minutes to chelate calcium and prevent the formation of pectin macromolecules. Subsequently, the juice was passaged through 11 μm, 1 μm and 0.45 μm filters to remove large particles. Filtered juice was concentrated and washed with 400 ml PBS, pH 7.4 by Tangential Flow Filtration (TFF) to a final volume of 400 ml (2.5× concentrated) and dialyzed overnight in PBS pH 7.4 using a 300 kDa dialysis membrane to remove contaminants. Subsequently, the dialyzed juice was further concentrated by TFF to a final concentration of 60 ml (˜17×). Next, we used size exclusion chromatography to elute the PMP-containing fractions, which were analyzed by absorbance at 280 nm (SpectraMax®) and a protein concentration assay (Pierce™ BCA assay, ThermoFisher) to verify the PMP-containing fractions and late fractions containing contaminants. SEC fractions 4-6 contained purified PMPs (fractions 8-14 contained contaminants), were pooled together, and were filter sterilized by sequential filtration using 0.8 μm, 0.45 μm and 0.22 μm syringe filters. The final PMP concentration (6.92×10¹² PMPs/mL) in the combined sterilized PMP containing fractions was determined by NanoFCM, using concentration and size standards provided by the manufacturer.

Lemon PMPs were frozen at −20° C. or −80° C. for one week, thawed at room temperature, and the concentration was measured by NanoFCM (FIG. 5I). The data indicate that lemon PMPs are stable after 1 freeze-thaw cycle after storage for one week at −20° C. or −80° C.

### Example 20: PMP Production from Plant Cell Culture Medium

This example demonstrates that PMPs can be produced from plant cell culture. In this example, the Zea mays Black Mexican Sweet (BMS) cell line is used as a model plant cell line.

a) Production of Zea mays BMS Cell Line PMPs

The Zea mays Black Mexican sweet (BMS) cell line was purchased from the ABRC and was grown in Murashige and Skoog basal medium pH 5.8, containing 4.3 g/L Murashige and Skoog Basal Salt Mixture (Sigma M5524), 2% sucrose (S0389, Millipore Sigma), 1× MS vitamin solution (M3900, Millipore Sigma), 2 mg/L 2,4-dichlorophenoxyacetic acid (D7299, Millipore Sigma) and 250 ug/L thiamine HCL (V-014, Millipore Sigma), at 24° C. with agitation (110 rpm), and was passaged 20% volume/volume every 7 days.

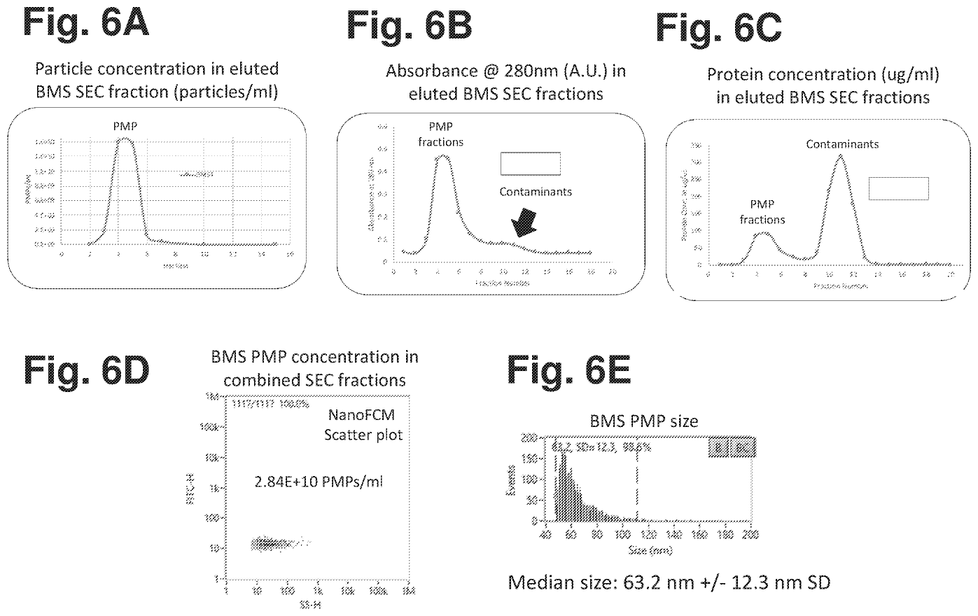

Three days after passaging, 160 ml BMS cells was collected and spun down at 500×g for 5 min to remove cells, and 10,000×g for 40 min to remove large debris. Medium was passed through a 0.45 μm filter to remove large particles, and filtered medium was concentrated and washed (100 ml MES buffer, 20 mM MES, 100 mM NaCL, pH 6) by TFF (5 nm pore size) to 4 mL (40×). Next, we used size exclusion chromatography to elute the PMP-containing fractions, which were analyzed by NanoFCM for PMP concentration, by absorbance at 280 nm (SpectraMax®), and by a protein concentration assay (Pierce™ BCA assay, ThermoFisher) to verify the PMP-containing fractions and late fractions containing contaminants (FIGS. 6A-6C). SEC fractions 4-6 contained purified PMPs (fractions 9-13 contained contaminants), and were pooled together. The final PMP concentration (2.84×10¹⁰ PMPs/ml) and median PMP size (63.2 nm+/−12.3 nm SD) in the combined PMP containing fractions were determined by NanoFCM, using concentration and size standards provided by the manufacturer (FIGS. 6D-6E).

These data show that PMPs can be isolated, purified, and concentrated from plant liquid culture media.

### Example 21: Uptake of PMPs by Bacteria and Fungi

This example demonstrates the ability of PMPs to associate with and be taken up by bacteria and fungi. In this example, grapefruit and lemon PMPs are used as a model PMP, Escherichia coli, Pseudomonas syringae, and Pseudomonas aeruginosa are used as model pathogenic bacteria, and the yeast Saccharomyces cerevisiae is used as a model pathogenic fungus.

a) Labeling of Grapefruit and Lemon PMPs with DyLight 800 NHS Ester

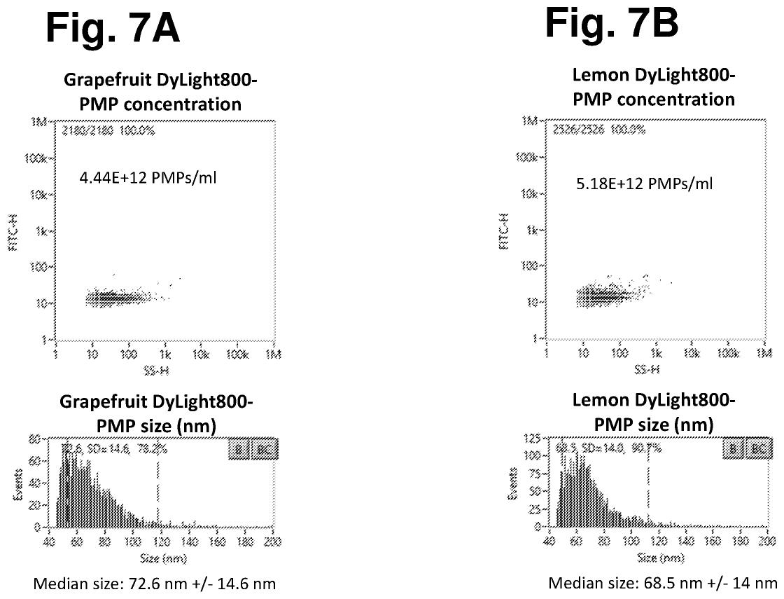

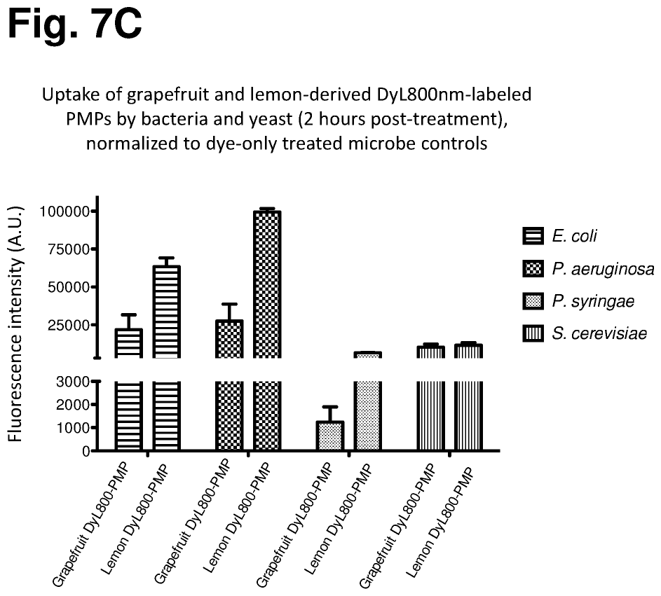

Grapefruit and lemon PMPs were produced as described in Examples 19a and 19b. PMPs were labeled with the DyLight 800 NHS Ester (Life Technologies, #46421) covalent membrane dye (DyL800). Briefly, DyL800 was dissolved in DMSO to a final concentration of 10 mg/ml, and 200 μl of PMPs were mixed with 5 μl dye and incubated for 1 h at room temperature on a shaker. Labeled PMPs were washed 2-3 times by ultracentrifuge at 100,000×g for 1 hr at 4° C., and pellets were resuspended with 1.5 ml UltraPure water. To control for the presence of potential dye aggregates, a dye-only control sample was prepared according to the same procedure, adding 200 μl of UltraPure water instead of PMPs. The final DyL800-labeled PMP pellet and DyL800 dye-only control were resuspended in a minimal amount of UltraPure water and characterized by NanoFCM. The final concentration of grapefruit DyL800-labeled PMPs was 4.44×10¹² PMPs/ml, with a median DyL800-PMP size of 72.6 nm+/−14.6 nm (FIG. 7A), and the final concentration of lemon DyL800-labeled PMPs was 5.18×10¹² PMPs/ml with an average DyL800-PMP size of 68.5 nm+/−14 nm (FIG. 7B).

b. Uptake of DyL800-Labeled Grapefruit and Lemon PMPs by Yeast

Saccharomyces cerevisiae (ATCC, #9763) was grown on yeast extract peptone dextrose broth (YPD) and maintained at 30° C. To determine whether PMPs can be taken up by yeast, a fresh 5 ml yeast culture was grown overnight at 30° C., and cells were pelleted at 1500×g for 5 min and resuspended in 10 ml water. Yeast cells were washed once with 10 ml water, resuspended in 10 ml water, and incubated for 2 h at 30° C. with shaking to nutrient starve the cells. Next, 95 ul of yeast cells were mixed with either 5 ul water (negative control), DyL800 dye only control (dye aggregate control), or DyL800-PMPs to a final concentration of 5×10¹⁰ DyL800-PMPs/ml in a 1.5 ml tube. Samples were incubated for 2 h at 30° C. with shaking. Next, treated cells were washed with 1 ml wash buffer (water supplemented with 0.5% Triton X-100), incubated for 5 min, and spun down at 1500×g for 5 min. The supernatant was removed and the yeast cells were washed an additional 3 times to remove PMPs that are not taken up by the cells and a final time with water to remove the detergent. Yeast cells were resuspended in 100 ul water and transferred to a clear bottom 96 well plate, and the relative fluorescence intensity (A.U.) at 800 nm excitation was measured on an Odyssey® CLx scanner (Li-Cor).

To assess DyL800-PMP uptake by yeast, samples were normalized to the DyL800 dye only control, and the grapefruit and lemon DyL800-PMP relative fluorescence intensities were compared. Our data indicates that Saccharomyces cerevisiae takes up PMPs, and no uptake difference was observed between lemon and grapefruit DyL800-PMPs (FIG. 7C).

c) Uptake of DyL800-Labeled Grapefruit and Lemon PMPs by Bacteria

Bacteria and yeast strains were maintained as indicated by the supplier: E. coli(Ec, ATCC, #25922) was grown on Trypticase Soy Agar/broth at 37° C., Pseudomonas aeruginosa (Pa, ATCC) was grown on Tryptic soy Agar/broth with 50 mg/ml rifampicin at 37° C., and Pseudomonas syringae pv. tomato str. DC3000 bacteria (Ps, ATCC, #BAA-871) was grown on King's Medium B agar with 50 mg/ml rifampicin at 30° C.

To determine whether PMPs can be taken up by bacteria, fresh 5 ml bacterial cultures were grown overnight, and cells were pelleted at 3000×g for 5 min, resuspended in 5 ml 10 mM MgCl₂, washed once with 5 ml 10 mM MgCl₂, and resuspended in 5 ml 10 mM MgCl₂. Cells were incubated for 2 h at 37° C. (Ec) or 30° C. (Pa, Ps) in a shaking incubator at ˜200 rpm to nutrient starve the cells. The OD600 was measured and cell densities were adjusted to ˜10×10⁹ CFU/ml. Next, 95 ul of bacterial cells were mixed with either 5 ul water (negative control), DyL800 dye only control (dye aggregate control), or DyL800-PMPs at a final concentration of 5×10¹⁰ DyL800-PMPs/ml in a 1.5 ml tube. Samples were incubated for 2 h at 30° C. with shaking. Next, treated cells were washed with 1 ml wash buffer (10 mM MgCl₂ with 0.5% Triton X-100), incubated for 5 min, and spun down at 3000× g for 5 min. The supernatant was removed and the yeast cells were washed an additional 3 times to remove PMPs that are not taken up by the cells, and once more with 1 ml 10 mM MgCl₂ to remove detergent. Bacterial cells were resuspended in 100 ul 10 mM MgCl₂ and transferred to a clear bottom 96 well plate, and the relative fluorescence intensity (A.U.) at 800 nm excitation was measured on an Odyssey® CLx scanner (Li-Cor).

To assess DyL800-PMP uptake by bacteria, samples were normalized to the DyL800 dye only control, and the grapefruit and lemon DyL800-PMP relative fluorescence intensities were compared. Our data indicates that all bacteria species tested take up PMPs (FIG. 7C). In general, lemon PMPs were preferentially taken up (higher signal intensity than grapefruit PMPs). E. coli and P. aeruginosa displayed the highest DyL800-PMP uptake.

### Example 22: Uptake of PMPs by Insect Cells

This example demonstrates the ability of PMPs to associate with and be taken up by insect cells. In this example, sf9 Spodoptera frugiperda (insect) cells and S2 Drosophila melanogaster(insect) cell lines are used as model insect cells, and lemon PMPs are used as model PMPs.

a) Production of Lemon PMPs

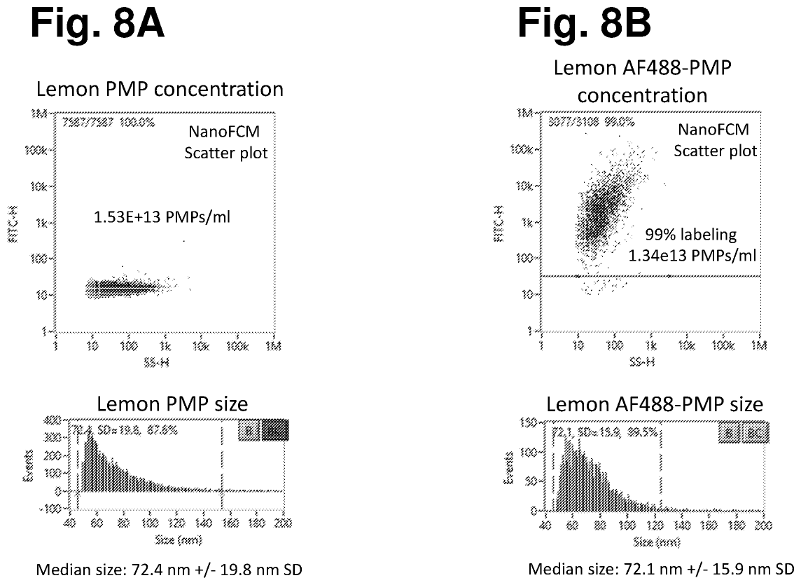

Lemons were obtained from a local Whole Foods Market®. Lemon juice (3.3 L) was collected using a juice press, pH adjusted to pH4 with NaOH, and incubated with 0.5 U/ml pectinase (Sigma, 17389) to remove pectin contaminants. Juice was incubated for one hour at room temperature with stirring, and stored overnight at 4 C, and subsequently centrifuged at 3000 g for 20 minutes, followed by 10,000 g for 40 minutes to remove large debris. Next, the processed juice was incubated with 500 mM EDTA pH8.6, to a final concentration of 50 mM EDTA, pH7.5 for 30 minutes at room temperature to chelate calcium and prevent the formation of pectin macromolecules. Subsequently, the EDTA-treated juice was passaged through an 11 μm, 1 μm and 0.45 μm filter to remove large particles. Filtered juice was washed (300 ml PBS during TFF procedure) and concentrated 2× to a total volume of 1350 ml by Tangential Flow Filtration (TFF), and dialyzed overnight using a 300 kDa dialysis membrane. Subsequently, the dialyzed juice was further washed (500 ml PMS during TFF procedure) and concentrated by TFF to a final concentration of 160 ml (˜20×). Next, we used size exclusion chromatography to elute the PMP-containing fractions, and analyzed the 280 nm absorbance (SpectraMax®) to determine the PMP-containing fractions from late elution fractions containing contaminants. SEC fractions 4-7 containing purified PMPs were pooled together, filter sterilized by sequential filtration using 0.85 μm, 0.4 μm and 0.22 μm syringe filters, and concentrated further by pelleting PMPs for 1.5 hrs at 40,000× g and finally the pellet is resuspended in Ultrapure water. The final PMP concentration (1.53×10¹³ PMPs/ml) and median PMP size (72.4 nm+/−19.8 nm SD) (FIG. 8A) were determined by nano-flow cytometry (NanoFCM) using concentration and size standards provided by the manufacturer, and PMP protein concentration (12.317 mg/ml) was determined using a Pierce™ BCA assay (ThermoFisher) according to the manufacturer's instructions.

b) Labeling of lemon PMPs with Alexa Fluor 488 NHS Ester

Lemon PMPs were labeled with the Alexa Fluor 488 NHS Ester (Life Technologies, covalent membrane dye (AF488). Briefly, AF488 was dissolved in DMSO to a final concentration of 10 mg/ml, 200 μl of PMPs (1.53×10¹³ PMPs/ml) were mixed with 5 μl dye, incubated for 1 h at room temperature on a shaker, and labeled PMPs were washed 2-3 times by ultracentrifuge at 100,000×g for 1 hr at 4° C. and pellets were resuspended with 1.5 ml UltraPure water. To control for the presence of potential dye aggregates, a dye-only control sample was prepared according to the same procedure, adding 200 ul of UltraPure water instead of PMPs. The final AF488-labeled PMP pellet and AF488 dye-only control were resuspended in a minimal amount of UltraPure water and characterized by NanoFCM. The final concentration of AF488-labeled PMPs was 1.33×10¹³ PMPs/ml with a median AF488-PMP size of 72.1 nm+/−15.9 nm SD, and a labeling efficiency of 99% was achieved (FIG. 8B).

c) Treatment of Insect Cells with Lemon AF488-PMPs

Lemon PMPs were produced and labeled as described in Examples 22a and 22b. The sf9 Spodoptera frugiperda cell line was obtained from ThermoFisher Scientific (#B82501), and maintained in TNM-FH insect medium (Sigma Aldrich, T1032) supplemented with 10% heat inactivated fetal bovine serum. The S2 Drosophila melanogaster cell line was obtained from the ATCC (#CRL-1963) and maintained in Schneider's Drosophila medium (Gibco/ThermoFisher Scientific #21720024) supplemented with 10% heat inactivated fetal bovine serum. Both cell lines were grown at 26° C. For PMP treatment, S2/Sf9 cells were seeded at 50% confluency on sterile 0.01% poly-l-lysine-coated glass coverslips in a 24 well plate in 2 ml of complete medium, and allowed to adhere to the coverslip overnight. Next, cells were treated by adding 10 ul AF488 dye only (dye aggregate control), lemon PMPs (PMP only control), or AF488-PMPs to duplicate samples, which were incubated for 2 h at 26° C. The final concentration was 1.33×10¹¹ PMPs/AF488-PMPs per well. The cells were then washed twice with 1 ml PBS, and fixed for 15 min with 4% formaldehyde in PBS. Cells were subsequently permeabilized with PBS+0.02% triton X-100 for 15 min, and nuclei were stained with a 1:1000 DAPI solution for 30 min. Cells were washed once with PBS and coverslips were mounted on a glass slides with ProLong™ Gold Antifade (ThermoFisher Scientific) to reduce photobleaching. The resin was set overnight and the cells were examined on an Olympus epifluorescence microscope using a 100× objective, and Z-stack images of 10 um with 0.25 um increments were taken. Similar results were obtained for both S2 D. melanogaster and S9 L. frugiperda cells. While no green foci were observed in the AF488 dye only control, and the PMP only control, nearly all insect cells treated with AF488-PMPs displayed green foci within the insect cells. There was a strong signal in the cytoplasm with several bright larger foci indicative on endosomal compartments. Due to bleed through of DAPI in the 488 channel, it was not possible to assess for the presence of AF488-PMP signal in the nucleus. For sf9 cells, 94.4% (n=38) of the examined cells displayed green foci, while this was not observed in the control samples AF488 dye only (n=68) or PMP only (n=42) controls.

Our data indicate that PMPs can associate with insect cell membranes, and can be efficiently taken up by insect cells.

### Example 23: Loading of PMPs with a Small Molecule

This example demonstrates loading of PMPs with a model small molecule for the purpose of delivering an agent using different PMP sources and encapsulation methods. In this example, doxorubicin is used as a model small molecule, and lemon and grapefruit PMPs are used as model PMPs.

We show that PMPs can be efficiently loaded with doxorubicin, and that loaded PMPs are stable for at least 8 weeks at 4° C.

a) Production of Grapefruit PMPs Using TFF Combined with SEC

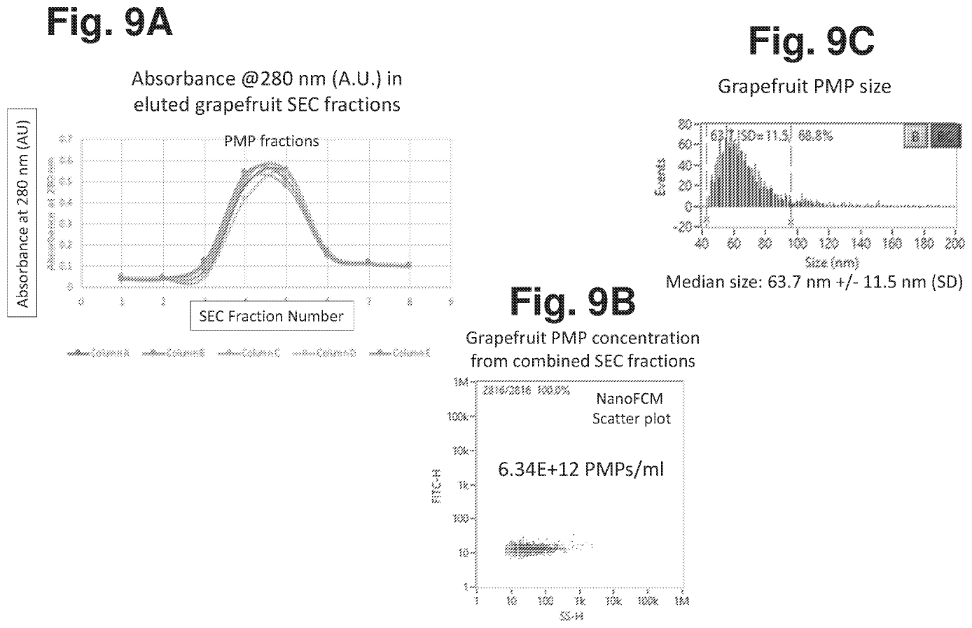

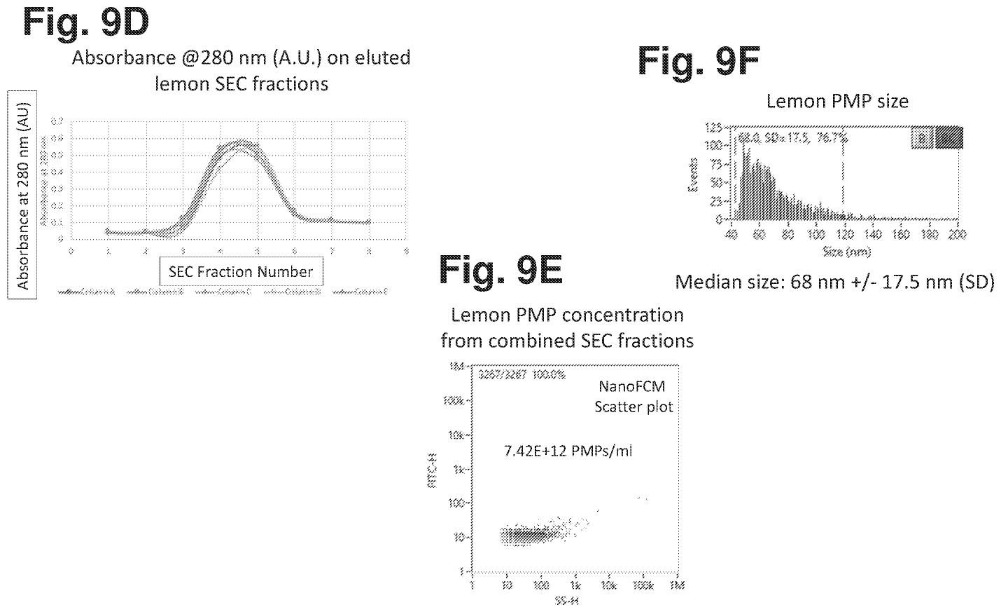

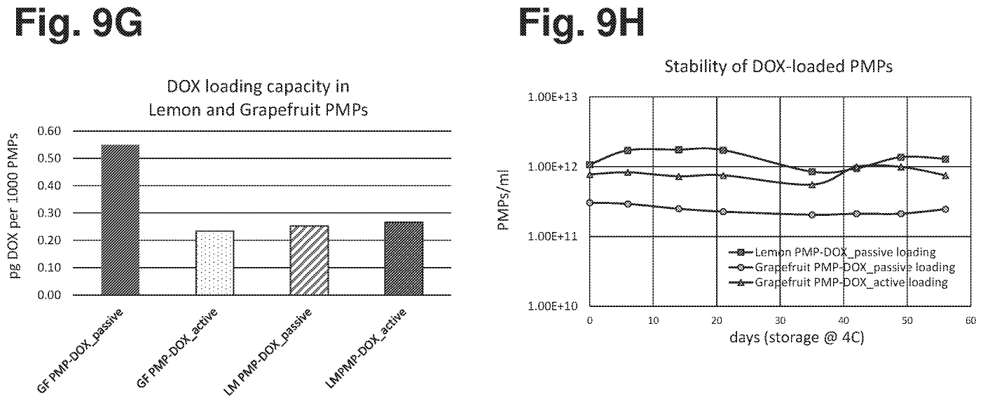

White grapefruits (Florida) were obtained from a local Whole Foods Market®. One liter of grapefruit juice was collected using a juice press, and was subsequently centrifuged at 3000×g for 20 minutes, followed by 10,000×g for 40 minutes to remove large debris. Next, 500 mM EDTA pH8.6 was added to final concentration of 50 mM EDTA, pH7 and incubated for 30 minutes to chelate calcium and prevent the formation of pectin macromolecules. Subsequently the juice was passaged through a coffee filter and 1 μm and 0.45 μm filters to remove large particles. Filtered juice was concentrated by Tangential Flow Filtration (TFF, 5 nm pore size) to 400 ml and dialyzed overnight in PBS pH 7.4 using a 300 kDa dialysis membrane to remove contaminants. Subsequently, the dialyzed juice was further concentrated by TFF to a final concentration of 50 ml (20×). Next, we used size exclusion chromatography to elute the PMP-containing fractions, which were analyzed by 280 nm absorbance (SpectraMax®) to verify the PMP-containing fractions and late fractions containing contaminants (FIG. 9A). SEC fractions 4-6 containing purified PMPs were pooled together, and concentrated further by pelleting PMPs for 1.5 hrs at 40,000×g and resuspending the pellet in Ultrapure water. The final PMP concentration (6.34×10¹² PMPs/ml) and median PMP size (63.7 nm+/−11.5 nm (SD)) were determined by NanoFCM, using concentration and size standards provided by the manufacturer (FIGS. 9B and 9C).

b) Production of Lemon PMPs Using TFF Combined with SEC

Lemons were obtained from a local Whole Foods Market®. One liter of lemon juice was collected using a juice press, and was subsequently centrifuged at 3000 g for 20 minutes, followed by 10,000 g for 40 minutes to remove large debris. Next, 500 mM EDTA pH8.6 was added to final concentration of 50 mM EDTA, pH7 and incubated for 30 minutes to chelate calcium and prevent the formation of pectin macromolecules. Subsequently the juice was passaged through a coffee filter, 1 um and 0.45 um filters to remove large particles. Filtered juice was concentrated by Tangential Flow Filtration (TFF, 5 nm pore size) to 400 ml and dialyzed overnight in PBS pH 7.4 using a 300 kDa dialysis membrane to remove contaminants. Subsequently, the dialyzed juice was further concentrated by TFF to a final concentration of 50 ml (20×). Next, we used size exclusion chromatography to elute the PMP-containing fractions, which were analyzed by 280 nm absorbance (SpectraMax®) to verify the PMP-containing fractions and late fractions containing contaminants (FIG. 9D). SEC fractions 4-6 containing purified PMPs were pooled together, and concentrated further by pelleting PMPs for 1.5 hrs at 40,000×g and resuspending the pellet in Ultrapure water. Final PMP concentration (7.42×10¹² PMPs/ml) and median PMP size (68 nm+/−17.5 nm (SD)) were determined by NanoFCM, using concentration and size standards provided by the manufacturer (FIGS. 9E and 9F).

c) Passive Loading of Doxorubicin in Lemon and Grapefruit PMPs

Grapefruit (Example 23a) and lemon (Example 23b) PMPs were used for loading doxorubicin (DOX). A stock solution of doxorubicin (DOX, Sigma PHR1789) was prepared at a concentration of 10 mg/mL in Ultrapure water (UltraPure™ DNase/RNase-Free Distilled Water, ThermoFisher, 10977023), filter sterilized (0.22 μm), and stored at 4° C. 0.5 mL of PMPs were mixed with 0.25 mL of DOX solution. The final DOX concentration in the mixture was 3.3 mg/mL. The initial particle concentration for grapefruit (GF) PMPs was 9.8×10¹² PMPs/mL and for lemon (LM) PMPs was 1.8×10¹³ PMPs/mL. The mixture was agitated for 4 hours at 25° C., 100 rpm, in the dark. Then the mixture was diluted 3.3 times with UltraPure water (the final concentration of DOX in the mixture was 1 mg/ml) and split into two equals parts (1.25 mL for passive loading, and 1.25 mL for active loading (Example 23d). Both samples were incubated for an additional 23 h at 25° C., 100 rpm, in the dark. All steps were carried out under sterile conditions.

For passive loading of DOX, to remove unloaded or weakly bound DOX, the sample was purified by ultracentrifugation. The mixture was split into 6 equal parts (200 uL each) and sterile water (1.3 mL) was added. Samples were spun down (40,000×g, 1.5 h, 4 C) in 1.5 mL ultracentrifuge tubes. The PMP-DOX pellets were resuspended in sterile water and spun down twice. Samples were kept at 4° C. for three days. Prior to use, DOX-loaded PMPs were washed one more time by ultracentrifugation (40,000×g, 1.5 h, 4° C.). The final pellet was resuspended in sterile UltraPure water and stored at 4° C. until further use. The concentration of DOX in PMPs was determined by a SpectraMax spectrophotometer (Ex/Em=485/550 nm) and concentration of the total number of particles was determined by nano-flow cytometry (NanoFCM).

d) Active Loading of Doxorubicin in Lemon and Grapefruit PMPs

Grapefruit (Example 23a) and lemon (Example 23b) PMPs were used for loading doxorubicin (DOX). A stock solution of doxorubicin (DOX, Sigma PHR1789) was prepared at a concentration of 10 mg/mL in UltraPure water (ThermoFisher, 10977023), sterilized (0.22 um), and stored at 4° C. 0.5 mL of PMPs were mixed with 0.25 mL of DOX solution. The final DOX concentration in the mixture was 3.3 mg/mL. The initial particle concentration for grapefruit (GF) PMPs was 9.8×10¹² PMPs/mL and for lemon (LM) PMPs was 1.8×10¹³ PMPs/mL. The mixture was agitated for 4 hours at 25° C., 100 rpm, in the dark. Then the mixture was diluted 3.3 times with UltraPure water (the final concentration of DOX in the mixture was 1 mg/ml) and split into two equals parts (1.25 mL for passive loading (Example 23c), and 1.25 mL for active loading). Both samples were incubated for additional 23 h at 25° C., 100 rpm, in the dark. All steps were carried out under sterile conditions.

After incubation at 25° C. for a day, the mixture was kept at 4° C. for 4 days. Then the mixture was sonicated for 30 min in a sonication bath (Branson 2800) at 42° C., vortexed, and sonicated once more for 20 min. Next, the mixture was diluted two times with sterile water and extruded using an Avanti Mini Extruder (Avanti Lipids). To reduce the number of lipid bilayers and overall particle size, the DOX-loaded PMPs were extruded in a decreasing stepwise fashion: 800 nm, 400 nm and 200 nm for grapefruit (GF) PMPs; and 800 nm, 400 nm for lemon (LM) PMPs. To remove unloaded or weakly bound DOX, the samples were washed using an ultracentrifugation approach. Specifically, the sample (1.5 mL) was diluted with sterile UltraPure water (6.5 mL total) and was spun down twice at 40,000×g for 1 h at 4° C. in 7 mL ultracentrifuge tubes. The final pellet was resuspended in sterile UltraPure water and kept at 4° C. until further use.

e) Determination of the Loading Capacity of DOX-Loaded PMPs Prepared by Passive and Active Loading

To assess the loading capacity of DOX in PMPs, DOX concentration was assessed by fluorescence intensity measurement (Ex/Em=485/550 nm) using a SpectraMax® spectrophotometer. A calibration curve of free DOX from 0 to 83.3 ug/mL was used. To dissociate DOX-loaded PMPs and DOX complexes (π-π stacking), samples and standards were incubated with 1% SDS at 37° C. for 30 min prior to fluorescence measurements. Loading capacity (pg DOX per 1000 particles) was calculated as concentration of DOX (pg/mL) divided by the total concentration of PMPs (PMPs/mL) (FIG. 9G). The loading capacity for passively loaded PMPs was 0.55 pg DOX (GF PMP-DOX) and 0.25 pg DOX (LM PMP-DOX) for 1000 PMPs. The loading capacity for actively loaded PMPs was 0.23 pg DOX (GF PMP-DOX) and 0.27 pg DOX (LM PMP-DOX) for 1000 PMPs.

f) Stability of Doxorubicin-Loaded Grapefruit and Lemon PMPs

The stability of DOX-loaded PMPs was assessed by measurement of concentration of total PMPs (PMP/ml) in the sample over time using NanoFCM. The stability study was carried out at 4° C. for eight weeks in the dark. Aliquots of PMP-DOX were stored at 4° C. and analyzed by NanoFCM on predetermined days. The particle size of PMP-DOX did not change significantly. Thus, for passively loaded GF PMPs the range of average particle sizes was 70-80 nm over two months. Concentrations of total PMPs in the sample were analyzed (FIG. 9H). The range of concentrations for passively loaded GF PMPs was from 2.06×10¹¹ to 3.06×10¹¹ PMPs/ml, for actively loaded GF PMPs was from 5.55×10¹¹ to 9.97×10¹¹ PMPs/ml, and for passively loaded LM PMPs was from 8.52×10¹¹ to 1.76×10¹² PMPs/ml over eight weeks at 4° C. Our data indicate that DOX-loaded PMPs are stable for 8 weeks at 4° C.

### Example 24: Treatment of Bacteria and Fungi with Small Molecule-Loaded PMPs

This example demonstrates the ability of PMPs to be loaded with a small molecule with the purpose of decreasing the fitness of pathogenic bacteria and fungi. In this example, grapefruit PMPs are used as a model PMP, E. coli, P. syringae and P. aeruginosa are used as model pathogenic bacteria, the yeast S. cerevisiae is used as a model pathogenic fungus, and doxorubicin is used as a model small molecule. Doxorubicin is a cytotoxic anthracycline antibiotic isolated from cultures of Streptomyces peucetius var caesius. Doxorubicin interacts with DNA by intercalation and inhibits both DNA replication and RNA transcription. Doxorubicin has been shown to have antibiotic activity (Westman et al., Chem Biol, 19(10): 1255-1264, 2012.)

a) Production of Grapefruit PMPs Using TFF Combined with SEC

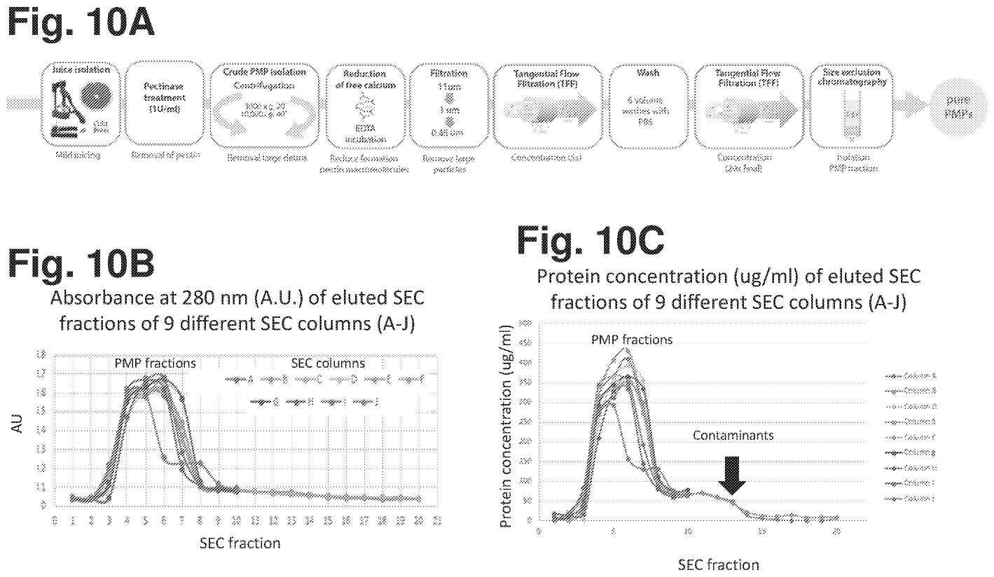

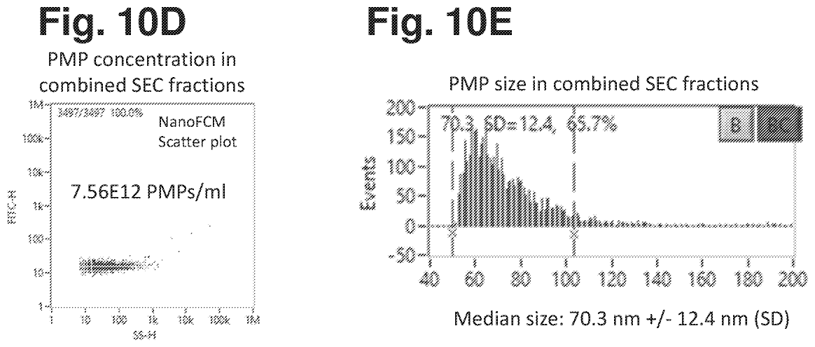

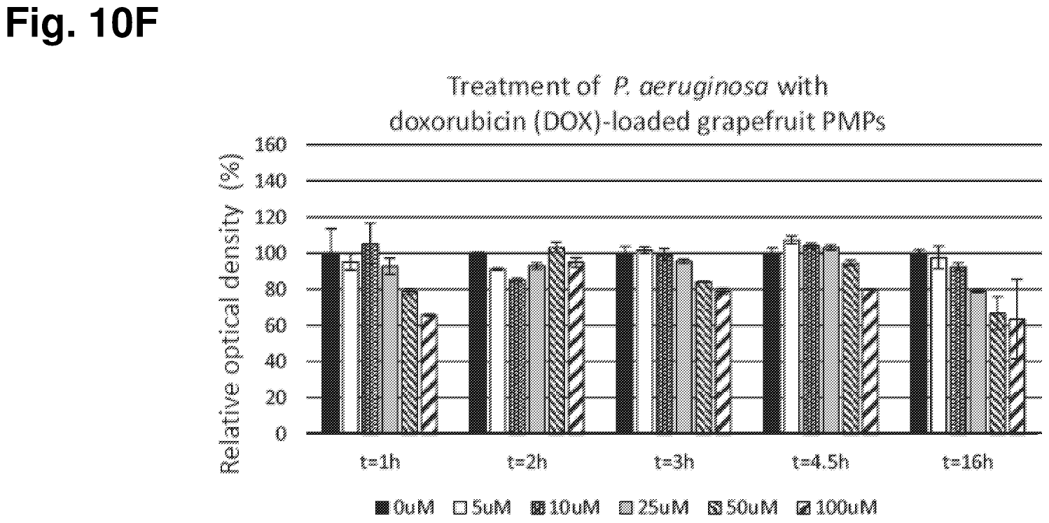

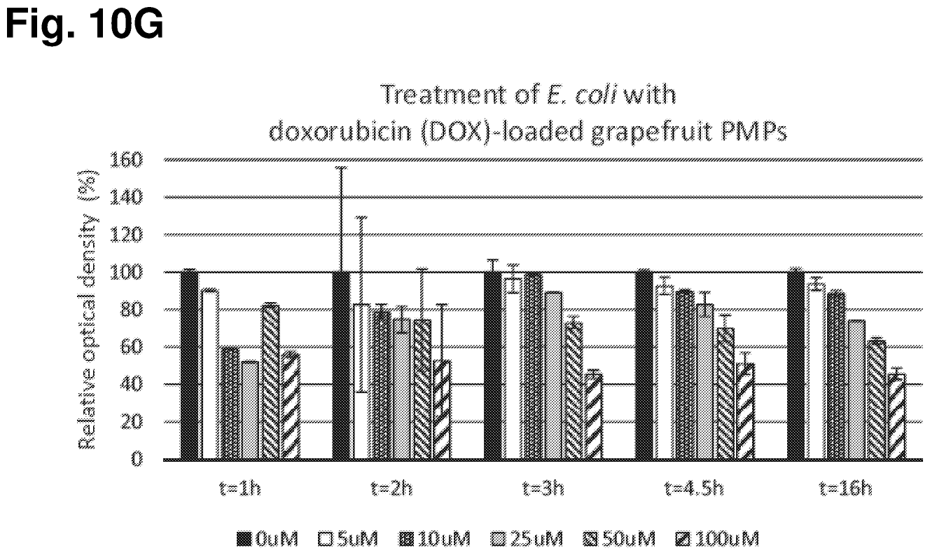

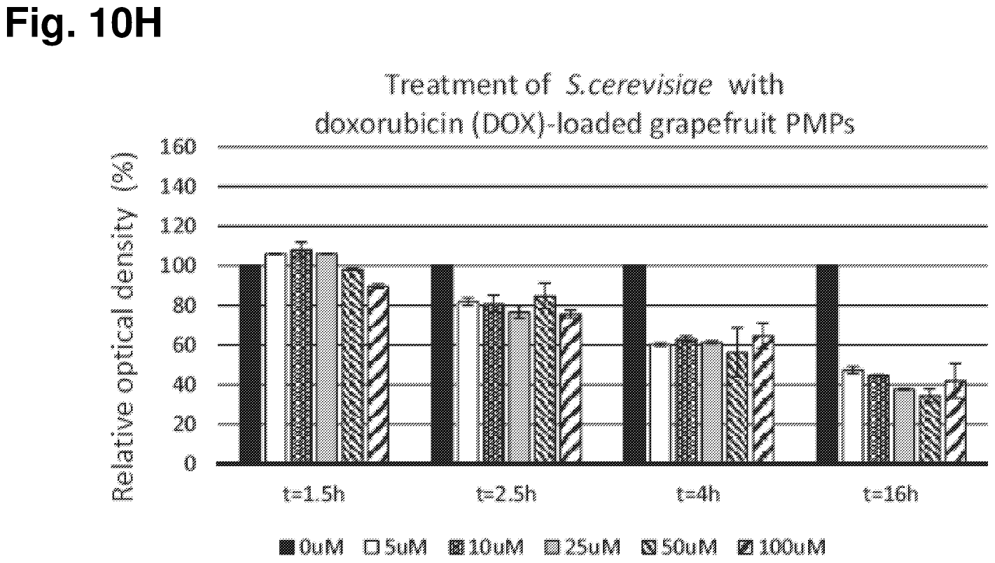

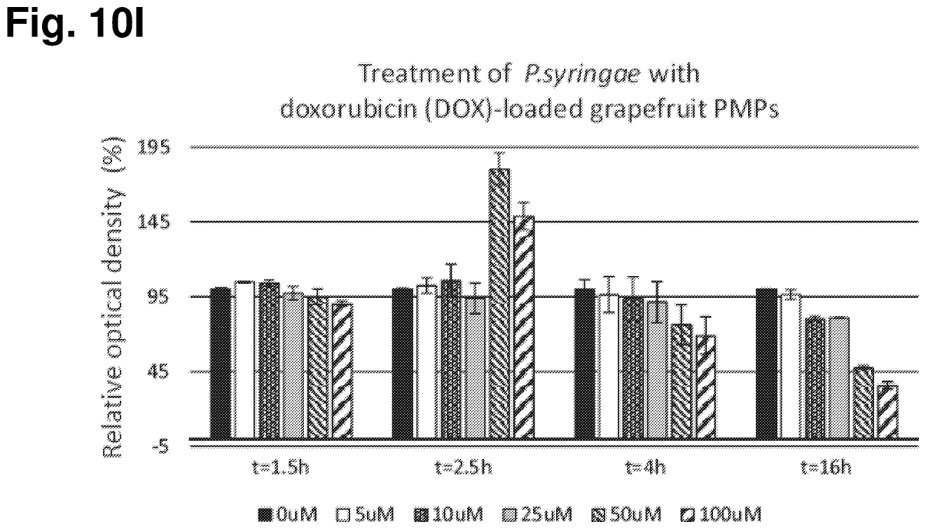

Red organic grapefruits were obtained from a local Whole Foods Market®. An overview of the PMP production workflow is given in FIG. 10A. Four liters of grapefruit juice were collected using a juice press, pH adjusted to pH4 with NaOH, incubated with 1 U/ml pectinase (Sigma, 17389) to remove pectin contaminants, and subsequently centrifuged at 3,000 g for 20 minutes, followed by 10,000 g for 40 minutes to remove large debris. Next, the processed juice was incubated with 500 mM EDTA pH8.6, to a final concentration of 50 mM EDTA, pH7.7 for 30 minutes to chelate calcium and prevent the formation of pectin macromolecules. Subsequently, the EDTA-treated juice was passaged through an 11 m, 1 Prn and 0.45 prn filter to remove large particles. Filtered juice was washed and concentrated by Tangential Flow Filtration (TFF) using a 300 kDa TFF. Juice was concentrated 5×, followed by a 6 volume exchange wash with PBS, and further filtrated to a final concentration 198 mL (20×). Next, we used size exclusion chromatography to elute the PMP-containing fractions, which were analyzed by absorbance at 280 nm (SpectraMax®) and protein concentration (Pierce™ BCA assay, ThermoFisher) to verify the PMP-containing fractions and late fractions containing contaminants (FIGS. 10B and 10C). SEC fractions 3-7 contained purified PMPs (fractions 9-12 contained contaminants), were pooled together, were filter sterilized by sequential filtration using 0.8 μm, 0.45 μm and 0.22 μm syringe filters, and were concentrated further by pelleting PMPs for 1.5 hrs at 40,000× g and resuspending the pellet in 4 ml UltraPure™ DNase/RNase-Free Distilled Water (ThermoFisher, 10977023). Final PMP concentration (7.56×10¹² PMPs/ml) and average PMP size (70.3 nm+/−12.4 nm SD) were determined by NanoFCM, using concentration and size standards provided by the manufacturer (FIGS. 10D and 10E). The produced grapefruit PMPs were used for loading doxorubicin.

b) Loading of Doxorubicin in Grapefruit PMPs

Grapefruit PMPs produced in Example 24a were used for loading doxorubicin (DOX). A stock solution of doxorubicin (Sigma PHR1789) was prepared at a concentration of 10 mg/mL in UltraPure water and filter sterilized (0.22 prn). Sterile grapefruit PMPs (3 mL at particle concentration of 7.56×10¹² PMPs/ml) were mixed with the 1.29 mL of DOX solution. The final DOX concentration in the mixture was 3 mg/mL. The mixture was sonicated for 20 min in a sonication bath (Branson 2800) with temperature rising to 40° C. and kept an additional 15 minutes in the bath without sonication. The mixture was agitated for 4 hours at 24° C., 100 rpm, in the dark. Next, the mixture was extruded using Avanti Mini Extruder (Avanti Lipids). To reduce the number of lipid bilayers and overall particle size, the DOX-loaded PMPs were extruded in a decreasing stepwise fashion: 800 nm, 400 nm and 200 nm. The extruded sample was filter sterilized by subsequent passage through a 0.8 μm and 0.45 μm filter (Millipore, diameter 13 mm) in a TC hood. To remove unloaded or weakly-bound DOX, the sample was purified using an ultracentrifugation approach. Specifically, the sample was spun down at 100,000× g for 1 h at 4° C. in 1.5 mL ultracentrifuge tubes. The supernatant was collected for further analysis and stored at 4° C. The pellet was resuspended in sterile water and ultracentrifuged under the same conditions. This step was repeated four times. The final pellet was resuspended in sterile UltraPure water and kept at 4° C. until further use.

Next, the concentration of particles and the loading capacity of PMPs was determined. The total number of PMPs in the sample (4.76×10¹² PMP/ml) and the median particle size (72.8 nm+/−21 nm SD) were determined using a NanoFCM. The DOX concentration was assessed by fluorescence intensity measurement (Ex/Em=485/550 nm) using a SpectraMax® spectrophotometer. A calibration curve of free DOX from 0 to 50 ug/mL was prepared in sterile water. To dissociate DOX-loaded PMPs and DOX complexes (π-π stacking), samples and standards were incubated with 1% SDS at 37° C. for 45 min prior to fluorescence measurements. The loading capacity (pg DOX per 1000 particles) was calculated as the concentration of DOX (pg/ml) divided by the total number of PMPs (PMPs/ml). The PMP-DOX loading capacity was 1.2 pg DOX per 1000 PMPs. However, it should be noted that the loading efficiency (the % of DOX-loaded PMPs compared to the total number of PMPs) could not be assessed as the DOX fluorescence spectrum could not be detected on the NanoFCM.

Our results indicate that PMPs can be efficiently loaded with a small molecule.

c) Treatment of Bacteria and Yeast with Dox-Loaded Grapefruit PMPs

To establish that PMPs can deliver a cytotoxic agent, several microbe species were treated with Doxorubicin-loaded grapefruit PMPs (PMP-DOX) from Example 24b.

Bacteria and yeast strains were maintained as indicated by the supplier: E. coli(ATCC, #25922) was grown on Trypticase Soy Agar/broth at 37° C., Pseudomonas aeruginosa (ATCC) was grown on Tryptic soy Agar/broth with 50 mg/ml rifampicin at 37° C., Pseudomonas syringae pv. tomato str. DC3000 bacteria (ATCC, #BAA-871) was grown on King's Medium B agar with 50 mg/ml rifampicin at 30° C., and Saccharomyces cerevisiae (ATCC, #9763) was grown on yeast extract peptone dextrose broth (YPD) and maintained at 30° C. Prior to treatment, fresh one day cultures were grown overnight, the OD (600 nm) was adjusted to 0.1 OD with medium prior to use, and bacteria/yeast were transferred to a 96 well plate for treatment (duplicate samples, 100 μl/well). Bacteria/yeast were treated with a 50 μl PMP-DOX solution in Ultrapure water to an effective DOX concentration of 0 (negative control), 5 μM, 10 μM, 25 μM, 50 μM and 100 μM (final volume per well was 150 μl). Plates were covered with aluminum foil, and incubated at 37° C. (E. coli, P. aeruginosa), or 30° C. (S. cerevisiae, P. syringae) and agitated at 220 rpm.

A kinetic Absorbance measurement at 600 nm was performed on a SpectraMax® spectrophotometer to monitor the OD of the cultures at t=0 h, t=1 h, t=2 h, t=3 h, t=4.5 h, t=16 h (E. coli, P. aeruginosa) or t=0.5 h, t=1.5 h, t=2.5 h, t=3.5 h, t=4 h, t=16 h (P. syringae, S. cerevisiae). Since doxorubicin has a broad fluorescence spectrum that partially bleeds into the 600 nm absorbance at a high DOX concentration, all OD values per treatment dose were first normalized to the OD of the first time point at that dose (t=0 for E. coli, P. aeruginosa, t=0.5 for P. syringae, S. cerevisiae).

To compare the cytotoxic effect of PMP-DOX treatment on different bacterial and yeast strains, within each treatment group the relative OD was determined as compared to the untreated control (set to 100%). All microbe species tested showed a varying degree of cytotoxixity induced by PMP-DOX (FIGS. 10F-101), which was dose dependent except in S. cerevisiae. S. cerevisiae was the most sensitive to PMP-DOX, already showing a cytotoxic response after 2.5 hrs of treatment, and reaching an IC50 at the lowest effective dose tested (5 uM), 16 hours post-treatment, which is 10× more sensitive than any other microbe tested in this series. P. syringae reached an IC50 at 50 μM and 100 μM 16 hours after incubation. From 3 hours after treatment, E. coli reached an IC50 only for 100 μM. P. aeruginosa was the least sensitive to PMP-DOX, showing a maximum growth reduction of 37% at effective DOX dosages of 50 and 100 μM. We also tested free doxorubicin and found that using the same dosages, cytotoxicity is induced earlier than with PMP-DOX delivery. This indicates that the small doxorubicin molecule readily diffuses into the unicellular organisms, compared to lipid membrane PMPs which, to release their cargo, need to cross the microbial cell wall and fuse with target cell membranes either directly with the plasma membrane or with the endosomal membrane after endocytic uptake.

Our data shows that PMPs loaded with a small molecule can negatively impact the fitness of a variety of bacteria and yeast.

### Example 25: Treatment of a Microbe with Protein Loaded PMPs

This example demonstrates that PMPs can be exogenously loaded with a protein, PMPs can protect their cargo from degradation, and PMPs can deliver their functional cargo to an organism. In this example, grapefruit PMPs are used as model PMP, Pseudomonas aeruginosa bacteria is used as a model organism, and luciferase protein is used as a model protein.

While protein and peptide-based drugs have great potential to impact the fitness of a wide variety pathogenic bacteria and fungi that are resistant or hard to treat, their deployment has been unsuccessful due to their instability and formulation challenges.

a) Loading of Luciferase Protein into Grapefruit PMPs

Grapefruit PMPs were produced as described in Example 24a. Luciferase (Luc) protein was purchased from LSBio (cat. no. LS-G5533-150) and dissolved in PBS, pH7.4 to a final concentration of 300 μg/mL. Filter-sterilized PMPs were loaded with luciferase protein by electroporation, using a protocol adapted from Rachael W. Sirianni and Bahareh Behkam (eds.), Targeted Drug Delivery: Methods and Protocols, Methods in Molecular Biology, vol. 1831. PMPs alone (PMP control), luciferase protein alone (protein control), or PMP+luciferase protein (protein-loaded PMPs), were mixed with 4.8× electroporation buffer (100% Optiprep (Sigma, D1556) in UltraPure water) to have a final 21% Optiprep concentration in the reaction mix (see Table 9). Protein control was made by mixing luciferase protein with UltraPure water instead of Optiprep (protein control), as the final PMP-Luc pellet was diluted in water. Samples were transferred into chilled cuvettes and electroporated at 0.400 kV, 125 μF (0.125 mF), resistance low 100Ω-high 600Ω with two pulses (4-10 ms) using a Biorad GenePulser. The reaction was put on ice for 10 minutes, and transferred to a pre-ice chilled 1.5 ml ultracentrifuge tube. All samples containing PMPs were washed 3 times by adding 1.4 ml ultrapure water, followed by ultracentrifugation (100,000×g for 1.5 h at 4° C.). The final pellet was resuspended in a minimal volume of UltraPure water (50 μL) and kept at 4° C. until use. After electroporation, samples containing luciferase protein only were not washed by centrifugation and were stored at 4° C. until use.

To determine the PMP loading capacity, one microliter of Luciferase-loaded PMPs (PMP-Luc) and one microliter of unloaded PMPs were used. To determine the amount of Luciferase protein loaded in the PMPs, a Luciferase protein (LSBio, LS-G5533-150) standard curve was made (10, 30, 100, 300, and 1000 ng). Luciferase activity in all samples and standards was assayed using the ONE-Glo™ luciferase assay kit (Promega, E6110) and measuring luminescence using a SpectraMax® spectrophotometer. The amount of luciferase protein loaded in PMPs was determined using a standard curve of Luciferase protein (LSBio, LS-G5533-150) and normalized to the luminescence in the unloaded PMP sample. The loading capacity (ng luciferase protein per 1E+9 particles) was calculated as the luciferase protein concentration (ng) divided by the number of loaded PMPs (PMP-Luc). The PMP-Luc loading capacity was 2.76 ng Luciferase protein/1×10⁹ PMPs.

Our results indicate that PMPs can be loaded with a model protein that remains active after encapsulation.

TABLE 9
Luciferase protein loading strategy using electroporation.

| | Luciferase PMP | Luciferase | PMP |
|---|---|---|---|
| | (protein- | (protein | (PMP |
| | loaded PMPs) | control) | control) |
| Luciferase protein | 25 | 25 | 0 |
| (300 μg/mL (μL) | | | |
| Optiprep 100% (μL) | 14.7 | 0 | 14.7 |
| UltraPure water (μL) | 10.3 | 45 | 35.3 |
| PMP GF (PMP stock | 20 | 0 | 20 |
| concentration = | | | |
| 7.56 × 10¹² PMP/mL) | | | |
| Final volume | 70 | 70 | 70 |

Note:
25 μL luciferase is equivalent to 7.5 μg luciferase protein.

b) Treatment of Pseudomonas aeruginosa with Luciferase Protein-Loaded Grapefruit PMPs

Pseudomonas aeruginosa (ATCC) was grown overnight at 30° C. in tryptic soy broth supplemented with 50 ug/ml Rifampicin, according to the supplier's instructions. Pseudomonas aeruginosa cells (total volume of 5 ml) were collected by centrifugation at 3,000×g for 5 min. Cells were washed twice with 10 ml 10 mM MgCl₂ and resuspended in 5 ml 10 mM MgCl₂. The OD600 was measured and adjusted to 0.5.

Treatments were performed in duplicate in 1.5 ml Eppendorf tubes, containing 50 μl of the resuspended Pseudomonas aeruginosa cells supplemented with either 3 ng of PMP-Luc (diluted in Ultrapure water), 3 ng free luciferase protein (protein only control; diluted in Ultrapure water), or Ultrapure water (negative control). Ultrapure water was added to 75 μl in all samples. Samples were mixed and incubated at room temperature for 2 h and covered with aluminum foil. Samples were next centrifuged at 6,000×g for 5 min, and 70 μl of the supernatant was collected and saved for luciferase detection. The bacterial pellet was subsequently washed three times with 500 μl 10 mM MgCl₂ containing 0.5% Triton X-100 to remove/burst PMPs that were not taken up. A final wash with 1 ml 10 mM MgCl₂ was performed to remove residual Triton X-100. 970 μl of the supernatant was removed (leaving the pellet in 30 ul wash buffer) and 20 μl 10 mM MgCl₂ and 25 μl Ultrapure water were added to resuspend the Pseudomonas aeruginosa pellets. Luciferase protein was measured by luminescence using the ONE-Glo™ luciferase assay kit (Promega, E6110), according to the manufacturer's instructions. Samples (bacterial pellet and supernatant samples) were incubated for 10 minutes, and luminescence was measured on a SpectraMax® spectrophotometer.

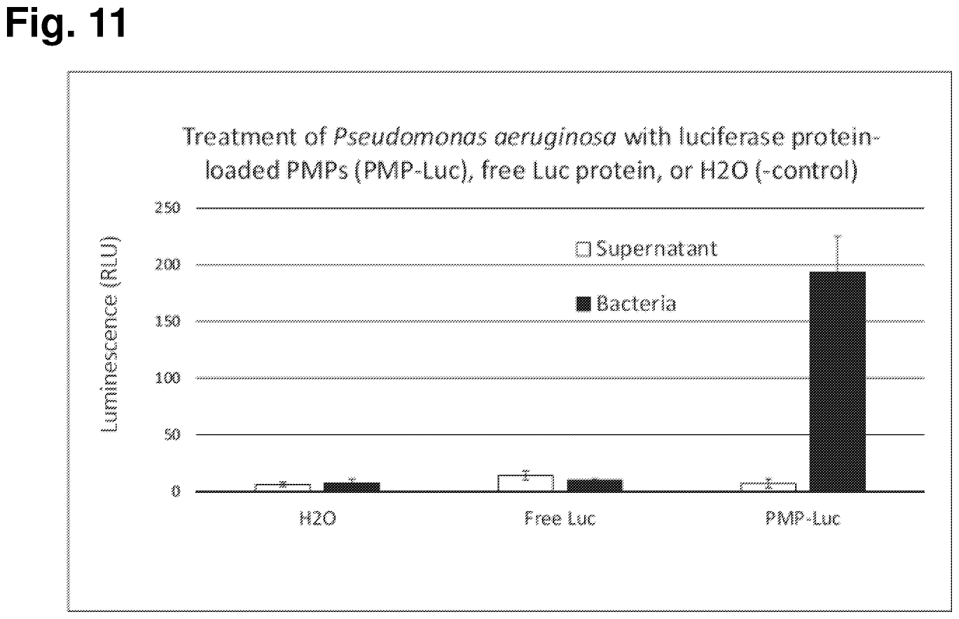

Pseudomonas aeruginosa treated with Luciferase protein-loaded grapefruit PMPs had a 19.3 fold higher luciferase expression than treatment with free luciferase protein alone or the Ultrapure water control (negative control), indicating that PMPs are able to efficiently deliver their protein cargo into bacteria (FIG. 11). In addition, PMPs appear to protect luciferase protein from degradation, as free luciferase protein levels in both the supernatant and bacterial pellets are very low. Considering the treatment dose was 3 ng luciferase protein, based on the luciferase protein standard curve, free luciferase protein in supernatant or bacterial pellets after 2 hours of RT incubation in water corresponds to <0.1 ng luciferase protein, indicating protein degradation.

Our data shows that PMPs can deliver a protein cargo into organisms, and that PMPs can protect their cargo from degradation by the environment.

### Example 26: Uptake of PMPs by Plant Cells

This example demonstrates the ability of PMPs to associate with and be taken up by plant cells. In this example, lemon PMPs are used as a model PMP, and soy, wheat and corn cell lines are used as model plant cells.

a) Labeling of Lemon PMPs with Alexa Fluor 488 NHS Ester

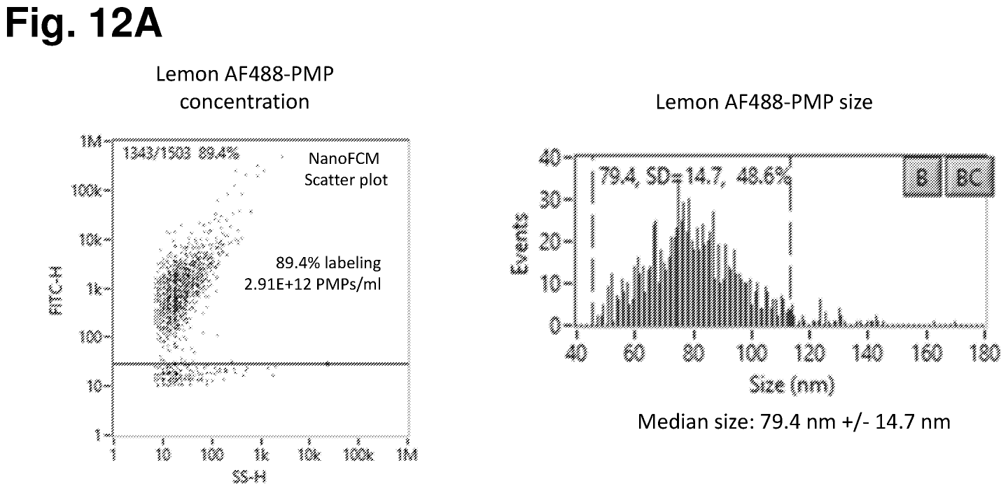

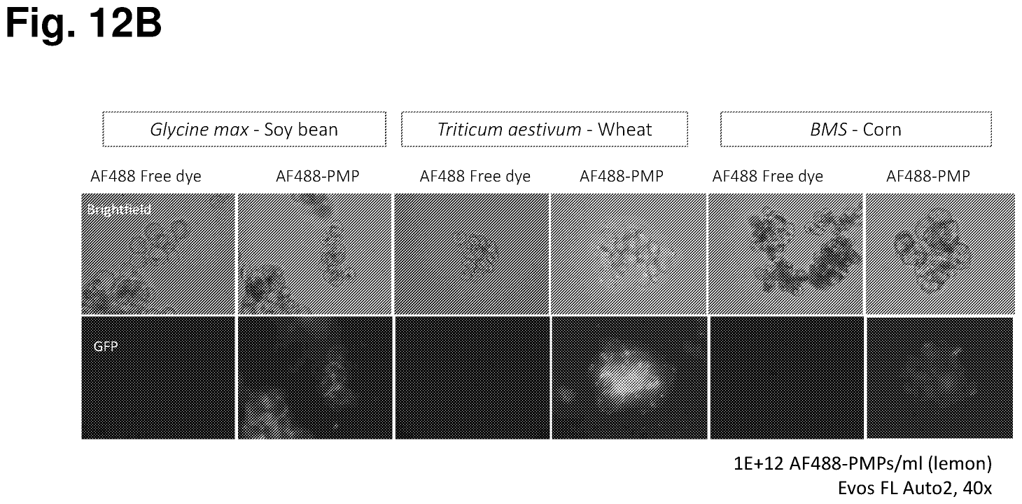

Lemon PMPs were produced as described in Example 19b. PMPs were labeled with the Alexa Fluor 488® NHS Ester (Life Technologies, covalent membrane dye (AF488)). Briefly, AF488 was dissolved in DMSO to a final concentration of 10 mg/ml, 200 ul of PMPs (1.53E+13 PMPs/ml) were mixed with 5 ul dye, incubated for 1 h at room temperature on a shaker, and labeled PMPs were washed 2-3 times by ultracentrifuge at 100,000×g for 1 hr at 4° C. Pellets were resuspended with 1.5 ml UltraPure water. To control for the presence of potential dye aggregates, a dye-only control sample was prepared according to the same procedure, adding 200 ul of UltraPure water instead of PMPs. The final AF488-labeled PMP pellet and AF488 dye-only control were resuspended in a minimal amount of UltraPure water and characterized by NanoFCM. The final concentration of lemon 488-labeled PMPs was 2.91×10¹² PMPs/ml with a median AF488-PMP size of 79.4 nm+/−14.7 nm and a labeling efficiency of 89.4% (FIG. 12A).

b) Uptake of AF488-Labeled Lemon PMPs by Plant Cells

Plant cell lines were purchased from the Deutsche Sammlung von Mikroorganismen und Zellkulturen (DSMZ) (Glycine max, #PC-1026; Triticum aestivum, #PC-998) and ABRC (Zea mays, Black Mexican sweet (BMS), and were grown in baffled vented 250 mL flasks in the dark, at 24° C. with agitation (110 rpm). Glycine max and Triticum aestivum were grown in 3.2 g/L Gamborg's B-5 Basal Medium with Minimal Organics supplemented (G5893, Millipore Sigma) pH 5.5, supplemented with 2% sucrose, and 2 mg/L 2,4-dichlorophenoxyacetic acid (2,4D) (D7299, Millipore Sigma) according to the supplier's instructions. BMS cells were grown in Murashige and Skoog basal medium pH 5.8, containing 4.3 g/L Murashige and Skoog Basal Salt Mixture (Sigma M5524), 2% sucrose (S0389, Millipore Sigma), 1× MS vitamin solution (M3900, Millipore Sigma), 2 mg/L 2,4-dichlorophenoxyacetic acid (D7299, Millipore Sigma) and 250 ug/L thiamine HCL (V-014, Millipore Sigma).

For treatment with AF488-PMPs, 5 mL of the cell suspensions was taken to determine the percent Pack Cell Volume (PCV). The PCV is defined as the volume of cells divided by the total volume of the cell culture aliquot, and expressed as a percentage. Cells were centrifuged for 5 min at 3900 rpm, and the volume of the cell pellet was determined. The % PCV for BMS, Glycine max, and Triticum aestivum were 20%, 15%, and 18%, respectively. For the uptake experiment, the % PCV of the cultures was adjusted to 2%, by diluting cells in their appropriate medium. Next, 125 μl of the plant cell suspensions was added to a 24 well plate, and duplicate samples were treated with 125 μl MES buffer (200 mM MES+10 mM NaCl, pH6) alone (negative control), AF488 dye only (dye only control) or a final concentration of 1×10¹² AF488-PMPs/mL diluted in MES buffer to 125 μl. Cells were incubated for 2 hours at 24° C. in the dark, washed three times with 1 mL MES buffer to remove AF488-PMPs or free dye that had not been taken up, and resuspended in 300 μL of MES buffer for imaging on an epifluorescence microscope (EVOS FL Auto 2, Invitrogen). Compared to the AF488 dye only control which had no detectable fluorescence, a variable fluorescent signal could be detected in all plant cell lines, indicating PMP uptake (FIG. 12B). Triticum aestivum cells displayed the strongest fluorescence signal, indicating that out of the three plant cell lines tested, they had the highest uptake of AF488-labeled lemon PMPs.

Our data shows that PMPs can be taken up by plant cells in vitro.

### Example 27: Uptake of PMPs in Plants

This example demonstrates the ability of PMPs to be taken up and systemically transported in planta. In this example, grapefruit, lemon and Arabidopsis thaliana seedling PMPs are used as model PMPs, and Arabidopsis seedlings and alfalfa sprouts are used as model plants.

a) Labeling of Lemon and Grapefruit PMPs with DyLight 800 NHS Ester

Grapefruit and lemon PMPs were produced as described in Examples 19a and 19b. PMPs were labeled with the DyLight 800 NHS Ester (Life Technologies, #46421) covalent membrane dye (DyL800). Briefly, Dy800 was dissolved in DMSO to a final concentration of 10 mg/ml, 200 μl of PMPs were mixed with 5 μl dye, incubated for 1 h at room temperature on a shaker, and labeled PMPs were washed 2-3 times by ultracentrifugation at 100,000×g for 1 hr at 4° C. and pellets were resuspended with 1.5 ml UltraPure water. To control for the presence of potential dye aggregates, a dye-only control sample was prepared according to the same procedure, adding 200 μl of UltraPure water instead of PMPs. The final DyL800-labeled PMP pellet and DyL800 dye-only control were resuspended in a minimal amount of UltraPure water and characterized by NanoFCM. The final concentration of grapefruit DyL800-labeled PMPs was 4.44×10¹² PMPs/ml, and of lemon DyL800-labeled PMPs was 5.18×10¹² PMPs/ml. The labeling efficiency could not be determined using the NanoFCM, as it cannot detect infrared.

b) Germination and Growth of Arabidopsis thaliana Seedlings

Wild type Arabidopsis thaliana Col-0 seeds were obtained from the ABRC and were surface sterilized with 70% ethanol, incubation with 50% bleach/0.1% triton X-100 for 10 minutes, and 4 sterile ddH2O washes to remove the bleach solution. Seeds were stratified for 1 d at 4° C. in the dark. Approximately 250 seeds were germinated per 100 cm² plate (pre-coated with 0.5% fetal calf serum in water), containing 20 mL 0.5× MS medium (2.15 g/L Murashige and Skoog salts, 1% sucrose, pH 5.8), sealed with 3M surgical tape and grown in an incubator with a photoperiod of 16 h light at 23° C./8 h dark at 21° C.

c) Uptake of DyL800-Labeled Grapefruit, Lemon and Ats PMPs by Arabidopsis thaliana and Alfalfa

To assess whether PMPs can be taken up and transported systemically in planta, Arabidopsis seedlings were germinated in liquid culture as described in Example 27b on top of a mesh filter, to allow the roots to grow through the mesh, and to allow partial exposure of At seedlings to a PMP solution. Alfalfa sprouts were obtained from a local supermarket. 9 day-old Arabidopsis seedlings and Alfalfa sprouts were treated with a 0.5 ml solution of water (negative control), DyL800 dye only (dye control) DyL800-labeled grapefruit PMPs (1.6×10¹⁰ PMPs/ml), or lemon (5.1×10¹⁰ PMPs/ml) PMPs in 0.5× MS medium by partial root exposure (At seedlings in a mesh floating in a PMP solution, or in Alfalfa sprouts by partial root exposure in a 1.5 ml Eppendorf tube) for 22 or 24 hours, respectively, at 23° C. Plants where then washed 3 times in MS medium and imaged using an Odyssey® CLx infrared imager (Li-Cor).

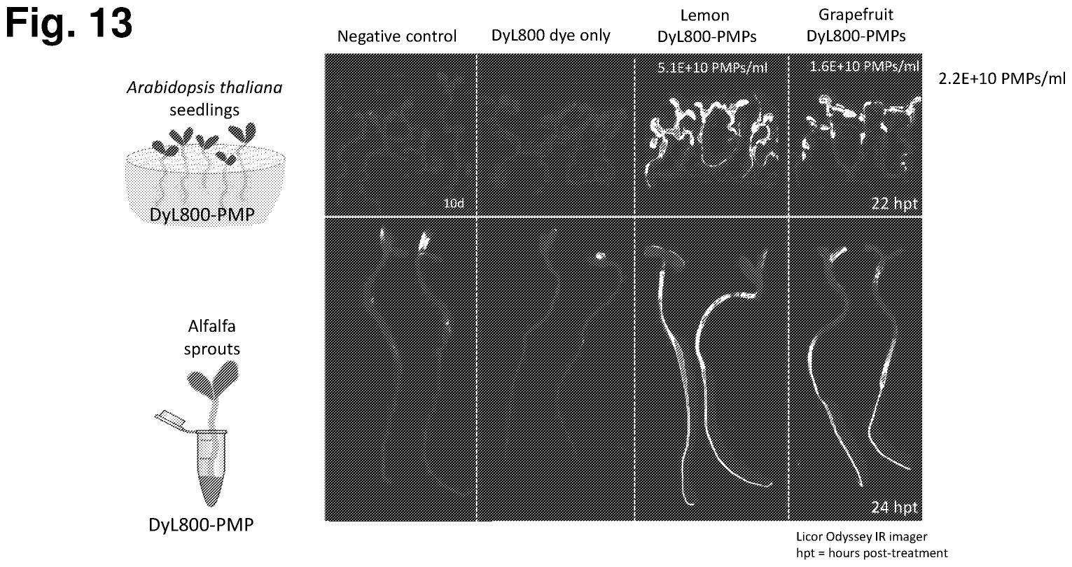

Compared to the negative (some autofluorescence in Alfalfa sprout leafs) and dye only control, all PMP sources showed a fluorescence signal (white is high fluorescent signal, black is no signal) in both Arabidopsis seedlings and Alfalfa sprouts, indicating that PMPs are taken up by both plants (FIG. 13). The presence of fluorescence signal in Arabidopsis leafs or Alfalfa stem areas that were not exposed to the PMP solution indicates active transport of the PMPs in planta. As the DyL800 treatment concentrations were not normalized in this experiment, it is not possible to assess source/target uptake efficiency differences.

Our data indicate that PMPs derived from various plant sources can be taken up and transported in planta.

### Example 28: Treatment of Arabidopsis thaliana Seedlings with DOX-Loaded Grapefruit PMPs

This example demonstrates the ability of PMPs to be loaded with a small molecule with the purpose of decreasing the fitness of a plant. In this example, doxorubicin is used as a model small molecule, and Arabidopsis thaliana is used as a model plant. Doxorubicin is a cytotoxic anthracycline antibiotic isolated from cultures of Streptomyces peucetius var. caesius. Doxorubicin interacts with DNA by intercalation and inhibits both DNA replication and RNA transcription. Doxorubicin has been shown to be cytotoxic in plants (Culiarez-Mac et al, Plant Growth Regulation. (5): 155-164, 1987.

Effective and safe herbicides are necessary to prevent major crop yield loss due to weeds, while protecting the environment from the toxic side-effects of herbicide over-use.

a) Treatment of Arabidopsis Thaliana Seedlings with Doxorubicin-Loaded PMPs

Grapefruit PMPs were produced and loaded with doxorubicin as described in Examples 24a and 24b. Wild type Arabidopsis thaliana Col-0 seeds were obtained from the ABRC, surface sterilized with 50% bleach, stratified for 1-3 d at 4° C., and germinated on half-strength (0.5×) Murashige and Skoog (MS) medium supplemented with 0.5% sucrose, 2.5 mM MES, pH 5.6, containing 0.8% agar, with a photoperiod of 16 h light 23° C./8 h dark 21° C.

To test whether PMPs can deliver a small molecule cargo in planta, 7-day old Arabidopsis thaliana seedlings were transferred to 0.5× liquid MS medium in a 24 well plate (1 seedling per well), and treated with free DOX or DOX-loaded PMPs with an encapsulated DOX dose of 0 (negative control), 25 μM, 50 μM and 100 μM. The plate was covered with aluminum foil and incubated for 24 hours. DOX-containing medium was removed, the seedlings were washed two times with ½X MS medium, and fresh medium was added. Seedlings were incubated for an additional 3 days under a normal photoperiod (16 h light 23° C./8 h dark 21° C.). Next, seedlings were removed from the plate and towel-dried for imaging and cytotoxicity was assessed by analyzing leaf vigor, leaf color and root length. Cytotoxicity is defined by shortening of the roots, loss of leaf vigor, and leaf discoloration (yellow instead of green) when compared to an untreated seedling control. Compared to free DOX that only showed cytotoxicity at 100 μM DOX (root shortening and leaf discoloration), PMPs loaded with DOX were cytotoxic at 50 μM and 100 μM DOX. 50 μM PMP-DOX treated seedlings showed severe leaf yellowing with reduced leaf vigor, and shortening of the roots. Our data indicates that PMPs can be loaded with and can deliver a small molecule in planta, and that PMPs loaded with doxorubicin are twice as efficient in inducing a cytotoxic response than free doxorubicin.

### OTHER EMBODIMENTS

Some embodiments of the invention are within the following numbered paragraphs.

1. A pest control composition comprising a plurality of plant messenger packs (PMPs), wherein the composition is formulated for delivery to a plant and wherein the composition comprises at least 5% PMPs.
2. A pest control composition comprising a plurality of PMPs, wherein the composition is formulated for delivery to a plant pest and wherein the composition comprises at least 5% PMPs.
3. The pest control composition of paragraph 1 or 2, wherein the composition is stable for at least one day at room temperature, and/or stable for at least one week at 4° C.
4. A pest control composition comprising a plurality of PMPs, wherein the composition is stable for at least one day at room temperature, and/or stable for at least one week at 4° C.
5. The pest control composition of paragraph 4, wherein the composition is formulated for delivery to a plant.
6. The pest control composition of paragraph 4, wherein the composition is formulated for delivery to a plant pest.
7. The pest control composition of any one of paragraphs 1-6, wherein the PMPs are stable for at least 24 hours, 48 hours, seven days, or 30 days.
8. The pest control composition of paragraph 7, wherein the PMPs are stable at a temperature of at least 24° C., 20° C., or 4° C.
9. The pest control composition of any one of paragraphs 1-8, wherein the plurality of PMPs in the composition is at a concentration effective to decrease the fitness of a plant pest.
10. A pest control composition comprising a plurality of PMPs, and wherein the plurality of PMPs in the composition is at a concentration effective to decrease the fitness of a plant pest.
11. The pest control composition of paragraph 10, wherein the composition is formulated for delivery to a plant.
12. The pest control composition of paragraph 10, wherein the composition is formulated for delivery to a plant pest.
13. The pest control composition of any one of paragraphs 10-12, wherein the composition is stable for at least one day at room temperature, and/or stable for at least one week at 4° C.
14. The pest control composition of any one of paragraphs 9-13, wherein the PMP comprises a plurality of PMP proteins, and the concentration of PMPs is the concentration of PMP proteins therein.
15. The pest control composition of any one of paragraphs 9-14, wherein the plurality of PMPs in the composition is at a concentration of at least 0.01 ng, 0.1 ng, 1 ng, 2 ng, 3 ng, 4 ng, 5 ng, 10 ng, 50 ng, 100 ng, 250 ng, 500 ng, 750 ng, 1 μg, 10 μg, 50 μg, 100 μg, or 250 μg PMP protein/ml.
16. The pest control composition of any one of paragraphs 1-15, wherein each of the plurality of PMPs comprises a purified plant extracellular vesicle (EV), or a segment or extract thereof.
17. A pest control composition comprising a plurality of PMPs, wherein each of the PMPs is a plant EV, or a segment or extract thereof, and wherein the composition is formulated for delivery to a plant.
18. A pest control composition comprising a plurality of PMPs, wherein the PMP is a plant EV, or a segment or extract thereof, and wherein the composition is formulated for delivery to a pest.
19. The pest control composition of paragraph 17 or 18, wherein the composition is stable for at least one day at room temperature, and/or stable for at least one week at 4° C.
20. The pest control composition of any one of paragraphs 17-19, wherein the plurality of PMPs in the composition is at a concentration effective to decrease the fitness of a plant pest.
21. The pest control composition of any one of paragraphs 16-20, wherein the plant EV is a modified plant extracellular vesicle (EV).
22. The pest control composition of paragraph 21, wherein the isolated plant EV is a plant exosome or a plant microvesicle.
23. The pest control composition of any one of paragraphs 1-22, wherein the plurality of PMPs further comprises a pest repellent.
24. A pest control composition comprising a plurality of PMPs, wherein each of the plurality of PMPs comprises a heterologous pesticidal agent and wherein the composition is formulated for delivery to a plant or a plant pest.
25. The pest control composition of paragraph 1, wherein the heterologous pesticidal agent is an herbicidal agent, an antibacterial agent, an antifungal agent, an insecticidal agent, a molluscicidal agent, or a nematicidal agent.
26. The pest control composition of paragraph 2, wherein the herbicidal agent is doxorubicin.
27. The pest control composition of paragraph 2, wherein the herbicidal agent is glufosinate, glyphosate, propaquizafop, metamitron, metazachlor, pendimethalin, flufenacet, diflufenican, clomazone, nicosulfuron, mesotrione, pinoxaden, sulcotrione, prosulfocarb, sulfentrazone, bifenox, quinmerac, triallate, terbuthylazine, atrazine, oxyfluorfen, diuron, trifluralin, or chlorotoluron.
28. The pest control composition of paragraph 2, wherein the antibacterial agent is doxorubicin.
29. The pest control composition of paragraph 2, wherein the antibacterial agent is an antibiotic.
30. The pest control composition of paragraph 6, wherein the antibiotic is vancomycin.
31. The pest control composition of paragraph 6, wherein the antibiotic is a penicillin, a cephalosporin, a tetracycline, a macrolide, a sulfonamide, vancomycin, polymixin, gramicidin, chloramphenicol, clindamycin, spectinomycin, ciprofloxacin, isoniazid, rifampicin, pyrazinamide, ethambutol, myambutol, or streptomycin.
32. The pest control composition of paragraph 2, wherein the antifungal agent is azoxystrobin, mancozeb, prothioconazole, folpet, tebuconazole, difenoconazole, captan, bupirimate, or fosetyl-Al.
33. The pest control composition of paragraph 2, wherein the insecticidal agent is a chloronicotinyl, a neonicotinoid, a carbamate, an organophosphate, a pyrethroid, an oxadiazine, a spinosyn, a cyclodiene, an organochlorine, a fiprole, a mectin, a diacylhydrazine, a benzoylurea, an organotin, a pyrrole, a dinitroterpenol, a METI, a tetronic acid, a tetramic acid, or a pthalamide.
34. The pest control composition of paragraph 1, wherein the heterologous pesticidal agent is a small molecule, a nucleic acid, or a polypeptide.
35. The pest control composition of paragraph 11, wherein the small molecule is an antibiotic or a secondary metabolite.
36. The pest control composition of paragraph 11, wherein the nucleic acid is an inhibitory RNA.
37. The pest control composition of any one of paragraphs 1-13, wherein the heterologous pesticidal agent is encapsulated by each of the plurality of PMPs.
38. The pest control composition of any one of paragraphs 1-13, wherein the heterologous pesticidal agent is embedded on the surface of each of the plurality of PMPs.
39. The pest control composition of any one of paragraphs 1-13, wherein the heterologous pesticidal agent is conjugated to the surface of each of the plurality of PMPs.
40. The pest control composition of any one of paragraphs 1-16, wherein each of the plurality of PMPs further comprises a pest repellent.
41. The pest control composition of any one of paragraphs 1-17, wherein each of the plurality of PMPs further comprises an additional heterologous pesticidal agent.
42. The pest control composition of any one of paragraphs 1-18, wherein the plant pest is a bacterium or a fungus.
43. The pest control composition of paragraph 19, wherein the bacterium is a Pseudomonas species.
44. The pest control composition of paragraph 20, wherein the Pseudomonas species is Pseudomonas aeruginosa or Pseudomonas syringae.
45. The method of paragraph 19, wherein the fungus is a Sclerotinia species, a Botrytis species, an Aspergillus species, a Fusarium species, or a Penicillium species.
46. The pest control composition of any one of paragraphs 1-28, wherein the plant pest is an insect, a mollusk, or a nematode.
47. The pest control composition of paragraph 23, wherein the insect is an aphid or a lepidopteran.
48. The pest control composition of paragraph 23, wherein the nematode is a corn root-knot nematode.
49. The pest control composition of any one of paragraphs 1-25, wherein the composition is stable for at least one day at room temperature, and/or stable for at least one week at 4° C.
50. The pest control composition of any one of paragraphs 1-25, wherein the PMPs are stable for at least 24 hours, 48 hours, seven days, or 30 days at 4° C.
51. The pest control composition of paragraph 27, wherein the PMPs are stable at a temperature of at least 20° C., 24° C., or 37° C.
52. The pest control composition of any one of paragraphs 1-28, wherein the plurality of PMPs in the composition is at a concentration effective to decrease the fitness of a plant pest.
53. The pest control composition of any one of paragraphs 1-29, wherein the plurality of PMPs in the composition is at a concentration of at least 0.01 ng, 0.1 ng, 1 ng, 2 ng, 3 ng, 4 ng, 5 ng, 10 ng, 50 ng, 100 ng, 250 ng, 500 ng, 750 ng, 1 μg, 10 μg, 50 μg, 100 μg, or 250 μg PMP protein/mL.
54. The pest control composition of any one of paragraphs 1-30, wherein the composition comprises an agriculturally acceptable carrier.
55. The pest control composition of any one of paragraphs 1-31, wherein the composition is formulated to stabilize the PMPs.
56. The pest control composition of any one of paragraphs 1-32, wherein the composition is formulated as a liquid, a solid, an aerosol, a paste, a gel, or a gas composition.
57. The pest control composition of paragraph 1, wherein the composition comprises at least 5% PMPs.
58. A pest control composition comprising a plurality of PMPs, wherein the PMPs are isolated from a plant by a process which comprises the steps of (a) providing an initial sample from a plant, or a part thereof, wherein the plant or part thereof comprises EVs; (b) isolating a crude PMP fraction from the initial sample, wherein the crude PMP fraction has a decreased level of at least one contaminant or undesired component from the plant or part thereof relative to the level in the initial sample; (c) purifying the crude PMP fraction, thereby producing a plurality of pure PMPs, wherein the plurality of pure PMPs have a decreased level of at least one contaminant or undesired component from the plant or part thereof relative to the level in the crude EV fraction; (d) loading the plurality of pure PMPs with a pest control agent; and (e) formulating the PMPs of step (d) for delivery to a plant or a plant pest.
59. A plant comprising the pest control composition of any one of paragraphs 1-35.
60. A plant pest comprising the pest control composition of any one of paragraphs 1-35.
61. A method of delivering a pest control composition to a plant comprising contacting the plant with the composition of any one of paragraphs 1-35.
62. A method of increasing the fitness of a plant, the method comprising delivering to the plant the composition of any one of paragraphs 1-35, wherein the method increases the fitness of the plant relative to an untreated plant.
63. The method of paragraph 38 or 39, wherein the plant has an infestation by a plant pest.
64. The method of paragraph 40, wherein the method decreases the infestation relative to the infestation in an untreated plant.
65. The method of paragraph 40, wherein the method substantially eliminates the infestation relative to the infestation in an untreated plant.
66. The method of paragraph 38 or 39, wherein the plant is susceptible to infestation by a plant pest.
67. The method of paragraph 43, wherein the method decreases the likelihood of infestation in the plant relative to the likelihood of infestation in an untreated plant.
68. The method of any one of paragraphs 40-44, wherein the plant pest is a bacterium or a fungus.
69. The method of paragraph 45, wherein the bacterium is a Pseudomonas species.
70. The method of paragraph 45, wherein the fungus is a Sclerotinia species, a Botrytis species, an Aspergillus species, a Fusarium species, or a Penicillium species.
71. The method of any one of paragraphs 40-44, wherein the plant pest is an insect, a mollusk, or a nematode.
72. The method of paragraph 48, wherein the insect is an aphid or a lepidopteran.
73. The method of paragraph 48, wherein the nematode is a corn root-knot nematode.
74. The method of any one of paragraphs 38-50, wherein the pest control composition is delivered as a liquid, a solid, an aerosol, a paste, a gel, or a gas.
75. A method of delivering a pest control composition to a plant pest comprising contacting the plant pest with the composition of any one of paragraphs 1-36.
76. A method of decreasing the fitness of a plant pest, the method comprising delivering to the plant pest the composition of any one of paragraphs 1-36, wherein the method decreases the fitness of the plant pest relative to an untreated plant pest.
77. The method of paragraph 52 or 53, wherein the method comprises delivering the composition to at least one habitat where the plant pest grows, lives, reproduces, feeds, or infests.
78. The method of any one of paragraphs 52-54, wherein the composition is delivered as a plant pest comestible composition for ingestion by the plant pest.
79. The method of any one of paragraphs 52-55, wherein the plant pest is a bacterium or a fungus.
80. The method of any one of paragraphs 52-55, wherein the plant pest is an insect, a mollusk, or a nematode.
81. The method of paragraph 57, wherein the insect is an aphid or a lepidopteran.
82. The method of paragraph 57, wherein the nematode is a corn root-knot nematode.
83. The method of any one of paragraphs 52-59, wherein the composition is delivered as a liquid, a solid, an aerosol, a paste, a gel, or a gas.
84. A method of treating a plant having a fungal infection, wherein the method comprises delivering to the plant a pest control composition comprising a plurality of PMPs.
85. A method of treating a plant having a fungal infection, wherein the method comprises delivering to the plant a pest control composition comprising a plurality of PMPs, and wherein each of the plurality of PMPs comprises an antifungal agent.
86. The method of paragraph 62, wherein the antifungal agent is a nucleic acid that inhibits expression of a gene in a fungus that causes the fungal infection.
87. The method of paragraph 63, wherein the gene is dcl1 and/or dcl2.
88. The method of any one of paragraphs 61-64, wherein the fungal infection is caused by a fungus belonging to a Sclerotinia species, a Botrytis species, an Aspergillus species, a Fusarium species, or a Penicillium species.
89. The method of paragraph 65, wherein the Sclerotinia species is Sclerotinia sclerotiorum.
90. The method of paragraph 65, wherein the Botrytis species is Botrytis cinerea.
91. The method of any one of paragraphs 61-67, wherein the composition comprises a PMP derived from Arabidopsis.
92. The method of any one of paragraphs 61-68, wherein the method decreases or substantially eliminates the fungal infection.
93. A method of treating a plant having a bacterial infection, wherein the method comprises delivering to the plant a pest control composition comprising a plurality of PMPs.
94. A method of treating a plant having a bacterial infection, wherein the method comprises delivering to the plant a pest control composition comprising a plurality of PMPs, and wherein each of the plurality of PMPs comprises an antibacterial agent.
95. The method of paragraph 71, wherein the antibacterial agent is doxorubicin.
96. The method of any one of paragraphs 70-72, wherein the bacterial infection is caused by a bacterium belonging to a Pseudomonas spp.
97. The method of paragraph 73, wherein the Pseudomonas spp. is Pseudomonas syringae.
98. The method of any one of paragraphs 70-74, wherein the composition comprises a PMP derived from Arabidopsis.
99. The method of any one of paragraphs 70-75, wherein the method decreases or substantially eliminates the bacterial infection.
100. A method of decreasing the fitness of an insect plant pest, wherein the method comprises delivering to the insect plant pest a pest control composition comprising a plurality of PMPs.
101. A method of decreasing the fitness of an insect plant pest, wherein the method comprises delivering to the insect plant pest a pest control composition comprising a plurality of PMPs, and wherein each of the plurality of PMPs comprises an insecticidal agent.
102. The method of paragraph 78, where in the insecticidal agent is a peptide nucleic acid.
103. The method of any one of paragraphs 77-79, wherein the insect plant pest is an aphid.
104. The method of any one of paragraphs 77-79, wherein the insect plant pest is a lepidopteran.
105. The method of paragraph 81, wherein the lepidopteran is Spodoptera frugiperda.
106. The method of any one of paragraphs 77-82, wherein the method decreases the fitness of the insect plant pest relative to an untreated insect plant pest.
107. A method of decreasing the fitness of a nematode plant pest, wherein the method comprises delivering to the nematode plant pest a pest control composition comprising a plurality of PMPs.
108. A method of decreasing the fitness of a nematode plant pest, wherein the method comprises delivering to the nematode plant pest a pest control composition comprising a plurality of PMPs, and wherein each of the plurality of PMPs comprises a nematicidal agent.
109. The method of paragraph 85, wherein the nematicidal agent is a peptide.
110. The method of paragraph 86, wherein the peptide is Mi-NLP-15b.
111. The method of any one of paragraphs 84-88, wherein the nematode plant pest is a corn root-knot nematode.
112. The method of any one of paragraphs 84-88, wherein the method decreases the fitness of the nematode plant pest relative to an untreated nematode plant pest.
113. A method of decreasing the fitness of a weed, wherein the method comprises delivering to the weed a pest control composition comprising a plurality of PMPs.
114. A method of decreasing the fitness of a weed, wherein the method comprises delivering to the weed a pest control composition comprising a plurality of PMPs, and wherein each of the plurality of PMPs comprises an herbicidal agent.
115. The method of paragraph 90 or 91, wherein the method decreases the fitness of the weed relative to an untreated weed.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

Other embodiments are within the claims.

### APPENDIX

TABLE 1
Plant EV-Markers

| Example Species | Accession No. | Protein Name |
|---|---|---|
| Arabidopsis thaliana | C0LGG8 | Probable LRR receptor-like serine/threonine-protein kinase |
| | | At1g53430 (EC 2.7.11.1) |
| Arabidopsis thaliana | F4HQT8 | Uncharacterized protein |
| Arabidopsis thaliana | F4HWU0 | Protein kinase superfamily protein |
| Arabidopsis thaliana | F4I082 | Bifunctional inhibitor/lipid-transfer protein/seed storage 2S |
| | | albumin superfamily protein |
| Arabidopsis thaliana | F4I3M3 | Kinase with tetratricopeptide repeat domain-containing |
| | | protein |
| Arabidopsis thaliana | F4IB62 | Leucine-rich repeat protein kinase family protein |
| Arabidopsis thaliana | O03042 | Ribulose bisphosphate carboxylase large chain (RuBisCO |
| | | large subunit) (EC 4.1.1.39) |
| Arabidopsis thaliana | O03986 | Heat shock protein 90-4 (AtHSP90.4) (AtHsp90-4) (Heat |
| | | shock protein 81-4) (Hsp81-4) |
| Arabidopsis thaliana | O04023 | Protein SRC2 homolog (AtSRC2) |
| Arabidopsis thaliana | O04309 | Jacalin-related lectin 35 (JA-responsive protein 1) |
| | | (Myrosinase-binding protein-like At3g16470) |
| Arabidopsis thaliana | O04314 | PYK10-binding protein 1 (Jacalin-related lectin 30) (Jasmonic |
| | | acid-induced protein) |
| Arabidopsis thaliana | O04922 | Probable glutathione peroxidase 2 (EC 1.11.1.9) |
| Arabidopsis thaliana | O22126 | Fasciclin-like arabinogalactan protein 8 (AtAGP8) |
| Arabidopsis thaliana | O23179 | Patatin-like protein 1 (AtPLP1 (EC 3.1.1.—) (Patatin-related |
| | | phospholipase A IIgamma) (pPLAIIg) (Phospholipase A IVA) |
| | | (AtPLAIVA) |
| Arabidopsis thaliana | O23207 | Probable NAD(P)H dehydrogenase (quinone) FQR1-like 2 |
| | | (EC 1.6.5.2) |
| Arabidopsis thaliana | O23255 | Adenosylhomocysteinase 1 (AdoHcyase 1) (EC 3.3.1.1) |
| | | (Protein EMBRYO DEFECTIVE 1395) (Protein |
| | | HOMOLOGY-DEPENDENT GENE SILENCING 1) |
| | | (S-adenosyl-L-homocysteine hydrolase 1) |
| | | (SAH hydrolase 1) |
| Arabidopsis thaliana | O23482 | Oligopeptide transporter 3 (AtOPT3) |
| Arabidopsis thaliana | O23654 | V-type proton ATPase catalytic subunit A (V-ATPase subunit |
| | | A) (EC 3.6.3.14) (V-ATPase 69 kDa subunit) (Vacuolar H(+)-ATPase |
| | | subunit A) (Vacuolar proton pump subunit alpha) |
| Arabidopsis thaliana | O48788 | Probable inactive receptor kinase At2g26730 |
| Arabidopsis thaliana | O48963 | Phototropin-1 (EC 2.7.11.1) (Non-phototropic hypocotyl |
| | | protein 1) (Root phototropism protein 1) |
| Arabidopsis thaliana | O49195 | Vegetative storage protein 1 |
| Arabidopsis thaliana | O50008 | 5-methyltetrahydropteroyltriglutamate--homocysteine |
| | | methyltransferase 1 (EC 2.1.1.14) (Cobalamin-independent |
| | | methionine synthase 1) (AtMS1) (Vitamin-B12-independent |
| | | methionine synthase 1) |
| Arabidopsis thaliana | O64696 | Putative uncharacterized protein At2g34510 |
| Arabidopsis thaliana | O65572 | Carotenoid 9,10(9′,10′)-cleavage dioxygenase 1 (EC |
| | | 1.14.99.n4) (AtCCD1) (Neoxanthin cleavage enzyme NC1) |
| | | (AtNCED1) |
| Arabidopsis thaliana | O65660 | PLAT domain-containing protein 1 (AtPLAT1) (PLAT domain |
| | | protein 1) |
| Arabidopsis thaliana | O65719 | Heat shock 70 kDa protein 3 (Heat shock cognate 70 kDa |
| | | protein 3) (Heat shock cognate protein 70-3) (AtHsc70-3) |
| | | (Heat shock protein 70-3) (AtHsp70-3) |
| Arabidopsis thaliana | O80517 | Uclacyanin-2 (Blue copper-binding protein II) (BCB II) |
| | | (Phytocyanin 2) (Uclacyanin-II) |
| Arabidopsis thaliana | O80576 | At2g44060 (Late embryogenesis abundant protein, group 2) |
| | | (Similar to late embryogenesis abundant proteins) |
| Arabidopsis thaliana | O80725 | ABC transporter B family member 4 (ABC transporter |
| | | ABCB.4) (AtABCB4) (Multidrug resistance protein 4) |
| | | (P-glycoprotein 4) |
| Arabidopsis thaliana | O80837 | Remorin (DNA-binding protein) |
| Arabidopsis thaliana | O80852 | Glutathione S-transferase F9 (AtGSTF9) (EC 2.5.1.18) |
| | | (AtGSTF7) (GST class-phi member 9) |
| Arabidopsis thaliana | O80858 | Expressed protein (Putative uncharacterized protein |
| | | At2g30930) (Putative uncharacterized protein At2g30930; |
| | | F7F1.14) |
| Arabidopsis thaliana | O80939 | L-type lectin-domain containing receptor kinase IV.1 |
| | | (Arabidopsis thaliana lectin-receptor kinase e) (AthlecRK-e) |
| | | (LecRK-IV.1) (EC 2.7.11.1) (Lectin Receptor Kinase 1) |
| Arabidopsis thaliana | O80948 | Jacalin-related lectin 23 (Myrosinase-binding protein-like |
| | | At2g39330) |
| Arabidopsis thaliana | O82628 | V-type proton ATPase subunit G1 (V-ATPase subunit G1) |
| | | (Vacuolar H(+)-ATPase subunit G isoform 1) (Vacuolar |
| | | proton pump subunit G1) |
| Arabidopsis thaliana | P10795 | Ribulose bisphosphate carboxylase small chain 1A, |
| | | chloroplastic (RuBisCO small subunit 1A) (EC 4.1.1.39) |
| Arabidopsis thaliana | P10896 | Ribulose bisphosphate carboxylase/oxygenase activase, |
| | | chloroplastic (RA) (RuBisCO activase) |
| Arabidopsis thaliana | P17094 | 60S ribosomal protein L3-1 (Protein EMBRYO DEFECTIVE |
| | | 2207) |
| Arabidopsis thaliana | P19456 | ATPase 2, plasma membrane-type (EC 3.6.3.6) (Proton |
| | | pump 2) |
| Arabidopsis thaliana | P20649 | ATPase 1, plasma membrane-type (EC 3.6.3.6) (Proton |
| | | pump 1) |
| Arabidopsis thaliana | P22953 | Probable mediator of RNA polymerase II transcription subunit |
| | | 37e (Heat shock 70 kDa protein 1) (Heat shock cognate 70 |
| | | kDa protein 1) (Heat shock cognate protein 70-1) (AtHsc70-1) |
| | | (Heat shock protein 70-1) (AtHsp70-1) (Protein |
| | | EARLY-RESPONSIVE TO DEHYDRATION 2) |
| Arabidopsis thaliana | P23586 | Sugar transport protein 1 (Glucose transporter) (Hexose |
| | | transporter 1) |
| Arabidopsis thaliana | P24636 | Tubulin beta-4 chain (Beta-4-tubulin) |
| Arabidopsis thaliana | P25696 | Bifunctional enolase 2/transcriptional activator (EC 4.2.1.11) |
| | | (2-phospho-D-glycerate hydro-lyase 2) (2-phosphoglycerate |
| | | dehydratase 2) (LOW EXPRESSION OF OSMOTICALLY |
| | | RESPONSIVE GENES 1) |
| Arabidopsis thaliana | P25856 | Glyceraldehyde-3-phosphate dehydrogenase GAPA1, |
| | | chloroplastic (EC 1.2.1.13) (NADP-dependent |
| | | glyceraldehydephosphate dehydrogenase A subunit 1) |
| Arabidopsis thaliana | P28186 | Ras-related protein RABE1c (AtRABE1c) (Ras-related |
| | | protein Ara-3) (Ras-related protein Rab8A) (AtRab8A) |
| Arabidopsis thaliana | P30302 | Aquaporin PIP2-3 (Plasma membrane intrinsic protein 2-3) |
| | | (AtPIP2; 3) (Plasma membrane intrinsic protein 2c) (PIP2c) |
| | | (RD28-PIP) (TMP2C) (Water stress-induced tonoplast |
| | | intrinsic protein) (WSI-TIP) [Cleaved into: Aquaporin PIP2-3, |
| | | N-terminally processed] |
| Arabidopsis thaliana | P31414 | Pyrophosphate-energized vacuolar membrane proton pump |
| | | 1 (EC 3.6.1.1) (Pyrophosphate-energized inorganic |
| | | pyrophosphatase 1) (H(+)-PPase 1) (Vacuolar proton |
| | | pyrophosphatase 1) (Vacuolar proton pyrophosphatase 3) |
| Arabidopsis thaliana | P32961 | Nitrilase 1 (EC 3.5.5.1) |
| Arabidopsis thaliana | P38666 | 60S ribosomal protein L24-2 (Protein SHORT VALVE 1) |
| Arabidopsis thaliana | P39207 | Nucleoside diphosphate kinase 1 (EC 2.7.4.6) (Nucleoside |
| | | diphosphate kinase I) (NDK I) (NDP kinase I) (NDPK I) |
| Arabidopsis thaliana | P42643 | 14-3-3-like protein GF14 chi (General regulatory factor 1) |
| Arabidopsis thaliana | P42737 | Beta carbonic anhydrase 2, chloroplastic (AtbCA2) |
| | | (AtbetaCA2) (EC 4.2.1.1) (Beta carbonate dehydratase 2) |
| Arabidopsis thaliana | P42759 | Dehydrin ERD10 (Low-temperature-induced protein LTI45) |
| Arabidopsis thaliana | P42761 | Glutathione S-transferase F10 (AtGSTF10) (EC 2.5.1.18) |
| | | (AtGSTF4) (GST class-phi member 10) (Protein EARLY |
| | | RESPONSE TO DEHYDRATION 13) |
| Arabidopsis thaliana | P42763 | Dehydrin ERD14 |
| Arabidopsis thaliana | P42791 | 60S ribosomal protein L18-2 |
| Arabidopsis thaliana | P43286 | Aquaporin PIP2-1 (Plasma membrane intrinsic protein 2-1) |
| | | (AtPIP2; 1) (Plasma membrane intrinsic protein 2a) (PIP2a) |
| | | [Cleaved into: Aquaporin PIP2-1, N-terminally processed] |
| Arabidopsis thaliana | P46286 | 60S ribosomal protein L8-1 (60S ribosomal protein L2) |
| | | (Protein EMBRYO DEFECTIVE 2296) |
| Arabidopsis thaliana | P46422 | Glutathione S-transferase F2 (AtGSTF2) (EC 2.5.1.18) (24 |
| | | kDa auxin-binding protein) (AtPM24) (GST class-phi member |
| | | 2) |
| Arabidopsis thaliana | P47998 | Cysteine synthase 1 (EC 2.5.1.47) (At.OAS.5-8) (Beta-substituted |
| | | Ala synthase 1; 1) (ARAth-Bsas1; 1) (CSase A) |
| | | (AtCS-A) (Cys-3A) (O-acetylserine (thiol)-lyase 1) (OAS-TL |
| | | A) (O-acetylserine sulfhydrylase) (Protein ONSET OF LEAF |
| | | DEATH 3) |
| Arabidopsis thaliana | P48347 | 14-3-3-like protein GF14 epsilon (General regulatory factor |
| | | 10) |
| Arabidopsis thaliana | P48491 | Triosephosphate isomerase, cytosolic (TIM) (Triose-phosphate |
| | | isomerase) (EC 5.3.1.1) |
| Arabidopsis thaliana | P50318 | Phosphoglycerate kinase 2, chloroplastic (EC 2.7.2.3) |
| Arabidopsis thaliana | P53492 | Actin-7 (Actin-2) |
| Arabidopsis thaliana | P54144 | Ammonium transporter 1 member 1 (AtAMT1; 1) |
| Arabidopsis thaliana | P92963 | Ras-related protein RABB1c (AtRABB1c) (Ras-related |
| | | protein Rab2A) (AtRab2A) |
| Arabidopsis thaliana | P93004 | Aquaporin PIP2-7 (Plasma membrane intrinsic protein 2-7) |
| | | (AtPIP2; 7) (Plasma membrane intrinsic protein 3) (Salt |
| | | stress-induced major intrinsic protein) [Cleaved into: |
| | | Aquaporin PIP2-7, N-terminally processed] |
| Arabidopsis thaliana | P93025 | Phototropin-2 (EC 2.7.11.1) (Defective in chloroplast |
| | | avoidance protein 1) (Non-phototropic hypocotyl 1-like |
| | | protein 1) (AtKin7) (NPH1-like protein 1) |
| Arabidopsis thaliana | P93819 | Malate dehydrogenase 1, cytoplasmic (EC 1.1.1.37) |
| | | (Cytosolic NAD-dependent malate dehydrogenase 1) |
| | | (cNAD-MDH1) (Cytosolic malate dehydrogenase 1) |
| | | (Cytosolic MDH1) |
| Arabidopsis thaliana | Q03250 | Glycine-rich RNA-binding protein 7 (AtGR-RBP7) (AtRBG7) |
| | | (Glycine-rich protein 7) (AtGRP7) (Protein COLD, |
| | | CIRCADIAN RHYTHM, AND RNA BINDING 2) (Protein |
| | | CCR2) |
| Arabidopsis thaliana | Q05431 | L-ascorbate peroxidase 1, cytosolic (AP) (AtAPx01) (EC |
| | | 1.11.1.11) |
| Arabidopsis thaliana | Q06611 | Aquaporin PIP1-2 (AtPIP1; 2) (Plasma membrane intrinsic |
| | | protein 1b) (PIP1b) (Transmembrane protein A) (AthH2) |
| | | (TMP-A) |
| Arabidopsis thaliana | Q07488 | Blue copper protein (Blue copper-binding protein) (AtBCB) |
| | | (Phytocyanin 1) (Stellacyanin) |
| Arabidopsis thaliana | Q0WLB5 | Clathrin heavy chain 2 |
| Arabidopsis thaliana | Q0WNJ6 | Clathrin heavy chain 1 |
| Arabidopsis thaliana | Q1ECE0 | Vesicle-associated protein 4-1 (Plant VAP homolog 4-1) |
| | | (AtPVA41) (Protein MEMBRANE-ASSOCIATED MANNITOL-INDUCED) |
| | | (AtMAMI) (VAMP-associated protein 4-1) |
| Arabidopsis thaliana | Q38882 | Phospholipase D alpha 1 (AtPLDalpha1) (PLD alpha 1) (EC |
| | | 3.1.4.4) (Choline phosphatase 1) (PLDalpha) |
| | | (Phosphatidylcholine-hydrolyzing phospholipase D 1) |
| Arabidopsis thaliana | Q38900 | Peptidyl-prolyl cis-trans isomerase CYP19-1 (PPIase CYP19-1) |
| | | (EC 5.2.1.8) (Cyclophilin of 19 kDa 1) (Rotamase |
| | | cyclophilin-3) |
| Arabidopsis thaliana | Q39033 | Phosphoinositide phospholipase C 2 (EC 3.1.4.11) |
| | | (Phosphoinositide phospholipase PLC2) (AtPLC2) (PI-PLC2) |
| Arabidopsis thaliana | Q39085 | Delta(24)-sterol reductase (EC 1.3.1.72) (Cell elongation |
| | | protein DIMINUTO) (Cell elongation protein Dwarf1) (Protein |
| | | CABBAGE1) (Protein ENHANCED VERY-LOW-FLUENCE |
| | | RESPONSE 1) |
| Arabidopsis thaliana | Q39228 | Sugar transport protein 4 (Hexose transporter 4) |
| Arabidopsis thaliana | Q39241 | Thioredoxin H5 (AtTrxh5) (Protein LOCUS OF |
| | | INSENSITIVITY TO VICTORIN 1) (Thioredoxin 5) (AtTRX5) |
| Arabidopsis thaliana | Q39258 | V-type proton ATPase subunit E1 (V-ATPase subunit E1) |
| | | (Protein EMBRYO DEFECTIVE 2448) (Vacuolar |
| | | H(+)-ATPase subunit E isoform 1) (Vacuolar proton pump subunit |
| | | E1) |
| Arabidopsis thaliana | Q42112 | 60S acidic ribosomal protein P0-2 |
| Arabidopsis thaliana | Q42403 | Thioredoxin H3 (AtTrxh3) (Thioredoxin 3) (AtTRX3) |
| Arabidopsis thaliana | Q42479 | Calcium-dependent protein kinase 3 (EC 2.7.11.1) |
| | | (Calcium-dependent protein kinase isoform CDPK6) (AtCDPK6) |
| Arabidopsis thaliana | Q42547 | Catalase-3 (EC 1.11.1.6) |
| Arabidopsis thaliana | Q56WH1 | Tubulin alpha-3 chain |
| Arabidopsis thaliana | Q56WK6 | Patellin-1 |
| Arabidopsis thaliana | Q56X75 | CASP-like protein 4D2 (AtCASPL4D2) |
| Arabidopsis thaliana | Q56ZI2 | Patellin-2 |
| Arabidopsis thaliana | Q7Y208 | Glycerophosphodiester phosphodiesterase GDPDL1 (EC |
| | | 3.1.4.46) (Glycerophosphodiester phosphodiesterase-like 1) |
| | | (ATGDPDL1) (Glycerophosphodiesterase-like 3) (Protein |
| | | SHV3-LIKE 2) |
| Arabidopsis thaliana | Q84VZ5 | Uncharacterized GPI-anchored protein At5g19240 |
| Arabidopsis thaliana | Q84WU7 | Eukaryotic aspartyl protease family protein (Putative |
| | | uncharacterized protein At3g51330) |
| Arabidopsis thaliana | Q8GUL8 | Uncharacterized GPI-anchored protein At5g19230 |
| Arabidopsis thaliana | Q8GYA4 | Cysteine-rich receptor-like protein kinase 10 (Cysteine-rich |
| | | RLK10) (EC 2.7.11.—) (Receptor-like protein kinase 4) |
| Arabidopsis thaliana | Q8GYN5 | RPM1-interacting protein 4 |
| Arabidopsis thaliana | Q8GZ99 | At5g49760 (Leucine-rich repeat protein kinase family protein) |
| | | (Leucine-rich repeat receptor-like protein kinase) (Putative |
| | | receptor protein kinase) |
| Arabidopsis thaliana | Q8L636 | Sodium/calcium exchanger NCL (Na(+)/Ca(2+)-exchange |
| | | protein NCL) (Protein NCX-like) (AtNCL) |
| Arabidopsis thaliana | Q8L7S1 | At1g45200 (At1g45200/At1g45200) (Triacylglycerol |
| | | lipase-like 1) |
| Arabidopsis thaliana | Q8LAA6 | Probable aquaporin PIP1-5 (AtPIP1; 5) (Plasma membrane |
| | | intrinsic protein 1d) (PIP1d) |
| Arabidopsis thaliana | Q8LCP6 | Endoglucanase 10 (EC 3.2.1.4) (Endo-1,4-beta glucanase |
| | | 10) |
| Arabidopsis thaliana | Q8RWV0 | Transketolase-1, chloroplastic (TK) (EC 2.2.1.1) |
| Arabidopsis thaliana | Q8S8Q6 | Tetraspanin-8 |
| Arabidopsis thaliana | Q8VZG8 | MDIS1-interacting receptor like kinase 2 (AtMIK2) (Probable |
| | | LRR receptor-like serine/threonine-protein kinase |
| | | At4g08850) (EC 2.7.11.1) |
| Arabidopsis thaliana | Q8VZU2 | Syntaxin-132 (AtSYP132) |
| Arabidopsis thaliana | Q8W4E2 | V-type proton ATPase subunit B3 (V-ATPase subunit B3) |
| | | (Vacuolar H(+)-ATPase subunit B isoform 3) (Vacuolar |
| | | proton pump subunit B3) |
| Arabidopsis thaliana | Q8W4S4 | V-type proton ATPase subunit a3 (V-ATPase subunit a3) |
| | | (V-type proton ATPase 95 kDa subunit a isoform 3) (V-ATPase |
| | | 95 kDa isoform a3) (Vacuolar H(+)-ATPase subunit a isoform |
| | | 3) (Vacuolar proton pump subunit a3) (Vacuolar proton |
| | | translocating ATPase 95 kDa subunit a isoform 3) |
| Arabidopsis thaliana | Q93VG5 | 40S ribosomal protein S8-1 |
| Arabidopsis thaliana | Q93XY5 | Tetraspanin-18 (TOM2A homologous protein 2) |
| Arabidopsis thaliana | Q93YS4 | ABC transporter G family member 22 (ABC transporter |
| | | ABCG.22) (AtABCG22) (White-brown complex homolog |
| | | protein 23) (AtWBC23) |
| Arabidopsis thaliana | Q93Z08 | Glucan endo-1,3-beta-glucosidase 6 (EC 3.2.1.39) |
| | | ((1 −> 3)-beta-glucan endohydrolase 6) |
| | | ((1 −> 3)-beta-glucanase 6) (Beta-1,3-endoglucanase |
| | | 6) (Beta-1,3-glucanase 6) |
| Arabidopsis thaliana | Q940M8 | 3-oxo-5-alpha-steroid 4-dehydrogenase (DUF1295) |
| | | (At1g73650/F25P22_7) |
| Arabidopsis thaliana | Q944A7 | Probable serine/threonine-protein kinase At4g35230 (EC |
| | | 2.7.11.1) |
| Arabidopsis thaliana | Q944G5 | Protein NRT1/PTR FAMILY 2.10 (AtNPF2.10) (Protein |
| | | GLUCOSINOLATE TRANSPORTER-1) |
| Arabidopsis thaliana | Q94AZ2 | Sugar transport protein 13 (Hexose transporter 13) |
| | | (Multicopy suppressor of snf4 deficiency protein 1) |
| Arabidopsis thaliana | Q94BT2 | Auxin-induced in root cultures protein 12 |
| Arabidopsis thaliana | Q94CE4 | Beta carbonic anhydrase 4 (AtbCA4) (AtbetaCA4) (EC |
| | | 4.2.1.1) (Beta carbonate dehydratase 4) |
| Arabidopsis thaliana | Q94KI8 | Two pore calcium channel protein 1 (Calcium channel protein |
| | | 1) (AtCCH1) (Fatty acid oxygenation up-regulated protein 2) |
| | | (Voltage-dependent calcium channel protein TPC1) (AtTPC1) |
| Arabidopsis thaliana | Q96262 | Plasma membrane-associated cation-binding protein 1 |
| | | (AtPCAP1) (Microtubule-destabilizing protein 25) |
| Arabidopsis thaliana | Q9C5Y0 | Phospholipase D delta (AtPLDdelta) (PLD delta) (EC 3.1.4.4) |
| Arabidopsis thaliana | Q9C7F7 | Non-specific lipid transfer protein GPI-anchored 1 |
| | | (AtLTPG-1) (Protein LTP-GPI-ANCHORED 1) |
| Arabidopsis thaliana | Q9C821 | Proline-rich receptor-like protein kinase PERK15 (EC |
| | | 2.7.11.1) (Proline-rich extensin-like receptor kinase 15) |
| | | (AtPERK15) |
| Arabidopsis thaliana | Q9C8G5 | CSC1-like protein ERD4 (Protein EARLY-RESPONSIVE TO |
| | | DEHYDRATION STRESS 4) |
| Arabidopsis thaliana | Q9C9C5 | 60S ribosomal protein L6-3 |
| Arabidopsis thaliana | Q9CAR7 | Hypersensitive-induced response protein 2 (AtHIR2) |
| Arabidopsis thaliana | Q9FFH6 | Fasciclin-like arabinogalactan protein 13 |
| Arabidopsis thaliana | Q9FGT8 | Temperature-induced lipocalin-1 (AtTIL1) |
| Arabidopsis thaliana | Q9FJ62 | Glycerophosphodiester phosphodiesterase GDPDL4 (EC |
| | | 3.1.4.46) (Glycerophosphodiester phosphodiesterase-like 4) |
| | | (ATGDPDL4) (Glycerophosphodiesterase-like 1) (Protein |
| | | SHV3-LIKE 1) |
| Arabidopsis thaliana | Q9FK68 | Ras-related protein RABA1c (AtRABA1c) |
| Arabidopsis thaliana | Q9FKS8 | Lysine histidine transporter 1 |
| Arabidopsis thaliana | Q9FM65 | Fasciclin-like arabinogalactan protein 1 |
| Arabidopsis thaliana | Q9FNH6 | NDR1/HIN1-like protein 3 |
| Arabidopsis thaliana | Q9FRL3 | Sugar transporter ERD6-like 6 |
| Arabidopsis thaliana | Q9FWR4 | Glutathione S-transferase DHAR1, mitochondrial (EC |
| | | 2.5.1.18) (Chloride intracellular channel homolog 1) (CLIC |
| | | homolog 1) (Glutathione-dependent dehydroascorbate |
| | | reductase 1) (AtDHAR1) (GSH-dependent dehydroascorbate |
| | | reductase 1) (mtDHAR) |
| Arabidopsis thaliana | Q9FX54 | Glyceraldehyde-3-phosphate dehydrogenase GAPC2, |
| | | cytosolic (EC 1.2.1.12) (NAD-dependent |
| | | glyceraldehydephosphate dehydrogenase C subunit 2) |
| Arabidopsis thaliana | Q9LE22 | Probable calcium-binding protein CML27 (Calmodulin-like |
| | | protein 27) |
| Arabidopsis thaliana | Q9LEX1 | At3g61050 (CaLB protein) (Calcium-dependent lipid-binding |
| | | (CaLB domain) family protein) |
| Arabidopsis thaliana | Q9LF79 | Calcium-transporting ATPase 8, plasma membrane-type (EC |
| | | 3.6.3.8) (Ca(2+)-ATPase isoform 8) |
| Arabidopsis thaliana | Q9LJG3 | GDSL esterase/lipase ESM1 (EC 3.1.1.—) (Extracellular lipase |
| | | ESM1) (Protein EPITHIOSPECIFIER MODIFIER 1) |
| | | (AtESM1) |
| Arabidopsis thaliana | Q9LJI5 | V-type proton ATPase subunit d1 (V-ATPase subunit d1) |
| | | (Vacuolar H(+)-ATPase subunit d isoform 1) (Vacuolar proton |
| | | pump subunit d1) |
| Arabidopsis thaliana | Q9LME4 | Probable protein phosphatase 2C 9 (AtPP2C09) (EC |
| | | 3.1.3.16) (Phytochrome-associated protein phosphatase 2C) |
| | | (PAPP2C) |
| Arabidopsis thaliana | Q9LNP3 | At1g17620/F11A6_23 (F1L3.32) (Late embryogenesis |
| | | abundant (LEA) hydroxyproline-rich glycoprotein family) |
| | | (Putative uncharacterized protein At1g17620) |
| Arabidopsis thaliana | Q9LNW1 | Ras-related protein RABA2b (AtRABA2b) |
| Arabidopsis thaliana | Q9LQU2 | Protein PLANT CADMIUM RESISTANCE 1 (AtPCR1) |
| Arabidopsis thaliana | Q9LQU4 | Protein PLANT CADMIUM RESISTANCE 2 (AtPCR2) |
| Arabidopsis thaliana | Q9LR30 | Glutamate--glyoxylate aminotransferase 1 (AtGGT2) (EC |
| | | 2.6.1.4) (Alanine aminotransferase GGT1) (EC 2.6.1.2) |
| | | (Alanine--glyoxylate aminotransferase GGT1) (EC 2.6.1.44) |
| | | (Alanine-2-oxoglutarate aminotransferase 1) (EC 2.6.1.—) |
| Arabidopsis thaliana | Q9LSI9 | Inactive LRR receptor-like serine/threonine-protein kinase |
| | | BIR2 (Protein BAK1-INTERACTING RECEPTOR-LIKE |
| | | KINASE 2) |
| Arabidopsis thaliana | Q9LSQ5 | NAD(P)H dehydrogenase (quinone) FQR1 (EC 1.6.5.2) |
| | | (Flavodoxin-like quinone reductase 1) |
| Arabidopsis thaliana | Q9LUT0 | Protein kinase superfamily protein (Putative uncharacterized |
| | | protein At3g17410) (Serine/threonine protein kinase-like |
| | | protein) |
| Arabidopsis thaliana | Q9LV48 | Proline-rich receptor-like protein kinase PERK1 (EC 2.7.11.1) |
| | | (Proline-rich extensin-like receptor kinase 1) (AtPERK1) |
| Arabidopsis thaliana | Q9LX65 | V-type proton ATPase subunit H (V-ATPase subunit H) |
| | | (Vacuolar H(+)-ATPase subunit H) (Vacuolar proton pump |
| | | subunit H) |
| Arabidopsis thaliana | Q9LYG3 | NADP-dependent malic enzyme 2 (AtNADP-ME2) |
| | | (NADP-malic enzyme 2) (EC 1.1.1.40) |
| Arabidopsis thaliana | Q9M088 | Glucan endo-1,3-beta-glucosidase 5 (EC 3.2.1.39) |
| | | ((1 −> 3)-beta-glucan endohydrolase 5) |
| | | ((1 −> 3)-beta-glucanase 5) (Beta-1,3-endoglucanase |
| | | 5) (Beta-1,3-glucanase 5) |
| Arabidopsis thaliana | Q9M2D8 | Uncharacterized protein At3g61260 |
| Arabidopsis thaliana | Q9M386 | Late embryogenesis abundant (LEA) hydroxyproline-rich |
| | | glycoprotein family (Putative uncharacterized protein |
| | | At3g54200) (Putative uncharacterized protein F24B22.160) |
| Arabidopsis thaliana | Q9M390 | Protein NRT1/PTR FAMILY 8.1 (AtNPF8.1) (Peptide |
| | | transporter PTR1) |
| Arabidopsis thaliana | Q9M5P2 | Secretory carrier-associated membrane protein 3 (AtSC3) |
| | | (Secretory carrier membrane protein 3) |
| Arabidopsis thaliana | Q9M8T0 | Probable inactive receptor kinase At3g02880 |
| Arabidopsis thaliana | Q9SDS7 | V-type proton ATPase subunit C (V-ATPase subunit C) |
| | | (Vacuolar H(+)-ATPase subunit C) (Vacuolar proton pump |
| | | subunit C) |
| Arabidopsis thaliana | Q9SEL6 | Vesicle transport v-SNARE 11 (AtVTI11) (Protein SHOOT |
| | | GRAVITROPISM 4) (Vesicle soluble NSF attachment protein |
| | | receptor VTI1a) (AtVTI1a) (Vesicle transport v-SNARE |
| | | protein VTI1a) |
| Arabidopsis thaliana | Q9SF29 | Syntaxin-71 (AtSYP71) |
| Arabidopsis thaliana | Q9SF85 | Adenosine kinase 1 (AK 1) (EC 2.7.1.20) (Adenosine |
| | | 5′-phosphotransferase 1) |
| Arabidopsis thaliana | Q9SIE7 | PLAT domain-containing protein 2 (AtPLAT2) (PLAT domain |
| | | protein 2) |
| Arabidopsis thaliana | Q9SIM4 | 60S ribosomal protein L14-1 |
| Arabidopsis thaliana | Q9SIU8 | Probable protein phosphatase 2C 20 (AtPP2C20) (EC |
| | | 3.1.3.16) (AtPPC3; 1.2) |
| Arabidopsis thaliana | Q9SJ81 | Fasciclin-like arabinogalactan protein 7 |
| Arabidopsis thaliana | Q9SKB2 | Leucine-rich repeat receptor-like serine/threonine/tyrosine-protein |
| | | kinase SOBIR1 (EC 2.7.10.1) (EC 2.7.11.1) (Protein |
| | | EVERSHED) (Protein SUPPRESSOR OF BIR1-1) |
| Arabidopsis thaliana | Q9SKR2 | Synaptotagmin-1 (NTMC2T1.1) (Synaptotagmin A) |
| Arabidopsis thaliana | Q9SLF7 | 60S acidic ribosomal protein P2-2 |
| Arabidopsis thaliana | Q9SPE6 | Alpha-soluble NSF attachment protein 2 (Alpha-SNAP2) |
| | | (N-ethylmaleimide-sensitive factor attachment protein alpha 2) |
| Arabidopsis thaliana | Q9SRH6 | Hypersensitive-induced response protein 3 (AtHIR3) |
| Arabidopsis thaliana | Q9SRY5 | Glutathione S-transferase F7 (EC 2.5.1.18) (AtGSTF8) (GST |
| | | class-phi member 7) (Glutathione S-transferase 11) |
| Arabidopsis thaliana | Q9SRZ6 | Cytosolic isocitrate dehydrogenase [NADP] (EC 1.1.1.42) |
| Arabidopsis thaliana | Q9SSK5 | MLP-like protein 43 |
| Arabidopsis thaliana | Q9SU13 | Fasciclin-like arabinogalactan protein 2 |
| Arabidopsis thaliana | Q9SU40 | Monocopper oxidase-like protein SKU5 (Skewed roots) |
| Arabidopsis thaliana | Q9SUR6 | Cystine lyase CORI3 (EC 4.4.1.35) (Protein CORONATINE |
| | | INDUCED 3) (Protein JASMONIC ACID RESPONSIVE 2) |
| | | (Tyrosine aminotransferase CORI3) |
| Arabidopsis thaliana | Q9SVC2 | Syntaxin-122 (AtSYP122) (Synt4) |
| Arabidopsis thaliana | Q9SVF0 | Putative uncharacterized protein AT4g38350 (Putative |
| | | uncharacterized protein F22I13.120) |
| Arabidopsis thaliana | Q9SW40 | Major facilitator superfamily protein (Putative uncharacterized |
| | | protein AT4g34950) (Putative uncharacterized protein |
| | | T11I11.190) |
| Arabidopsis thaliana | Q9SYT0 | Annexin D1 (AnnAt1) (Annexin A1) |
| Arabidopsis thaliana | Q9SZ11 | Glycerophosphodiester phosphodiesterase GDPDL3 (EC |
| | | 3.1.4.46) (Glycerophosphodiester phosphodiesterase-like 3) |
| | | (ATGDPDL3) (Glycerophosphodiesterase-like 2) (Protein |
| | | MUTANT ROOT HAIR 5) (Protein SHAVEN 3) |
| Arabidopsis thaliana | Q9SZN1 | V-type proton ATPase subunit B2 (V-ATPase subunit B2) |
| | | (Vacuolar H(+)-ATPase subunit B isoform 2) (Vacuolar |
| | | proton pump subunit B2) |
| Arabidopsis thaliana | Q9SZP6 | AT4g38690/F20M13_250 (PLC-like phosphodiesterases |
| | | superfamily protein) (Putative uncharacterized protein |
| | | AT4g38690) (Putative uncharacterized protein F20M13.250) |
| Arabidopsis thaliana | Q9SZR1 | Calcium-transporting ATPase 10, plasma membrane-type |
| | | (EC 3.6.3.8) (Ca(2+)-ATPase isoform 10) |
| Arabidopsis thaliana | Q9T053 | Phospholipase D gamma 1 (AtPLDgamma1) (PLD gamma 1) |
| | | (EC 3.1.4.4) (Choline phosphatase) (Lecithinase D) |
| | | (Lipophosphodiesterase II) |
| Arabidopsis thaliana | Q9T076 | Early nodulin-like protein 2 (Phytocyanin-like protein) |
| Arabidopsis thaliana | Q9T0A0 | Long chain acyl-CoA synthetase 4 (EC 6.2.1.3) |
| Arabidopsis thaliana | Q9T0G4 | Putative uncharacterized protein AT4g10060 (Putative |
| | | uncharacterized protein T5L19.190) |
| Arabidopsis thaliana | Q9XEE2 | Annexin D2 (AnnAt2) |
| Arabidopsis thaliana | Q9XGM1 | V-type proton ATPase subunit D (V-ATPase subunit D) |
| | | (Vacuolar H(+)-ATPase subunit D) (Vacuolar proton pump |
| | | subunit D) |
| Arabidopsis thaliana | Q9XI93 | At1g13930/F16A14.27 (F16A14.14) (F7A19.2 protein) |
| | | (Oleosin-B3-like protein) |
| Arabidopsis thaliana | Q9XIE2 | ABC transporter G family member 36 (ABC transporter |
| | | ABCG.36) (AtABCG36) (Pleiotropic drug resistance protein |
| | | 8) (Protein PENETRATION 3) |
| Arabidopsis thaliana | Q9ZPZ4 | Putative uncharacterized protein (Putative uncharacterized |
| | | protein At1g09310) (T31J12.3 protein) |
| Arabidopsis thaliana | Q9ZQX4 | V-type proton ATPase subunit F (V-ATPase subunit F) |
| | | (V-ATPase 14 kDa subunit) (Vacuolar H(+)-ATPase subunit F) |
| | | (Vacuolar proton pump subunit F) |
| Arabidopsis thaliana | Q9ZSA2 | Calcium-dependent protein kinase 21 (EC 2.7.11.1) |
| Arabidopsis thaliana | Q9ZSD4 | Syntaxin-121 (AtSYP121) (Syntaxin-related protein At-Syr1) |
| Arabidopsis thaliana | Q9ZV07 | Probable aquaporin PIP2-6 (Plasma membrane intrinsic |
| | | protein 2-6) (AtPIP2; 6) (Plasma membrane intrinsic protein |
| | | 2e) (PIP2e) [Cleaved into: Probable aquaporin PIP2-6, |
| | | N-terminally processed] |
| Arabidopsis thaliana | Q9ZVF3 | MLP-like protein 328 |
| Arabidopsis thaliana | Q9ZWA8 | Fasciclin-like arabinogalactan protein 9 |
| Arabidopsis thaliana | Q9ZSD4 | SYR1, Syntaxin Related Protein 1, also known as SYP121, |
| | | PENETRATION1/PEN1 (Protein PENETRATION 1) |
| Citrus lemon | A1ECK0 | Putative glutaredoxin |
| Citrus lemon | A9YVC9 | Pyrophosphate--fructose 6-phosphate 1-phosphotransferase |
| | | subunit beta (PFP) (EC 2.7.1.90) (6-phosphofructokinase, |
| | | pyrophosphate dependent) (PPi-PFK) |
| | | (Pyrophosphate-dependent 6-phosphofructose-1-kinase) |
| Citrus lemon | B2YGY1 | Glycosyltransferase (EC 2.4.1.—) |
| Citrus lemon | B6DZD3 | Glutathione S-transferase Tau2 (Glutathione transferase |
| | | Tau2) |
| Citrus lemon | C3VIC2 | Translation elongation factor |
| Citrus lemon | C8CPS0 | Importin subunit alpha |
| Citrus lemon | D3JWB5 | Flavanone 3-hydroxylase |
| Citrus lemon | E0ADY2 | Putative caffeic acid O-methyltransferase |
| Citrus lemon | E5DK62 | ATP synthase subunit alpha (Fragment) |
| Citrus lemon | E9M5S3 | Putative L-galactose-1-phosphate phosphatase |
| Citrus lemon | F1CGQ9 | Heat shock protein 90 |
| Citrus lemon | F8WL79 | Aminopeptidase (EC 3.4.11.—) |
| Citrus lemon | F8WL86 | Heat shock protein |
| Citrus lemon | K9JG59 | Abscisic acid stress ripening-related protein |
| Citrus lemon | Q000W4 | Fe(III)-chelate reductase |
| Citrus lemon | Q39538 | Heat shock protein (Fragment) |
| Citrus lemon | Q5UEN6 | Putative signal recognition particle protein |
| Citrus lemon | Q8GV08 | Dehydrin |
| Citrus lemon | Q8L893 | Cytosolic phosphoglucomutase (Fragment) |
| Citrus lemon | Q8S990 | Polygalacturonase-inhibiting protein |
| Citrus lemon | Q8W3U6 | Polygalacturonase-inhibitor protein |
| Citrus lemon | Q93XL8 | Dehydrin COR15 |
| Citrus lemon | Q941Q1 | Non-symbiotic hemoglobin class 1 |
| Citrus lemon | Q9MBF3 | Glycine-rich RNA-binding protein |
| Citrus lemon | Q9SP55 | V-type proton ATPase subunit G (V-ATPase subunit G) |
| | | (Vacuolar proton pump subunit G) |
| Citrus lemon | Q9THJ8 | Ribulose bisphosphate carboxylase large chain (EC 4.1.1.39) |
| | | (Fragment) |
| Citrus lemon | Q9ZST2 | Pyrophosphate--fructose 6-phosphate 1-phosphotransferase |
| | | subunit alpha (PFP) (6-phosphofructokinase, pyrophosphate |
| | | dependent) (PPi-PFK) (Pyrophosphate-dependent |
| | | 6-phosphofructose-1-kinase) |
| Citrus lemon | Q9ZWH6 | Polygalacturonase inhibitor |
| Citrus lemon | S5DXI9 | Nucleocapsid protein |
| Citrus lemon | S5NFC6 | GTP cyclohydrolase |
| Citrus lemon | V4RG42 | Uncharacterized protein |
| Citrus lemon | V4RGP4 | Uncharacterized protein |
| Citrus lemon | V4RHN8 | Uncharacterized protein |
| Citrus lemon | V4RJ07 | Uncharacterized protein |
| Citrus lemon | V4RJK9 | Adenosylhomocysteinase (EC 3.3.1.1) |
| Citrus lemon | V4RJM1 | Uncharacterized protein |
| Citrus lemon | V4RJX1 | 40S ribosomal protein S6 |
| Citrus lemon | V4RLB2 | Uncharacterized protein |
| Citrus lemon | V4RMX8 | Uncharacterized protein |
| Citrus lemon | V4RNA5 | Uncharacterized protein |
| Citrus lemon | V4RP81 | Glycosyltransferase (EC 2.4.1.—) |
| Citrus lemon | V4RPZ5 | Adenylyl cyclase-associated protein |
| Citrus lemon | V4RTN9 | Histone H4 |
| Citrus lemon | V4RUZ4 | Phosphoserine aminotransferase (EC 2.6.1.52) |
| Citrus lemon | V4RVF6 | Uncharacterized protein |
| Citrus lemon | V4RXD4 | Uncharacterized protein |
| Citrus lemon | V4RXG2 | Uncharacterized protein |
| Citrus lemon | V4RYA0 | Uncharacterized protein |
| Citrus lemon | V4RYE3 | Uncharacterized protein |
| Citrus lemon | V4RYH3 | Uncharacterized protein |
| Citrus lemon | V4RYX8 | Uncharacterized protein |
| Citrus lemon | V4RZ12 | Coatomer subunit beta′ |
| Citrus lemon | V4RZ89 | Uncharacterized protein |
| Citrus lemon | V4RZE3 | Uncharacterized protein |
| Citrus lemon | V4RZF3 | 1,2-dihydroxy-3-keto-5-methylthiopentene dioxygenase (EC |
| | | 1.13.11.54) (Acireductone dioxygenase (Fe(2+)-requiring)) |
| | | (ARD) (Fe-ARD) |
| Citrus lemon | V4RZM7 | Uncharacterized protein |
| Citrus lemon | V4RZX6 | Uncharacterized protein |
| Citrus lemon | V4S1V0 | Uncharacterized protein |
| Citrus lemon | V4S2B6 | Uncharacterized protein |
| Citrus lemon | V4S2N1 | Uncharacterized protein |
| Citrus lemon | V4S2S5 | Uncharacterized protein (Fragment) |
| Citrus lemon | V4S346 | Uncharacterized protein |
| Citrus lemon | V4S3T8 | Uncharacterized protein |
| Citrus lemon | V4S409 | Cyanate hydratase (Cyanase) (EC 4.2.1.104) (Cyanate |
| | | hydrolase) (Cyanate lyase) |
| Citrus lemon | V4S4E4 | Histone H2B |
| Citrus lemon | V4S4F6 | Flavin-containing monooxygenase (EC 1.—.—.—) |
| Citrus lemon | V4S4J1 | Uncharacterized protein |
| Citrus lemon | V4S4K9 | Uncharacterized protein |
| Citrus lemon | V4S535 | Proteasome subunit alpha type (EC 3.4.25.1) |
| Citrus lemon | V4S5A8 | Isocitrate dehydrogenase [NADP] (EC 1.1.1.42) |
| Citrus lemon | V4S5G8 | Uncharacterized protein |
| Citrus lemon | V4S5I6 | Uncharacterized protein |
| Citrus lemon | V4S5N4 | Uncharacterized protein (Fragment) |
| Citrus lemon | V4S5Q3 | Uncharacterized protein |
| Citrus lemon | V4S5X8 | Uncharacterized protein |
| Citrus lemon | V4S5Y1 | Uncharacterized protein |
| Citrus lemon | V4S6P4 | Calcium-transporting ATPase (EC 3.6.3.8) |
| Citrus lemon | V4S6W0 | Uncharacterized protein |
| Citrus lemon | V4S6W7 | Uncharacterized protein (Fragment) |
| Citrus lemon | V4S6Y4 | Uncharacterized protein |
| Citrus lemon | V4S773 | Ribosomal protein L19 |
| Citrus lemon | V4S7U0 | Uncharacterized protein |
| Citrus lemon | V4S7U5 | Uncharacterized protein |
| Citrus lemon | V4S7W4 | Pyruvate kinase (EC 2.7.1.40) |
| Citrus lemon | V4S885 | Uncharacterized protein |
| Citrus lemon | V4S8T3 | Peptidyl-prolyl cis-trans isomerase (PPIase) (EC 5.2.1.8) |
| Citrus lemon | V4S920 | Uncharacterized protein |
| Citrus lemon | V4S999 | Uncharacterized protein |
| Citrus lemon | V4S9G5 | Phosphoglycerate kinase (EC 2.7.2.3) |
| Citrus lemon | V4S9Q6 | Beta-amylase (EC 3.2.1.2) |
| Citrus lemon | V4SA44 | Serine/threonine-protein phosphatase (EC 3.1.3.16) |
| Citrus lemon | V4SAE0 | Alpha-1,4 glucan phosphorylase (EC 2.4.1.1) |
| Citrus lemon | V4SAF6 | Uncharacterized protein |
| Citrus lemon | V4SAI9 | Eukaryotic translation initiation factor 3 subunit M (eIF3m) |
| Citrus lemon | V4SAJ5 | Ribosomal protein |
| Citrus lemon | V4SAR3 | Uncharacterized protein |
| Citrus lemon | V4SB37 | Uncharacterized protein |
| Citrus lemon | V4SBI0 | Elongation factor 1-alpha |
| Citrus lemon | V4SBI8 | D-3-phosphoglycerate dehydrogenase (EC 1.1.1.95) |
| Citrus lemon | V4SBL9 | Polyadenylate-binding protein (PABP) |
| Citrus lemon | V4SBR1 | S-formylglutathione hydrolase (EC 3.1.2.12) |
| Citrus lemon | V4SBR6 | Uncharacterized protein |
| Citrus lemon | V4SCG7 | Uncharacterized protein |
| Citrus lemon | V4SCJ2 | Uncharacterized protein |
| Citrus lemon | V4SCQ6 | Peptidyl-prolyl cis-trans isomerase (PPIase) (EC 5.2.1.8) |
| Citrus lemon | V4SDJ8 | Uncharacterized protein |
| Citrus lemon | V4SE41 | Protein DETOXIFICATION (Multidrug and toxic compound |
| | | extrusion protein) |
| Citrus lemon | V4SE90 | Uncharacterized protein |
| Citrus lemon | V4SED1 | Succinate dehydrogenase [ubiquinone] flavoprotein subunit, |
| | | mitochondrial (EC 1.3.5.1) |
| Citrus lemon | V4SEI1 | Uncharacterized protein |
| Citrus lemon | V4SEN9 | Uncharacterized protein |
| Citrus lemon | V4SEX8 | Uncharacterized protein |
| Citrus lemon | V4SF31 | Uncharacterized protein |
| Citrus lemon | V4SF69 | 40S ribosomal protein S24 |
| Citrus lemon | V4SF76 | Cysteine synthase (EC 2.5.1.47) |
| Citrus lemon | V4SFK3 | Uncharacterized protein |
| Citrus lemon | V4SFL4 | Uncharacterized protein |
| Citrus lemon | V4SFW2 | Uncharacterized protein |
| Citrus lemon | V4SGC9 | Uncharacterized protein |
| Citrus lemon | V4SGJ4 | Uncharacterized protein |
| Citrus lemon | V4SGN4 | Uncharacterized protein |
| Citrus lemon | V4SGV6 | Uncharacterized protein |
| Citrus lemon | V4SGV7 | Uncharacterized protein |
| Citrus lemon | V4SHH1 | Plasma membrane ATPase (EC 3.6.3.6) (Fragment) |
| Citrus lemon | V4SHI2 | Uncharacterized protein |
| Citrus lemon | V4SHJ3 | Uncharacterized protein |
| Citrus lemon | V4SI86 | Uncharacterized protein |
| Citrus lemon | V4SI88 | Uncharacterized protein |
| Citrus lemon | V4SIA2 | Uncharacterized protein |
| Citrus lemon | V4SIC1 | Phospholipase D (EC 3.1.4.4) |
| Citrus lemon | V4SJ14 | Uncharacterized protein |
| Citrus lemon | V4SJ48 | Uncharacterized protein |
| Citrus lemon | V4SJ69 | Uncharacterized protein |
| Citrus lemon | V4SJD9 | Uncharacterized protein |
| Citrus lemon | V4SJS7 | Uncharacterized protein |
| Citrus lemon | V4SJT5 | Uncharacterized protein |
| Citrus lemon | V4SKA2 | Uncharacterized protein |
| Citrus lemon | V4SKG4 | Glucose-6-phosphate isomerase (EC 5.3.1.9) |
| Citrus lemon | V4SKJ1 | Uncharacterized protein |
| Citrus lemon | V4SL90 | Uncharacterized protein |
| Citrus lemon | V4SLC6 | Proteasome subunit beta type (EC 3.4.25.1) |
| Citrus lemon | V4SLI7 | Uncharacterized protein |
| Citrus lemon | V4SLQ6 | Uncharacterized protein |
| Citrus lemon | V4SMD8 | Uncharacterized protein |
| Citrus lemon | V4SMN7 | Uncharacterized protein |
| Citrus lemon | V4SMV5 | Uncharacterized protein |
| Citrus lemon | V4SN00 | Uncharacterized protein |
| Citrus lemon | V4SNA9 | Uncharacterized protein |
| Citrus lemon | V4SNC1 | Uncharacterized protein |
| Citrus lemon | V4SNC4 | Aconitate hydratase (Aconitase) (EC 4.2.1.3) |
| Citrus lemon | V4SNZ3 | Uncharacterized protein |
| Citrus lemon | V4SP86 | Uncharacterized protein |
| Citrus lemon | V4SPM1 | 40S ribosomal protein S12 |
| Citrus lemon | V4SPW4 | 40S ribosomal protein S4 |
| Citrus lemon | V4SQ71 | Uncharacterized protein |
| Citrus lemon | V4SQ89 | Uncharacterized protein |
| Citrus lemon | V4SQ92 | Uncharacterized protein |
| Citrus lemon | V4SQC7 | Peroxidase (EC 1.11.1.7) |
| Citrus lemon | V4SQG3 | Uncharacterized protein |
| Citrus lemon | V4SR15 | Uncharacterized protein |
| Citrus lemon | V4SRN3 | Transmembrane 9 superfamily member |
| Citrus lemon | V4SS09 | Uncharacterized protein |
| Citrus lemon | V4SS11 | Uncharacterized protein |
| Citrus lemon | V4SS50 | Uncharacterized protein |
| Citrus lemon | V4SSB6 | Uncharacterized protein |
| Citrus lemon | V4SSB8 | Proteasome subunit alpha type (EC 3.4.25.1) |
| Citrus lemon | V4SSL7 | Uncharacterized protein |
| Citrus lemon | V4SSQ1 | Uncharacterized protein |
| Citrus lemon | V4SST6 | Uncharacterized protein |
| Citrus lemon | V4SSW9 | Uncharacterized protein |
| Citrus lemon | V4SSX5 | Uncharacterized protein |
| Citrus lemon | V4SU82 | Uncharacterized protein |
| Citrus lemon | V4SUD3 | Uncharacterized protein |
| Citrus lemon | V4SUL7 | Uncharacterized protein |
| Citrus lemon | V4SUP3 | Uncharacterized protein |
| Citrus lemon | V4SUT4 | UDP-glucose 6-dehydrogenase (EC 1.1.1.22) |
| Citrus lemon | V4SUY5 | Uncharacterized protein |
| Citrus lemon | V4SV60 | Serine/threonine-protein phosphatase (EC 3.1.3.16) |
| Citrus lemon | V4SV61 | Uncharacterized protein |
| Citrus lemon | V4SVI5 | Proteasome subunit alpha type (EC 3.4.25.1) |
| Citrus lemon | V4SVI6 | Uncharacterized protein |
| Citrus lemon | V4SW04 | Uncharacterized protein (Fragment) |
| Citrus lemon | V4SWD9 | Uncharacterized protein |
| Citrus lemon | V4SWJ0 | 40S ribosomal protein S3a |
| Citrus lemon | V4SWQ9 | Uncharacterized protein |
| Citrus lemon | V4SWR9 | Uncharacterized protein |
| Citrus lemon | V4SWU9 | Fructose-bisphosphate aldolase (EC 4.1.2.13) |
| Citrus lemon | V4SX11 | Uncharacterized protein |
| Citrus lemon | V4SX99 | Uncharacterized protein |
| Citrus lemon | V4SXC7 | Proteasome subunit alpha type (EC 3.4.25.1) |
| Citrus lemon | V4SXQ5 | Uncharacterized protein |
| Citrus lemon | V4SXW1 | Beta-adaptin-like protein |
| Citrus lemon | V4SXY9 | Uncharacterized protein |
| Citrus lemon | V4SY74 | Uncharacterized protein |
| Citrus lemon | V4SY90 | Uncharacterized protein |
| Citrus lemon | V4SY93 | Uncharacterized protein |
| Citrus lemon | V4SYH9 | Uncharacterized protein |
| Citrus lemon | V4SYK6 | Uncharacterized protein |
| Citrus lemon | V4SZ03 | Uncharacterized protein |
| Citrus lemon | V4SZ73 | Uncharacterized protein |
| Citrus lemon | V4SZI9 | Uncharacterized protein |
| Citrus lemon | V4SZX7 | Uncharacterized protein |
| Citrus lemon | V4T057 | Ribosomal protein L15 |
| Citrus lemon | V4T0V5 | Eukaryotic translation initiation factor 3 subunit A (eIF3a) |
| | | (Eukaryotic translation initiation factor 3 subunit 10) |
| Citrus lemon | V4T0Y1 | Uncharacterized protein |
| Citrus lemon | V4T1Q6 | Uncharacterized protein |
| Citrus lemon | V4T1U7 | Uncharacterized protein |
| Citrus lemon | V4T2D9 | Uncharacterized protein |
| Citrus lemon | V4T2M6 | Tubulin beta chain |
| Citrus lemon | V4T3G2 | Uncharacterized protein |
| Citrus lemon | V4T3P3 | 6-phosphogluconate dehydrogenase, decarboxylating (EC |
| | | 1.1.1.44) |
| Citrus lemon | V4T3V9 | Uncharacterized protein |
| Citrus lemon | V4T3Y6 | Uncharacterized protein |
| Citrus lemon | V4T4H3 | Uncharacterized protein |
| Citrus lemon | V4T4I7 | Uncharacterized protein |
| Citrus lemon | V4T4M7 | Superoxide dismutase [Cu—Zn] (EC 1.15.1.1) |
| Citrus lemon | V4T539 | Uncharacterized protein |
| Citrus lemon | V4T541 | Uncharacterized protein |
| Citrus lemon | V4T576 | Uncharacterized protein |
| Citrus lemon | V4T5E1 | Uncharacterized protein |
| Citrus lemon | V4T5I3 | Uncharacterized protein |
| Citrus lemon | V4T5W7 | Uncharacterized protein |
| Citrus lemon | V4T6T5 | 60S acidic ribosomal protein P0 |
| Citrus lemon | V4T722 | Uncharacterized protein |
| Citrus lemon | V4T785 | Uncharacterized protein |
| Citrus lemon | V4T7E2 | Uncharacterized protein |
| Citrus lemon | V4T7I7 | Uncharacterized protein |
| Citrus lemon | V4T7N0 | Proteasome subunit beta type (EC 3.4.25.1) |
| Citrus lemon | V4T7N4 | Uncharacterized protein |
| Citrus lemon | V4T7T2 | Uncharacterized protein |
| Citrus lemon | V4T7W5 | Uncharacterized protein |
| Citrus lemon | V4T825 | Uncharacterized protein |
| Citrus lemon | V4T846 | Uncharacterized protein |
| Citrus lemon | V4T8E9 | S-acyltransferase (EC 2.3.1.225) (Palmitoyltransferase) |
| Citrus lemon | V4T8G2 | Uncharacterized protein |
| Citrus lemon | V4T8G9 | Chorismate synthase (EC 4.2.3.5) |
| Citrus lemon | V4T8Y6 | Uncharacterized protein |
| Citrus lemon | V4T8Y8 | Uncharacterized protein |
| Citrus lemon | V4T939 | Carboxypeptidase (EC 3.4.16.—) |
| Citrus lemon | V4T957 | Uncharacterized protein |
| Citrus lemon | V4T998 | Uncharacterized protein |
| Citrus lemon | V4T9B9 | Uncharacterized protein |
| Citrus lemon | V4T9Y7 | Uncharacterized protein |
| Citrus lemon | V4TA70 | Uncharacterized protein |
| Citrus lemon | V4TAF6 | Uncharacterized protein |
| Citrus lemon | V4TB09 | Uncharacterized protein |
| Citrus lemon | V4TB32 | Uncharacterized protein |
| Citrus lemon | V4TB89 | Uncharacterized protein |
| Citrus lemon | V4TBN7 | Phosphoinositide phospholipase C (EC 3.1.4.11) |
| Citrus lemon | V4TBQ3 | Uncharacterized protein |
| Citrus lemon | V4TBS4 | Uncharacterized protein |
| Citrus lemon | V4TBU3 | Uncharacterized protein |
| Citrus lemon | V4TCA6 | Uncharacterized protein |
| Citrus lemon | V4TCL3 | Uncharacterized protein |
| Citrus lemon | V4TCS5 | Pectate lyase (EC 4.2.2.2) |
| Citrus lemon | V4TD99 | Uncharacterized protein |
| Citrus lemon | V4TDB5 | Uncharacterized protein |
| Citrus lemon | V4TDI2 | Uncharacterized protein |
| Citrus lemon | V4TDY3 | Serine/threonine-protein kinase (EC 2.7.11.1) |
| Citrus lemon | V4TE72 | Uncharacterized protein |
| Citrus lemon | V4TE95 | Uncharacterized protein |
| Citrus lemon | V4TEC0 | Uncharacterized protein |
| Citrus lemon | V4TED8 | Uncharacterized protein |
| Citrus lemon | V4TES4 | Uncharacterized protein |
| Citrus lemon | V4TEY9 | Uncharacterized protein |
| Citrus lemon | V4TF24 | Proteasome subunit alpha type (EC 3.4.25.1) |
| Citrus lemon | V4TF52 | Uricase (EC 1.7.3.3) (Urate oxidase) |
| Citrus lemon | V4TFV8 | Catalase (EC 1.11.1.6) |
| Citrus lemon | V4TGU1 | Uncharacterized protein |
| Citrus lemon | V4TH28 | Uncharacterized protein |
| Citrus lemon | V4TH78 | Reticulon-like protein |
| Citrus lemon | V4THM9 | Uncharacterized protein |
| Citrus lemon | V4TIU2 | Ribulose-phosphate 3-epimerase (EC 5.1.3.1) |
| Citrus lemon | V4TIW6 | Uncharacterized protein |
| Citrus lemon | V4TIY6 | Uncharacterized protein |
| Citrus lemon | V4TIZ5 | Uncharacterized protein |
| Citrus lemon | V4TJ75 | Uncharacterized protein |
| Citrus lemon | V4TJC3 | Uncharacterized protein |
| Citrus lemon | V4TJQ9 | Uncharacterized protein |
| Citrus lemon | V4TK29 | NEDD8-activating enzyme E1 regulatory subunit |
| Citrus lemon | V4TL04 | Uncharacterized protein |
| Citrus lemon | V4TLL5 | Uncharacterized protein |
| Citrus lemon | V4TLP6 | Uncharacterized protein |
| Citrus lemon | V4TM00 | Uncharacterized protein |
| Citrus lemon | V4TM19 | Uncharacterized protein |
| Citrus lemon | V4TMB7 | Uncharacterized protein (Fragment) |
| Citrus lemon | V4TMD1 | Uncharacterized protein |
| Citrus lemon | V4TMD6 | Uncharacterized protein |
| Citrus lemon | V4TMV4 | Uncharacterized protein |
| Citrus lemon | V4TN30 | Uncharacterized protein |
| Citrus lemon | V4TN38 | Uncharacterized protein |
| Citrus lemon | V4TNY8 | Uncharacterized protein |
| Citrus lemon | V4TP87 | Carbonic anhydrase (EC 4.2.1.1) (Carbonate dehydratase) |
| Citrus lemon | V4TPM1 | Homoserine dehydrogenase (HDH) (EC 1.1.1.3) |
| Citrus lemon | V4TQB6 | Uncharacterized protein |
| Citrus lemon | V4TQM7 | Uncharacterized protein |
| Citrus lemon | V4TQR2 | Uncharacterized protein |
| Citrus lemon | V4TQV9 | Uncharacterized protein |
| Citrus lemon | V4TS21 | Proteasome subunit beta type (EC 3.4.25.1) |
| Citrus lemon | V4TS28 | Annexin |
| Citrus lemon | V4TSD8 | Uncharacterized protein (Fragment) |
| Citrus lemon | V4TSF8 | Uncharacterized protein |
| Citrus lemon | V4TSI9 | Uncharacterized protein |
| Citrus lemon | V4TT89 | Uncharacterized protein |
| Citrus lemon | V4TTA0 | Uncharacterized protein |
| Citrus lemon | V4TTR8 | Uncharacterized protein |
| Citrus lemon | V4TTV4 | Uncharacterized protein |
| Citrus lemon | V4TTZ7 | Uncharacterized protein |
| Citrus lemon | V4TU54 | Uncharacterized protein |
| Citrus lemon | V4TVB6 | Uncharacterized protein |
| Citrus lemon | V4TVG1 | Eukaryotic translation initiation factor 5A (eIF-5A) |
| Citrus lemon | V4TVJ4 | Profilin |
| Citrus lemon | V4TVM6 | Uncharacterized protein |
| Citrus lemon | V4TVM9 | Uncharacterized protein |
| Citrus lemon | V4TVP7 | Uncharacterized protein |
| Citrus lemon | V4TVT8 | Uncharacterized protein |
| Citrus lemon | V4TW14 | Uncharacterized protein |
| Citrus lemon | V4TWG9 | T-complex protein 1 subunit delta |
| Citrus lemon | V4TWU1 | Probable bifunctional methylthioribulose-1-phosphate |
| | | dehydratase/enolase-phosphatase E1 [Includes: Enolase-phosphatase |
| | | E1 (EC 3.1.3.77) (2,3-diketo-5-methylthio-1-phosphopentane |
| | | phosphatase); Methylthioribulose-1-phosphate dehydratase |
| | | (MTRu-1-P dehydratase) (EC 4.2.1.109)] |
| Citrus lemon | V4TWX8 | Uncharacterized protein |
| Citrus lemon | V4TXH0 | Glutamate decarboxylase (EC 4.1.1.15) |
| Citrus lemon | V4TXK9 | Uncharacterized protein |
| Citrus lemon | V4TXU9 | Thiamine thiazole synthase, chloroplastic (Thiazole |
| | | biosynthetic enzyme) |
| Citrus lemon | V4TY40 | Uncharacterized protein |
| Citrus lemon | V4TYJ6 | Uncharacterized protein |
| Citrus lemon | V4TYP5 | 60S ribosomal protein L13 |
| Citrus lemon | V4TYP6 | Uncharacterized protein |
| Citrus lemon | V4TYR6 | Uncharacterized protein |
| Citrus lemon | V4TYZ8 | Tubulin alpha chain |
| Citrus lemon | V4TZ91 | Guanosine nucleotide diphosphate dissociation inhibitor |
| Citrus lemon | V4TZA8 | Uncharacterized protein |
| Citrus lemon | V4TZJ1 | Uncharacterized protein |
| Citrus lemon | V4TZK5 | Uncharacterized protein |
| Citrus lemon | V4TZP2 | Uncharacterized protein |
| Citrus lemon | V4TZT8 | Uncharacterized protein |
| Citrus lemon | V4TZU3 | Mitogen-activated protein kinase (EC 2.7.11.24) |
| Citrus lemon | V4TZU5 | Dihydrolipoyl dehydrogenase (EC 1.8.1.4) |
| Citrus lemon | V4TZZ0 | Uncharacterized protein |
| Citrus lemon | V4U003 | Eukaryotic translation initiation factor 3 subunit K (eIF3k) |
| | | (eIF-3 p25) |
| Citrus lemon | V4U068 | Uncharacterized protein |
| Citrus lemon | V4U088 | Uncharacterized protein |
| Citrus lemon | V4U0J7 | Uncharacterized protein |
| Citrus lemon | V4U133 | Uncharacterized protein |
| Citrus lemon | V4U1A8 | Uncharacterized protein |
| Citrus lemon | V4U1K1 | Xylose isomerase (EC 5.3.1.5) |
| Citrus lemon | V4U1M1 | Uncharacterized protein |
| Citrus lemon | V4U1V0 | Uncharacterized protein |
| Citrus lemon | V4U1X7 | Uncharacterized protein |
| Citrus lemon | V4U1X9 | Proteasome subunit beta type (EC 3.4.25.1) |
| Citrus lemon | V4U251 | Uncharacterized protein |
| Citrus lemon | V4U283 | Uncharacterized protein |
| Citrus lemon | V4U2E4 | Uncharacterized protein |
| Citrus lemon | V4U2F7 | Uncharacterized protein |
| Citrus lemon | V4U2H8 | Uncharacterized protein |
| Citrus lemon | V4U2L0 | Malate dehydrogenase (EC 1.1.1.37) |
| Citrus lemon | V4U2L2 | Uncharacterized protein |
| Citrus lemon | V4U2W4 | V-type proton ATPase subunit C |
| Citrus lemon | V4U3L2 | Uncharacterized protein |
| Citrus lemon | V4U3W8 | Uncharacterized protein |
| Citrus lemon | V4U412 | Uncharacterized protein |
| Citrus lemon | V4U4K2 | Uncharacterized protein |
| Citrus lemon | V4U4M4 | Uncharacterized protein |
| Citrus lemon | V4U4N5 | Eukaryotic translation initiation factor 6 (eIF-6) |
| Citrus lemon | V4U4S9 | Uncharacterized protein |
| Citrus lemon | V4U4X3 | Serine hydroxymethyltransferase (EC 2.1.2.1) |
| Citrus lemon | V4U4Z9 | Uncharacterized protein |
| Citrus lemon | V4U500 | Uncharacterized protein |
| Citrus lemon | V4U5B0 | Eukaryotic translation initiation factor 3 subunit E (eIF3e) |
| | | (Eukaryotic translation initiation factor 3 subunit 6) |
| Citrus lemon | V4U5B8 | Glutathione peroxidase |
| Citrus lemon | V4U5R5 | Citrate synthase |
| Citrus lemon | V4U5Y8 | Uncharacterized protein |
| Citrus lemon | V4U6I5 | ATP synthase subunit beta (EC 3.6.3.14) |
| Citrus lemon | V4U6Q8 | Uncharacterized protein |
| Citrus lemon | V4U706 | Uncharacterized protein |
| Citrus lemon | V4U717 | Uncharacterized protein |
| Citrus lemon | V4U726 | Uncharacterized protein |
| Citrus lemon | V4U729 | Uncharacterized protein |
| Citrus lemon | V4U734 | Serine/threonine-protein phosphatase (EC 3.1.3.16) |
| Citrus lemon | V4U7G7 | Uncharacterized protein |
| Citrus lemon | V4U7H5 | Uncharacterized protein |
| Citrus lemon | V4U7R1 | Potassium transporter |
| Citrus lemon | V4U7R7 | Mitogen-activated protein kinase (EC 2.7.11.24) |
| Citrus lemon | V4U833 | Malic enzyme |
| Citrus lemon | V4U840 | Uncharacterized protein |
| Citrus lemon | V4U8C3 | Uncharacterized protein |
| Citrus lemon | V4U8J1 | 3-phosphoshikimate 1-carboxyvinyltransferase (EC 2.5.1.19) |
| Citrus lemon | V4U8J8 | T-complex protein 1 subunit gamma |
| Citrus lemon | V4U995 | Uncharacterized protein |
| Citrus lemon | V4U999 | Uncharacterized protein |
| Citrus lemon | V4U9C7 | Eukaryotic translation initiation factor 3 subunit D (eIF3d) |
| | | (Eukaryotic translation initiation factor 3 subunit 7) (eIF-3-zeta) |
| Citrus lemon | V4U9G8 | Proline iminopeptidase (EC 3.4.11.5) |
| Citrus lemon | V4U9L1 | Uncharacterized protein |
| Citrus lemon | V4UA63 | Phytochrome |
| Citrus lemon | V4UAC8 | Uncharacterized protein |
| Citrus lemon | V4UAR4 | Uncharacterized protein |
| Citrus lemon | V4UB30 | Uncharacterized protein |
| Citrus lemon | V4UBK8 | V-type proton ATPase subunit a |
| Citrus lemon | V4UBL3 | Coatomer subunit alpha |
| Citrus lemon | V4UBL5 | Uncharacterized protein (Fragment) |
| Citrus lemon | V4UBM0 | Uncharacterized protein |
| Citrus lemon | V4UBZ8 | Aspartate aminotransferase (EC 2.6.1.1) |
| Citrus lemon | V4UC72 | Uncharacterized protein |
| Citrus lemon | V4UC97 | Beta-glucosidase (EC 3.2.1.21) |
| Citrus lemon | V4UCE2 | Uncharacterized protein |
| Citrus lemon | V4UCT9 | Acetyl-coenzyme A synthetase (EC 6.2.1.1) |
| Citrus lemon | V4UCZ1 | Uncharacterized protein |
| Citrus lemon | V4UE34 | Uncharacterized protein |
| Citrus lemon | V4UE78 | Uncharacterized protein |
| Citrus lemon | V4UER3 | Uncharacterized protein |
| Citrus lemon | V4UET6 | Uncharacterized protein |
| Citrus lemon | V4UEZ6 | Uncharacterized protein |
| Citrus lemon | V4UFD0 | Uncharacterized protein |
| Citrus lemon | V4UFG8 | Uncharacterized protein |
| Citrus lemon | V4UFK1 | Uncharacterized protein |
| Citrus lemon | V4UG68 | Eukaryotic translation initiation factor 3 subunit I (eIF3i) |
| Citrus lemon | V4UGB0 | Uncharacterized protein |
| Citrus lemon | V4UGH4 | Uncharacterized protein |
| Citrus lemon | V4UGL9 | Uncharacterized protein |
| Citrus lemon | V4UGQ0 | Ubiquitinyl hydrolase 1 (EC 3.4.19.12) |
| Citrus lemon | V4UH00 | Uncharacterized protein |
| Citrus lemon | V4UH48 | Uncharacterized protein |
| Citrus lemon | V4UH77 | Proteasome subunit alpha type (EC 3.4.25.1) |
| Citrus lemon | V4UHD8 | Uncharacterized protein |
| Citrus lemon | V4UHD9 | Uncharacterized protein |
| Citrus lemon | V4UHF1 | Uncharacterized protein |
| Citrus lemon | V4UHZ5 | Uncharacterized protein |
| Citrus lemon | V4UI07 | 40S ribosomal protein S8 |
| Citrus lemon | V4UI34 | Eukaryotic translation initiation factor 3 subunit L (eIF3I) |
| Citrus lemon | V4UIF1 | Uncharacterized protein |
| Citrus lemon | V4UIN5 | Uncharacterized protein |
| Citrus lemon | V4UIX8 | Uncharacterized protein |
| Citrus lemon | V4UJ12 | Uncharacterized protein |
| Citrus lemon | V4UJ42 | Uncharacterized protein |
| Citrus lemon | V4UJ63 | Uncharacterized protein |
| Citrus lemon | V4UJB7 | Uncharacterized protein (Fragment) |
| Citrus lemon | V4UJC4 | Uncharacterized protein |
| Citrus lemon | V4UJX0 | Phosphotransferase (EC 2.7.1.—) |
| Citrus lemon | V4UJY5 | Uncharacterized protein |
| Citrus lemon | V4UK18 | Uncharacterized protein |
| Citrus lemon | V4UK52 | Uncharacterized protein |
| Citrus lemon | V4UKM9 | Uncharacterized protein |
| Citrus lemon | V4UKS4 | Uncharacterized protein |
| Citrus lemon | V4UKV6 | 40S ribosomal protein SA |
| Citrus lemon | V4UL30 | Pyrophosphate-fructose 6-phosphate 1-phosphotransferase |
| | | subunit beta (PFP) (EC 2.7.1.90) (6-phosphofructokinase, |
| | | pyrophosphate dependent) (PPi-PFK) |
| | | (Pyrophosphate-dependent 6-phosphofructose-1-kinase) |
| Citrus lemon | V4UL39 | Uncharacterized protein |
| Citrus lemon | V4ULH9 | Uncharacterized protein |
| Citrus lemon | V4ULL2 | Uncharacterized protein |
| Citrus lemon | V4ULS0 | Uncharacterized protein |
| Citrus lemon | V4UMU7 | Uncharacterized protein |
| Citrus lemon | V4UN36 | Uncharacterized protein |
| Citrus lemon | V4UNT5 | Uncharacterized protein |
| Citrus lemon | V4UNW1 | Uncharacterized protein |
| Citrus lemon | V4UP89 | Uncharacterized protein |
| Citrus lemon | V4UPE4 | Uncharacterized protein |
| Citrus lemon | V4UPF7 | Uncharacterized protein |
| Citrus lemon | V4UPK0 | Uncharacterized protein |
| Citrus lemon | V4UPX5 | Uncharacterized protein |
| Citrus lemon | V4UQ58 | Uncharacterized protein |
| Citrus lemon | V4UQF6 | Uncharacterized protein |
| Citrus lemon | V4UR21 | Uncharacterized protein |
| Citrus lemon | V4UR80 | Uncharacterized protein |
| Citrus lemon | V4URK3 | Uncharacterized protein |
| Citrus lemon | V4URT3 | Uncharacterized protein |
| Citrus lemon | V4US96 | Uncharacterized protein |
| Citrus lemon | V4USQ8 | Uncharacterized protein |
| Citrus lemon | V4UT16 | Uncharacterized protein |
| Citrus lemon | V4UTC6 | Uncharacterized protein |
| Citrus lemon | V4UTC8 | Uncharacterized protein |
| Citrus lemon | V4UTP6 | Uncharacterized protein |
| Citrus lemon | V4UTY0 | Proteasome subunit alpha type (EC 3.4.25.1) |
| Citrus lemon | V4UU96 | Uncharacterized protein |
| Citrus lemon | V4UUB6 | Uncharacterized protein |
| Citrus lemon | V4UUJ9 | Aminopeptidase (EC 3.4.11.—) |
| Citrus lemon | V4UUK6 | Uncharacterized protein |
| Citrus lemon | V4UV09 | Uncharacterized protein |
| Citrus lemon | V4UV83 | Lysine--tRNA ligase (EC 6.1.1.6) (Lysyl-tRNA synthetase) |
| Citrus lemon | V4UVJ5 | Diacylglycerol kinase (DAG kinase) (EC 2.7.1.107) |
| Citrus lemon | V4UW03 | Uncharacterized protein |
| Citrus lemon | V4UW04 | Uncharacterized protein |
| Citrus lemon | V4UWR1 | Uncharacterized protein |
| Citrus lemon | V4UWV8 | Uncharacterized protein |
| Citrus lemon | V4UX36 | Uncharacterized protein |
| Citrus lemon | V4V003 | Uncharacterized protein |
| Citrus lemon | V4V0J0 | 40S ribosomal protein S26 |
| Citrus lemon | V4V1P8 | Uncharacterized protein |
| Citrus lemon | V4V4V0 | Uncharacterized protein |
| Citrus lemon | V4V5T8 | Ubiquitin-fold modifier 1 |
| Citrus lemon | V4V600 | Uncharacterized protein |
| Citrus lemon | V4V622 | Aldehyde dehydrogenase |
| Citrus lemon | V4V6W1 | Uncharacterized protein |
| Citrus lemon | V4V6Z2 | Uncharacterized protein |
| Citrus lemon | V4V738 | Uncharacterized protein |
| Citrus lemon | V4V8H5 | Vacuolar protein sorting-associated protein 35 |
| Citrus lemon | V4V9P6 | Eukaryotic translation initiation factor 3 subunit F (eIF3f) |
| | | (eIF-3-epsilon) |
| Citrus lemon | V4V9V7 | Clathrin heavy chain |
| Citrus lemon | V4V9X3 | Uncharacterized protein |
| Citrus lemon | V4VAA3 | Superoxide dismutase (EC 1.15.1.1) |
| Citrus lemon | V4VAF3 | Uncharacterized protein |
| Citrus lemon | V4VBQ0 | Uncharacterized protein (Fragment) |
| Citrus lemon | V4VCL1 | Proteasome subunit beta type (EC 3.4.25.1) |
| Citrus lemon | V4VCZ9 | Uncharacterized protein |
| Citrus lemon | V4VDK1 | Peptidylprolyl isomerase (EC 5.2.1.8) |
| Citrus lemon | V4VEA1 | Uncharacterized protein |
| Citrus lemon | V4VEB3 | Alanine--tRNA ligase (EC 6.1.1.7) (Alanyl-tRNA synthetase) |
| | | (AlaRS) |
| Citrus lemon | V4VEE3 | Glutamine synthetase (EC 6.3.1.2) |
| Citrus lemon | V4VFM3 | Uncharacterized protein |
| Citrus lemon | V4VFN5 | Proteasome subunit beta type (EC 3.4.25.1) |
| Citrus lemon | V4VGD6 | Uncharacterized protein |
| Citrus lemon | V4VGL9 | Uncharacterized protein |
| Citrus lemon | V4VHI6 | Uncharacterized protein |
| Citrus lemon | V4VIP4 | Uncharacterized protein |
| Citrus lemon | V4VJT4 | Uncharacterized protein |
| Citrus lemon | V4VK14 | Uncharacterized protein |
| Citrus lemon | V4VKI5 | Protein-L-isoaspartate O-methyltransferase (EC 2.1.1.77) |
| Citrus lemon | V4VKP2 | Glyceraldehyde-3-phosphate dehydrogenase (EC 1.2.1.—) |
| Citrus lemon | V4VL73 | Acyl-coenzyme A oxidase |
| Citrus lemon | V4VLL7 | Uncharacterized protein |
| Citrus lemon | V4VN43 | Uncharacterized protein (Fragment) |
| Citrus lemon | V4VQH3 | Methylenetetrahydrofolate reductase (EC 1.5.1.20) |
| Citrus lemon | V4VTC9 | Uncharacterized protein (Fragment) |
| Citrus lemon | V4VTT4 | Uncharacterized protein |
| Citrus lemon | V4VTY7 | Uncharacterized protein |
| Citrus lemon | V4VU14 | Uncharacterized protein |
| Citrus lemon | V4VU32 | Uncharacterized protein |
| Citrus lemon | V4VUK6 | S-(hydroxymethyl)glutathione dehydrogenase (EC 1.1.1.284) |
| Citrus lemon | V4VVR8 | Uncharacterized protein |
| Citrus lemon | V4VXE2 | Uncharacterized protein |
| Citrus lemon | V4VY37 | Phosphomannomutase (EC 5.4.2.8) |
| Citrus lemon | V4VYC0 | Uncharacterized protein |
| Citrus lemon | V4VYV1 | Uncharacterized protein |
| Citrus lemon | V4VZ80 | Uncharacterized protein |
| Citrus lemon | V4VZJ7 | Uncharacterized protein |
| Citrus lemon | V4W2P2 | Alpha-mannosidase (EC 3.2.1.—) |
| Citrus lemon | V4W2Z9 | Chloride channel protein |
| Citrus lemon | V4W378 | Uncharacterized protein |
| Citrus lemon | V4W4G3 | Uncharacterized protein |
| Citrus lemon | V4W5F1 | Uncharacterized protein |
| Citrus lemon | V4W5N8 | Uncharacterized protein |
| Citrus lemon | V4W5U2 | Uncharacterized protein |
| Citrus lemon | V4W6G1 | Uncharacterized protein |
| Citrus lemon | V4W730 | Uncharacterized protein |
| Citrus lemon | V4W7J4 | Obg-like ATPase 1 |
| Citrus lemon | V4W7L5 | Uncharacterized protein |
| Citrus lemon | V4W8C5 | Uncharacterized protein |
| Citrus lemon | V4W8C9 | Uncharacterized protein |
| Citrus lemon | V4W8D3 | Uncharacterized protein |
| Citrus lemon | V4W951 | Uncharacterized protein |
| Citrus lemon | V4W9F6 | 60S ribosomal protein L18a |
| Citrus lemon | V4W9G2 | Uncharacterized protein (Fragment) |
| Citrus lemon | V4W9L3 | Uncharacterized protein |
| Citrus lemon | V4W9Y8 | Uncharacterized protein |
| Citrus lemon | V4WAP9 | Coatomer subunit beta (Beta-coat protein) |
| Citrus lemon | V4WBK6 | Cytochrome b-c1 complex subunit 7 |
| Citrus lemon | V4WC15 | Malic enzyme |
| Citrus lemon | V4WC19 | Uncharacterized protein |
| Citrus lemon | V4WC74 | Uncharacterized protein |
| Citrus lemon | V4WC86 | Serine/threonine-protein phosphatase 2A 55 kDa regulatory |
| | | subunit B |
| Citrus lemon | V4WCS4 | GTP-binding nuclear protein |
| Citrus lemon | V4WD80 | Aspartate aminotransferase (EC 2.6.1.1) |
| Citrus lemon | V4WDK0 | Uncharacterized protein |
| Citrus lemon | V4WDK3 | ATP-dependent 6-phosphofructokinase (ATP-PFK) |
| | | (Phosphofructokinase) (EC 2.7.1.11) (Phosphohexokinase) |
| Citrus lemon | V4WE00 | Uncharacterized protein |
| Citrus lemon | V4WEE3 | Uncharacterized protein |
| Citrus lemon | V4WEN2 | Uncharacterized protein |
| Citrus lemon | V4WG97 | Autophagy-related protein |
| Citrus lemon | V4WGV2 | Uncharacterized protein |
| Citrus lemon | V4WGW5 | Uridine kinase (EC 2.7.1.48) |
| Citrus lemon | V4WHD4 | Uncharacterized protein |
| Citrus lemon | V4WHF8 | Sucrose synthase (EC 2.4.1.13) |
| Citrus lemon | V4WHK2 | Pectinesterase (EC 3.1.1.11) |
| Citrus lemon | V4WHQ4 | Uncharacterized protein |
| Citrus lemon | V4WHT6 | Uncharacterized protein |
| Citrus lemon | V4WJ93 | Uncharacterized protein |
| Citrus lemon | V4WJA9 | Uncharacterized protein |
| Citrus lemon | V4WJB1 | Uncharacterized protein |
| Citrus lemon | V9HXG3 | Protein disulfide-isomerase (EC 5.3.4.1) |
| Citrus lemon | W8Q8K1 | Putative inorganic pyrophosphatase |
| Citrus lemon | W8QJL0 | Putative isopentenyl pyrophosphate isomerase |

| Grape | Accession Number | Identified Proteins |
|---|---|---|
| Grape | A5C5K3 (+2) | Adenosylhomocysteinase |
| Grape | Q9M6B5 | Alcohol dehydrogenase 6 |
| Grape | A3FA65 (+1) | Aquaporin PIP1; 3 |
| Grape | Q0MX13 (+2) | Aquaporin PIP2; 2 |
| Grape | A3FA69 (+4) | Aquaporin PIP2; 4 |
| Grape | A5AFS1 (+2) | Elongation factor 1-alpha |
| Grape | UPI0001985702 | elongation factor 2 |
| Grape | D7T227 | Enolase |
| Grape | D7TJ12 | Enolase |
| Grape | A5B118 (+1) | Fructose-bisphosphate aldolase |
| Grape | E0CQ39 | Glucose-6-phosphate isomerase |
| Grape | D7TW04 | Glutathione peroxidase |
| Grape | A1YW90 (+3) | Glutathione S-transferase |
| Grape | A5BEW0 | Histone H4 |
| Grape | UPI00015C9A6A | HSC70-1 (heat shock cognate 70 kDa protein 1); ATP |
| | | binding isoform 1 |
| Grape | D7FBC0 (+1) | Malate dehydrogenase |
| Grape | D7TBH4 | Malic enzyme |
| Grape | A5ATB7 (+1) | Methylenetetrahydrofolate reductase |
| Grape | A5JPK7 (+1) | Monodehydroascorbate reductase |
| Grape | A5AKD8 | Peptidyl-prolyl cis-trans isomerase |
| Grape | A5BQN6 | Peptidyl-prolyl cis-trans isomerase |
| Grape | A5CAF6 | Phosphoglycerate kinase |
| Grape | Q09VU3 (+1) | Phospholipase D |
| Grape | D7SK33 | Phosphorylase |
| Grape | A5AQ89 | Profilin |
| Grape | C5DB50 (+2) | Putative 2,3-bisphosphoglycerate-independent |
| | | phosphoglycerate mutase |
| Grape | D7TIZ5 | Pyruvate kinase |
| Grape | A5BV65 | Triosephosphate isomerase |
| Grapefruit | G8Z362 (+1) | (E)-beta-farnesene synthase |
| Grapefruit | Q5CD81 | (E)-beta-ocimene synthase |
| Grapefruit | D0UZK1 (+2) | 1,2 rhamnosyltransferase |
| Grapefruit | A7ISD3 | 1,6-rhamnosyltransferase |
| Grapefruit | Q80H98 | 280 kDa protein |
| Grapefruit | Q15GA4 (+2) | 286 kDa polyprotein |
| Grapefruit | D7NHW9 | 2-phospho-D-glycerate hydrolase |
| Grapefruit | D0EAL9 | 349 kDa polyprotein |
| Grapefruit | Q9DTG5 | 349-kDa polyprotein |
| Grapefruit | O22297 | Acidic cellulase |
| Grapefruit | Q8H986 | Acidic class I chitinase |
| Grapefruit | D3GQL0 | Aconitate hydratase 1 |
| Grapefruit | K7N8A0 | Actin |
| Grapefruit | A8W8Y0 | Alcohol acyl transferase |
| Grapefruit | Q84V85 | Allene oxide synthase |
| Grapefruit | F8WL79 | Aminopeptidase |
| Grapefruit | Q09MG5 | Apocytochrome f |
| Grapefruit | J7EIR8 | Ascorbate peroxidase |
| Grapefruit | B9VRH6 | Ascorbate peroxidase |
| Grapefruit | G9I820 | Auxin-response factor |
| Grapefruit | J7ICW8 | Beta-amylase |
| Grapefruit | Q8L5Q9 | Beta-galactosidase |
| Grapefruit | A7BG60 | Beta-pinene synthase |
| Grapefruit | C0KLD1 | Beta-tubulin |
| Grapefruit | Q91QZ1 | Capsid protein |
| Grapefruit | Q3SAK9 | Capsid protein |
| Grapefruit | D2U833 | Cation chloride cotransporter |
| Grapefruit | C3VPJ0 (+3) | Chaicone synthase |
| Grapefruit | D5LM39 | Chloride channel protein |
| Grapefruit | Q9M4U0 | Cinnamate 4-hydroxylase CYP73 |
| Grapefruit | Q39627 | Citrin |
| Grapefruit | G2XKD3 | Coat protein |
| Grapefruit | Q3L2I6 | Coat protein |
| Grapefruit | D5FV16 | CRT/DRE binding factor |
| Grapefruit | Q8H6S5 | CTV.2 |
| Grapefruit | Q8H6Q8 | CTV.20 |
| Grapefruit | Q8H6Q7 | CTV.22 |
| Grapefruit | Q1I1D7 | Cytochrome P450 |
| Grapefruit | Q7Y045 | Dehydrin |
| Grapefruit | F8WLD2 | DNA excision repair protein |
| Grapefruit | Q09MI8 | DNA-directed RNA polymerase subunit beta″ |
| Grapefruit | D2WKC9 | Ethylene response 1 |
| Grapefruit | D2WKD2 | Ethylene response sensor 1 |
| Grapefruit | D7PVG7 | Ethylene-insensitive 3-like 1 protein |
| Grapefruit | G3CHK8 | Eukaryotic translation initiation factor 3 subunit E |
| Grapefruit | A9NJG4 (+3) | Fatty acid hydroperoxide lyase |
| Grapefruit | B8Y9B5 | F-box family protein |
| Grapefruit | Q000W4 | Fe(III)-chelate reductase |
| Grapefruit | Q6Q3H4 | Fructokinase |
| Grapefruit | F8WL95 | Gag-pol polyprotein |
| Grapefruit | Q8L5K4 | Gamma-terpinene synthase, chloroplastic |
| Grapefruit | Q9SP43 | Glucose-1-phosphate adenylyltransferase |
| Grapefruit | Q3HM93 | Glutathione S-transferase |
| Grapefruit | D0VEW6 | GRAS family transcription factor |
| Grapefruit | F8WL87 | Heat shock protein |
| Grapefruit | H9NHK0 | Hsp90 |
| Grapefruit | Q8H6R4 | Jp18 |
| Grapefruit | G3CHK6 | Leucine-rich repeat family protein |
| Grapefruit | B2YGX9 (+1) | Limonoid UDP-glucosyltransferase |
| Grapefruit | Q05KK0 | MADS-box protein |
| Grapefruit | F8WLB4 | Mechanosensitive ion channel domain-containing protein |
| Grapefruit | Q5CD82 | Monoterpene synthase |
| Grapefruit | F8WLC4 | MYB transcription factor |
| Grapefruit | A5YWA9 | NAC domain protein |
| Grapefruit | Q09MC9 | NAD(P)H-quinone oxidoreductase subunit 5, chloroplastic |
| Grapefruit | Q8H6R9 | NBS-LRR type disease resistance protein |
| Grapefruit | Q8H6S0 | NBS-LRR type disease resistance protein |
| Grapefruit | Q8H6R6 | NBS-LRR type disease resistance protein |
| Grapefruit | J9WR93 | p1a |
| Grapefruit | Q1X8V8 | P23 |
| Grapefruit | E7DSS0 (+4) | P23 |
| Grapefruit | G0Z9I6 | p27 |
| Grapefruit | I3XHN0 | p33 |
| Grapefruit | B8YDL3 | p33 protein |
| Grapefruit | B9VB22 | p33 protein |
| Grapefruit | P87587 | P346 |
| Grapefruit | B9VB56 | p349 protein |
| Grapefruit | I3RWW7 | p349 protein |
| Grapefruit | B9VB20 | p349 protein |
| Grapefruit | Q9WID7 | p349 protein |
| Grapefruit | Q2XP16 | P353 |
| Grapefruit | O04886 (+1) | Pectinesterase 1 |
| Grapefruit | F8WL74 | Peptidyl-prolyl cis-trans isomerase |
| Grapefruit | Q0ZA67 | Peroxidase |
| Grapefruit | F1CT41 | Phosphoenolpyruvate carboxylase |
| Grapefruit | B1PBV7 (+2) | Phytoene synthase |
| Grapefruit | Q9ZWQ8 | Plastid-lipid-associated protein, chloroplastic |
| Grapefruit | Q94FM1 | Pol polyprotein |
| Grapefruit | Q94FM0 | Pol polyprotein |
| Grapefruit | G9I825 | Poly C-binding protein |
| Grapefruit | O64460 (+7) | Polygalacturonase inhibitor |
| Grapefruit | I3XHM8 | Polyprotein |
| Grapefruit | C0STR9 | Polyprotein |
| Grapefruit | H6U1F0 | Polyprotein |
| Grapefruit | B8QHP8 | Polyprotein |
| Grapefruit | I3V6C0 | Polyprotein |
| Grapefruit | C0STS0 | Polyprotein |
| Grapefruit | K0FGH5 | Polyprotein |
| Grapefruit | Q3HWZ1 | Polyprotein |
| Grapefruit | F8WLA5 | PPR containing protein |
| Grapefruit | Q06652 (+1) | Probable phospholipid hydroperoxide glutathione |
| | | peroxidase |
| Grapefruit | P84177 | Profilin |
| Grapefruit | Q09MB4 | Protein ycf2 |
| Grapefruit | A8C183 | PSI reaction center subunit II |
| Grapefruit | A5JVP6 | Putative 2b protein |
| Grapefruit | D0EFM2 | Putative eukaryotic translation initiation factor 1 |
| Grapefruit | Q18L98 | Putative gag-pol polyprotein |
| Grapefruit | B5AMI9 | Putative movement protein |
| Grapefruit | A1ECK5 | Putative multiple stress-responsive zinc-finger protein |
| Grapefruit | B5AMJ0 | Putative replicase polyprotein |
| Grapefruit | I7CYN5 | Putative RNA-dependent RNA polymerase |
| Grapefruit | Q8RVR2 | Putative terpene synthase |
| Grapefruit | B5TE89 | Putative uncharacterized protein |
| Grapefruit | Q8JVF3 | Putative uncharacterized protein |
| Grapefruit | F8WLB0 | Putative uncharacterized protein ORF43 |
| Grapefruit | A5JVP4 | Putative viral replicase |
| Grapefruit | M1JAW3 | Replicase |
| Grapefruit | H6VXK8 | Replicase polyprotein |
| Grapefruit | J9UF50 (+1) | Replicase protein 1a |
| Grapefruit | J9RV45 | Replicase protein 2a |
| Grapefruit | Q5EGG5 | Replicase-associated polyprotein |
| Grapefruit | G9I823 | RNA recognition motif protein 1 |
| Grapefruit | J7EPC0 | RNA-dependent RNA polymerase |
| Grapefruit | Q6DN67 | RNA-directed RNA polymerase L |
| Grapefruit | A9CQM4 | SEPALLATA1 homolog |
| Grapefruit | Q9SLS2 | Sucrose synthase |
| Grapefruit | Q9SLV8 (+1) | Sucrose synthase |
| Grapefruit | Q38JC1 | Temperature-induced lipocalin |
| Grapefruit | D0ELH6 | Tetratricopeptide domain-containing thioredoxin |
| Grapefruit | D2KU75 | Thaumatin-like protein |
| Grapefruit | C3VIC2 | Translation elongation factor |
| Grapefruit | D5LY07 | Ubiquitin/ribosomal fusion protein |
| Grapefruit | C6KI43 | UDP-glucosyltransferase family 1 protein |
| Grapefruit | A0FKR1 | Vacuolar citrate/H+ symporter |
| Grapefruit | Q944C8 | Vacuolar invertase |
| Grapefruit | Q9MB46 | V-type proton ATPase subunit E |
| Grapefruit | F8WL82 | WD-40 repeat family protein |
| Helianthuus annuus | HanXRQChr03g0080391 | Hsp90 |
| Helianthuus annuus | HanXRQChr13g0408351 | Hsp90 |
| Helianthuus annuus | HanXRQChr13g0408441 | Hsp90 |
| Helianthuus annuus | HanXRQChr14g0462551 | Hsp90 |
| Helianthuus annuus | HanXRQChr02g0044471 | Hsp70 |
| Helianthuus annuus | HanXRQChr02g0044481 | Hsp70 |
| Helianthuus annuus | HanXRQChr05g0132631 | Hsp70 |
| Helianthuus annuus | HanXRQChr05g0134631 | Hsp70 |
| Helianthuus annuus | HanXRQChr05g0134801 | Hsp70 |
| Helianthuus annuus | HanXRQChr10g0299441 | glutathione S-transferase |
| Helianthuus annuus | HanXRQChr16g0516291 | glutathione S-transferase |
| Helianthuus annuus | HanXRQChr03g0091431 | lactate/malate dehydrogenase |
| Helianthuus annuus | HanXRQChr13g0421951 | lactate/malate dehydrogenase |
| Helianthuus annuus | HanXRQChr10g0304821 | lactate/malate dehydrogenase |
| Helianthuus annuus | HanXRQChr12g0373491 | lactate/malate dehydrogenase |
| Helianthuus annuus | HanXRQChr01g0031071 | small GTPase superfamily, Rab type |
| Helianthuus annuus | HanXRQChr01g0031091 | small GTPase superfamily, Rab type |
| Helianthuus annuus | HanXRQChr02g0050791 | small GTPase superfamily, Rab type |
| Helianthuus annuus | HanXRQChr11g0353711 | small GTPase superfamily, Rab type |
| Helianthuus annuus | HanXRQChr13g0402771 | small GTPase superfamily, Rab type |
| Helianthuus annuus | HanXRQChr07g0190171 | isocitrate/isopropylmalate dehydrogenase |
| Helianthuus annuus | HanXRQChr16g0532251 | isocitrate/isopropylmalate dehydrogenase |
| Helianthuus annuus | HanXRQChr03g0079131 | phosphoenolpyruvate carboxylase |
| Helianthuus annuus | HanXRQChr15g0495261 | phosphoenolpyruvate carboxylase |
| Helianthuus annuus | HanXRQChr13g0388931 | phosphoenolpyruvate carboxylase |
| Helianthuus annuus | HanXRQChr14g0442731 | phosphoenolpyruvate carboxylase |
| Helianthuus annuus | HanXRQChr15g0482381 | UTP--glucose-1-phosphate uridylyltransferase |
| Helianthuus annuus | HanXRQChr16g0532261 | UTP--glucose-1-phosphate uridylyltransferase |
| Helianthuus annuus | HanXRQChr05g0135591 | tubulin |
| Helianthuus annuus | HanXRQChr06g0178921 | tubulin |
| Helianthuus annuus | HanXRQChr08g0237071 | tubulin |
| Helianthuus annuus | HanXRQChr11g0337991 | tubulin |
| Helianthuus annuus | HanXRQChr13g0407921 | tubulin |
| Helianthuus annuus | HanXRQChr05g0145191 | tubulin |
| Helianthuus annuus | HanXRQChr07g0187021 | tubulin |
| Helianthuus annuus | HanXRQChr07g0189811 | tubulin |
| Helianthuus annuus | HanXRQChr09g0253681 | tubulin |
| Helianthuus annuus | HanXRQChr10g0288911 | tubulin |
| Helianthuus annuus | HanXRQChr11g0322631 | tubulin |
| Helianthuus annuus | HanXRQChr12g0367231 | tubulin |
| Helianthuus annuus | HanXRQChr13g0386681 | tubulin |
| Helianthuus annuus | HanXRQChr13g0393261 | tubulin |
| Helianthuus annuus | HanXRQChr12g0371591 | ubiquitin |
| Helianthuus annuus | HanXRQChr12g0383641 | ubiquitin |
| Helianthuus annuus | HanXRQChr17g0569881 | ubiquitin |
| Helianthuus annuus | HanXRQChr06g0171511 | photosystem II HCF136, stability/assembly factor |
| Helianthuus annuus | HanXRQChr17g0544921 | photosystem II HCF136, stability/assembly factor |
| Helianthuus annuus | HanXRQChr16g0526461 | proteasome B-type subunit |
| Helianthuus annuus | HanXRQChr17g0565551 | proteasome B-type subunit |
| Helianthuus annuus | HanXRQChr05g0149801 | proteasome B-type subunit |
| Helianthuus annuus | HanXRQChr09g0241421 | proteasome B-type subunit |
| Helianthuus annuus | HanXRQChr11g0353161 | proteasome B-type subunit |
| Helianthuus annuus | HanXRQChr16g0506311 | proteinase inhibitor family I3 (Kunitz) |
| Helianthuus annuus | HanXRQChr16g0506331 | proteinase inhibitor family I3 (Kunitz) |
| Helianthuus annuus | HanXRQChr09g0265401 | metallopeptidase (M10 family) |
| Helianthuus annuus | HanXRQChr09g0265411 | metallopeptidase (M10 family) |
| Helianthuus annuus | HanXRQChr05g0154561 | ATPase, AAA-type |
| Helianthuus annuus | HanXRQChr08g0235061 | ATPase, AAA-type |
| Helianthuus annuus | HanXRQChr09g0273921 | ATPase, AAA-type |
| Helianthuus annuus | HanXRQChr16g0498881 | ATPase, AAA-type |
| Helianthuus annuus | HanXRQChr02g0058711 | oxoacid dehydrogenase acyltransferase |
| Helianthuus annuus | HanXRQChr08g0214191 | oxoacid dehydrogenase acyltransferase |
| Helianthuus annuus | HanXRQChr08g0208631 | small GTPase superfamily, SAR1-type |
| Helianthuus annuus | HanXRQChr11g0331441 | small GTPase superfamily, SAR1-type |
| Helianthuus annuus | HanXRQChr12g0371571 | small GTPase superfamily, SAR1-type |
| Helianthuus annuus | HanXRQChr12g0383571 | small GTPase superfamily, SAR1-type |
| Helianthuus annuus | HanXRQChr14g0446771 | small GTPase superfamily, SAR1-type |
| Helianthuus annuus | HanXRQChr17g0539461 | small GTPase superfamily, SAR1-type |
| Helianthuus annuus | HanXRQChr17g0548271 | small GTPase superfamily, SAR1-type |
| Helianthuus annuus | HanXRQChr17g0569871 | small GTPase superfamily, SAR1-type |
| Helianthuus annuus | HanXRQChr10g0311201 | ATPase, V1 complex, subunit A |
| Helianthuus annuus | HanXRQChr12g0359711 | ATPase, V1 complex, subunit A |
| Helianthuus annuus | HanXRQChr04g0124671 | fructose-1,6-bisphosphatase |
| Helianthuus annuus | HanXRQChr06g0176631 | fructose-1,6-bisphosphatase |
| Helianthuus annuus | HanXRQCPg0579861 | photosystem II PsbD/D2, reaction centre |
| Helianthuus annuus | HanXRQChr00c0439g0574731 | photosystem II PsbD/D2, reaction centre |
| Helianthuus annuus | HanXRQChr04g0099321 | photosystem II PsbD/D2, reaction centre |
| Helianthuus annuus | HanXRQChr08g0210231 | photosystem II PsbD/D2, reaction centre |
| Helianthuus annuus | HanXRQChr11g0326671 | photosystem II PsbD/D2, reaction centre |
| Helianthuus annuus | HanXRQChr17g0549121 | photosystem II PsbD/D2, reaction centre |
| Helianthuus annuus | HanXRQCPg0579731 | photosystem II protein D1 |
| Helianthuus annuus | HanXRQChr00c0126g0571821 | photosystem II protein D1 |
| Helianthuus annuus | HanXRQChr00c0165g0572191 | photosystem II protein D1 |
| Helianthuus annuus | HanXRQChr00c0368g0574171 | photosystem II protein D1 |
| Helianthuus annuus | HanXRQChr00c0454g0574931 | photosystem II protein D1 |
| Helianthuus annuus | HanXRQChr00c0524g0575441 | photosystem II protein D1 |
| Helianthuus annuus | HanXRQChr00c0572g0575941 | photosystem II protein D1 |
| Helianthuus annuus | HanXRQChr09g0257281 | photosystem II protein D1 |
| Helianthuus annuus | HanXRQChr11g0326571 | photosystem II protein D1 |
| Helianthuus annuus | HanXRQChr11g0327051 | photosystem II protein D1 |
| Helianthuus annuus | HanXRQChr16g0503941 | photosystem II protein D1 |
| Helianthuus annuus | HanXRQCPg0580061 | photosystem II cytochrome b559 |
| Helianthuus annuus | HanXRQChr01g0020331 | photosystem II cytochrome b559 |
| Helianthuus annuus | HanXRQChr10g0283581 | photosystem II cytochrome b559 |
| Helianthuus annuus | HanXRQChr10g0284271 | photosystem II cytochrome b559 |
| Helianthuus annuus | HanXRQChr10g0289291 | photosystem II cytochrome b559 |
| Helianthuus annuus | HanXRQChr10g0318171 | photosystem II cytochrome b559 |
| Helianthuus annuus | HanXRQChr11g0326851 | photosystem II cytochrome b559 |
| Helianthuus annuus | HanXRQChr16g0529011 | photosystem II cytochrome b559 |
| Helianthuus annuus | HanXRQChr08g0219051 | chlorophyll A-B binding protein |
| Helianthuus annuus | HanXRQChr12g0370841 | chlorophyll A-B binding protein |
| Helianthuus annuus | HanXRQChr02g0053151 | chlorophyll A-B binding protein |
| Helianthuus annuus | HanXRQChr02g0053161 | chlorophyll A-B binding protein |
| Helianthuus annuus | HanXRQCPg0580051 | cytochrome f |
| Helianthuus annuus | HanXRQChr01g0020341 | cytochrome f |
| Helianthuus annuus | HanXRQChr10g0283571 | cytochrome f |
| Helianthuus annuus | HanXRQChr10g0284261 | cytochrome f |
| Helianthuus annuus | HanXRQChr10g0289281 | cytochrome f |
| Helianthuus annuus | HanXRQChr10g0318181 | cytochrome f |
| Helianthuus annuus | HanXRQChr11g0326841 | cytochrome f |
| Helianthuus annuus | HanXRQChr15g0497521 | cytochrome f |
| Helianthuus annuus | HanXRQChr06g0163851 | ribosomal protein |
| Helianthuus annuus | HanXRQChr09g0252071 | ribosomal protein |
| Helianthuus annuus | HanXRQChr12g0374041 | ribosomal protein |
| Helianthuus annuus | HanXRQChr04g0128141 | ribosomal protein |
| Helianthuus annuus | HanXRQChr05g0163131 | ribosomal protein |
| Helianthuus annuus | HanXRQChr03g0076971 | ribosomal protein |
| Helianthuus annuus | HanXRQChr05g0159851 | ribosomal protein |
| Helianthuus annuus | HanXRQChr05g0159971 | ribosomal protein |
| Helianthuus annuus | HanXRQChr11g0324631 | ribosomal protein |
| Helianthuus annuus | HanXRQChr13g0408051 | ribosomal protein |
| Helianthuus annuus | HanXRQChr03g0089331 | ribosomal protein |
| Helianthuus annuus | HanXRQChr13g0419951 | ribosomal protein |
| Helianthuus annuus | HanXRQChr15g0497041 | ribosomal protein |
| Helianthuus annuus | HanXRQChr16g0499761 | ribosomal protein |
| Helianthuus annuus | HanXRQChr04g0106961 | ribosomal protein |
| Helianthuus annuus | HanXRQChr06g0175811 | ribosomal protein |
| Helianthuus annuus | HanXRQChr04g0122771 | ribosomal protein |
| Helianthuus annuus | HanXRQChr09g0245691 | ribosomal protein |
| Helianthuus annuus | HanXRQChr16g0520021 | ribosomal protein |
| Helianthuus annuus | HanXRQChr03g0060471 | ribosomal protein |
| Helianthuus annuus | HanXRQChr14g0429531 | ribosomal protein |
| Helianthuus annuus | HanXRQChr06g0171911 | ribosomal protein |
| Helianthuus annuus | HanXRQChr15g0479091 | ribosomal protein |
| Helianthuus annuus | HanXRQChr15g0479101 | ribosomal protein |
| Helianthuus annuus | HanXRQChr17g0543641 | ribosomal protein |
| Helianthuus annuus | HanXRQChr17g0543661 | ribosomal protein |
| Helianthuus annuus | HanXRQChr04g0105831 | ribosomal protein |
| Helianthuus annuus | HanXRQChr09g0258341 | ribosomal protein |
| Helianthuus annuus | HanXRQChr10g0287141 | ribosomal protein |
| Helianthuus annuus | HanXRQChr15g0463911 | ribosomal protein |
| Helianthuus annuus | HanXRQChr03g0076171 | ribosomal protein |
| Helianthuus annuus | HanXRQChr05g0159291 | ribosomal protein |
| Helianthuus annuus | HanXRQChr13g0407551 | ribosomal protein |
| Helianthuus annuus | HanXRQChr12g0380701 | ribosomal protein |
| Helianthuus annuus | HanXRQChr15g0477271 | ribosomal protein |
| Helianthuus annuus | HanXRQChr17g0545211 | ribosomal protein |
| Helianthuus annuus | HanXRQChr17g0570741 | ribosomal protein |
| Helianthuus annuus | HanXRQChr17g0570761 | ribosomal protein |
| Helianthuus annuus | HanXRQChr02g0044021 | ribosomal protein |
| Helianthuus annuus | HanXRQChr05g0152871 | ribosomal protein |
| Helianthuus annuus | HanXRQChr01g0012781 | ribosomal protein |
| Helianthuus annuus | HanXRQChr08g0230861 | ribosomal protein |
| Helianthuus annuus | HanXRQChr13g0391831 | ribosomal protein |
| Helianthuus annuus | HanXRQChr11g0337791 | bifunctional trypsin/alpha-amylase inhibitor |
| Helianthuus annuus | HanXRQChr10g0312371 | 2-oxoacid dehydrogenase acyltransferase |
| Helianthuus annuus | HanXRQChr09g0276191 | acid phosphatase (class B) |
| Helianthuus annuus | HanXRQChr05g0142271 | aldose-1-epimerase |
| Helianthuus annuus | HanXRQChr14g0439791 | alpha-D-phosphohexomutase |
| Helianthuus annuus | HanXRQChr09g0251071 | alpha-L-fucosidase |
| Helianthuus annuus | HanXRQChr05g0147371 | annexin |
| Helianthuus annuus | HanXRQChr09g0247561 | Asp protease (Peptidase family A1) |
| Helianthuus annuus | HanXRQChr13g0409681 | berberine-bridge enzyme (S)-reticulin: oxygen oxido-reductase |
| Helianthuus annuus | HanXRQChr10g0295971 | beta-hydroxyacyl-(acyl-carrier-protein) dehydratase |
| Helianthuus annuus | HanXRQChr13g0412571 | carbohydrate esterase family 13 - CE13 (pectin acylesterase - PAE) |
| Helianthuus annuus | HanXRQChr12g0360101 | carbohydrate esterase family 8 - CE8 (pectin methylesterase - PME) |
| Helianthuus annuus | HanXRQChr01g0019231 | carbonic anhydrase |
| Helianthuus annuus | HanXRQChr02g0036611 | cellular retinaldehyde binding/alpha-tocopherol transport |
| Helianthuus annuus | HanXRQChr10g0313581 | chaperonin Cpn60 |
| Helianthuus annuus | HanXRQChr09g0251791 | chlathrin |
| Helianthuus annuus | HanXRQChr11g0329811 | chlorophyll A-B binding protein |
| Helianthuus annuus | HanXRQChr13g0398861 | cobalamin (vitamin B12)-independent methionine synthase |
| Helianthuus annuus | HanXRQChr10g0298981 | cyclophilin |
| Helianthuus annuus | HanXRQChr04g0103281 | Cys protease (papain family) |
| Helianthuus annuus | HanXRQChr09g0268361 | cytochrome P450 |
| Helianthuus annuus | HanXRQChr17g0535591 | dirigent protein |
| Helianthuus annuus | HanXRQChr03g0065901 | expansin |
| Helianthuus annuus | HanXRQChr11g0336761 | expressed protein (cupin domain, seed storage protein |
| | | domain) |
| Helianthuus annuus | HanXRQChr10g0280931 | expressed protein (cupin domain, seed storage protein |
| | | domain) |
| Helianthuus annuus | HanXRQChr10g0288971 | expressed protein (cupin domain, seed storage protein |
| | | domain) |
| Helianthuus annuus | HanXRQChr12g0380361 | expressed protein (cupin domain, seed storage protein |
| | | domain) |
| Helianthuus annuus | HanXRQChr09g0254381 | expressed protein (cupin domain, seed storage protein |
| | | domain) |
| Helianthuus annuus | HanXRQChr04g0112711 | expressed protein (cupin domain, seed storage protein |
| | | domain) |
| Helianthuus annuus | HanXRQChr07g0196131 | expressed protein (cupin domain, seed storage protein |
| | | domain) |
| Helianthuus annuus | HanXRQChr10g0301281 | expressed protein (cupin domain, seed storage protein |
| | | domain) |
| Helianthuus annuus | HanXRQChr10g0301931 | expressed protein (cupin domain, seed storage protein |
| | | domain) |
| Helianthuus annuus | HanXRQChr13g0404461 | expressed protein (cupin domain) |
| Helianthuus annuus | HanXRQChr01g0015821 | expressed protein (DUF642) |
| Helianthuus annuus | HanXRQChr03g0065301 | expressed protein (Gnk2-homologous domain, antifungal |
| | | protein of Ginkgo seeds) |
| Helianthuus annuus | HanXRQChr03g0068311 | expressed protein (LRR domains) |
| Helianthuus annuus | HanXRQChr10g0291371 | expressed protein (LRR domains) |
| Helianthuus annuus | HanXRQChr03g0075061 | fasciclin-like arabinogalactan protein (FLA) |
| Helianthuus annuus | HanXRQChr08g0221961 | ferritin |
| Helianthuus annuus | HanXRQChr09g0257521 | FMN-dependent dehydrogenase |
| Helianthuus annuus | HanXRQChr14g0441641 | fructose-bisphosphate aldolase |
| Helianthuus annuus | HanXRQChr10g0312621 | germin |
| Helianthuus annuus | HanXRQChr09g0244271 | glucose-methanol-choline oxidoreductase |
| Helianthuus annuus | HanXRQChr03g0061571 | glutamate synthase |
| Helianthuus annuus | HanXRQChr05g0144801 | glyceraldehyde 3-phosphate dehydrogenase |
| Helianthuus annuus | HanXRQChr17g0550211 | glycerophosphoryl diester phosphodiesterase |
| Helianthuus annuus | HanXRQChr06g0175391 | glycoside hydrolase family 16 - GH16 (endoxyloglucan |
| | | transferase) |
| Helianthuus annuus | HanXRQChr11g0351571 | glycoside hydrolase family 17 - GH17 (beta-1,3-glucosidase) |
| Helianthuus annuus | HanXRQChr05g0141461 | glycoside hydrolase family 18 - GH18 |
| Helianthuus annuus | HanXRQChr09g0276721 | glycoside hydrolase family 19 - GH19 |
| Helianthuus annuus | HanXRQChr02g0046191 | glycoside hydrolase family 2 - GH2 |
| Helianthuus annuus | HanXRQChr16g0524981 | glycoside hydrolase family 20 - GH20 |
| | | (N-acetyl-beta-glucosaminidase) |
| Helianthuus annuus | HanXRQChr11g0322851 | glycoside hydrolase family 27 - GH27 |
| | | (alpha-galactosidase/melibiase) |
| Helianthuus annuus | HanXRQChr10g0293191 | glycoside hydrolase family 3 - GH3 |
| Helianthuus annuus | HanXRQChr16g0511881 | glycoside hydrolase family 31 - GH31 (alpha-xylosidase) |
| Helianthuus annuus | HanXRQChr14g0461441 | glycoside hydrolase family 32 - GH32 (vacuolar invertase) |
| Helianthuus annuus | HanXRQChr13g0423671 | glycoside hydrolase family 35 - GH35 (beta-galactosidase) |
| Helianthuus annuus | HanXRQChr10g0319301 | glycoside hydrolase family 35 - GH35 (beta-galactosidase) |
| Helianthuus annuus | HanXRQChr09g0256531 | glycoside hydrolase family 38 - GH38 (alpha-mannosidase) |
| Helianthuus annuus | HanXRQChr11g0320901 | glycoside hydrolase family 5 - GH5 (glucan-1,3-beta |
| | | glucosidase) |
| Helianthuus annuus | HanXRQChr05g0130491 | glycoside hydrolase family 51 - GH51 |
| | | (alpha-arabinofuranosidase) |
| Helianthuus annuus | HanXRQChr10g0314191 | glycoside hydrolase family 79 - GH79 |
| | | (endo-beta-glucuronidase/heparanase |
| Helianthuus annuus | HanXRQChr13g0397411 | homologous to A. thaliana PMR5 (Powdery Mildew |
| | | Resistant) (carbohydrate acylation) |
| Helianthuus annuus | HanXRQChr14g0444681 | inhibitor family I3 (Kunitz-P family) |
| Helianthuus annuus | HanXRQChr14g0445181 | lactate/malate dehydrogenase |
| Helianthuus annuus | HanXRQChr17g0564111 | lectin (D-mannose) |
| Helianthuus annuus | HanXRQChr17g0558861 | lectin (PAN-2 domain) |
| Helianthuus annuus | HanXRQChr02g0039251 | lipase acylhydrolase (GDSL family) |
| Helianthuus annuus | HanXRQChr01g0000161 | lipid transfer protein/trypsin-alpha amylase inhibitor |
| Helianthuus annuus | HanXRQChr02g0047121 | mannose-binding lectin |
| Helianthuus annuus | HanXRQChr10g0303361 | mitochondrial carrier protein |
| Helianthuus annuus | HanXRQChr15g0489551 | multicopper oxidase |
| Helianthuus annuus | HanXRQChr05g0135581 | neutral/alkaline nonlysosomal ceramidase |
| Helianthuus annuus | HanXRQChr01g0017621 | nucleoside diphosphate kinase |
| Helianthuus annuus | HanXRQChr10g0295991 | peroxidase |
| Helianthuus annuus | HanXRQChr13g0398251 | peroxiredoxin |
| Helianthuus annuus | HanXRQChr11g0333171 | phosphate-induced (phi) protein 1 |
| Helianthuus annuus | HanXRQChr03g0060421 | phosphodiesterase/nucleotide pyrophosphatase/phosphate |
| | | transferase |
| Helianthuus annuus | HanXRQChr03g0078011 | phosphofructokinase |
| Helianthuus annuus | HanXRQChr13g0408831 | phosphoglycerate kinase |
| Helianthuus annuus | HanXRQChr10g0286701 | phosphoglycerate mutase |
| Helianthuus annuus | HanXRQChr06g0171591 | photosystem II PsbP, oxygen evolving complex |
| Helianthuus annuus | HanXRQChr14g0434951 | plastid lipid-associated protein/fibrillin conserved domain |
| Helianthuus annuus | HanXRQChr05g0146621 | plastocyanin (blue copper binding protein) |
| Helianthuus annuus | HanXRQChr11g0330251 | polyphenol oxidase |
| Helianthuus annuus | HanXRQChr04g0094541 | proteasome A-type subunit |
| Helianthuus annuus | HanXRQChr03g0081271 | proteasome B-type subunit |
| Helianthuus annuus | HanXRQChr12g0356851 | purple acid phosphatase |
| Helianthuus annuus | HanXRQChr15g0485781 | pyridoxal phosphate-dependent transferase |
| Helianthuus annuus | HanXRQChr11g0336791 | ribosomal protein |
| Helianthuus annuus | HanXRQChr11g0330521 | ribosomal protein |
| Helianthuus annuus | HanXRQChr11g0326801 | ribulose bisphosphate carboxylase, large subunit |
| Helianthuus annuus | HanXRQChr16g0523951 | ribulose-1,5-bisphosphate carboxylase small subunit |
| Helianthuus annuus | HanXRQChr01g0022151 | S-adenosyl-L-homocysteine hydrolase |
| Helianthuus annuus | HanXRQChr14g0454811 | S-adenosylmethionine synthetase |
| Helianthuus annuus | HanXRQChr04g0109991 | SCP-like extracellular protein (PR-1) |
| Helianthuus annuus | HanXRQChr03g0072241 | Ser carboxypeptidase (Peptidase family S10) |
| Helianthuus annuus | HanXRQChr12g0377221 | Ser protease (subtilisin) (Peptidase family S8) |
| Helianthuus annuus | HanXRQChr02g0055581 | superoxide dismutase |
| Helianthuus annuus | HanXRQChr15g0493261 | thaumatin (PR5) |
| Helianthuus annuus | HanXRQChr16g0532531 | transketolase |
| Helianthuus annuus | HanXRQChr07g0197421 | translation elongation factor EFTu/EF1A |
| Helianthuus annuus | HanXRQChr06g0173951 | translationally controlled tumour protein |

What is claimed is:

1. A pest control composition comprising a plurality of PMPs, wherein each of the plurality of PMPs comprises a heterologous pesticidal agent and wherein the composition is formulated for delivery to a plant or a plant pest.

2. The pest control composition of claim 1, wherein the heterologous pesticidal agent is an herbicidal agent, an antibacterial agent, an antifungal agent, an insecticidal agent, a molluscicidal agent, or a nematicidal agent.

3. The pest control composition of claim 2, wherein the herbicidal agent is doxorubicin.

4. The pest control composition of claim 2, wherein the herbicidal agent is glufosinate, glyphosate, propaquizafop, metamitron, metazachlor, pendimethalin, flufenacet, diflufenican, clomazone, nicosulfuron, mesotrione, pinoxaden, sulcotrione, prosulfocarb, sulfentrazone, bifenox, quinmerac, triallate, terbuthylazine, atrazine, oxyfluorfen, diuron, trifluralin, or chlorotoluron.

5. The pest control composition of claim 2, wherein the antibacterial agent is doxorubicin.

6. The pest control composition of claim 2, wherein the antibacterial agent is an antibiotic.

7. The pest control composition of claim 6, wherein the antibiotic is vancomycin.

8. The pest control composition of claim 6, wherein the antibiotic is a penicillin, a cephalosporin, a tetracycline, a macrolide, a sulfonamide, vancomycin, polymixin, gramicidin, chloramphenicol, clindamycin, spectinomycin, ciprofloxacin, isoniazid, rifampicin, pyrazinamide, ethambutol, myambutol, or streptomycin.

9. The pest control composition of claim 2, wherein the antifungal agent is azoxystrobin, mancozeb, prothioconazole, folpet, tebuconazole, difenoconazole, captan, bupirimate, or fosetyl-Al.

10. The pest control composition of claim 2, wherein the insecticidal agent is a chloronicotinyl, a neonicotinoid, a carbamate, an organophosphate, a pyrethroid, an oxadiazine, a spinosyn, a cyclodiene, an organochlorine, a fiprole, a mectin, a diacylhydrazine, a benzoylurea, an organotin, a pyrrole, a dinitroterpenol, a METI, a tetronic acid, a tetramic acid, or a pthalamide.

11. The pest control composition of claim 1, wherein the heterologous pesticidal agent is a small molecule, a nucleic acid, or a polypeptide.

12. The pest control composition of claim 11, wherein the small molecule is an antibiotic or a secondary metabolite.

13. The pest control composition of claim 11, wherein the nucleic acid is an inhibitory RNA.

14. The pest control composition of claim 1, wherein the heterologous pesticidal agent is encapsulated by each of the plurality of PMPs.

15. The pest control composition of claim 1, wherein the heterologous pesticidal agent is embedded on the surface of each of the plurality of PMPs.

16. The pest control composition of claim 1, wherein the heterologous pesticidal agent is conjugated to the surface of each of the plurality of PMPs.

17. The pest control composition of claim 1, wherein each of the plurality of PMPs further comprises a pest repellent.

18. The pest control composition of claim 1, wherein each of the plurality of PMPs further comprises an additional heterologous pesticidal agent.

19. The pest control composition of claim 1, wherein the plant pest is a bacterium or a fungus.

20. The pest control composition of claim 19, wherein the bacterium is a Pseudomonas species.

21. The pest control composition of claim 20, wherein the Pseudomonas species is Pseudomonas aeruginosa or Pseudomonas syringae.

22. The method of claim 19, wherein the fungus is a Sclerotinia species, a Botrytis species, an Aspergillus species, a Fusarium species, or a Penicillium species.

23. The pest control composition of claim 1, wherein the plant pest is an insect, a mollusk, or a nematode.

24. The pest control composition of claim 23, wherein the insect is an aphid or a lepidopteran.

25. The pest control composition of claim 23, wherein the nematode is a corn root-knot nematode.

26. The pest control composition of claim 1, wherein the composition is stable for at least one day at room temperature, and/or stable for at least one week at 4° C.

27. The pest control composition of claim 1, wherein the PMPs are stable for at least 24 hours, 48 hours, seven days, or 30 days at 4° C.

28. The pest control composition of claim 27, wherein the PMPs are stable at a temperature of at least 20° C., 24° C., or 37° C.

29. The pest control composition of claim 1, wherein the plurality of PMPs in the composition is at a concentration effective to decrease the fitness of a plant pest.

30. The pest control composition of claim 1, wherein the plurality of PMPs in the composition is at a concentration of at least 1, 10, 50, 100, or 250 μg PMP protein/mL.

31. The pest control composition of claim 1, wherein the composition comprises an agriculturally acceptable carrier.

32. The pest control composition of claim 1 wherein the composition is formulated to stabilize the PMPs.

33. The pest control composition of claim 1, wherein the composition is formulated as a liquid, a solid, an aerosol, a paste, a gel, or a gas composition.

34. The pest control composition of claim 1, wherein the composition comprises at least 5% PMPs.

35. A pest control composition comprising a plurality of PMPs, wherein the PMPs are isolated from a plant by a process which comprises the steps of:
(a) providing an initial sample from a plant, or a part thereof, wherein the plant or part thereof comprises EVs;
(b) isolating a crude PMP fraction from the initial sample, wherein the crude PMP fraction has a decreased level of at least one contaminant or undesired component from the plant or part thereof relative to the level in the initial sample;
(c) purifying the crude PMP fraction, thereby producing a plurality of pure PMPs, wherein the plurality of pure PMPs have a decreased level of at least one contaminant or undesired component from the plant or part thereof relative to the level in the crude EV fraction;
(d) loading the plurality of pure PMPs with a pest control agent; and
(e) formulating the PMPs of step (d) for delivery to a plant or a plant pest.

36. A plant comprising the pest control composition of claim 1.

37. A plant pest comprising the pest control composition of claim 1.

38. A method of delivering a pest control composition to a plant comprising contacting the plant with the composition of claim 1.

39. A method of increasing the fitness of a plant, the method comprising delivering to the plant the composition of claim 1, wherein the method increases the fitness of the plant relative to an untreated plant.

40. The method of claim 38, wherein the plant has an infestation by a plant pest.

41. The method of claim 40, wherein the method decreases the infestation relative to the infestation in an untreated plant.

42. The method of claim 40, wherein the method substantially eliminates the infestation relative to the infestation in an untreated plant.

43. The method of claim 38, wherein the plant is susceptible to infestation by a plant pest.

44. The method of claim 43, wherein the method decreases the likelihood of infestation in the plant relative to the likelihood of infestation in an untreated plant.

45. The method of claim 40, wherein the plant pest is a bacterium or a fungus.

46. The method of claim 45, wherein the bacterium is a Pseudomonas species.

47. The method of claim 45, wherein the fungus is a Sclerotinia species, a Botrytis species, an Aspergillus species, a Fusarium species, or a Penicillium species.

48. The method of claim 40, wherein the plant pest is an insect, a mollusk, or a nematode.

49. The method of claim 48, wherein the insect is an aphid or a lepidopteran.

50. The method of claim 48, wherein the nematode is a corn root-knot nematode.

51. The method of claim 38, wherein the pest control composition is delivered as a liquid, a solid, an aerosol, a paste, a gel, or a gas.

52. A method of delivering a pest control composition to a plant pest comprising contacting the plant pest with the composition of claim 1.

53. A method of decreasing the fitness of a plant pest, the method comprising delivering to the plant pest the composition of claim 1, wherein the method decreases the fitness of the plant pest relative to an untreated plant pest.

54. The method of claim 52, wherein the method comprises delivering the composition to at least one habitat where the plant pest grows, lives, reproduces, feeds, or infests.

55. The method of claim 52, wherein the composition is delivered as a plant pest comestible composition for ingestion by the plant pest.

56. The method of claim 52, wherein the plant pest is a bacterium or a fungus.

57. The method of claim 52, wherein the plant pest is an insect, a mollusk, or a nematode.

58. The method of claim 57, wherein the insect is an aphid or a lepidopteran.

59. The method of claim 57, wherein the nematode is a corn root-knot nematode.

60. The method of claim 52, wherein the composition is delivered as a liquid, a solid, an aerosol, a paste, a gel, or a gas.

61. A method of treating a plant having a fungal infection, wherein the method comprises delivering to the plant a pest control composition comprising a plurality of PMPs.

62. A method of treating a plant having a fungal infection, wherein the method comprises delivering to the plant a pest control composition comprising a plurality of PMPs, and wherein each of the plurality of PMPs comprises an antifungal agent.

63. The method of claim 62, wherein the antifungal agent is a nucleic acid that inhibits expression of a gene in a fungus that causes the fungal infection.

64. The method of claim 63, wherein the gene is dcl1 and/or dcl2.

65. The method of claim 61, wherein the fungal infection is caused by a fungus belonging to a Sclerotinia species, a Botrytis species, an Aspergillus species, a Fusarium species, or a Penicillium species.

66. The method of claim 65, wherein the Sclerotinia species is Sclerotinia sclerotiorum.

67. The method of claim 65, wherein the Botrytis species is Botrytis cinerea.

68. The method of claim 61, wherein the composition comprises a PMP derived from Arabidopsis.

69. The method of claim 61, wherein the method decreases or substantially eliminates the fungal infection.

70. A method of treating a plant having a bacterial infection, wherein the method comprises delivering to the plant a pest control composition comprising a plurality of PMPs.

71. A method of treating a plant having a bacterial infection, wherein the method comprises delivering to the plant a pest control composition comprising a plurality of PMPs, and wherein each of the plurality of PMPs comprises an antibacterial agent.

72. The method of claim 71, wherein the antibacterial agent is doxorubicin.

73. The method of claim 70, wherein the bacterial infection is caused by a bacterium belonging to a Pseudomonas spp.

74. The method of claim 73, wherein the Pseudomonas spp. is Pseudomonas syringae.

75. The method of claim 70, wherein the composition comprises a PMP derived from Arabidopsis.

76. The method of claim 70, wherein the method decreases or substantially eliminates the bacterial infection.

77. A method of decreasing the fitness of an insect plant pest, wherein the method comprises delivering to the insect plant pest a pest control composition comprising a plurality of PMPs.

78. A method of decreasing the fitness of an insect plant pest, wherein the method comprises delivering to the insect plant pest a pest control composition comprising a plurality of PMPs, and wherein each of the plurality of PMPs comprises an insecticidal agent.

79. The method of claim 78, where in the insecticidal agent is a peptide nucleic acid.

80. The method of claim 77, wherein the insect plant pest is an aphid.

81. The method of claim 77, wherein the insect plant pest is a lepidopteran.

82. The method of claim 81, wherein the lepidopteran is Spodoptera frugiperda.

83. The method of claim 77, wherein the method decreases the fitness of the insect plant pest relative to an untreated insect plant pest.

84. A method of decreasing the fitness of a nematode plant pest, wherein the method comprises delivering to the nematode plant pest a pest control composition comprising a plurality of PMPs.

85. A method of decreasing the fitness of a nematode plant pest, wherein the method comprises delivering to the nematode plant pest a pest control composition comprising a plurality of PMPs, and wherein each of the plurality of PMPs comprises a nematicidal agent.

86. The method of claim 85, wherein the nematicidal agent is a peptide.

87. The method of claim 86, wherein the peptide is Mi-NLP-15b.

88. The method of claim 84, wherein the nematode plant pest is a corn root-knot nematode.

89. The method of claim 84, wherein the method decreases the fitness of the nematode plant pest relative to an untreated nematode plant pest.

90. A method of decreasing the fitness of a weed, wherein the method comprises delivering to the weed a pest control composition comprising a plurality of PMPs.

91. A method of decreasing the fitness of a weed, wherein the method comprises delivering to the weed a pest control composition comprising a plurality of PMPs, and wherein each of the plurality of PMPs comprises an herbicidal agent.

92. The method of claim 90 or 91, wherein the method decreases the fitness of the weed relative to an untreated weed.


SEQUENCE LISTING (19 sequences)

SEQ ID NO: 1 - DNA, 3 bp, Artificial Sequence
  Synthetic Construct
  misc_feature (1)..(1): n is a, c, g, or t
ngg

SEQ ID NO: 2 - DNA, 6 bp, Artificial Sequence
  Synthetic Construct
  misc_feature (1)..(2): n is a, c, g, or t
nnagaa

SEQ ID NO: 3 - DNA, 5 bp, Artificial Sequence
  Synthetic Construct
  misc_feature (1)..(1): n is a, c, g, or t
  misc_feature (4)..(4): n is a, c, g, or t
nggng

SEQ ID NO: 4 - DNA, 7 bp, Artificial Sequence
  Synthetic Construct
  misc_feature (1)..(3): n is a, c, g, or t
nnngatt

SEQ ID NO: 5 - DNA, 3 bp, Artificial Sequence
  Synthetic Construct
  misc_feature (3)..(3): n is a, c, g, or t
ttn

SEQ ID NO: 6 - DNA, 3 bp, Artificial Sequence
  Synthetic Construct
cta

SEQ ID NO: 7 - DNA, 23 bp, Artificial Sequence
  Synthetic Construct
cctacattctacttgatctagta

SEQ ID NO: 8 - DNA, 18 bp, Artificial Sequence
  Synthetic Construct
gttggtagttgtgggtta

SEQ ID NO: 9 - DNA, 22 bp, Artificial Sequence
  Synthetic Construct
gagctgaagtggcttccatgac

SEQ ID NO: 10 - DNA, 21 bp, Artificial Sequence
  Synthetic Construct
ggtccgacatacccatgatcc

SEQ ID NO: 11 - DNA, 20 bp, Artificial Sequence
  Synthetic Construct
aactgaaaaacaccttgggc

SEQ ID NO: 12 - DNA, 20 bp, Artificial Sequence
  Synthetic Construct
cctgggttgttgaagtggta

SEQ ID NO: 13 - DNA, 21 bp, Artificial Sequence
  Synthetic Construct
acaatcctatctttcggaagc

SEQ ID NO: 14 - DNA, 22 bp, Artificial Sequence
  Synthetic Construct
agactcttcttcttgaagacag

SEQ ID NO: 15 - DNA, 20 bp, Artificial Sequence
  Synthetic Construct
gattgtgcaaagtctcaaca

SEQ ID NO: 16 - DNA, 23 bp, Artificial Sequence
  Synthetic Construct
attgggtttgactatatgtctta

SEQ ID NO: 17 - DNA, 25 bp, Artificial Sequence
  Synthetic Construct
gctgtaatttcaatgtgcagaatcc

SEQ ID NO: 18 - DNA, 23 bp, Artificial Sequence
  Synthetic Construct
ggagcaacaattaatcgcatttc

SEQ ID NO: 19 - protein, 14 aa, Meloidogyne incognita
SFDSFTGPGFTGLD